(19) United States
(12) Patent Application Publication
(10) Pub. No.: US 2015/0336943 A1
(43) Pub. Date: Nov. 26, 2015

(54) NOVEL BENZODIOXANE-PIPERIDINE DERIVATIVES AND THEIR THERAPEUTIC APPLICATIONS FOR TREATING NEUROPSYCHIATRIC DISORDERS

(71) Applicant: PIERRE FABRE MEDICAMENT, Boulogne-Billancourt (FR)

(72) Inventors: Pierre SOKOLOFF, Belleserre (FR); Philippe MAILLOS, Labastide Saint Georges (FR); Florence CUISIAT, Castres (FR); Jean-Louis VIDALUC, Castres (FR); Thierry IMBERT, Viviers les Montagnes (FR)

(73) Assignee: PIERRE FABRE MEDICAMENT, Boulogne-Billancourt (FR)

(21) Appl. No.: 14/759,337

(22) PCT Filed: Jan. 7, 2014
(86) PCT No.: PCT/EP2014/050131

§ 371 Date: Jul. 6, 2015

(30) Foreign Application Priority Data
Jan. 7, 2013 (FR) 1350088

(51) Int. Cl. C07D 413/14

(52) U.S. Cl. CPC C07D 413/14; C07B 2200/07

(57) ABSTRACT

The present invention concerns benzodioxane-piperidine with general formula I:

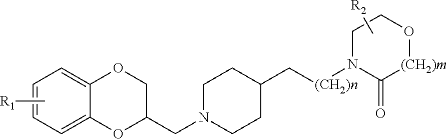

wherein notably:
   R1 represents one or more identical or different substituent(s) on the benzene ring, each independently representing a hydrogen or halogen atom, or a C₁₋₄ alkyl group, or a C₁₋₄ alkoxy group or a C₁₋₄ hydroxyalkyl group or a C₁₋₄ alkylcarbonyl or an alkoxycarbonyl group or an OH group or an SO2R group with R alkyl, or a CN group, or a CF3 group, or an OCF 3 group;
      n=1, 2 or 3;
      m=0 or 1, and
   R2 represents one or more identical or different substituent(s) on the oxazolidinone or morpholinone ring, each independently representing:
a hydrogen atom, a C₁₋₄ alkyl group, or a C₁₋₄ alkoxy group, or a C₁₋₄ hydroxyalkyl group, or an alkylcarbonyl group, or an alkoxycarbonyl group, or an alkoxyphenyl group.

The invention concerns benzodioxane-piperidine-oxazolidinone and benzodioxane-piperidine-morpholinone derivatives, pharmaceutical compositions containing them and their therapeutic applications, as partial agonists or antagonists of the D2 and D3 dopamine receptors and antagonists of α2C adrenergic receptors for the treatment of various neurological and psychiatric disorders. According to the invention, these disorders include, in a non-limiting manner, schizophrenia, bipolar disorder, autism and cognitive problems associated with various neurological disorders.

Schizophrenia is a term used to describe a group of pathologies of unclear origin that affect approximately 1% of the general population. This pathology is characterized by a variety of symptoms, which are classified as positive symptoms (hallucinations, delirium, disorganized thinking) and negative symptoms (social withdrawal and affective blunting), with an onset age in adolescence or early adulthood that can persist in the chronic form with episodes of exacerbation for many years. Cognitive deficits, such as problems with executive function, memory and verbal expression make up an integral part of the symptomotology throughout the course of this disorder.

Patients with schizophrenia may be treated with drugs called neuroleptics, also known as antipsychotics. The therapeutic effect of antipsychotics is generally recognized as resulting from blockage of receptors for the neurotransmitter dopamine in the brain. There are five known subtypes of the dopamine receptor, called D1, D2, D3, D4 and D5 (Sokoloff, P. et al., Novel dopamine receptor subtypes as targets for antipsychotic drugs. Annals New-York Academy of Sciences, 1995, 757, 278-292) and conventional antipsychotics are antagonists (or partial agonists, for Example aripiprazole) of the D2 and D3 receptors. The importance of blocking the D3 subtype for treating schizophrenia and other conditions has been emphasized (Sokoloff P, Diaz J, Le Foil B, Guillin O, Leriche L, Bezard E, Gross C. The dopamine D3 receptor: a therapeutic target for the treatment of neuropsychiatric disorders. 2006, 5: 25-43).

Antipsychotics based on the concomitant blockage of the D2 and D3 receptors are relatively active in improving the positive symptoms of schizophrenia. In return, they are only slightly active or inactive against the negative symptoms and for improving cognitive deficits. There is therefore a need to find new ingredients for antipsychotics with improved efficacy.

The literature also mentions that alpha2-adrenergic antagonist products potentiate the effect of antipsychotics (Litman R E, Hong W W, Weissman E M, Su T P, Potter W Z, Pickar D. Idazoxan, an alpha 2 antagonist, augments fluphenazine in schizophrenic patients: a pilot study. J Clin Psychopharmacol. 1993, 13:264-267) and act favorably on cognitive deficits (Marien M R, Colpaert F C, Rosenquist A C. Noradrenergic mechanisms in neurodegenerative diseases: a theory Brain Res Brain Res Rev. 2004; 45:38-78; Svensson T H. Alpha-adrenoceptor modulation hypothesis of antipsychotic atypicality. Prog Neuropsychopharmacol Biol Psychiatry. 2003 October; 27 (7):1145-58).

Alpha2-adrenergic receptors play a critical role in regulating neuronal activity and neurotransmitter release. Alpha2-adrenergic receptors are made up of three genetically and functionally distinct subtypes: alpha2A, alpha2B and alpha2C.

Comparative studies with transgenic mice have shown that the alpha2A subtype is the primary mediator of certain effects induced by alpha2-adrenergic agonists not selective for subtype, such as sedation, analgesia, hypothermia, sympathetic inhibition and reduction of blood pressure. Likewise, neuropsychopharmacological tests on KO (“knock-out”) mice that do not express the receptor or OE (over-expressing) mice that over-express the receptor show that the alpha2C-adrenergic subtype has a distinct inhibitor role in the development of sensory information, and in the control of cognitive, motor and emotional activities and those related to central nervous system stress (Scheinin M, Sallinen J, Haapalinna A (2001) Evaluation of the alpha2C-adrenoceptor as a neuropsychiatric drug target; studies in transgenic mouse models. Life Sci. 68: 2277-2285).

It has therefore been proposed that compounds selective for alpha2C receptors may be therapeutically useful in various disorders or conditions of the central nervous system, for example, to improve the cognitive symptoms associated with attention deficit-hyperactivity disorder or neurodegenerative diseases and their progression. These diseases include, as non-exhaustive examples, Parkinson's disease, Alzheimer's disease, Huntington's chorea, amyotrophic lateral sclerosis, progressive supranuclear palsy, Down syndrome, corticobasal degeneration, pugilistic dementia, multiple system atrophy, Lewy body disease, Pick's disease), or age-related disorders or mood, depression, anxiety disorders and mental diseases propagated by stress. Ischemic and post-ischemic brain disorders, strokes and their consequences and narcolepsy are also concerned (MacDonald E, Kobilka B K, Scheinin M. Gene targeting: homing in on alpha 2-adrenoceptor-subtype function. Trends Pharmacol Sci. 1997, 18: 211-219; Arnsten A F. Adrenergic targets for the treatment of cognitive deficits in schizophrenia. Psychopharmacology (Berl) 2004, 174:25-31).

Disorders and diseases in which patients are generally or occasionally treated by antipsychotics, such as bipolar disorder, autism spectrum disorders or major depression are also concerned by the therapeutic use of D2 and D3 receptor antagonists or partial agonists and alpha2C receptor antagonists, the subject of the present invention.

Bipolar disorder is a mood disorder, historically known as manic-depression, where patients live alternatingly in a state where excitation is abnormally elevated (manic or hypomanic state), and, in many cases, in an unusually depressed state. This disorder takes many forms. Bipolar disorder may appear as unipolar depression: a cyclic depressive state without the presence of mania (or hypomania). Bipolar disorder starts in childhood or adulthood for the majority of patients; most often the first episodes are depression. Estimates of the lifetime prevalence of bipolar disorder vary, with studies generally giving values of around 1%. The DSM-IV-TR (Diagnostic and Statistical Manual of Mental Disorders, Fourth Edition, American Psychiatric Association, 2004) lists three specific subtypes and one unspecified one:
Bipolar disorder I: one or more manic episodes.
Bipolar disorder II: no manic episodes, but one or more hypomanic episodes and one or more episodes of major depression.
Cyclothymia: history of hypomanic episodes with periods of depression that do not meet the criteria for major depressive episodes.

Antipsychotics are generally used to treat the manic or hypomanic phase. Some, like quetiapine, are also used to treat the depressive episodes of bipolar disorder.

Some antipsychotics are also used to treat autism, which consists of learning and socialization problems, as well as stress-related disorders in children and adolescents, whether they are associated with specified or unspecified pervasive developmental disorders, childhood and adolescent disintegrative disorder, early deficit psychoses, developmental disharmony of psychotic type, autisms or atypical form mood disorders, according to the ICD-10 classifications (10th edition of “International Statistical Classification of Diseases and Related Health Problems”, ICD-10, Classification of Mental and Behavioral Disorders: diagnostic criteria for research, World Health Organization, 1993 revised 1997), in the DSM-IV-TR (Diagnostic and Statistical Manual of Mental Disorders, Fourth Edition, American Psychiatric Association, 2004) and the CFTMEA-R Classification Française des Troubles Mentaux de l'Enfant and de l'Adolescent [French Classification of Mental Disorders in Children and Adolescents](Mises, Quemada, CFTMEA R 2000. CTNERHI, Paris 2002; C. Bursztejn, P. Jeammet. Autism and psychoses in children in the CFTMEA R-2000, Annales medico-psychologiques, 160; 216-219, 2002).

Finally, antipsychotics are used in severe forms of major depression, in patients with very high anxiety with delusions.

Cardiovascular tolerance is a key factor in selecting a candidate alpha2-adrenergic receptor antagonist for clinical development. Therefore, understanding the respective role of alpha2-adrenergic receptors and other adrenergic receptors in regulating cardiovascular function is fundamentally important. Some clarifications on this subject were provided by a KO mice study (MacDonald et al., 1997, already cited) showing that activation of alpha2B adrenergic receptors counteracts the hypotensive effects of alpha2 agonists, involving the alpha2A subtype. These studies also indicate that alpha2C adrenergic receptors, despite their distribution in strategic positions in the central and peripheral nervous system, which could theoretically change cardiovascular homeostasis, do not seem to have significant functional impact, at least not in transgenic animal models. Thus, an alpha2-adrenergic receptor that was selective for subtype C could have reduced cardiovascular impact or better tolerance compared to an alpha2 antagonist not selective for subtype.

The prior art mentions, for benzodioxane-piperidine derivatives bearing an amide, a urea or an imidazolidinone, as non-selective alpha2-antagonists (WO 97/28157, WO 98/02435); some of these products also have a weak affinity for the D2 dopaminergic receptor (T. Imbert, J. Med. Chem, 2000, 43: 3653-3664). During the development of the present invention, the inventors showed that these products already described act as effective or very effective agonists (full agonists) for D2 and D3 dopamine receptors. Activity as a D2 and D3 receptor agonist was determined according to the described methods (Bruins Slot L A, De Vries L, Newman-Tancredi A, Cussac D. Differential profile of antipsychotics at serotonin 5-HT1A and dopamine D2S receptors coupled to extracellular signal-regulated kinase. Eur J Pharmacol, 2006, 534; 63-70; Bruins Slot L A, Palmier C, Tardif S, Cussac D. Action of novel antipsychotics at human dopamine D3 receptors coupled to G protein and ERK1/2 activation. Neuropharmacology, 2007, 53: 232-241) for the following compounds, such as described in WO 97/28157 and WO 98/02435;
1-benzyl-3-(2-(1-((2,3-dihydrobenzo[b][1,4]dioxin-2-yl)methyl)piperidin-4-yl)ethyl)imidazolidin-2-one
1-(2-(1-((2,3-dihydrobenzo[b][1,4]dioxin-2-yl)methyl)piperidin-4-yl)ethyl)-3-methylimidazolidin-2-one
1-(2,6-dichlorophenyl)-3-(2-(1-((2,3-dihydrobenzo[b][1,4]dioxin-2-yl)methyl)piperidin-4-yl)ethyl)imidazolidin-2-one
1-(2-(1-((2,3-dihydrobenzo[b][1,4]dioxin-2-yl)methyl)piperidin-4-yl)ethyl)-3-(pyridin-4-yl)imidazolidin-2-one
3-(2-(1-((2,3-dihydrobenzo[b][1,4]dioxin-2-yl)methyl)piperidin-4-yl)ethyl)-1-phenylimidazolidine-2,4-dione
(S)—N-(2-(1-((2,3-dihydrobenzo[b][1,4]dioxin-2-yl)methyl)piperidin-4-yl)ethyl)-[1,1′-biphenyl]-4-carboxamide
(S)—N-(2-(1-((2,3-dihydrobenzo[b][1,4]dioxin-2-yl)methyl)piperidin-4-yl)ethyl)-4-(1H-pyrrol-1-yl)benzamide
(S)—N-(2-(1-((2,3-dihydrobenzo[b][1,4]dioxin-2-yl)methyl)piperidin-4-yl)ethyl)-4-(pyridin-4-yl)benzamide
(S)—N-(2-(1-((2,3-dihydrobenzo[b][1,4]dioxin-2-yl)methyl)piperidin-4-yl)ethyl)-4-(pyridin-3-yl)benzamide
(S)—N-(2-(1-((2,3-dihydrobenzo[b][1,4]dioxin-2-yl)methyl)piperidin-4-yl)ethyl)-9-oxo-9H-fluorene-1-carboxamide.

For these compounds, the intrinsic activity (in percentage of that for dopamine) varied from 20 to 88% for receptor D2 and from 85 to 102% for receptor D3. The power of the products (concentration producing half the maximum response) varied from 1.9 to 320 nanomoles/liter at receptor D2 and from 3.3 to 43 nanomoles/liter at receptor D3. These products therefore behave as effective to very effective agonists on receptors D2 and D3, which is not desirable to produce antipsychotics.

Surprisingly and unexpectedly, the introduction of substituents on the benzodioxane part and/or replacement of the amide parts, urea or imidazolidinone by a oxazolidinone or morpholinone heterocycle have a major and multiple pharmacological impact that renders these products much more interesting for producing new antipsychotics. Indeed, these chemical modulations:
increase affinity with dopamine receptor D2
give these new products a higher affinity for receptor D3
reduce their agonist power on receptors D2 and D3
render these products more powerful alpha2 antagonists on subtype alpha2C than on subtypes alpha2A and alpha2B.

The compounds of the invention can therefore be used for preparing pharmaceutical compositions and medicaments for the treatment of neurological or psychiatric diseases, conditions or disorders, such as described above, for which products are sought that are both D2 and D3 antagonists, with a preferential affinity for receptor D3, and alpha2C antagonists.

As used here, the term “salts” designates acid and base inorganic addition salts of the compounds of the present invention. Preferably, the salts are pharmaceutically acceptable, i.e., they are nontoxic for the patient to whom they are administered.

The term “pharmaceutically acceptable” refers to molecular entities and compositions that do not produce any adverse, allergic or other undesirable reaction when they are administered to an animal or human.

When used here, the term “pharmaceutically-acceptable excipient” includes any diluent, adjuvant or excipient, such as preservatives, fillers, disintegrating agents, wetting agents, emulsifiers, dispersants, antibacterial or antifungal agents or agents that would delay absorption and intestinal and digestive absorption. The use of these media or vectors is well known in the art. Unless the agent is chemically incompatible with a chromone derivative, its use in pharmaceutical compositions with the compounds according to the invention is considered.

In the context of the invention, the term “treatment” as used here means preventing or inhibiting the onset or progression of the disorder to which the term applies, or even to one or more symptoms of this disorder.

“Therapeutically-active quantity” means a quantity of benzodioxane derivative effective for obtaining the desired therapeutic effect according to the invention.

According to the invention, the term “patient” refers to a human or non-human mammal affected or even potentially affected by a disease. Preferentially the patient is a human.

In the present invention, C₁₋₄ alkyl group means a linear or branched hydrocarbon chain with 1 to 4 carbon atoms.

In the present invention, C₁₋₄ alkoxyl group means a linear or branched hydrocarbon chain with 1 to 4 carbon atoms and an oxygen atom.

In the present invention, halogen group means fluorine, chlorine, or bromine.

In the present invention, C₁₋₄ hydroxyalkyl group means an alkyl as defined previously in which a hydrogen atom is replaced by a hydroxyl group.

In the present invention, C₁₋₄ alkylcarbonyl group means a group such as C(═O)R with R alkyl as defined previously.

In the present invention, C₁₋₄ alkoxycarbonyl group means a group such as C(═O)OR with R alkyl as defined previously.

In the present invention, C₁₋₄ phenylalkyl group means a phenyl bound by a carbon atom to an alkyl group as defined previously.

In the present invention, C₁₋₄ alkoxyphenyl group means a phenyl bound by an oxygen atom to an alkyl group as defined previously.

In the present invention, C₁₋₄ alkoxybenzyl group means a benzyl bound by an oxygen atom to an alkyl group as defined previously.

In the present invention, stereoisomer forms mean both enantiomers and diastereomers.

The present invention concerns benzodioxane-piperidine-oxazolidinone and benzodioxane-piperidine-morpholinone derivatives and therapeutic applications thereof as partial agonists or antagonists of dopamine receptors D2 and D3 and antagonists of α2C adrenergic receptors for the treatment of various neurological and psychiatric disorders.

These compounds have general formula 1.

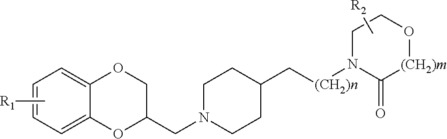

wherein:

R1 represents one or more identical or different substituent(s) on the benzene ring, each independently representing a hydrogen or halogen atom, or a C₁₋₄ alkyl group, or a C₁₋₄ alkoxy group or a C₁₋₄ hydroxyalkyl group or an alkylcarbonyl group or an alkoxycarbonyl group or an OH group or an SO2R group with R alkyl, or a CN group, or a CF3 group, or an OCF3 group;

n=1, 2 or 3;

R2 represents one or more identical or different substituent(s) on the oxazolidinone or morpholinone ring, each independently representing:

a hydrogen atom or a C₁₋₄ alkyl group, or C₁₋₄ alkoxy group, or C₁₋₄ hydroxyalkyl group or an alkylcarbonyl group, or an alkoxycarbonyl group or an alkoxyphenyl group—optionally substituted by one or more identical or different substituent(s) each independently representing a C₁₋₄ alkoxy group or C₁₋₄ alkyl group or halogen, or a hydroxy—or an alkoxybenzyl group—optionally substituted by one or more identical or different substituent (s) each independently representing a C₁₋₄ alkoxy group or a C₁₋₄ alkyl group or halogen, or a hydroxy—or a hydroxybenzyl group, or a benzyl group—optionally substituted with one or more identical or different substituent(s) each independently representing a C₁₋₄ alkoxy group or C₁₋₄ alkyl group or a halogen, or a hydroxy—or a phenyl group—optionally substituted with one or more identical or different substituent (s) each independently representing a C₁₋₄ alkoxy group or a C₁₋₄ alkyl group or halogen, or a hydroxy—while R2 is a ring fused with the oxazolidinone or morpholinone group that bears it, consisting of an unsubstituted benzene or benzene substituted by a C₁₋₄ alkoxy, hydroxy, halogen or cyano;

m=0 or 1.

The invention also concerns stereoisomer forms, including enantiomers of the compounds of general formula 1, pharmaceutically-acceptable salts thereof, and pharmaceutically-acceptable compositions containing them, and their use as a medicament intended to treat central nervous system disorders.

Compounds of general formula 1 are prepared:
according to diagram 1.

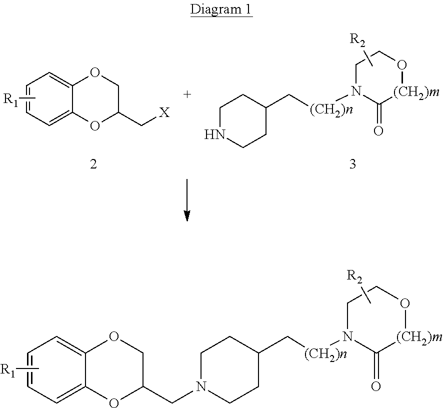

Compounds of general formula 1 are obtained by nucleophilic substitution or coupling between type 3 piperidine derivatives and type 2 benzodioxane derivatives where X is a halogen, mesylate, tosylate or brosylate leaving group.

Type 2 derivatives are prepared from corresponding hydroxyl compounds 4 according to diagram 2. Hydroxyl compounds 4 are prepared by the method described in J. Med. Chem, 1997, 40, 4235-4256, or according to the process described in J. Am. Chem. Soc, 2001, 123, 12202-12206.

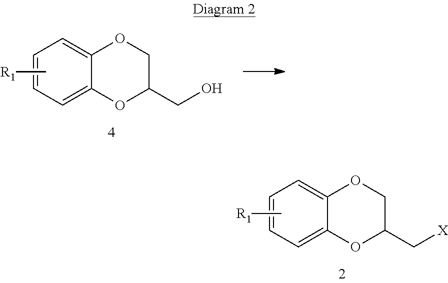

Compounds of type 3 are prepared:
by coupling of the corresponding type 5 oxazolidinone or morpholinone with a protected piperidine alkyl derivative—where P is a protecting group and X a leaving group as defined previously—followed by deprotection according to diagram 3.

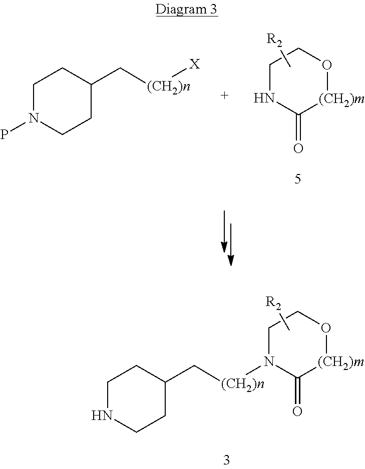

or for m=0 by construction of the oxazolidinone ring, according to diagram 4, from piperidine amino alcohol derivatives 6 by reaction with phosgene or a chemical analogue such as carbonyl diimidazole, followed by deprotection of the amine function of the piperidine moiety.
or for m=1 by construction of the morpholinone ring, according to diagram 4, from piperidine amino alcohol derivatives 6 by reaction with chloroacetyl chloride, and then deprotection of the amine function of the piperidine moiety.

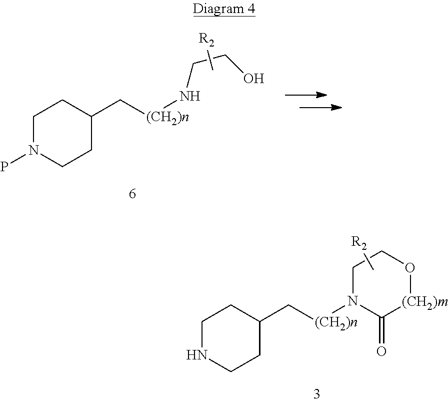

The preceding compounds of type 6 are obtained:

When R2 is borne by the carbon atom in a of the nitrogen atom of oxazolidinone or morpholinone, from a piperidine alcohol product after oxidation into aldehyde and reductive amination with amino alcohol derivatives 10 according to diagram 5

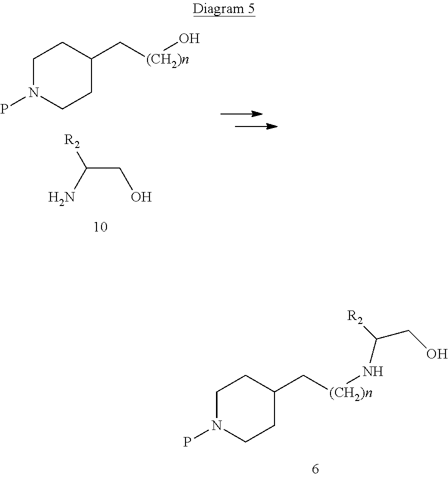

or when R2 is borne by the carbon atom in a of the oxygen atom of oxazolidinone or morpholinone, by opening of epoxides 11 by piperidine alkylamine itself prepared from piperidine alkyl alcohol according to diagram 6

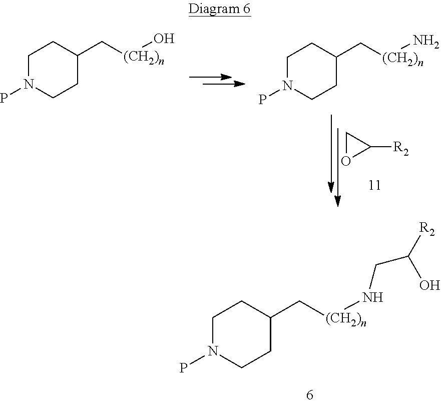

For compounds of type 3 where R2 is an ester located at a of the nitrogen of oxazolidinone or morpholinone, it is subjected to chemical transformations such as reduction into alcohol and transformation into alkoxy 7, or such as reduction into aldehyde. The initial aldehyde or ester subjected to the action of nucleophiles respectively give the hydroxylated mono-addition compound 8 and di-addition compound 9 where R3 is an alkyl or phenyl or benzyl group, then the amine function of compounds 7, 8 and 9 is deprotected to give compounds 3 as described in diagram 7

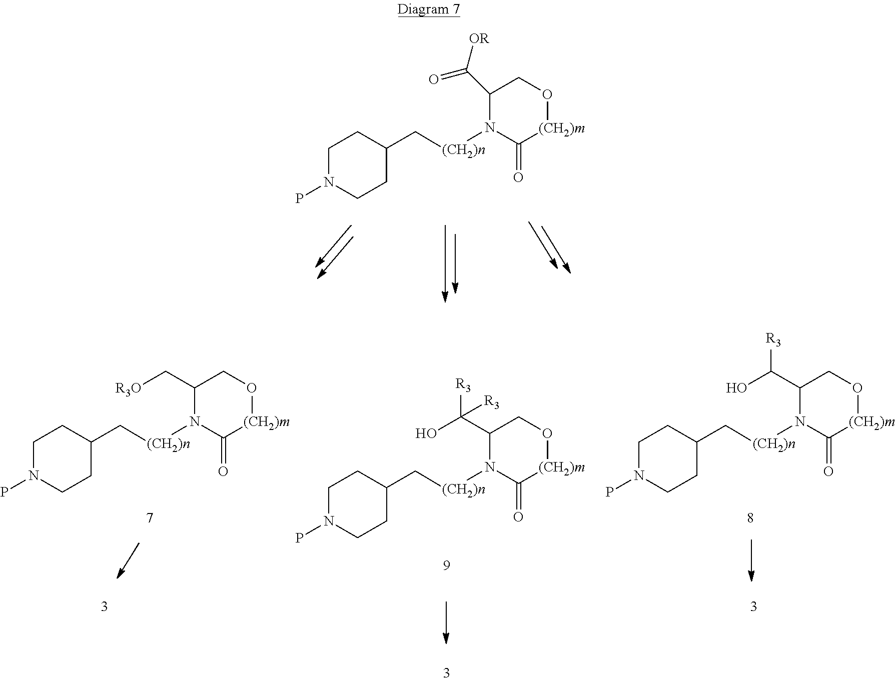

The preceding compounds of type 5 are prepared for m=0, when they are not commercial:

either when R2 is a single substituent borne by the carbon at a of the oxazolidinone nitrogen
for phenyl substituted R2, from the corresponding aldehyde, which leads by Strecker reaction to type 12 aminobenzyl cyanides which are transformed to the corresponding type 13 amino alcohol by transformation of the nitrile function to amide and then to acid, which is then reduced to alcohol and the amine function is released by debenzylation, the amino alcohol 10 thus obtained is transformed into oxazolidinone 5 by reaction with phosgene or a chemical analog such as carbonyl diimidazole according to diagram 8

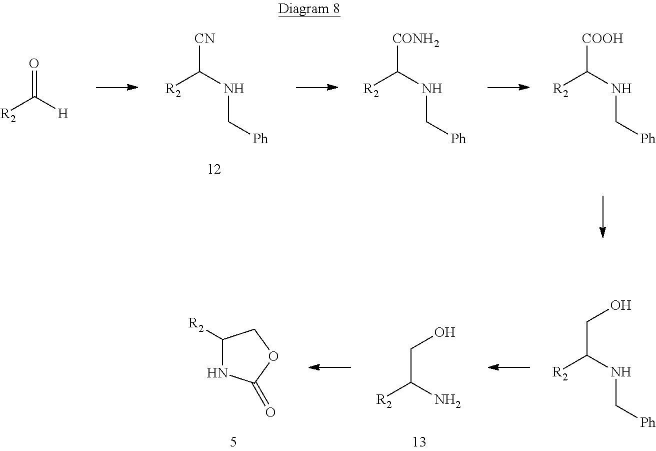

or for benzyl substituted R2, from amino acid or corresponding ester 14, which is reduced to amino alcohol then transformed to the corresponding oxazolidinone by reaction with phosgene or a chemical analog such as carbonyl diimidazole according to diagram 9.

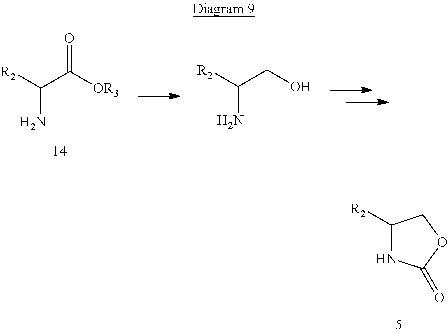

or for R2 alkoxy, or alkoxybenzyl or alkoxyphenyl from serine ester which is cyclized into the corresponding oxazolidinone by the action of phosgene or a chemical analog such as diimidazole carbonyl, then the ester function is reduced to the corresponding alcohol, the hydroxy function is transformed to the leaving group which is then substituted by the action of an alkoxide to give compound 3 as described in diagram 10.

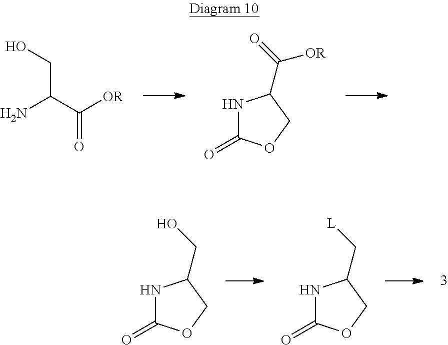

either when R2 represents several identical or different substituents on the oxazolidinone rings each independently representing:
a C₁₋₄ alkyl group
a phenyl group
a benzyl group
a C₁₋₄ hydroxyalkyl group
   from serine ester (S or R)—or the corresponding hydroxy amino acid for longer chains—the amine and alcohol functional groups are protected, the di-protected product thus obtained is treated with an organic anion—for Example an organomagnesium—to yield corresponding di-substituted compound 15 (R4=alkyl, phenyl or benzyl), or first reduced to an aldehyde and then also treated with an anion to give corresponding monosubstituted compound 16 (R4=alkyl, phenyl or benzyl). Compounds 15 and 16 then undergo the cyclization/deprotection of the amine steps or the reverse, which is cyclized by sodium hydride into oxazolidinone whose nitrogen atom is then deprotected to provide corresponding compounds 5 after deprotection of the alcohol function according to diagram 11.

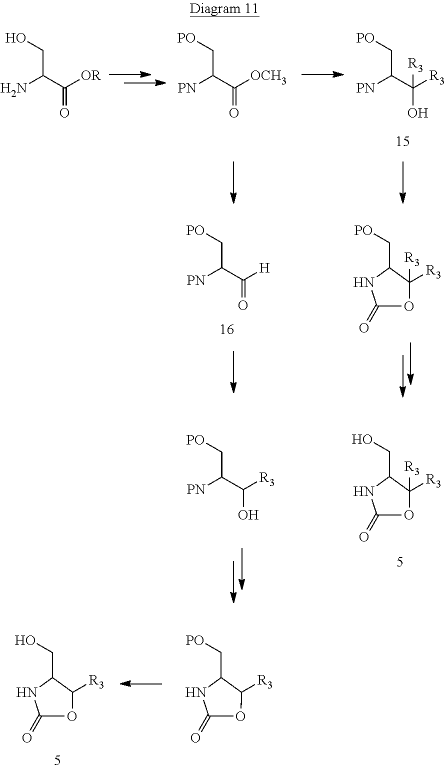

For example, but non-limiting, preparations of the compounds of the invention are illustrated in the following examples:

### EXAMPLE 1

### (S)-3-(2-(1-((2,3-dihydrobenzo[b][1,4]dioxin-2-yl)methyl)piperidin-4-yl)ethyl)oxazolidin-2-one

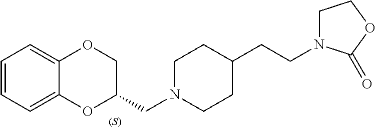

In a 100-mL single-necked flask, 0.8 g (3.58 mmol) of (R)-2-(bromomethyl)-2,3-dihydrobenzo[b][1,4]dioxin 2.1 are added to a solution of 0.71 g (3.58 mmol) of 3-(2-(piperidin-4-yl)ethyl)oxazolidin-2-one 3.1 in 15 ml of acetonitrile. Then 0.65 g (4.7 mmol) of K₂CO₃ and a spatula tip of KI are introduced. This is heated at 80° C. for 8 hours. After return to room temperature, the medium is concentrated, then washed with water and extracted with dichloromethane. The organic phase is dried on MgSO₄, filtered, evaporated and purified by flash chromatography on silica gel (eluent: dichloromethane/ethyl acetate=85/15%). 0.3 g (0.87 mmol) of compound 1 are recovered in the form of a colorless oil.

Yield: 24%

¹H NMR (CDCl₃) δ: 1.29 (ls, 3H); 1.48 (m, 2H); 1.71 (is, 2H); 2.09 (m, 2H); 2.55 (m, 1H); 2.65 (m, 1H); 2.89 (m, 1H); 2.99 (m, 1H); 3.31 (t, 2H, J=7.2 Hz); 3.54 (t, 2H, J=8.4 Hz); 3.97 (dd, 1H, J=7.8 and 8 Hz); 4.31 (m, 4H); 6.85 (m, 4H).

0.3 g (0.87 mmol) of compound 1 are dissolved in a minimal amount of methanol and 0.4 ml (2 mmol) of a 5 N solution of HCl in isopropanol are added. The medium is concentrated and triturated with ethyl ether and acetone. The precipitate is filtered and left to dry in the vacuum chamber. 0.19 g (0.50 mmol) of compound 1 hydrochloride are obtained in the form of a white solid.

F: 120-123° C.

[α]=−56.07 (c=0.135, methanol)

¹H NMR (DMSOd₆) δ: 1.51 (m, 5H); 1.9 (m, 2H); 3.02 (m, 2H); 3.19 (t, 2H, J=6.8 Hz); 3.29 (m, 1H); 3.48 (m, 2H); 3.52 (t, 2H, J=8 Hz); 3.66 (m, 1H); 4.05 (m, 1H); 4.25 (t, 2H, J=8 Hz); 4.32 (m, 1H); 4.86 (m, 1H); 6.91 (m, 4H); 10.48 (ls, 1H).

MS m/z 347 (M+1).

HPLC (XBRIDGE C8, acetonitrile/water/KH₂PO₄ (20/80/6.8 g pH 4), 1 ml/min), retention time=10.08 min, chemical purity=99.4%

HPLC (CHIRALPACK AD-H, heptane/ethanol/diethylamine (40/60/0.1), 0.8 ml/min); compound 1, retention time=6.53 min, compound 2, retention time=5.92 min, ratio of AUCs (1)/(2)=99.3/0.7.

### EXAMPLE 2

### (R)-3-(2-(1-((2,3-dihydrobenzo[b][1,4]dioxin-2-yl)methyl)piperidin-4-yl)ethyl)oxazolidin-2-one

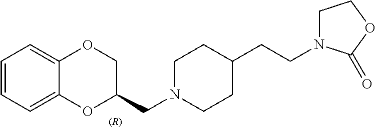

By proceeding as in Example 1 but replacing (R)-2-(bromomethyl)-2,3-dihydrobenzo[b][1,4]dioxine 2.1 by (S)-2-(bromomethyl)-2,3-dihydrobenzo[b][1,4]dioxine 2.2, 0.8 g (2.31 mmol) of compound 2 are obtained in the form of a pale yellow oil, after purification by flash chromatography on silica gel (eluent: dichloromethane/ethyl acetate=85/15%).

Yield: 40%.

¹H NMR (CDCl₃) δ: see NMR Example 1

0.8 g (2.31 mmol) of compound 2 are dissolved in a minimal amount of methanol and 0.9 ml (4.52 mmol) of a 5 N solution of HCl in isopropanol are added. The medium is concentrated and triturated with ethyl ether and acetone. The precipitate is filtered and dried in the vacuum chamber. 0.75 g (1.82 mmol) of compound 2 hydrochloride are obtained in the form of a white solid.

[α]=+57.60 (c=0.155, methanol)

¹H NMR (DMSOd₆) δ: see NMR Example 1

MS m/z 347 (M+1).

HPLC (CHIRALPACK AD-H, heptane/ethanol/diethylamine (40/60/0.1), 0.8 ml/min); compound 1, retention time=6.50 min, compound 2, retention time=5.88 min, ratio of AUCs (1)/(2)=1.2/98.8.

### EXAMPLE 3

### (S)-2-((4-(2-(2-oxooxazolidin-3-yl)ethyl)piperidin-1-yl)methyl)-2,3-dihydrobenzo[b][1,4]dioxine-6-carbonitrile

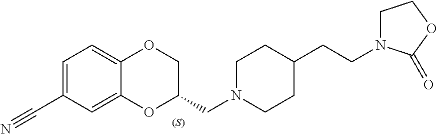

By proceeding as in Example 1 but replacing (R)-2-(bromomethyl)-2,3-dihydrobenzo[b][1,4]dioxine 2.1 by (R)-3-(bromomethyl)-2,3-dihydrobenzo[b][1,4]dioxine-6-carbonitrile 2.11, and the K₂CO₃/KI pair by diisopropylethylamine (1.4 eq), 0.83 g (2.23 mmol) of compound 3 are obtained in the form of a pale yellow oil after purification by flash chromatography on silica gel (eluent: dichloromethane/methanol=95/5%).

Yield: 68%.

¹H NMR (CDCl₃) δ: 1.28 (ls, 3H); 1.49 (m, 2H); 1.73 (m, 2H); 2.54 (m, 1H); 2.09 (m, 2H); 2.65 (m, 1H); 2.91 (m, 2H); 3.31 (t, 2H, J=7.6 Hz); 3.54 (t, 2H, J=8 Hz); 4.32 (m, 2H); 4.33 (t, 2H, J=8 Hz); 3.54 (t, 2H, J=8 Hz); 6.91 (d, 1H, J=8.4 Hz); 7.13 (dd, 1H, J=8.4 Hz and 1.6 Hz); 7.17 (d, 1H, J=1.6 Hz).

0.83 g (2.23 mmol) of compound 3 are dissolved in 5 ml of methanol and 0.18 g (2.01 mmol) of oxalic acid in solution in 5 ml of methanol are added. The medium is concentrated and triturated with ethyl ether and acetone. The solid that precipitates is filtered and then dried in the vacuum chamber. 0.8 g (1.73 mmol) of compound 3 oxalate are recovered in the form of a white solid.

F: 182° C.

[α]=−58.96 (c=0.081, methanol)

¹H NMR (DMSOd₆) δ: 1.37 (m, 5H); 1.80 (m, 2H); 2.60 (m, 2H); 3.06 (ls, 2H); 3.18 (t, 2H, J=6.8 Hz); 3.30 (m, 2H); 3.51 (t, 2H, J=8.4 Hz); 4.12 (dd, 1H, J=11.6 and 6.8 Hz); 4.24 (t, 2H, J=8.4 Hz); 4.41 (m, 1H); 4.68 (ls, 1H); 7.08 (d, 1H, J=8.4 Hz); 7.35 (dd, 1H, J=8.4 and 1.6 Hz); 7.44 (d, 1H, J=1.6 Hz).

MS m/z 372 (M+1).

HPLC (Acquity BEH C8, Phase A: 100% H₂O. 0.01 M pH 4 HCOONH₄, Phase B: 90% Acetonitrile 10% H₂O. 0.01 M pH 4 HCOONH₄, gradient B: 15 to 40% in 5 min, 0.75 ml/min), retention time=1.62 min, chemical purity: 99.3%.

HPLC (CHIRALPACK AD-H, acetonitrile/ethanol/diethylamine (90/10/0.1), 1 ml/min); compound 3, retention time=6.91 min, compound 4, retention time=6.03 min, ratio of AUCs (3)/(4)=99.6/0.4.

### EXAMPLE 4

### (R)-3-((4-(2-(2-oxooxazolidin-3-yl)ethyl)piperidin-1-yl)methyl)-2,3-dihydrobenzo[b][1,4]dioxine-6-carbonitrile

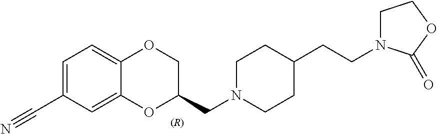

By proceeding as in Example 1 but replacing (R)-2-(bromomethyl)-2,3-dihydrobenzo[b][1,4]dioxine 2.1 by (S)-3-(bromomethyl)-2,3-dihydrobenzo[b][1,4]dioxine-6-carbonitrile 2.12, and the K₂CO₃/KI pair by diisopropylethylamine (1.4 eq), 1.8 g (4.85 mmol) of compound 4 are obtained in the form of a pale yellow oil, after purification by flash chromatography on silica gel (eluent: dichloromethane/methanol=95/5%).

Yield: 64%.

¹H NMR (CDCl₃) δ: see NMR Example 3

1.8 g (4.85 mmol) of compound 4 are dissolved in 10 ml of methanol and then 0.43 g (4.77 mmol) of oxalic acid in solution in 5 ml of methanol are added. The medium is concentrated and triturated with ethyl ether. The solid that precipitates is filtered and then dried in the vacuum chamber. 1.8 g (3.90 mmol) of compound 4 oxalate are recovered in the form of a white solid.

F: 190° C.

[α]=+51 (c=0.114, methanol)

¹H NMR (DMSOd₆) δ: see NMR Example 3

MS m/z 372 (M+1).

HPLC (Acquity BEH C8, Phase A: 100% H₂O. 0.01 M pH 4 HCOONH₄, Phase B: 90% Acetonitrile 10% H₂O. 0.01 M pH 4 HCOONH₄, gradient B: 15 to 40% in 5 min, 0.75 ml/min), retention time=1.62 min, chemical purity: 99.6%.

HPLC (CHIRALPACK AD-H, acetonitrile/ethanol/diethylamine (90/10/0.1), 1 ml/min); compound 4, retention time=6.01 min, compound 3, retention time=6.92 min, ratio of AUCs (4)/(3)=99.4/0.6.

### EXAMPLE 5

### (S)-3-((4-(2-(2-oxooxazolidin-3-yl)ethyl)piperidin-1-yl)methyl)-2,3-dihydrobenzo[b][1,4]dioxine-5-carbonitrile

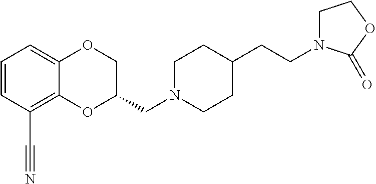 [SMILES: N#Cc1cccc2c1O[C@@H](CN1CCC(CCN3CCOC3=O)CC1)CO2]

By proceeding as in Example 1 but replacing (R)-2-(bromomethyl)-2,3-dihydrobenzo[b][1,4]dioxine 2.1 by (R)-(8-cyano-2,3-dihydrobenzo[b][1,4]dioxin-2-yl)methyl 4-bromobenzenesulfonate 2.13, and the K₂CO₃/KI pair by diisopropylethylamine (1.4 eq), 1.04 g (2.80 mmol) of compound 5 are obtained in the form of a pale yellow oil, after purification by flash chromatography on silica gel (gradient over 30 min dichloromethane=100% to dichloromethane/methanol/NH₄OH=96/3.5/0.5%).

Yield: 55%.

¹H NMR (CDCl₃) δ: 1.26 (m, 3H); 1.49 (m, 2H); 1.72 (m, 2H); 2.12 (m, 1H); 2.23 (m, 1H); 2.64 (dd, 1H, J=13.6 and 6.8 Hz); 2.72 (dd, 1H, J=13.6 and 5.2 Hz); 2.86 (m, 1H); 3.01 (m, 1H); 3.30 (t, 2H, J=7.2 Hz); 3.55 (t, 2H, J=8 Hz); 4.04 (dd, 1H, J=11.4 and 6.8 Hz); 4.34 (m, 4H); 6.87 (m, 1H); 7.07 (dd, 1H, J=8.4 and 1.6 Hz); 7.12 (dd, 1H, J=7.6 and 1.6 Hz).

1.04 g (2.80 mmol) of compound 5 are dissolved in 10 ml of ethyl acetate and 1 ml (5 mmol) of 5 N hydrochloric acid in isopropanol is added. The medium is stirred and then the solid is filtered and washed with ethyl ether. The solid is dried in the vacuum chamber. 1.05 g (2.57 mmol) of compound 5 hydrochloride are recovered in the form of a white solid.

F: 196° C.

¹H NMR (DMSOd₆) δ: 1.53 (m, 5H); 1.91 (m, 2H); 3.11 (m, 2H); 3.20 (t, 2H, J=6.8 Hz); 3.39 (m, 3H); 3.52 (t, 2H, J=8.4 Hz); 3.55 (m, 1H); 3.75 (m, 1H); 4.18 (dd, 1H, J=12 and 6.8 Hz); 4.25 (m, 1H); 4.45 (dd, 1H, 11.8 and 2 Hz); 5.14 (m, 1H); 7.05 (m, 1H); 7.28 (dd, 1H, J=8.2 and 1.2 Hz); 7.37 (dd, 1H, J=8 and 1.2 Hz); 10.98 (is, 1H).

MS m/z 372 (M+1).

HPLC (X-BRIDGE C8, acetonitrile/H₂O.KH₂PO₄ 6.8 g pH 4 (23/77), 1 ml/min), retention time=7.37 min, chemical purity: 100%.

### EXAMPLE 6

### (S)-2-((4-(2-(2-oxooxazolidin-3-yl)ethyl)piperidin-1-yl)methyl)-2,3-dihydrobenzo[b][1,4]dioxine-5-carbonitrile

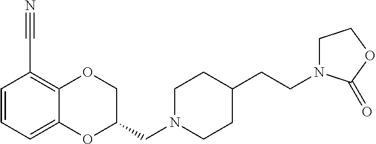 [SMILES: N#Cc1cccc2c1OC[C@H](CN1CCC(CCN3CCOC3=O)CC1)O2]

By proceeding as in Example 1 but replacing (R)-2-(bromomethyl)-2,3-dihydrobenzo[b][1,4]dioxine 2.1 by (R)-(5-cyano-2,3-dihydrobenzo[b][1,4]dioxin-2-yl)methyl 4-bromobenzenesulfonate 2.14, and the K₂CO₃/KI pair by diisopropylethylamine (1.4 eq), 0.14 g (0.37 mmol) of compound 6 are obtained in the form of a pale yellow oil after purification by flash chromatography on silica gel (eluent: dichloromethane/methanol=95/5%).

Yield: 50%.

¹H NMR (DMSOd₆) δ: 1.17 (m, 3H); 1.40 (m, 2H); 1.64 (m, 2H); 1.99 (m, 2H); 2.55 (m, 2H); 2.88 (m, 2H); 3.17 (t, 2H, J=7.2 Hz); 3.50 (t, 2H, J=8.4 Hz); 4.12 (dd, 1H, J=11.4 and 7.2 Hz); 4.24 (t, 2H, J=8 Hz); 4.43 (m, 2H); 6.97 (m, 1H); 7.21 (dd, 1H, J=8.2 and 1.2 Hz); 7.28 (dd, 1H, J=8.4 and 1.2 Hz).

0.14 g (0.37 mmol) of compound 6 are dissolved in 5 ml of acetonitrile and then 1 ml of water and 0.4 ml (0.40 mmol) of 1 N hydrochloric acid are added. After lyophilization, 0.15 g (0.37 mmol) of compound 6 hydrochloride are obtained in the form of a white solid.

¹H NMR (DMSOd₆) δ: 1.45 (m, 5H); 1.90 (m, 2H); 3.02 (m, 2H); 3.19 (t, 2H); 3.50 (m, 4H); 3.52 (t, 2H, J=7.6 Hz); 4.25 (m, 3H); 4.52 (m, 1H); 4.95 (m, 1H); 7.06 (m, 1H); 7.31 (m, 1H); 7.38 (m, 1H); 9.90 (ls, 1H).

MS m/z 372 (M+1).

HPLC (X-BRIDGE C18, Phase A: 100% H₂O. 0.1%, TFA, Phase B: 100% acetonitrile, gradient B: 0 to 100% in 6 min, 1.8 ml/min), retention time=4.26 min, chemical purity: 99.2%.

### EXAMPLE 7

### (S)-3-(2-(1-((7-fluoro-2,3-dihydrobenzo[b][1,4]dioxin-2-yl)methyl)piperidin-4-yl)ethyl)oxazolidin-2-one

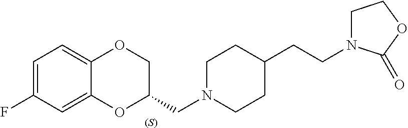

By proceeding as in Example 1 but replacing (R)-2-(bromomethyl)-2,3-dihydrobenzo[b][1,4]dioxine 2.1 by (R)-(7-fluoro-2,3-dihydrobenzo[b][1,4]dioxin-2-yl)methyl 4-bromobenzenesulfonate 2.17, the K₂CO₃/KI pair by diisopropylethylamine (1.4 eq) and acetonitrile by dimethylsulfoxide, 0.10 g (0.27 mmol) of compound 7 are obtained in the form of a pale yellow oil, after purification by flash chromatography on silica gel (gradient over 30 min 100% dichloromethane to dichloromethane/methanol=95/5%).

Yield: 37%.

¹H NMR (DMSOd₆) δ: 1.18 (m, 3H); 1.40 (m, 2H); 1.64 (m, 2H); 1.98 (m, 2H); 2.52 (m, 2H); 2.87 (m, 2H); 3.17 (t, 2H, J=7.2 Hz); 3.50 (t, 2H, J=8.4 Hz); 3.92 (dd, 1H, J=11.6 and 7.2 Hz); 4.23 (t, 2H, J=8.4 Hz); 4.25 (m, 1H); 4.35 (m, 1H); 6.65 (m, 1H); 6.76 (m, 1H); 6.87 (m, 1H).

0.10 g (0.27 mmol) of compound 7 are dissolved in 5 ml of acetonitrile and then 1 ml of water and 0.29 ml (0.29 mmol) of 1 N hydrochloric acid are added. After lyophilization, 0.11 g (0.27 mmol) of compound 7 hydrochloride are obtained in the form of a white solid.

¹H NMR (DMSOd₆) δ: 1.45 (m, 5H); 1.90 (m, 2H); 3.01 (m, 2H); 3.19 (m, 2H); 3.32 (m, 3H); 3.52 (t, 2H, J=8.4 Hz); 3.65 (m, 1H); 4.05 (dd, 1H, J=11.6 and 6.4 Hz); 4.25 (t, 2H, J=8.4 Hz); 4.28 (m, 1H); 4.89 (m, 1H); 6.75 (m, 1H); 6.87 (m, 1H); 6.95 (m, 1H); 10.14 (ls, 1H).

MS m/z 365 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.40 min, chemical purity: 97.8%.

### EXAMPLE 8

### (S)-3-(2-(1-((8-fluoro-2,3-dihydrobenzo[b][1,4]dioxin-2-yl)methyl)piperidin-4-yl)ethyl)oxazolidin-2-one

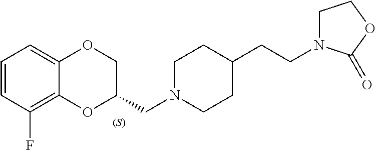

By proceeding as in Example 1 but replacing (R)-2-(bromomethyl)-2,3-dihydrobenzo[b][1,4]dioxine 2.1 by (R)-(8-fluoro-2,3-dihydrobenzo[b][1,4]dioxin-2-yl)methyl 4-bromobenzenesulfonate 2.16, the K₂CO₃/KI pair by diisopropylethylamine (1.4 eq) and acetonitrile by dimethylsulfoxide, 0.12 g (0.33 mmol) of compound 8 are obtained in the form of a beige solid, after purification by flash chromatography on silica gel (gradient over 30 min 100% dichloromethane to dichloromethane/methanol=95/5%).

Yield: 44%.

¹H NMR (DMSOd₆) δ: 1.16 (m, 3H); 1.40 (m, 2H); 1.65 (m, 2H); 2.01 (m, 2H); 2.56 (m, 2H); 2.83 (m, 1H); 2.94 (m, 1H); 3.17 (t, 2H, J=7.2 Hz); 3.50 (t, 2H, J=8 Hz); 4.00 (dd, 1H, J=11.6 and 7.2 Hz); 4.24 (t, 2H, J=8 Hz); 4.36 (m, 2H); 6.75 (m, 3H).

0.12 g (0.33 mmol) of compound 8 are dissolved in 5 ml of acetonitrile and then 1 ml of water and 0.35 ml (0.35 mmol) of 1 N hydrochloric acid are added. After lyophilization, 0.12 g (0.30 mmol) of compound 8 hydrochloride are obtained in the form of a pale yellow solid.

¹H NMR (DMSOd₆) δ: 1.46 (m, 5H); 1.91 (m, 2H); 3.04 (m, 2H); 3.19 (m, 2H); 3.34 (m, 1H); 3.51 (m, 4H); 3.66 (m, 1H); 4.13 (m, 1H); 4.25 (m, 2H); 4.39 (m, 1H); 4.96 (m, 1H); 6.78 (m, 1H); 6.87 (m, 2H); 10.43 (ls, 1H).

MS m/z 365 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.37 min, chemical purity: 100%.

### EXAMPLE 9

### (S)-3-(2-(1-((6-fluoro-2,3-dihydrobenzo[b][1,4]dioxin-2-yl)methyl)piperidin-4-yl)ethyl)oxazolidin-2-one

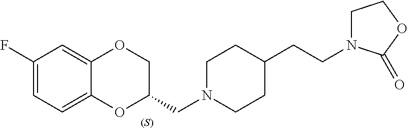

By proceeding as in Example 1 but replacing (R)-2-(bromomethyl)-2,3-dihydrobenzo[b][1,4]dioxine 2.1 by (R)-(6-fluoro-2,3-dihydrobenzo[b][1,4]dioxin-2-yl)methyl 4-bromobenzenesulfonate 2.18, the K₂CO₃/KI pair by diisopropylethylamine (1.4 eq) and acetonitrile by dimethylsulfoxide, 0.12 g (0.33 mmol) of compound 9 are obtained in the form of a pale yellow solid, after purification by flash chromatography on silica gel (gradient over 30 min 100% dichloromethane to dichloromethane/methanol=95/5%).

Yield: 52%.

¹H NMR (DMSOd₆) δ: 1.17 (m, 3H); 1.40 (m, 2H); 1.64 (m, 2H); 1.98 (m, 2H); 2.50 (m, 2H); 2.87 (m, 2H); 3.17 (t, 2H, J=7.2 Hz); 3.50 (t, 2H, J=8 Hz); 3.95 (dd, 1H, J=11.8 and 7.6 Hz); 4.26 (m, 4H); 6.65 (m, 1H); 6.76 (m, 1H); 6.87 (m, 1H).

0.12 g (0.33 mmol) of compound 9 are dissolved in 5 ml of acetonitrile and then 1 ml of water and 0.35 ml (0.35 mmol) of 1 N hydrochloric acid are added. After lyophilization, 0.13 g (0.32 mmol) of compound 9 hydrochloride are obtained in the form of a white solid.

¹H NMR (DMSOd₆) δ: 1.51 (m, 5H); 1.89 (m, 2H); 3.01 (m, 2H); 3.21 (m, 2H); 3.28 (m, 1H); 3.48 (m, 2H); 3.52 (t, 2H, J=8 Hz); 3.66 (m, 1H); 4.07 (dd, 1H, J=11.6 and 6.8 Hz); 4.25 (t, 2H, J=8 Hz); 4.37 (m, 1H); 4.89 (m, 1H); 6.74 (m, 1H); 6.85 (m, 1H); 6.97 (m, 1H,); 10.70 (ls, 1H).

MS m/z 365 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.41 min, chemical purity: 98.1%.

### EXAMPLE 10

### (S)-3-(2-(1-((5,7-difluoro-2,3-dihydrobenzo[b][1,4]dioxin-2-yl)methyl)piperidin-4-yl)ethyl)oxazolidin-2-one

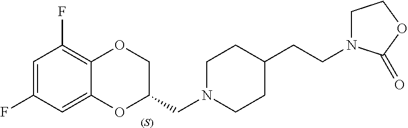

By proceeding as in Example 1 but replacing (R)-2-(bromomethyl)-2,3-dihydrobenzo[b][1,4]dioxine 2.1 by (R)-(5,7-difluoro-2,3-dihydrobenzo[b][1,4]dioxin-2-yl)methyl 4-bromobenzenesulfonate 2.19, the K₂CO₃/KI pair by diisopropylethylamine (1.4 eq) and acetonitrile by dimethylsulfoxide, 0.11 g (0.29 mmol) of compound 10 are obtained in the form of a pale yellow oil, after purification by flash chromatography on silica gel (gradient over 30 min 100% dichloromethane to dichloromethane/methanol=95/5%).

Yield: 40%.

¹H NMR (DMSOd₆) δ: 1.16 (m, 3H); 1.40 (m, 2H); 1.64 (m, 2H); 1.99 (m, 2H); 2.54 (m, 2H); 2.87 (m, 2H); 3.17 (t, 2H, J=7.2 Hz); 3.50 (t, 2H, J=8 Hz); 3.99 (dd, 1H, 11.6 Hz and 7.2 Hz); 4.24 (t, 2H, J=8 Hz); 4.34 (m, 1H); 4.44 (m, 1H); 6.69 (m, 1H); 6.84 (m, 1H).

0.11 g (0.29 mmol) of compound 10 are dissolved in 5 ml of acetonitrile and then 1 ml of water and 0.30 ml (0.30 mmol) of 1 N hydrochloric acid are added. After lyophilization, 0.11 g (0.26 mmol) of compound 10 hydrochloride are obtained in the form of a pale yellow oil.

¹H NMR (DMSOd₆) δ: 1.45 (m, 5H); 1.90 (m, 2H); 3.00 (m, 2H); 3.20 (m, 2H); 3.42 (m, 3H); 3.52 (t, 2H J=8 Hz); 3.65 (m, 1H); 4.13 (dd, 1H, J=11.8 and 6.4 Hz); 4.25 (t, 2H, J=8 Hz); 4.38 (m, 1H); 4.97 (m, 1H); 6.79 (m, 1H); 6.96 (m, 1H); 10.11 (ls, 1H).

MS m/z 383 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.50 min, chemical purity: 97.9%.

### EXAMPLE 11

### (S)-3-(2-(1-((6.7-difluoro-2,3-dihydrobenzo[b][1,4]dioxin-2-yl)methyl)piperidin-4-yl)ethyl)oxazolidin-2-one

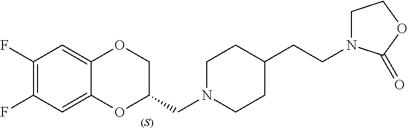

By proceeding as in Example 1 but replacing (R)-2-(bromomethyl)-2,3-dihydrobenzo[b][1,4]dioxine 2.1 by (R)-(6,7-difluoro-2,3-dihydrobenzo[b][1,4]dioxin-2-yl)methyl 4-bromobenzenesulfonate 2.22, the K₂CO₃/KI pair by diisopropylethylamine (1.4 eq) and acetonitrile by dimethylsulfoxide, 0.11 g (0.29 mmol) of compound 11 are obtained in the form of a pale yellow oil, after purification by flash chromatography on silica gel (gradient over 30 min 100% dichloromethane to dichloromethane/methanol=95/5%).

Yield: 40%.

¹H NMR (DMSOd₆) δ: 1.16 (m, 3H); 1.40 (m, 2H); 1.64 (m, 2H); 1.98 (m, 2H); 2.50 (m, 2H); 2.87 (m, 2H); 3.17 (t, 2H, J=7.6 Hz); 3.50 (t, 2H, J=8 Hz); 3.95 (dd, 1H, J=11.2 and 7.2 Hz); 4.24 (t, 2H, J=8 Hz); 4.28 (m, 2H); 7.04 (m, 2H).

0.11 g (0.29 mmol) of compound 11 are dissolved in 5 ml of acetonitrile and then 1 ml of water and 0.30 ml (0.30 mmol) of 1 N hydrochloric acid are added. After lyophilization, 0.12 g (0.28 mmol) of compound 11 hydrochloride are obtained in the form of a pale yellow gum.

¹H NMR (DMSOd₆) δ: 1.46 (m, 5H); 1.90 (m, 2H); 3.02 (m, 2H); 3.19 (m, 2H); 3.38 (m, 3H); 3.52 (t, 2H, J=8 Hz); 3.65 (m, 1H); 4.07 (dd, 1H, J=11.6 and 6.4 Hz); 4.25 (t, 2H, J=8 Hz); 4.33 (m, 1H); 4.87 (m, 1H); 7.14 (m, 2H); 10.1 (ls, 1H).

MS m/z 383 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.54 min, chemical purity: 98.1%.

### EXAMPLE 12

### (S)-3-(2-(1-((7-chloro-5-fluoro-2,3-dihydrobenzo[b][1,4]dioxin-2-yl)methyl)piperidin-4-yl)ethyl)oxazolidin-2-one

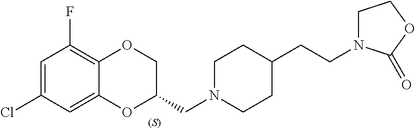

By proceeding as in Example 1 but replacing (R)-2-(bromomethyl)-2,3-dihydrobenzo[b][1,4]dioxine 2.1 by (R)-(7-chloro-5-fluoro-2,3-dihydrobenzo[b][1,4]dioxin-2-yl)methyl 4-bromobenzenesulfonate 2.20, the K₂CO₃/KI pair by diisopropylethylamine (1.4 eq) and acetonitrile by dimethylsulfoxide, 0.10 g (0.25 mmol) of compound 12 are obtained in the form of a pale yellow solid, after purification by flash chromatography on silica gel (gradient over 30 min 100% dichloromethane to dichloromethane/methanol=95/5%).

Yield: 38%.

¹H NMR (DMSOd₆) δ: 1.16 (m, 3H); 1.40 (m, 2H); 1.64 (m, 2H); 1.99 (m, 2H); 2.54 (m, 2H); 2.87 (m, 2H); 3.17 (t, 2H, J=7.2 Hz); 3.50 (t, 2H, J=8 Hz); 4.02 (dd, 1H, J=11.4 and 7.2 Hz); 4.24 (t, 2H, J=8 Hz); 4.37 (m, 1H); 4.45 (m, 1H); 6.87 (m, 1H); 7.01 (m, 1H).

0.10 g (0.25 mmol) of compound 12 are dissolved in 5 ml of acetonitrile and then 1 ml of water and 0.26 ml (0.26 mmol) of 1 N hydrochloric acid are added. After lyophilization, 0.11 g (0.25 mmol) of compound 12 hydrochloride are obtained in the form of a pale yellow solid.

¹H NMR (DMSOd₆) δ: 1.46 (m, 5H); 1.92 (m, 2H); 3.02 (m, 2H); 3.20 (t, 2H, J=7.2 Hz); 3.40 (m, 3H); 3.52 (t, 2H, J=8 Hz); 3.64 (m, 1H); 4.16 (dd, 1H, J=11.8 and 6 Hz); 4.25 (t, 2H, J=8 Hz); 4.40 (m, 1H); 4.97 (m, 1H); 6.97 (m, 1H); 7.12 (m, 1H); 10.01 (ls, 1H).

MS m/z 399 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.73 min, chemical purity: 98.6%.

### EXAMPLE 13

### (S)-3-(2-(1-((5-chloro-7-fluoro-2,3-dihydrobenzo[b][1,4]dioxin-2-yl)methyl)piperidin-4-yl)ethyl)oxazolidin-2-one

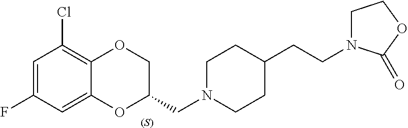

By proceeding as in Example 1 but replacing (R)-2-(bromomethyl)-2,3-dihydrobenzo[b][1,4]dioxine 2.1 by (R)-(5-chloro-7-fluoro-2,3-dihydrobenzo[b][1,4]dioxin-2-yl)methyl 4-bromobenzenesulfonate 2.21, the K₂CO₃/KI pair by diisopropylethylamine (1.4 eq) and acetonitrile by dimethylsulfoxide, 0.10 g (0.25 mmol) of compound 13 are obtained in the form of an orange oil after purification by flash chromatography on silica gel (gradient over 30 min 100% dichloromethane to dichloromethane/methanol=95/5%).

Yield: 37%.

¹H NMR (DMSOd₆) δ: 1.16 (m, 3H); 1.40 (m, 2H); 1.64 (m, 2H); 1.98 (m, 2H); 2.54 (m, 2H); 2.88 (m, 2H); 3.17 (t, 2H, J=6.8 Hz); 3.50 (t, 2H, J=8 Hz); 4.02 (dd, 1H, J=11.6 and 7.2 Hz); 4.24 (t, 2H, J=8 Hz); 4.41 (m, 2H); 6.85 (m, 1H); 6.97 (m, 1H).

0.10 g (0.25 mmol) of compound 13 are dissolved in 5 ml of acetonitrile and then 1 ml of water and 0.26 ml (0.26 mmol) of 1 N hydrochloric acid are added. After lyophilization, 0.11 g (0.25 mmol) of compound 13 hydrochloride are obtained in the form of a beige gum.

¹H NMR (DMSOd₆) δ: 1.46 (m, 5H); 1.91 (m, 2H); 3.01 (m, 2H); 3.20 (t, 2H); 3.37 (m, 3H); 3.52 (t, 2H, J=8 Hz); 3.62 (m, 1H); 4.18 (dd, 1H, J=12 and 5.6 Hz); 4.25 (t, 2H, J=8 Hz); 4.41 (m, 1H); 4.94 (m, 1H); 6.94 (m, 1H); 7.09 (m, 1H); 9.9 (ls, 1H).

MS m/z 399 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.68 min, chemical purity: 97.4%.

### EXAMPLE 14

### (S)-3-(2-(1-((7-chloro-2,3-dihydrobenzo[b][1,4]dioxin-2-yl)methyl)piperidin-4-yl)ethyl)oxazolidin-2-one

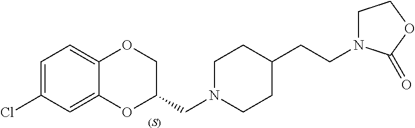

By proceeding as in Example 1 but replacing (R)-2-(bromomethyl)-2,3-dihydrobenzo[b][1,4]dioxine 2.1 by (R)-(7-chloro-2,3-dihydrobenzo[b][1,4]dioxin-2-yl)methyl 4-bromobenzenesulfonate 2.7, the K₂CO₃/KI pair by diisopropylethylamine (1.4 eq) and acetonitrile by dimethylsulfoxide, 0.12 g (0.31 mmol) of compound 14 are obtained in the form of a pale yellow oil, after purification by flash chromatography on silica gel (gradient over 30 min 100% dichloromethane to dichloromethane/methanol=95/5%).

Yield: 45%.

¹H NMR (DMSOd₆) δ: 1.17 (m, 3H); 1.40 (m, 2H); 1.64 (m, 2H); 1.98 (m, 2H); 2.52 (m, 2H); 2.87 (m, 2H); 3.17 (t, 2H, J=7.2 Hz); 3.50 (t, 2H, J=8 Hz); 3.96 (dd, 1H, J=11.6 and 6.8 Hz); 4.24 (t, 2H, J=8 Hz); 4.29 (m, 1H); 4.38 (m, 1H); 6.87 (m, 2H); 6.95 (d, 1H, J=2.4 Hz).

0.12 g (0.31 mmol) of compound 14 are dissolved in 5 ml of acetonitrile and then 1 ml of water and 0.32 ml (0.32 mmol) of 1 N hydrochloric acid are added. After lyophilization, 0.13 g (0.31 mmol) of compound 14 hydrochloride are obtained in the form of a pale yellow gum.

¹H NMR (DMSOd₆) δ: 1.46m, 5H); 1.92 (m, 2H); 3.02 (m, 2H); 3.20 (t, 2H); 3.38 (m, 3H); 3.52 (t, 2H, J=8 Hz); 3.63 (m, 1H); 4.08 (dd, 1H, J=11.6 and 6 Hz); 4.25 (t, 2H, J=8 Hz); 4.32 (m, 1H); 4.87 (m, 1H); 6.95 (is, 2H); 7.07 (ls, 1H); 9.9 (ls, 1H).

MS m/z 381 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.62 min, chemical purity: 98.7%.

### EXAMPLE 15

### (S)-3-(2-(1-((7-(methylsulfonyl)-2,3-dihydrobenzo[b][1,4]dioxin-2-yl)methyl)piperidin-4-yl)ethyl)oxazolidin-2-one

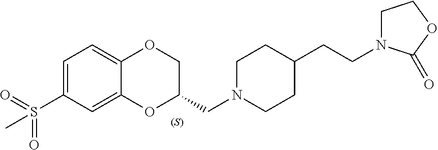

By proceeding as in Example 1 but replacing (R)-2-(bromomethyl)-2,3-dihydrobenzo[b][1,4]dioxine 2.1 by (R)-(7-(methylsulfonyl)-2,3-dihydrobenzo[b][1,4]dioxin-2-yl)methyl 4-bromobenzenesulfonate 2.23, the K₂CO₃/KI pair by diisopropylethylamine (1.4 eq) and acetonitrile by dimethylsulfoxide, 0.12 g (0.31 mmol) of compound 15 are obtained in the form of a colorless oil, after purification by flash chromatography on silica gel (gradient over 30 min 100% dichloromethane to dichloromethane/methanol=95/5%).

Yield: 14%.

¹H NMR (DMSOd₆) δ: 1.17 (m, 3H); 1.40 (m, 2H); 1.65 (m, 2H); 2.01 (m, 2H); 2.54 (m, 2H); 2.90 (m, 2H); 3.16 (s, 3H); 3.17 (t, 2H); 3.50 (t, 2H, J=8 Hz); 4.06 (dd, 1H, J=11.6 and 6.8 Hz); 4.24 (t, 2H, J=8 Hz); 4.40 (m, 2H); 7.10 (d, 1H, J=8.4 Hz); 7.36 (m, 2H).

0.12 g (0.31 mmol) of compound 15 are dissolved in 5 ml of acetonitrile and then 1 ml of water and 0.0028 g (0.31 mmol) of oxalic acid are added. After lyophilization, 0.13 g (0.31 mmol) of compound 15 oxalate are recovered in the form of a white solid.

¹H NMR (DMSOd₆) δ: 1.39 (m, 5H); 1.81 (m, 2H); 3.17 (s, 3H); 3.19 (t, 2H); 3.45 (lm, 6H); 3.51 (t, 2H, J=8 Hz); 4.12 (dd, 1H); 4.24 (t, 2H, J=8 Hz); 4.41 (m, 1H); 4.68 (ls, 1H); 7.15 (d, 1H, J=8.4 Hz); 7.43 (m, 2H) MS m/z 425 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=3.93 min, chemical purity: 100%.

### EXAMPLE 16

### (S)-methyl 3-((4-(2-(2-oxooxazolidin-3-yl)ethyl)piperidin-1-yl)methyl)-2,3-dihydrobenzo[b][1,4]dioxine-6-carboxylate

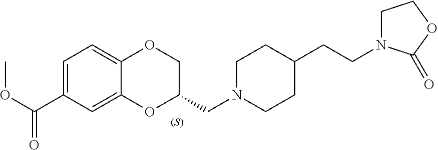

By proceeding as in Example 1 but replacing (R)-2-(bromomethyl)-2,3-dihydrobenzo[b][1,4]dioxine 2.1 by (R)-methyl 3-((((4-bromophenyl)sulfonyl)oxy)methyl)-2,3-dihydrobenzo[b][1,4]dioxine-6-carboxylate 2.24, the K₂CO₃/KI pair by diisopropylethylamine (1.4 eq) and acetonitrile by dimethylsulfoxide, 0.032 g (0.08 mmol) of compound 16 are obtained in the form of a white solid, after purification by flash chromatography on silica gel (gradient over 30 min 100% dichloromethane to dichloromethane/methanol=95/5%).

Yield: 12%.

¹H NMR (DMSOd₆) δ: 1.18 (m, 3H); 1.40 (m, 2H); 1.65 (m, 2H); 2.00 (m, 2H); 2.54 (m, 2H); 2.88 (m, 2H); 3.17 (t, 2H, J=7.2 Hz); 3.50 (t, 2H, J=8 Hz); 3.76 (s, 3H); 4.05 (m, 1H); 4.24 (t, 2H, J=8 Hz); 4.37 (m, 2H); 6.98 (d, 1H, J=8.4 Hz); 7.39 (d, 1H, J=2 Hz); 7.46 (dd, 1H, 8.4 and 2 Hz).

0.032 g (0.08 mmol) of compound 16 are dissolved in 5 ml of acetonitrile and then 0.0072 g (0.08 mmol) of oxalic acid and 1 ml of water are added. After lyophilization, 0.029 g (0.06 mmol) of compound 16 oxalate are recovered in the form of a white solid.

¹H NMR (DMSOd₆) δ: 1.42 (m, 5H); 1.85 (m, 2H); 2.81 (m, 2H); 3.20 (m, 4H); 3.40 (m, 2H); 3.52 (t, 2H J=8 Hz); 3.81 (s, 3H); 4.11 (dd, 1H, J=11.6 and 6.8 Hz); 4.23 (t, 2H, J=8 Hz); 4.40 (m, 1H); 4.73 (ls, 1H); 7.03 (m, 1H); 7.51 (m, 2H).

MS m/z 405 (M+1)

HPLC (X-BRIDGE C18, Phase A: 100% H₂O. 0.1% TFA, Phase B: 100% acetonitrile, gradient B: 0 to 100% in 6 min, 1.8 ml/min), retention time=4.36 min, chemical purity: 99.1%.

### EXAMPLE 17

### (S)-3-((4-(3-(2-oxooxazolidin-3-yl)propyl)piperidin-1-yl)methyl)-2,3-dihydrobenzo[b][1,4]dioxine-6-carbonitrile

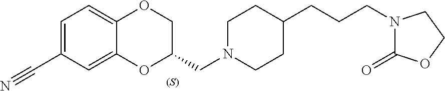

By proceeding as in Example 1 but replacing (R)-2-(bromomethyl)-2,3-dihydrobenzo[b][1,4]dioxine 2.1 by (R)-3-(bromomethyl)-2,3-dihydrobenzo[b][1,4]dioxine-6-carbonitrile 2.11, 3-(2-(piperidin-4-yl)ethyl)oxazolidin-2-one 3.1 by 3-(3-(piperidin-4-yl)propyl)oxazolidin-2-one 3.30, and the K₂CO₃/KI pair by diisopropylethylamine (2 eq), 1.12 g (2.90 mmol) of compound 17 are obtained in the form of a colorless oil, after purification by flash chromatography on silica gel (gradient: ethyl acetate=100% to ethyl acetate/(methanol/NH₄OH; 10/1)=98/2%).

Yield: 82%.

¹H NMR (CDCl₃) δ: 1.26 (m, 5H); 1.56 (m, 2H); 1.66 (m, 2H); 2.09 (m, 2H); 2.54 (dd, 1H, J=13.2 and 6.4 Hz); 2.65 (dd, 1H, J=13.2 and 5.6 Hz); 2.86 (m, 1H); 2.95 (m, 1H); 3.25 (t, 2H, J=7.2 Hz); 3.55 (m, 2H); 4.02 (dd, 1H, J=11.2 and 7.6 Hz); 4.29 (m, 4H); 6.91 (d, 1H, J=8.4 Hz); 7.14 (dd, 1H, J=8.4 and 2 Hz); 7.16 (d, 1H, J=2 Hz).

1.12 g (2.90 mmol) of compound 17 are dissolved in a minimal amount of methanol and 1.2 mL (6 mmol) of a 5 N solution of HCl in isopropanol are added. The medium is concentrated and triturated with ethyl ether. The precipitate, which tends to stick together, is filtered and left to dry in the vacuum chamber. 0.99 g (2.34 mmol) of compound 17 hydrochloride are obtained in the form of a white solid.

¹H NMR (DMSOd₆) δ: 1.20 (m, 2H); 1.52 (m, 5H); 1.84 (m, 2H); 3.03 (m, 2H); 3.13 (m, 2H); 3.40 (m, 5H); 3.67 (m, 1H); 4.22 (m, 3H); 4.46 (dd, 1H, J=12 and 2.4 Hz); 5.02 (m, 1H); 7.11 (d, 1H, J=8.4 Hz); 7.38 (dd, 1H, J=8.4 and 1.6 Hz); 7.49 (d, 1H, J=1.6 Hz).

MS m/z 386 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 15 to 40% in 5 min, 0.75 ml/min), retention time=1.88 min, chemical purity: 98.97%.

### EXAMPLE 18

### (S)-3-(2-(1-(((S)-2,3-dihydrobenzo[b][1,4]dioxin-2-yl)methyl)piperidin-4-yl)ethyl)-4-isopropyloxazolidin-2-one

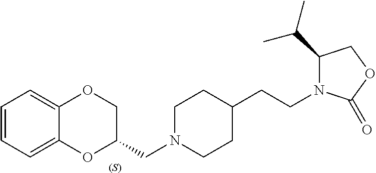

By proceeding as in Example 1 but replacing 3-(2-(piperidin-4-yl)ethyl)oxazolidin-2-one 3.1 by (S)-4-isopropyl-3-(2-(piperidin-4-yl)ethyl)oxazolidin-2-one 3.2, and acetonitrile by DMSO, 0.68 g (1.75 mmol) of compound 18 are obtained in the form of a pale yellow oil, after purification by flash chromatography on silica gel (gradient over 40 min 100% dichloromethane to dichloromethane/methanol=95/5%).

Yield: 70%.

0.68 g (1.75 mmol) of compound 18 are dissolved in a minimal amount of ethanol and 0.5 ml (2.5 mmol) of a 5 N solution of HCl in isopropanol are added. The medium is concentrated and triturated with ethyl ether. The precipitate is filtered and left to dry in the vacuum chamber. 0.53 g (1.25 mmol) of compound 18 hydrochloride are obtained in the form of a white solid.

F: 173° C.

¹H NMR (DMSOd₆) δ: 0.76 (d, 3H, J=6.8 Hz); 0.85 (d, 3H, J=6.8 Hz); 1.48 (m, 5H); 1.89 (m, 2H); 2.03 (m, 1H); 2.98 (m, 3H); 3.41 (m, 4H); 3.67 (m, 1H); 3.80 (m, 1H); 4.06 (m, 2H); 4.18 (m, 1H); 4.32 (m, 1H); 4.91 (m, 1H); 6.91 (m, 4H); 10.53 (ls, 1H).

MS m/z 389 (M+1).

HPLC (XBRIDGE C8, acetonitrile/water/KH₂PO₄ (30/70/6.8 g pH 4), 0.4 ml/min), retention time=6.62 min, chemical purity=97.8%

### EXAMPLE 19

### (S)-3-((4-(2-((S)-4-isopropyl-2-oxooxazolidin-3-yl)ethyl)piperidin-1-yl)methyl)-2,3-dihydrobenzo[b][1,4]dioxine-6-carbonitrile

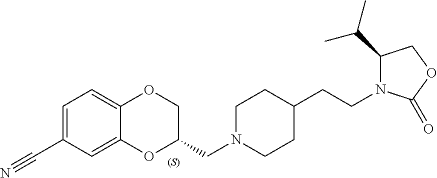

By proceeding as in Example 1 but replacing (R)-2-(bromomethyl)-2,3-dihydrobenzo[b][1,4]dioxine 2.1 by (R)-(7-cyano-2,3-dihydrobenzo[b][1,4]dioxin-2-yl)methyl 4-bromobenzenesulfonate 2.11, 3-(2-(piperidin-4-yl)ethyl)oxazolidin-2-one 3.1 by (S)-4-isopropyl-3-(2-(piperidin-4-yl)ethyl)oxazolidin-2-one 3.2, and the K₂CO₃/KI pair by diisopropylethylamine (1.4 eq), 0.17 g (0.41 mmol) of compound 19 are obtained in the form of a pale yellow solid, after purification by flash chromatography on silica gel (eluent: dichloromethane/ethyl acetate 70/30% then dichloromethane/methanol=95/5%).

Yield: 56%.

¹H NMR (CDCl₃) δ: 0.87 (d, 3H, J=6.8 Hz); 0.91 (d, 3H, J=7.2 Hz); 1.28 (m, 3H); 1.49 (m, 2H); 1.67 (m, 1H); 1.77 (m, 1H); 2.09 (m, 3H); 2.54 (m, 1H); 2.66 (m, 1H); 2.87 (m, 1H); 2.96 (m, 2H); 3.58 (m, 1H); 3.74 (m, 1H); 4.04 (m, 2H); 4.21 (m, 1H); 4.28 (m, 1H); 4.37 (m, 1H); 6.91 (d, 1H, J=8.4 Hz); 7.14 (m, 2H).

0.17 g (0.41 mmol) of compound 19 are dissolved in 5 ml of methanol and 0.37 g (0.41 mmol) of oxalic acid in solution in 5 ml of methanol are added. A solid precipitates; several drops of ethyl ether are added. The precipitate is filtered and then dried in the vacuum chamber. 0.14 g (0.28 mmol) of compound 19 oxalate are recovered in the form of a cream solid.

F: 195° C.

¹H NMR (DMSOd₆) δ: 0.76 (d, 3H, J=6.8 Hz); 0.84 (d, 3H, J=7.2 Hz); 1.40 (m, 5H); 1.80 (m, 2H); 2.02 (m, 1H); 2.60 (lm, 2H); 2.98 (m, 3H); 3.30 (m, 3H); 3.78 (m, 1H); 4.11 (m, 3H); 4.40 (m, 1H); 4.67 (m, 1H); 7.08 (d, 1H, J=8.4 Hz); 7.35 (dd, 1H, J=8.4 and 1.8 Hz); 7.44 (d, 1H, J=1.8 Hz).

MS m/z 414 (M+1).

HPLC (XBRIDGE C8, acetonitrile/water/KH₂PO₄ (30/70/6.8 g pH 4), 0.4 ml/min), retention time=6.61 min, chemical purity=99.05%

### EXAMPLE 20

### (S)-3-(2-(1-((2,3-dihydrobenzo[b][1,4]dioxin-2-yl)methyl)piperidin-4-yl)ethyl)-6-methoxybenzo[d]oxazol-2(3H)-one

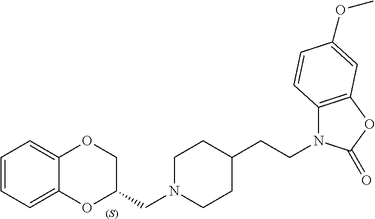

By proceeding as in Example 1 but replacing 3-(2-(piperidin-4-yl)ethyl)oxazolidin-2-one 3.1 by 6-methoxy-3-(2-(piperidin-4-yl)ethyl)benzo[d]oxazol-2(3H)-one 3.3, K₂CO₃ by Cs₂CO₃ and acetonitrile by DMF, 0.4 g (0.94 mmol) of compound 20 are obtained in the form of a cream solid, after purification by flash chromatography on silica gel (eluent n-heptane=100%, then dichloromethane/ethyl acetate=70/30% then dichloromethane/methanol=95/5%)

Yield: 47%.

¹H NMR (CDCl₃) δ: 1.32 (ls, 3H); 1.73 (m, 4H); 2.09 (m, 2H); 2.54 (m, 1H); 2.65 (m, 1H); 2.90 (m, 1H); 2.99 (m, 1H); 3.83 (m, 5H); 3.97 (dd, 1H J=11.6 and 7.6 Hz); 4.30 (m, 2H); 6.73 (dd, 1H, J=8.4 and 2.4 Hz); 6.85 (m, 6H).

0.4 g (0.94 mmol) of compound 20 are dissolved in 5 ml of methanol plus 5 ml of acetone and 0.085 g (0.94 mmol) of oxalic acid in solution in 5 ml of methanol are added. After stirring, a solid precipitates; several drops of ethyl ether are added. The precipitate is filtered and then dried in the vacuum chamber. 0.44 g (0.85 mmol) of compound 20 oxalate are recovered in the form of a white solid.

F: 220° C.

¹H NMR (DMSOd₆) δ: 1.41 (m, 3H); 1.64 (m, 2H); 1.86 (m, 2H); 2.67 (m, 2H); 3.08 (m, 2H); 3.28 (m, 1H); 3.36 (m, 1H); 3.75 (s, 3H); 3.83 (t, 2H, J=6.8 Hz); 3.99 (dd, 1H, J=11.4 and 6.4 Hz); 4.29 (m, 1H); 4.61 (m, 1H); 6.86 (m, 5H); 7.07 (dd, 1H, J=2 Hz); 7.22 (d, 1H, J=8.8 Hz).

MS m/z 425 (M+1).

HPLC (XBRIDGE C8, acetonitrile/water/KH₂PO₄ (35/65/6.8 g pH 4), 0.4 ml/min), retention time=6.63 min, chemical purity=100%

### EXAMPLE 21

### (S)-3-((4-(2-(6-methoxy-2-oxobenzo[d]oxazol-3(2H)-yl)ethyl)piperidin-1-yl)methyl)-2,3-dihydrobenzo[b][1,4]dioxine-6-carbonitrile

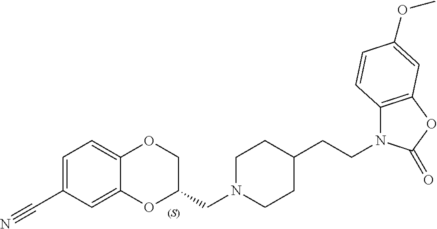

By proceeding as in Example 1 but replacing (R)-2-(bromomethyl)-2,3-dihydrobenzo[b][1,4]dioxine 2.1 by (S)-3-(bromomethyl)-2,3-dihydrobenzo[b][1,4]dioxine-6-carbonitrile 2.11, 3-(2-(piperidin-4-yl)ethyl)oxazolidin-2-one 3.1 by 6-methoxy-3-(2-(piperidin-4-yl)ethyl)benzo[d]oxazol-2(3H)-one 3.3, K₂CO₃ by Cs₂CO₃ and acetonitrile by DMF, 0.3 g (0.67 mmol) of compound 21 are obtained in the form of a cream solid, after purification by flash chromatography on silica gel (eluent; n-heptane/ethyl acetate=70/30% then dichloromethane/ethyl acetate=70/30%).

Yield: 31%.

¹H NMR (CDCl₃) δ: 1.32 (m, 3H); 1.70 (m, 2H); 1.77 (m, 2H); 2.10 (m, 2H); 2.54 (m, 1H); 2.65 (m, 1H); 2.87 (m, 1H); 2.95 (m, 1H); 3.83 (m, 5H); 4.02 (dd, 1H, J=11.6 and 7.6 Hz); 4.28 (m, 1H); 4.37 (dd, 1H, J=11.6 and 2 Hz); 6.73 (m, 1H); 6.84 (m, 2H); 6.91 (d, 1H, J=8.4 Hz); 7.14 (m, 2H).

0.3 g (0.67 mmol) of compound 21 are dissolved in 5 ml of methanol and 0.060 g (0.67 mmol) of oxalic acid in solution in 5 ml of methanol are added. After evaporation, the oil obtained is triturated in ethyl ether. The precipitate is filtered and then dried in the vacuum chamber. 0.3 g (0.56 mmol) of compound 21 oxalate are recovered in the form of a white solid.

F: 105° C.

¹H NMR (DMSOd₆) δ: 1.38 (m, 3H); 1.64 (m, 2H); 1.84 (m, 2H); 2.60 (m, 2H); 3.03 (m, 2H); 3.21 (m, 1H); 3.30 (m, 1H); 3.75 (s, 3H); 3.83 (t, 2H, J=7.2 Hz); 4.11 (dd, 1H, J=11.6 and 6.8 Hz); 4.40 (m, 1H); 4.67 (m, 1H); 6.81 (dd, 1H, J=8.4 and 2.4 Hz); 7.08 (m, 2H); 7.21 (d, 1H, J=8.4 Hz); 7.35 (dd, 1H, J=8.4 and 2 Hz); 7.44 (d, 1H, J=2 Hz).

MS m/z 450 (M+1).

HPLC (XBRIDGE C8, acetonitrile/water/KH₂PO₄ (40/60/6.8 g pH 4), 0.4 ml/min), retention time=4.35 min, chemical purity=97.64%

### EXAMPLE 22

### (S)-4-benzyl-3-(2-(1-(((S)-2,3-dihydrobenzo[b][1,4]dioxin-2-yl)methyl)piperidin-4-yl)ethyl)oxazolidin-2-one

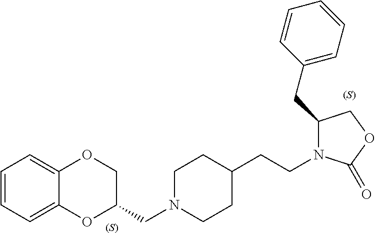

By proceeding as in Example 1 but replacing 3-(2-(piperidin-4-yl)ethyl)oxazolidin-2-one 3.1 by (S)-4-benzyl-3-(2-(piperidin-4-yl)ethyl)oxazolidin-2-one, 0.23 g (0.52 mmol) of compound 3.4 are obtained in the form of a colorless oil, after purification by flash chromatography on silica gel (eluent: dichloromethane/methanol=95/5%).

Yield: 34%.

The product is directly salified.

0.23 g (0.52 mmol) of compound 22 are dissolved in 5 ml of methanol and 0.043 g (0.47 mmol) of oxalic acid in solution in 5 ml of methanol are added. After stirring, a white precipitate forms; a little ether is added and the salt is filtered and then dried in the vacuum chamber. 0.2 g (0.38 mmol) of compound 22 oxalate are recovered in the form of a white solid.

F: 209° C.

¹H NMR (DMSOd₆) δ: 1.39 (m, 5H); 1.79 (m, 2H); 2.64 (m, 3H); 3.21 (m, 7H); 3.98 (m, 2H); 4.14 (m, 2H); 4.29 (d, 1H); 4.60 (m, 1H); 6.88 (m, 4H); 7.28 (m, 5H).

MS m/z 437 (M+1).

HPLC (XBRIDGE C8, acetonitrile/water/KH₂PO₄ (35/65/6.8 g pH 4), 1 ml/min), retention time=13.29 min, chemical purity=99.18%

HPLC (CHIRALPACK AD-H, heptane/ethanol/diethylamine (50/50/0.1), 1 ml/min); compound 22, retention time=9.34 min, compound 23, retention time=8.59 min, ratio of AUCs (22)/(23)=99.5/0.5.

### EXAMPLE 23

### (S)-4-benzyl-3-(2-(1-(((R)-2,3-dihydrobenzo[b][1,4]dioxin-2-yl)methyl)piperidin-4-yl)ethyl)oxazolidin-2-one

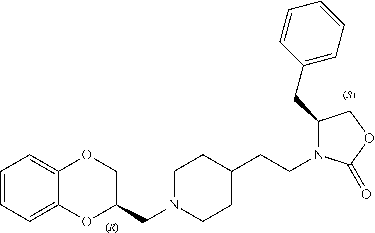

By proceeding as in Example 1 but replacing (R)-2-(bromomethyl)-2,3-dihydrobenzo[b][1,4]dioxine 2.1 by (S)-2-(bromomethyl)-2,3-dihydrobenzo[b][1,4]dioxine 2.2 and 3-(2-(piperidin-4-yl)ethyl)oxazolidin-2-one 3.1 by (S)-4-benzyl-3-(2-(piperidin-4-yl)ethyl)oxazolidin-2-one 3.4, 0.35 g (0.80 mmol) of compound 23 are obtained in the form of a colorless oil, after purification by flash chromatography on silica gel (eluent; dichloromethane/methanol=97/3%).

Yield: 34%.

The product is directly salified.

0.35 g (0.80 mmol) of compound 23 are dissolved in 5 ml of methanol and 0.07 g (0.77 mmol) of oxalic acid in solution in 5 ml of methanol are added. After evaporation, it is triturated with ethyl ether, a white precipitate forms, the salt is filtered and dried in the vacuum chamber. 0.26 g (0.49 mmol) of compound 23 oxalate are recovered in the form of a white solid.

F: 190° C.

¹H NMR (DMSOd₆) δ: 1.40 (m, 5H); 1.80 (m, 2H); 2.71 (m, 3H); 3.06 (m, 4H); 3.26 (m, 1H); 3.37 (m, 2H); 3.98 (m, 2H); 4.14 (m, 2H); 4.29 (d, 1H); 4.61 (m, 1H); 6.87 (m, 4H); 7.29 (m, 5H).

MS m/z 437 (M+1).

HPLC (XBRIDGE C8, acetonitrile/water/KH₂PO₄ (35/65/6.8 g pH 4), 1 ml/min), retention time=14.05 min, chemical purity=99.65%

HPLC (CHIRALPACK AD-H, heptane/ethanol/diethylamine (60/40/0.1), 1 ml/min); compound 23, retention time=10.17 min, compound 22, retention time=11.19 min, ratio of AUCs (22)/(23)=99.6/0.4.

### EXAMPLE 24

### (S)-3-((4-(2-((S)-4-benzyl-2-oxooxazolidin-3-yl)ethyl)piperidin-1-yl)methyl)-2,3-dihydrobenzo[b][1,4]dioxine-6-carbonitrile

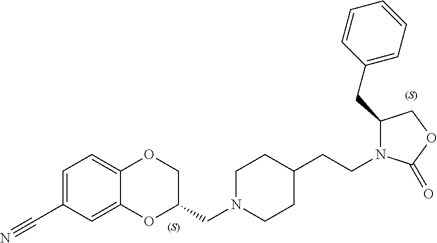

By proceeding as in Example 1 but replacing (R)-2-(bromomethyl)-2,3-dihydrobenzo[b][1,4]dioxine 2.1 by (R)-3-(bromomethyl)-2,3-dihydrobenzo[b][1,4]dioxine-6-carbonitrile 2.11, 3-(2-(piperidin-4-yl)ethyl)oxazolidin-2-one 3.1 by (S)-4-benzyl-3-(2-(piperidin-4-yl)ethyl)oxazolidin-2-one 3.4, K₂CO₃ by Cs₂CO₃ and acetonitrile by DMF, 0.6 g (1.3 mmol) of compound 24 are obtained in the form of a colorless oil, after purification by flash chromatography on silica gel (eluent: dichloromethane/ethyl acetate=70/30% then dichloromethane/methanol=95/5%).

Yield: 40%.

¹H NMR (CDCl₃) δ: 1.26 (m, 3H); 1.50 (m, 2H); 1.70 (m, 2H); 2.09 (m, 2H); 2.54 (m, 1H); 2.67 (m, 2H); 2.87 (m, 1H); 2.94 (m, 1H); 3.09 (m, 2H); 3.56 (m, 1H); 4.02 (m, 3H); 4.17 (m, 1H); 4.28 (m, 1H); 4.37 (dd, 1H, J=11.4 and 2.4 Hz); 6.91 (d, 1H, J=9.2 Hz); 7.15 (m, 4H); 7.31 (m, 3H).

0.6 g (1.3 mmol) of compound 24 are dissolved in a 5 ml of methanol and 0.5 ml (2.5 mmol) of a 5 N solution of HCl in isopropanol are added. The medium is concentrated and triturated with ethyl ether; a minimal amount of ethanol is added. The precipitate is filtered and left to dry in the vacuum chamber. 0.48 g (0.96 mmol) of compound 24 hydrochloride are obtained in the form of a white solid.

F: 68° C.

¹H NMR (DMSOd₆) δ: 1.47 (m, 5H); 1.90 (m, 2H); 2.50 (m, 2H); 2.74 (m, 1H); 3.08 (m, 4H); 3.45 (m, 2H); 3.66 (m, 1H); 3.97 (m, 1H); 4.14 (m, 3H); 4.44 (m, 1H); 4.97 (m, 1H); 7.12 (d, 1H, J=8.4 Hz); 7.29 (m, 5H); 7.39 (dd, 1H, J=8.4 and 2 Hz); 7.50 (d, 1H, J=2 Hz); 10.45 (ls, 1H).

MS m/z 462 (M+1).

HPLC (XBRIDGE C8, acetonitrile/water/KH₂PO₄ (35/65/6.8 g pH 4), 0.4 ml/min), retention time=6.73 min, chemical purity=97.43%

### EXAMPLE 25

### (S)-4-benzyl-3-(2-(1-(((S)-8-bromo-2,3-dihydrobenzo[b][1,4]dioxin-2-yl)methyl)piperidin-4-yl)ethyl)oxazolidin-2-one

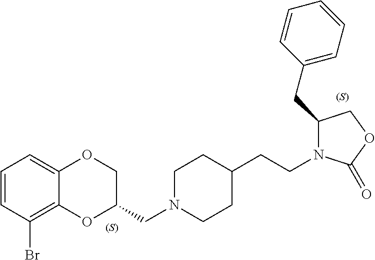

By proceeding as in Example 1 but replacing (R)-2-(bromomethyl)-2,3-dihydrobenzo[b][1,4]dioxine 2.1 by (R)-(8-bromo-2,3-dihydrobenzo[b][1,4]dioxin-2-yl)methyl 4-bromobenzenesulfonate 2.4, 3-(2-(piperidin-4-yl)ethyl)oxazolidin-2-one 3.1 by (S)-4-benzyl-3-(2-(piperidin-4-yl)ethyl)oxazolidin-2-one 3.4, K₂CO₃ by Cs₂CO₃ and acetonitrile by DMF, 0.74 g (1.43 mmol) of compound 25 are obtained in the form of a colorless oil, after purification by flash chromatography on silica gel (eluent: n-heptane/ethyl acetate=70/30% then dichloromethane/methanol=95/5%).

Yield: 54%.

¹H NMR (CDCl₃) δ: 1.27 (m, 3H); 1.50 (m, 2H); 1.68 (m, 2H); 2.1 (m, 1H); 2.23 (m, 1H); 2.68 (m, 3H); 2.87 (m, 1H); 3.09 (m, 3H); 3.57 (m, 1H); 4.01 (m, 3H); 4.17 (m, 1H); 4.30 (dd, 1H, J=11.4 and 2.8 Hz); 4.38 (m, 1H); 6.70 (m, 1H); 6.74 (m, 1H); 6.82 (dd, 1H, J=8 and 1.6 Hz); 7.16 (m, 2H); 7.32 (m, 3H).

0.74 g (1.43 mmol) of compound 25 are dissolved in 5 ml of methanol and 0.12 g (1.33 mmol) of oxalic acid in solution in 5 ml of methanol are added. A little ethyl ether is added; a white precipitate forms, the salt is filtered and dried in the vacuum chamber. 0.70 g (1.16 mmol) of compound 25 oxalate are recovered in the form of a white solid.

F: 177° C.

¹H NMR (DMSOd₆) δ: 1.40 (m, 5H); 1.81 (m, 2H); 2.74 (m, 3H); 3.14 (m, 5H); 3.42 (m, 2H); 3.96 (m, 1H); 4.12 (m, 3H); 4.33 (m, 1H); 4.72 (m, 1H); 6.82 (m, 1H); 6.93 (d, 1H, J=7.6 Hz); 7.16 (d, 1H, J=8 Hz); 7.28 (m, 5H).

MS m/z 515-517 (M+1).

HPLC (XBRIDGE C8, acetonitrile/water/KH₂PO₄ (40/60/6.8 g pH 4), 0.4 ml/min), retention time=6.25 min, chemical purity=94.66%

### EXAMPLE 26

### (S)-4-benzyl-3-(2-(1-(((S)-7-bromo-2,3-dihydrobenzo[b][1,4]dioxin-2-yl)methyl)piperidin-4-yl)ethyl)oxazolidin-2-one

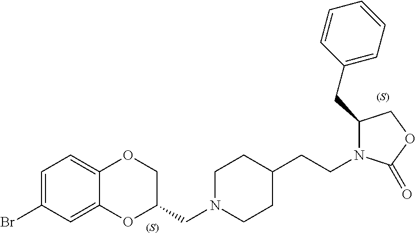

By proceeding as in Example 1 but replacing (R)-2-(bromomethyl)-2,3-dihydrobenzo[b][1,4]dioxine 2.1 by (R)-7-bromo-2-(bromomethyl)-2,3-dihydrobenzo[b][1,4]dioxine 2.5, 3-(2-(piperidin-4-yl)ethyl)oxazolidin-2-one 3.1 by (S)-4-benzyl-3-(2-(piperidin-4-yl)ethyl)oxazolidin-2-one 3.4, K₂CO₃ by Cs₂CO₃ and acetonitrile by DMF, 1.1 g (2.13 mmol) of compound 26 are obtained in the form of a colorless oil, after purification by flash chromatography on silica gel (eluent: n-heptane=100% then dichloromethane/methanol=95/5%).

Yield: 48%.

¹H NMR (CDCl₃) δ: 1.26 (m, 3H); 1.50 (m, 2H); 1.69 (m, 2H); 2.09 (m, 2H); 2.53 (m, 1H); 2.65 (m, 2H); 2.86 (m, 1H); 2.95 (m, 1H); 3.10 (m, 2H); 3.55 (m, 1H); 3.97 (m, 3H); 4.17 (m, 1H); 4.28 (m, 2H); 6.73 (d, 1H, J=8.8 Hz); 6.92 (dd, 1H, J=8.8 and 2.4 Hz); 7.03 (d, 1H, J=2.4 Hz); 7.16 (m, 2H); 7.32 (m, 3H).

1.1 g (2.13 mmol) of compound 26 are dissolved in 10 ml of methanol and 0.19 g (2.11 mmol) of oxalic acid in solution in 5 ml of methanol are added. A little ethyl ether is added; a white precipitate forms, the salt is filtered and dried in the vacuum chamber. 0.90 g (1.48 mmol) of compound 26 oxalate are recovered in the form of a white solid.

F: 147° C.

¹H NMR (DMSOd₆) δ: 1.40 (m, 5H); 1.79 (m, 2H); 2.72 (m, 3H); 3.07 (m, 4H); 3.25 (m, 1H); 3.38 (m, 2H); 3.97 (m, 1H); 4.04 (m, 1H); 4.14 (m, 2H); 4.30 (m, 1H); 4.70 (m, 1H); 6.88 (d, 1H, J=8.8 Hz); 7.03 (dd, 1H, J=8.8 and 2.4 Hz); 7.14 (d, 1H, J=2.4 Hz); 7.28 (m, 5H).

MS m/z 515-517 (M+1).

HPLC (XBRIDGE C8, acetonitrile/water/KH₂PO₄ (40/60/6.8 g pH 4), 0.4 ml/min), retention time=8.68 min, chemical purity=100%

### EXAMPLE 27

### (S)-4-benzyl-3-(2-(1-(((S)-6-bromo-2,3-dihydrobenzo[b][1,4]dioxin-2-yl)methyl)piperidin-4-yl)ethyl)oxazolidin-2-one

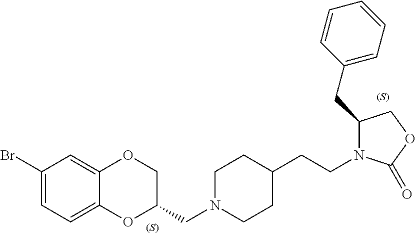

By proceeding as in Example 1 but replacing (R)-2-(bromomethyl)-2,3-dihydrobenzo[b][1,4]dioxine 2.1 by (R)-6-bromo-2-(bromomethyl)-2,3-dihydrobenzo[b][1,4]dioxine 2.6, 3-(2-(piperidin-4-yl)ethyl)oxazolidin-2-one 3.1 by (S)-4-benzyl-3-(2-(piperidin-4-yl)ethyl)oxazolidin-2-one 3.4, K₂CO₃ by Cs₂CO₃ and acetonitrile by DMF, 0.65 g (1.26 mmol) of compound 27 are obtained in the form of a colorless oil, after purification by flash chromatography on silica gel (eluent: n-heptane/ethyl acetate=70/30% then dichloromethane/methanol=95/5%).

Yield: 47%.

¹H NMR (CDCl₃) δ: 1.26 (m, 3H); 1.50 (m, 2H); 1.67 (m, 2H); 2.08 (m, 2H); 2.52 (m, 1H); 2.65 (m, 2H); 2.87 (m, 1H); 2.95 (m, 1H); 3.10 (m, 2H); 3.57 (m, 1H); 3.98 (m, 3H); 4.17 (m, 1H); 4.28 (m, 2H); 6.74 (d, 1H, J=8.4 Hz); 6.93 (dd, 1H, J=8.8 Hz and 2.4 Hz); 7.01 (d, 1H, J=2.4 Hz); 7.16 (m, 2H); 7.31 (m, 3H).

0.65 g (1.26 mmol) of compound 27 are dissolved in 5 ml of methanol and 0.11 g (1.22 mmol) of oxalic acid in solution in 5 ml of methanol are added. A little ethyl ether is added; a white precipitate forms, the salt is filtered and dried in the vacuum chamber. 0.90 g (1.48 mmol) of compound 27 oxalate are recovered in the form of a white solid.

F: 191° C.

¹H NMR (DMSOd₆) δ: 1.39 (m, 5H); 1.78 (m, 2H); 2.71 (m, 3H); 3.06 (m, 4H); 3.25 (m, 1H); 3.38 (m, 2H); 3.96 (dd, 1H, J=8 and 5.2 Hz); 4.1 (m, 3H); 4.31 (m, 1H); 4.64 (m, 1H); 6.89 (d, 1H, J=8.8); 7.03 (dd, 1H, J=8.4 and 2 Hz); 7.12 (d, 1H, J=2 Hz); 7.28 (m, 5H).

MS m/z 515-517 (M+1).

HPLC (XBRIDGE C8, acetonitrile/water/KH₂PO₄ (40/60/6.8 g pH 4), 0.4 ml/min), retention time=7.05 min, chemical purity=99.65%

### EXAMPLE 28

### (S)-4-benzyl-3-(2-(1-(((S)-5-bromo-2,3-dihydrobenzo[b][1,4]dioxin-2-yl)methyl)piperidin-4-yl)ethyl)oxazolidin-2-one

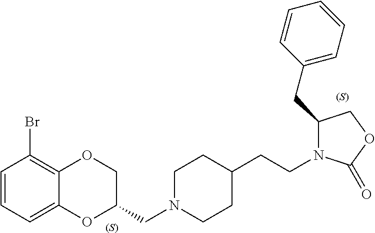

By proceeding as in Example 1 but replacing (R)-2-(bromomethyl)-2,3-dihydrobenzo[b][1,4]dioxine 2.1 by (R)-(5-bromo-2,3-dihydrobenzo[b][1,4]dioxin-2-yl)methyl 4-bromobenzenesulfonate 2.15, 3-(2-(piperidin-4-yl)ethyl)oxazolidin-2-one 3.1 by (S)-4-benzyl-3-(2-(piperidin-4-yl)ethyl)oxazolidin-2-one 3.4, the K₂CO₃/KI pair by diisopropylethylamine (1.4 eq) and acetonitrile by DMSO, 0.20 g (0.39 mmol) of compound 28 are obtained in the form of a colorless oil, after purification by flash chromatography on silica gel (eluent: n-heptane/ethyl acetate=70/30% then dichloromethane/methanol=95/5%).

Yield: 16%.

¹H NMR (CDCl₃) δ: 1.26 (m, 3H); 1.49 (m, 2H); 1.69 (m, 2H); 2.10 (m, 2H); 2.55 (m, 1H); 2.66 (m, 2H); 2.88 (m, 1H); 2.96 (m, 1H); 3.10 (m, 2H); 3.57 (m, 1H); 4.02 (m, 3H); 4.17 (m, 1H); 4.29 (m, 1H); 4.43 (dd, 1H, J=11.2 and 2.4 Hz); 6.17 (m, 1H); 6.84 (dd, 1H, J=8.2 and 1.6 Hz); 7.08 (dd, 1H, J=8 and 1.6 Hz); 7.16 (m, 2H); 7.32 (m, 3H).

0.20 g (0.39 mmol) of compound 28 are dissolved in 5 ml of methanol and 0.035 g (0.39 mmol) of oxalic acid in solution in 5 ml of methanol are added. A little ethyl ether is added; a white precipitate forms, the salt is filtered and dried in the vacuum chamber. 0.16 g (0.26 mmol) of compound 28 oxalate are obtained in the form of a white solid.

F: 174° C.

¹H NMR (DMSOd₆) δ: 1.39 (m, 5H); 1.78 (m, 2H); 2.62 (m, 2H); 2.73 (m, 1H); 3.06 (m, 4H); 3.32 (m, 3H); 3.96 (dd, 1H, J=8 and 4.8 Hz); 4.13 (m, 3H); 4.42 (dd, 1H, J=11.4 and 2 Hz); 4.66 (m, 1H); 6.82 (m, 1H); 6.95 (dd, 1H, J=8 and 1.6 Hz); 7.16 (dd, 1H, J=8 and 1.2 Hz); 7.28 (m, 5H).

MS m/z 515-517 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 45 to 70% in 5 min, 0.75 ml/min), retention time=1.13 min, chemical purity: 99.79%.

### EXAMPLE 29

### (S)-4-benzyl-3-(2-(1-(((S)-7-fluoro-2,3-dihydrobenzo[b][1,4]dioxin-2-yl)methyl)piperidin-4-yl)ethyl)oxazolidin-2-one

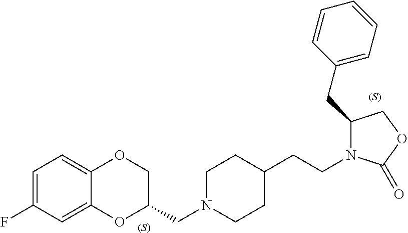

By proceeding as in Example 1 but replacing (R)-2-(bromomethyl)-2,3-dihydrobenzo[b][1,4]dioxine 2.1 by (R)-(7-fluoro-2,3-dihydrobenzo[b][1,4]dioxin-2-yl)methyl 4-bromobenzenesulfonate 2.17, 3-(2-(piperidin-4-yl)ethyl)oxazolidin-2-one 3.1 by (S)-4-benzyl-3-(2-(piperidin-4-yl)ethyl)oxazolidin-2-one 3.4, and the K₂CO₃/KI pair by diisopropylethylamine (1.4 eq), 1 g (2.2 mmol) of compound 29 is obtained in the form of a colorless oil, after purification by flash chromatography on silica gel (eluent: dichloromethane/ethyl acetate 70/30% then dichloromethane/methanol=95/5%).

Yield: 45%.

¹H NMR (CDCl₃) δ: 1.26 (m, 3H); 1.50 (m, 2H); 1.69 (m, 2H); 2.08 (m, 2H); 2.53 (m, 1H); 2.66 (m, 2H); 2.88 (m, 1H); 2.96 (m, 1H); 3.10 (m, 2H); 3.57 (m, 1H); 3.93 (dd, 1H, J=11.8 and 8 Hz); 4.00 (m, 2H); 4.15 (m, 1H); 4.27 (m, 2H); 6.53 (m, 1H); 6.61 (dd, 1H, J=9.2 and 2.8 Hz); 6.78 (dd, 1H, J=8.8 and 1.6 Hz); 7.16 (m, 2H); 7.31 (m, 3H).

1 g (2.2 mmol) of compound 29 are dissolved in 10 ml of methanol and 0.19 g (2.11 mmol) of oxalic acid in solution in 10 ml of methanol are added. A little ethyl ether is added; a white precipitate forms, the salt is filtered and dried in the vacuum chamber. 1.1 g (2.02 mmol) of compound 29 oxalate are recovered in the form of a white solid.

F: 160° C.

¹H NMR (DMSOd₆) δ: 1.39 (m, 5H); 1.79 (m, 2H); 2.67 (m, 3H); 3.06 (m, 4H); 3.25 (m, 1H); 3.38 (m, 3H); 3.98 (m, 2H); 4.11 (m, 1H); 4.29 (m, 1H); 4.65 (m, 1H); 6.71 (m, 1H); 6.83 (dd, 1H, J=9.6 and 3.2 Hz); 6.92 (dd, 1H, J=9.2 and 1.6 Hz); 7.28 (m, 5H).

MS m/z 455 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=1.82 min, chemical purity: 99.46%.

### EXAMPLE 30

### (S)-4-benzyl-3-(2-(1-(((S)-7-(trifluoromethyl)-2,3-dihydrobenzo[b][1,4]dioxin-2-yl)methyl)piperidin-4-yl)ethyl)oxazolidin-2-one

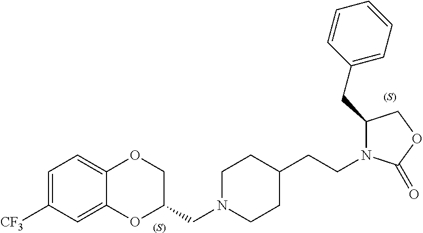

By proceeding as in Example 1 but replacing (R)-2-(bromomethyl)-2,3-dihydrobenzo[b][1,4]dioxine 2.1 by (R)-(7-(trifluoromethyl)-2,3-dihydrobenzo[b][1,4]dioxin-2-yl)methyl 4-bromobenzenesulfonate 2.10, 3-(2-(piperidin-4-yl)ethyl)oxazolidin-2-one 3.1 by (S)-4-benzyl-3-(2-(piperidin-4-yl)ethyl)oxazolidin-2-one 3.4 and acetonitrile by DMF, 0.24 g (0.48 mmol) of compound 30 are obtained in the form of a pale yellow oil, after purification by flash chromatography on silica gel (eluent:gradient dichloromethane=100% to dichloromethane/methanol=95/5% over 30 min).

Yield: 21%.

¹H NMR (CDCl₃) δ: 1.27 (m, 3H); 1.50 (m, 2H); 1.70 (m, 2H); 2.09 (m, 2H); 2.55 (m, 1H); 2.67 (m, 2H); 2.88 (m, 1H); 2.96 (m, 1H); 3.10 (m, 2H); 3.56 (m, 1H); 4.00 (m, 3H); 4.17 (m, 1H); 4.33 (m, 2H); 6.93 (d, 1H, J=8.4 Hz); 7.09 (m, 1H); 7.16 (m, 3H); 7.31 (m, 31H).

0.24 g (0.48 mmol) of compound 30 are dissolved in 5 ml of methanol and 0.039 g (0.43 mmol) of oxalic acid in solution in 5 ml of methanol are added. The medium is concentrated then it is triturated with ethyl ether; a white precipitate forms and the salt is filtered and dried in the vacuum chamber. 0.22 g (0.37 mmol) of compound 30 oxalate are recovered in the form of a white solid.

F: 139° C.

¹H NMR (DMSOd₆) δ: 1.30 (m, 3H); 1.44 (m, 2H); 1.78 (m, 2H); 2.50 (m, 1H); 2.73 (m, 1H); 3.06 (m, 4H); 3.35 (m, 4H); 3.96 (m, 1H); 4.13 (m, 3H); 4.39 (m, 1H); 4.66 (m, 1H); 7.10 (d, 1H, J=8.4 Hz); 7.28 (m, 7H).

MS m/z 505 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 55 to 100% in 6 min, 0.75 ml/min), retention time=0.91 min, chemical purity: 99.58%.

### EXAMPLE 31

### (S)-4-benzyl-3-(2-(1-(((S)-7-methyl-2,3-dihydrobenzo[b][1,4]dioxin-2-yl)methyl)piperidin-4-yl)ethyl)oxazolidin-2-one

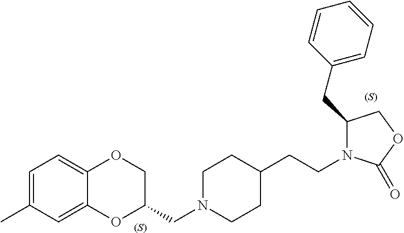

By proceeding as in Example 1 but replacing (R)-2-(bromomethyl)-2,3-dihydrobenzo[b][1,4]dioxine 2.1 by (R)-2-(bromomethyl)-7-methyl-2,3-dihydrobenzo[b][1,4]dioxine 2.3, 3-(2-(piperidin-4-yl)ethyl)oxazolidin-2-one 3.1 by (S)-4-benzyl-3-(2-(piperidin-4-yl)ethyl)oxazolidin-2-one 3.4 and acetonitrile by DMF, compound 31 is obtained in the form of a yellow oil. This base is solubilized in a minimal amount of ethanol and then 5 N HCl in isopropanol are added, this salified product is concentrated and purified on C18 reversed phase gel (eluent: 5 mM HCl/acetonitrile 90/10% over 30 min then gradient to 5 mM HCl/acetonitrile 50/50% over 20 min). The recovered fractions are lyophilized to obtain compound 31 hydrochloride are obtained in the form of a white solid.

Yield: 39%.

F: 65° C.

¹H NMR (DMSOd₆) δ: 1.45 (m, 3H); 1.62 (m, 2H); 1.91 (m, 2H); 2.50 (m, 1H); 2.74 (m, 1H); 3.07 (m, 4H); 3.4 (m, 2H); 3.52 (m, 1H); 3.62 (m, 1H); 4.00 (m, 2H); 4.12 (m, 2H); 4.26 (m, 1H); 4.79 (m, 1H); 6.69 (d, 1H, J=8 Hz); 6.78 (m, 2H); 7.29 (m, 5H); 9.79 (ls, 1H).

MS m/z 451 (M+1).

HPLC (XBRIDGE C8, acetonitrile/water/KH₂PO₄ (40/60/6.8 g pH 4), 0.4 ml/min), retention time=5.59 min, chemical purity=99.92%

### EXAMPLE 32

### (S)-4-benzyl-3-(2-(1-(((S)-7-(trifluoromethoxy)-2,3-dihydrobenzo[b][1,4]dioxin-2-yl)methyl)piperidin-4-yl)ethyl)oxazolidin-2-one

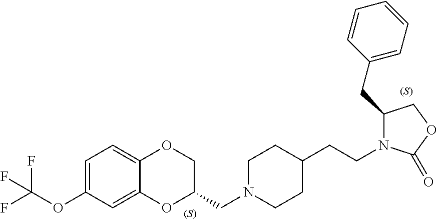

By proceeding as in Example 1 but replacing (R)-2-(bromomethyl)-2,3-dihydrobenzo[b][1,4]dioxine 2.1 by (R)-2-(bromomethyl)-7-(trifluoromethoxy)-2,3-dihydrobenzo[b][1,4]dioxine 2.8, 3-(2-(piperidin-4-yl)ethyl)oxazolidin-2-one 3.1 by (S)-4-benzyl-3-(2-(piperidin-4-yl)ethyl)oxazolidin-2-one 3.4 and acetonitrile by DMF, 0.58 g (1.11 mmol) of compound 32 are obtained in the form of a colorless oil, after purification on silica gel (gradient; =100% dichloromethane to dichloromethane/methanol=90/10% over 30 min).

Yield: 53%.

¹H NMR (CDCl₃) δ: 1.27 (m, 3H); 1.49 (m, 2H); 1.72 (m, 2H); 2.09 (m, 2H); 2.53 (dd, 1H, J=13.4 and 6 Hz); 2.66 (m, 2H); 2.88 (m, 1H); 2.96 (m, 1H); 3.10 (m, 2H); 3.57 (m, 1H); 3.98 (m, 3H); 4.17 (m, 1H); 4.30 (m, 2H); 6.70 (m, 1H); 6.78 (m, 1H); 6.84 (m, 1H); 7.16 (m, 2H); 7.32 (m, 3H).

0.58 g (1.11 mmol) of compound 32 are dissolved in 10 ml of methanol and 0.095 g (1.06 mmol) of oxalic acid in solution in 5 ml of methanol are added. A little ethyl ether is added; a white precipitate forms and the salt is filtered and dried in the vacuum chamber. 0.57 g (0.93 mmol) of compound 32 oxalate are recovered in the form of a white solid.

F: 194° C.

¹H NMR (DMSOd₆) δ: 1.34 (m, 3H); 1.44 (m, 2H); 1.78 (m, 2H); 2.60 (m, 2H); 2.73 (m, 1H); 3.06 (m, 4H); 3.23 (m, 1H); 3.38 (m, 2H); 3.96 (dd, 1H, J=7.8 and 5.2 Hz); 4.03 (dd, 1H, J=11.6 and 6.8 Hz); 4.14 (m, 2H); 4.33 (dd, 1H, J=11.6 and 2.4 Hz); 4.66 (m, 1H); 6.88 (m, 1H); 6.70 (m, 2H); 7.28 (m, 5H).

MS m/z 521 (M+1).

HPLC (XBRIDGE C8, acetonitrile/water/KH₂PO₄ (40/60/6.8 g pH 4), 0.4 ml/min), retention time=9.83 min, chemical purity=99.77%

### EXAMPLE 33

### (S)-4-benzyl-3-(2-(1-(((S)-6-bromo-7-methoxy-2,3-dihydrobenzo[b][1,4]dioxin-2-yl)methyl)piperidin-4-yl)ethyl)oxazolidin-2-one

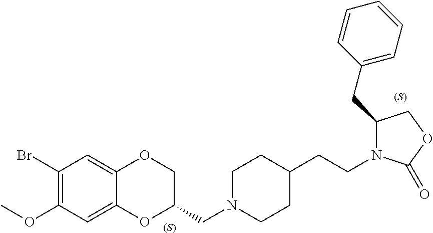

By proceeding as in Example 1 but replacing (R)-2-(bromomethyl)-2,3-dihydrobenzo[b][1,4]dioxine 2.1 by (R)-6-bromo-2-(bromomethyl)-7-methoxy-2,3-dihydrobenzo[b][1,4]dioxine 2.9, 3-(2-(piperidin-4-yl)ethyl)oxazolidin-2-one 3.1 by (S)-4-benzyl-3-(2-(piperidin-4-yl)ethyl)oxazolidin-2-one 3.4 and acetonitrile by DMF, 0.62 g (1.33 mmol) of compound 33 are obtained in the form of a colorless oil, after purification on silica gel (gradient; =100% dichloromethane to dichloromethane/methanol=90/10%).

Yield: 44%.

¹H NMR (CDCl₃) δ: 1.29 (m, 3H); 1.50 (m, 2H); 1.70 (m, 2H); 2.07 (m, 2H); 2.51 (m, 1H); 2.65 (m, 2H); 2.88 (m, 1H); 2.96 (m, 1H); 3.11 (m, 2H); 3.57 (m, 1H); 3.80 (s, 3H); 3.90 (dd, 1H, J=11.2 and 7.2 Hz); 4.01 (m, 2H); 4.17 (m, 1H); 4.25 (m, 2H); 6.50 (s, 1H); 7.06 (s, 1H); 7.16 (m, 2H); 7.40 (m, 3H).

0.62 g (1.33 mmol) of compound 33 are dissolved in 5 ml of methanol and 0.114 g (1.26 mmol) of oxalic acid in solution in 5 ml of methanol are added. A little ethyl ether is added; a white precipitate forms and the salt is filtered and dried in the vacuum chamber. 0.63 g (1.13 mmol) of compound 33 oxalate are recovered in the form of a white solid.

F: 204° C.

¹H NMR (DMSOd₆) δ: 1.31 (m, 3H); 1.43 (m, 2H); 1.77 (m, 2H); 2.50 (m, 1H); 2.73 (m, 1H); 3.07 (m, 4H); 3.42 (m, 4H); 3.75 (s, 3H); 3.96 (m, 1H); 4.14 (m, 3H); 4.25 (m, 1H); 4.60 (m, 1H); 6.71 (s, 1H); 7.14 (s, 1H); 7.29 (m, 5H).

MS m/z 545-547 (m+1).

HPLC (XBRIDGE C8, acetonitrile/water/KH₂PO₄ (40/60/6.8 g pH 4), 0.4 ml/min), retention time=6.47 min, chemical purity=99.72%

### EXAMPLE 34

### (S)-4-benzyl-3-(2-(1-(((S)-7-methoxy-2,3-dihydrobenzo[b][1,4]dioxin-2-yl)methyl)piperidin-4-yl)ethyl)oxazolidin-2-one

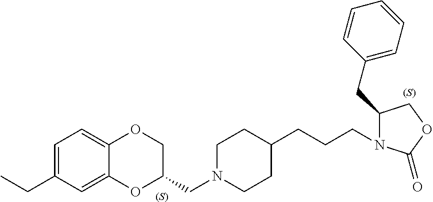

0.34 g (0.62 mmol) of the preceding compound (S)-4-benzyl-3-(2-(1-(((S)-6-bromo-7-methoxy-2,3-dihydrobenzo[b][1,4]dioxin-2-yl)methyl)piperidin-4-yl)ethyl)oxazolidin-2-one 33 are solubilized in 10 ml of methanol. A catalytic quantity of 10% Pd on carbon is added, and hydrogenation is performed at RT overnight. The medium is then filtered on Celite and concentrated. 0.15 g (0.32 mmol) of compound 34 are obtained in the form of a colorless oil, after purification on silica gel (gradient: dichloromethane=100% to dichloromethane/ethyl acetate=80/20% over 30 min).

Yield: 52%.

¹H NMR (CDCl₃) δ: 1.30 (m, 3H); 1.50 (m, 2H); 1.69 (m, 2H); 2.09 (m, 2H); 2.53 (dd, 1H, J=13.4 and 6 Hz); 2.66 (m, 2H); 2.89 (m, 1H); 2.97 (m, 1H); 3.10 (m, 2H); 3.58 (m, 1H); 3.73 (s, 3H); 3.92 (dd, 1H, J=11.2 and 7.2 Hz); 4.01 (m, 2H); 4.17 (m, 1H); 4.26 (m, 2H); 6.41 (dd, 1H, J=8.8 and 2.8 Hz); 6.47 (d, 1H, J=2.8 Hz); 6.77 (d, 1H, J=9.2 Hz); 7.16 (d, 1H, J=6.8 Hz); 7.31 (m, 4H).

0.15 g (0.32 mmol) of compound 34 are dissolved in a 5 ml, and then 0.1 ml (0.5 mmol) of a 5 N solution of HCl in isopropanol are added. This is concentrated; a 1/2 dioxane/water mixture is added, and then it is lyophilized. 0.12 g (0.24 mmol) of compound 34 hydrochloride are obtained in the form of a white solid.

F: 171° C.

¹H NMR (DMSOd₆) δ: 1.49 (m, 5H); 1.89 (m, 2H); 2.50 (m, 2H); 2.73 (m, 1H); 3.05 (m, 4H); 3.39 (m, 2H); 3.68 (is, 4H); 3.98 (m, 2H); 4.14 (m, 2H); 4.25 (m, 1H); 4.90 (m, 1H); 6.47 (m, 1H); 6.53 (m, 1H); 6.83 (m, 1H); 7.28 (m, 5H).

MS m/z 467 (M+1).

HPLC (XBRIDGE C8, acetonitrile/water/KH₂PO₄ (35/65/6.8 g pH 4), 1 ml/min), retention time=8.58 min, chemical purity=98.96%

### EXAMPLE 35

### (S)-4-benzyl-3-(2-(1-(((S)-7-hydroxy-2,3-dihydrobenzo[b][1,4]dioxin-2-yl)methyl)piperidin-4-yl)ethyl)oxazolidin-2-one

### Step 1: Preparation of (S)-4-benzyl-3-(2-(1-(((S)-7-phenoxy-2,3-dihydrobenzo[b][1,4]dioxin-2-yl)methyl)piperidin-4-yl)ethyl)oxazolidin-2-one

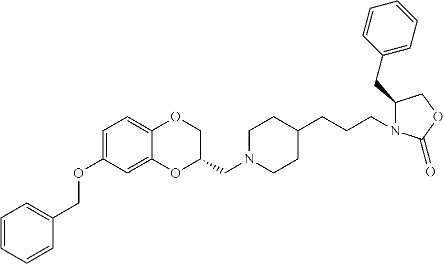 [SMILES: O=C1OC[C@H](Cc2ccccc2)N1CCCC1CCN(C[C@H]2COc3ccc(OCc4ccccc4)cc3O2)CC1]

By proceeding as in Example 1 but replacing (R)-2-(bromomethyl)-2,3-dihydrobenzo[b][1,4]dioxine 2.1 by (R)-(7-(benzyloxy)-2,3-dihydrobenzo[b][1,4]dioxin-2-yl)methyl methanesulfonate 2.25, 3-(2-(piperidin-4-yl)ethyl)oxazolidin-2-one 3.1 by (S)-4-benzyl-3-(2-(piperidin-4-yl)ethyl)oxazolidin-2-one 3.4, the K₂CO₃/KI pair by diisopropylethylamine (1.4 eq) and acetonitrile by DMSO, 0.7 g (1.29 mmol) of compound 35 are obtained in the form of a colorless oil, after purification on silica gel (gradient: dichloromethane/ethyl acetate=70/30% to dichloromethane/methanol=95/5%).

Yield: 24%.

¹H NMR (CDCl₃) δ: 1.26 (m, 3H); 1.50 (m, 2H); 1.67 (m, 2H); 2.08 (m, 2H); 2.53 (m, 1H); 2.65 (m, 2H); 2.92 (m, 1H); 3.03 (m, 1H); 3.11 (m, 2H); 3.57 (m, 1H); 3.92 (dd, 1H, J=11 and 6.8 Hz); 4.00 (m, 2H); 4.17 (m, 1H); 4.26 (m, 2H); 4.97 (s, 2H); 6.48 (dd, 1H, J=); 9 and 2.8 Hz); 6.55 (d, 1H, J=2.8 Hz); 6.77 (d, 1H, J=9 Hz); 7.16 (m, 2H); 7.35 (m, 8H)

### Step 2: Preparation of (S)-4-benzyl-3-(2-(1-(((S)-7-hydroxy-2,3-dihydrobenzo[b][1,4]dioxin-2-yl)methyl)piperidin-4-yl)ethyl)oxazolidin-2-one

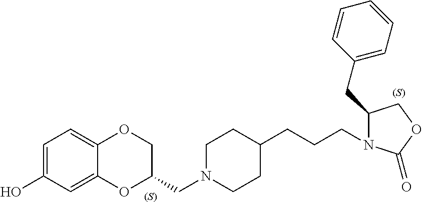

0.7 g (1.29 mmol) of the preceding compound (S)-4-benzyl-3-(2-(1-(((S)-7-phenoxy-2,3-dihydrobenzo[b][1,4]dioxin-2-yl)methyl)piperidin-4-yl)ethyl)oxazolidin-2-one are solubilized in 10 ml of methanol. A catalytic quantity of 5% Pd on carbon is added, and hydrogenation is performed at RT for 5 hours. The medium is then filtered on Celite and concentrated. The compound 35 is obtained in the form of a white solid, after purification on silica gel (eluent: dichloromethane/methanol=95/5%).

Yield: 58%.

¹H NMR (CDCl₃) δ: 1.29 (m, 3H); 1.49 (m, 2H); 1.66 (m, 2H); 2.1 (m, 2H); 2.53 (m, 1H); 2.65 (m, 2H); 2.90 (m, 1H); 3.08 (m, 3H); 3.57 (m, 1H); 3.90 (dd, 1H, 11.4 and 7.2 Hz); 4.00 (m, 2H); 4.18 (m, 2H); 4.30 (m, 1H); 5.30 (ls, 1H); 6.32 (dd, 1H, J=8.4 and 2.8 Hz); 6.40 (d, 1H, J=2.8 Hz); 6.71 (d, 1H, J=8.4 Hz); 7.16 (m, 2H); 7.31 (m, 3H).

0.34 g (0.75 mmol) of compound 35 are dissolved in 5 ml of methanol and 0.068 g (0.75 mmol) of oxalic acid in solution in 5 ml of methanol are added. The mixture is concentrated to dryness then it is triturated in ethyl ether and ethyl acetate; a white precipitate forms and the salt is filtered and dried in the vacuum chamber. 0.63 g (1.13 mmol) of compound 35 oxalate are recovered in the form of a white solid.

F: 122° C.

¹H NMR (DMSOd₆) δ: 1.40 (m, 5H); 1.79 (m, 2H); 2.71 (m, 3H); 3.06 (m, 4H); 3.26 (m, 1H); 3.37 (m, 2H); 3.93 (m, 2H); 4.14 (m, 3H); 4.58 (m, 1H); 6.28 (m, 2H); 6.68 (d, 1H, J=8.4 Hz); 7.28 (m, 5H).

MS m/z 453 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=1.13 min, chemical purity: 98.47%.

### EXAMPLE 36

### S)-4-benzyl-3-(2-(1-(((S)-6-hydroxy-2,3-dihydrobenzo[b][1,4]dioxin-2-yl)methyl)piperidin-4-yl)ethyl)oxazolidin-2-one

### Step 1: Preparation of (S)-4-benzyl-3-(2-(1-(((S)-7-phenoxy-2,3-dihydrobenzo[b][1,4]dioxin-2-yl)methyl)piperidin-4-yl)ethyl)oxazolidin-2-one

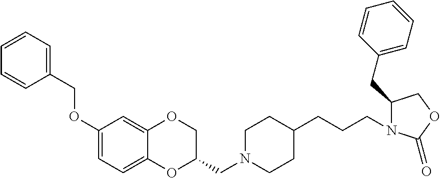 [SMILES: O=C1OC[C@H](Cc2ccccc2)N1CCCC1CCN(C[C@H]2COc3cc(OCc4ccccc4)ccc3O2)CC1]

By proceeding as in Example 1 but replacing (R)-2-(bromomethyl)-2,3-dihydrobenzo[b][1,4]dioxine 2.1 by (R)-(6-(benzyloxy)-2,3-dihydrobenzo[b][1,4]dioxin-2-yl)methyl methanesulfonate 2.26 and 3-(2-(piperidin-4-yl)ethyl)oxazolidin-2-one 3.1 by (S)-4-benzyl-3-(2-(piperidin-4-yl)ethyl)oxazolidin-2-one 3.4, 0.6 g (1.11 mmol) of compound 36 are obtained in the form of a colorless oil, after purification on silica gel (gradient: dichloromethane/ethyl acetate=70/30% to dichloromethane/methanol=95/5%).

Yield: 21%.

### Step 2: Preparation of (S)-4-benzyl-3-(2-(1-(((S)-7-hydroxy-2,3-dihydrobenzo[b][1,4]dioxin-2-yl)methyl)piperidin-4-yl)ethyl)oxazolidin-2-one

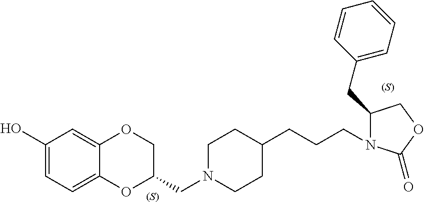

0.6 g (1.11 mmol) of the preceding compound ((S)-4-benzyl-3-(2-(1-(((S)-7-phenoxy-2,3-dihydrobenzo[b][1,4]dioxin-2-yl)methyl)piperidin-4-yl)ethyl)oxazolidin-2-one are solubilized in 5 ml of methanol and 5 ml of dichloromethane. A catalytic quantity of 5% Pd on carbon is added, and hydrogenation is performed at RT for 5 hours. The medium is then filtered on Celite and concentrated. 0.3 g (0.66 mmol) of compound 36 are obtained in the form of a cream residue, after purification on silica gel (gradient: dichloromethane/methanol=95/5% to dichloromethane/methanol=90/10% over 30 min).

Yield: 66%.

¹H NMR (CDCl₃) δ: 1.37 (m, 5H); 1.72 (m, 1H); 1.81 (m, 1H); 2.27 (m, 2H); 2.69 (m, 3H); 3.09 (m, 3H); 3.24 (m, 1H); 3.54 (m, 1H); 3.92 (dd, 1H, J=11.2 and 6.8 Hz); 4.02 (m, 2H); 4.18 (m, 2H); 4.43 (m, 1H); 6.35 (dd, 1H, J=8.4 and 2.8 Hz); 6.41 (d, 1H, J=2.8 Hz); 6.66 (d, 1H, J=8.8 Hz); 7.16 (m, 2H); 7.31 (m, 3H).

0.3 g (0.66 mmol) of compound 36 are dissolved in 5 ml of methanol and 0.060 g (0.66 mmol) of oxalic acid in solution in 5 ml of methanol are added. A white precipitate forms; the salt is filtered and dried in the vacuum chamber. 0.23 g (0.42 mmol) of compound 36 oxalate are obtained in the form of a white solid.

F: 122° C.

¹H NMR (DMSOd₆) δ: 1.41 (m, 5H); 1.79 (m, 2H); 2.50 (m, 1H); 2.73 (m, 2H); 3.06 (m, 4H); 3.27 (m, 1H); 3.37 (m, 2H); 3.95 (m, 2H); 4.17 (m, 3H); 4.52 (m, 1H); 6.27 (m, 2H); 6.70 (d, 1H, J=9.2 Hz); 7.28 (m, 5H).

MS m/z 453 (M+1).

HPLC (XBRIDGE C8, acetonitrile/water/KH₂PO₄ (30/70/6.8 g pH 4), 0.4 ml/min), retention time=7.61 min, chemical purity=99.91%

### EXAMPLE 37

### (S)-3-(2-(1-(((S)-2,3-dihydrobenzo[b][1,4]dioxin-2-yl)methyl)piperidin-4-yl)ethyl)-4-(4-methoxybenzyl)oxazolidin-2-one

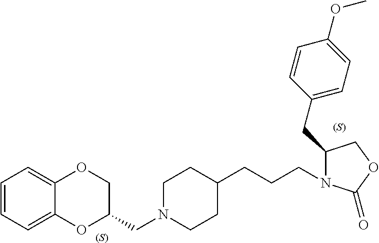

By proceeding as in Example 1 but replacing 3-(2-(piperidin-4-yl)ethyl)oxazolidin-2-one 3.1 by (S)-4-isopropyl-3-(2-(piperidin-4-yl)ethyl)oxazolidin-2-one 3.9, K₂CO₃ by Cs₂CO₃ and acetonitrile by DMF, 0.38 g (0.81 mmol) of compound 37 are obtained in the form of a colorless solid, after purification by on silica gel (eluent: gradient dichloromethane=100% to ethyl acetate=100%).

Yield: 41%

¹H NMR (CDCl₃) δ: 1.27 (m, 3H); 1.50 (m, 2H); 1.70 (m, 2H); 2.09 (m, 2H); 2.60 (m, 3H); 2.89 (m, 1H); 3.04 (m, 3H); 3.57 (m, 1H); 3.80 (s, 3H); 3.98 (m, 3H); 4.16 (m, 1H); 4.30 (m, 2H); 6.85 (m, 6H); 7.07 (m, 2H).

0.38 g (0.81 mmol) of compound 37 are dissolved in 5 ml of methanol and 0.073 g (0.81 mmol) of oxalic acid in solution in 5 ml of methanol are added. A little ethyl ether is added and a white precipitate forms. The salt is filtered and dried in the vacuum chamber. 0.37 g (0.66 mmol) of compound 37 oxalate are recovered in the form of a white solid.

F: 232° C.

¹H NMR (DMSOd₆) δ: 1.40 (m, 5H); 1.80 (m, 2H); 2.66 (m, 3H); 2.96 (m, 1H); 3.09 (m, 3H); 3.26 (m, 1H); 3.36 (m, 2H); 3.72 (s, 3H); 3.99 (m, 3H); 4.15 (m, 1H); 4.30 (m, 1H); 4.61 (m, 1H); 6.88 (m, 6H); 7.19 (m, 2H).

MS m/z 467 (M+1).

HPLC (XBRIDGE C8, acetonitrile/water/KH₂PO₄ (35/65/6.8 g pH 4), 1 ml/min), retention time=10.73 min, chemical purity=99.49%

### EXAMPLE 38

### (S)-3-(2-(1-(((S)-2,3-dihydrobenzo[b][1,4]dioxin-2-yl)methyl)piperidin-4-yl)ethyl)-4-(4-hydroxybenzyl)oxazolidin-2-one

### Step 1: (S)-4-(4-(benzyloxy)benzyl)-3-(2-(1-(((S)-2,3-dihydrobenzo[b][1,4]dioxin-2-yl)methyl)piperidin-4-yl)ethyl)oxazolidin-2-one

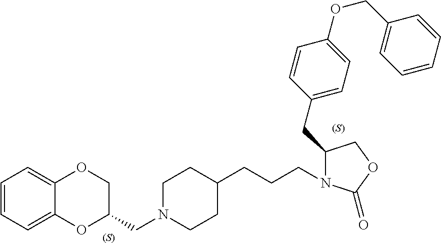

By proceeding as in Example 1 but replacing 3-(2-(piperidin-4-yl)ethyl)oxazolidin-2-one 3.1 by (S)-4-(4-(benzyloxy)benzyl)-3-(2-(piperidin-4-yl)ethyl)oxazolidin-2-one 3.10, K₂CO₃ by Cs₂CO₃ and acetonitrile by DMF, 0.7 g (1.29 mmol) of (S)-4-(4-(benzyloxy)benzyl)-3-(2-(1-(((S)-2,3-dihydrobenzo[b][1,4]dioxin-2-yl)methyl)piperidin-4-yl)ethyl)oxazolidin-2-one are obtained in the form of a colorless oil, after purification on silica gel (eluent: dichloromethane/methanol=95/5%)

Yield: 44%.

¹H NMR (CDCl₃) δ: 1.26 (m, 3H); 1.50 (m, 2H); 1.70 (m, 2H); 2.09 (m, 2H); 2.59 (m, 3H); 2.88 (m, 1H); 3.03 (m, 3H); 3.57 (m, 1H); 3.98 (m, 3H); 4.16 (m, 1H); 4.29 (m, 2H); 6.85 (m, 4H); 6.94 (m, 2H); 7.07 (m, 2H); 7.37 (m, 5H).

### Step 2: (S)-3-(2-(1-(((S)-2,3-dihydrobenzo[b][1,4]dioxin-2-yl)methyl)piperidin-4-yl)ethyl)-4-(4-hydroxybenzyl)oxazolidin-2-one

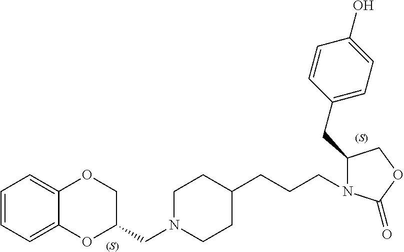

0.7 g (1.29 mmol) of the preceding compound ((S)-4-(4-(benzyloxy)benzyl)-3-(2-(1-(((S)-2,3-dihydrobenzo[b][1,4]dioxin-2-yl)methyl)piperidin-4-yl)ethyl)oxazolidin-2-one are solubilized in 10 ml of methanol. A catalytic quantity of 10% Pd on carbon is added, and hydrogenation is performed at RT for 5.50 hours. The medium is then filtered on Celite and concentrated. 0.32 g (0.070 mmol) of compound 38 are obtained in the form of a cream solid, after purification on silica gel (eluent: dichloromethane/methanol=95/5%).

Yield: 55%.

¹H NMR (CDCl₃) δ: 1.26 (m, 3H); 1.47 (m, 2H); 1.69 (m, 2H); 2.11 (m, 2H); 2.62 (m, 3H); 3.00 (m, 4H); 3.52 (m, 1H); 3.97 (m, 3H); 4.18 (m, 1H); 4.30 (m, 2H); 6.83 (m, 6H); 7.00 (m, 2H).

0.32 g (0.70 mmol) of compound 38 are dissolved in 5 ml of methanol and 0.060 g (0.70 mmol) of oxalic acid in solution in 5 ml of methanol are added. The medium is concentrated and triturated with ethyl ether and ethyl acetate; a white precipitate is formed. The salt is filtered and dried in the vacuum chamber. 0.32 g (0.59 mmol) of compound 38 oxalate are obtained in the form of a cream solid.

F: 107° C.

¹H NMR (DMSOd₆) δ: 1.41 (m, 5H); 1.81 (m, 2H); 2.66 (m, 3H); 2.91 (dd, 1H, J=13.6 and 4 Hz); 3.09 (m, 3H); 3.26 (m, 1H); 3.38 (m, 2H); 3.94 (dd, 1H, J=8 and 6 Hz); 4.00 (m, 2H); 4.14 (m, 1H); 4.29 (m, 1H); 4.63 (m, 1H); 6.69 (d, 2H, J=8.4 Hz); 6.89 (m, 4H); 7.05 (d, 2H, J=8.4 Hz).

MS m/z 453 (M+1).

HPLC (XBRIDGE C8, acetonitrile/water/KH₂PO₄ (30/70/6.8 g pH 4), 1 ml/min), retention time=8.11 min, chemical purity=98.65%

### EXAMPLE 39

### (S)-3-(2-(1-(((S)-2,3-dihydrobenzo[b][1,4]dioxin-2-yl)methyl)piperidin-4-yl)ethyl)-4-(3,4-dimethoxybenzyl)oxazolidin-2-one

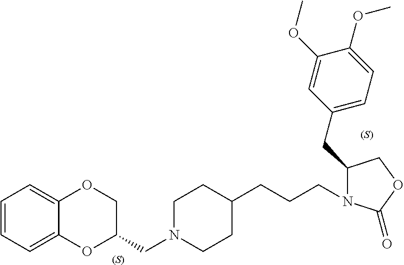

By proceeding as in Example 1 but replacing 3-(2-(piperidin-4-yl)ethyl)oxazolidin-2-one 3.1 by (S)-4-(3,4-dimethoxybenzyl)-3-(2-(piperidin-4-yl)ethyl)oxazolidin-2-one 3.11, K₂CO₃ by Cs₂CO₃ and acetonitrile by DMF, 0.39 g (0.78 mmol) of 39 are obtained in the form of a colorless oil, after purification on silica gel (eluent: dichloromethane/methanol=95/5%).

Yield: 42%

¹H NMR (CDCl₃) δ: 1.29 (m, 3H); 1.51 (m, 2H); 1.70 (m, 2H); 2.09 (m, 2H); 2.54 (dd, 1H, J=13.2 and 6 Hz); 2.64 (m, 2H); 2.90 (m, 1H); 3.05 (m, 3H); 3.56 (m, 1H); 3.87 (s, 3H); 3.88 (s, 3H); 3.99 (m, 3H); 4.17 (m, 1H); 4.30 (m, 2H); 6.64 (d, 1H, J=1.4 Hz); 6.70 (dd, 1H, J=5 and 1.4 Hz); 6.85 (m, 5H).

0.39 g (0.78 mmol) of compound 39 are dissolved in 5 ml of methanol and 0.070 g (0.78 mmol) of oxalic acid in solution in 5 ml of methanol are added. A white precipitate is formed. The salt is filtered and dried in the vacuum chamber. 0.34 g (0.58 mmol) of compound 39 oxalate are recovered in the form of a white solid.

F: 189° C.

¹H NMR (DMSOd₆) δ: 1.38 (m, 3H); 1.45 (m, 2H); 1.81 (m, 2H); 2.65 (m, 3H); 2.95 (dd, 1H, J=13.6 and 4.4 Hz); 3.10 (m, 3H); 3.26 (m, 1H); 3.37 (m, 2H); 3.72 (s 3H); 3.74 (s, 3H); 3.99 (m, 2H); 4.07 (m, 1H); 4.15 (m, 1H); 4.30 (m, 1H); 4.64 (m, 1H); 6.78 (m, 1H); 6.89 (m, 6H).

MS m/z 497 (M+1).

HPLC (XBRIDGE C8, acetonitrile/water/KH₂PO₄ (30/70/6.8 g pH 4), 0.4 ml/min), retention time=9.10 min, chemical purity=99.40%

### EXAMPLE 40

### (S)-3-((4-(2-((S)-4-(4-hydroxybenzyl)-2-oxooxazolidin-3-yl)ethyl)piperidin-1-yl)methyl)-2,3-dihydrobenzo[b][1,4]dioxine-6-carbonitrile

### Step 1: (S)-3-((4-(2-((S)-4-(4-(benzyloxy)benzyl)-2-oxooxazolidin-3-yl)ethyl)piperidin-1-yl)methyl)-2,3-dihydrobenzo[b][1,4]dioxine-6-carbonitrile

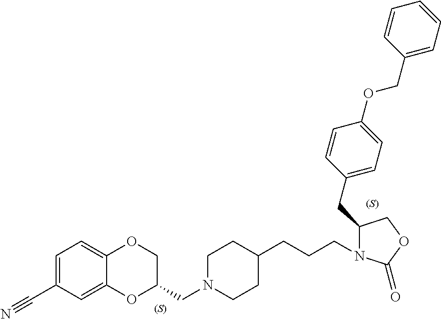

By proceeding as in Example 1 but replacing (R)-2-(bromomethyl)-2,3-dihydrobenzo[b][1,4]dioxine 2.1 by (S)-3-(bromomethyl)-2,3-dihydrobenzo[b][1,4]dioxine-6-carbonitrile 2.11, 3-(2-(piperidin-4-yl)ethyl)oxazolidin-2-one 3.1 by (S)-4-(4-(benzyloxy)benzyl)-3-(2-(piperidin-4-yl)ethyl)oxazolidin-2-one 3.10, the K₂CO₃/KI pair by DMAP (1 eq) and acetonitrile by dimethylsulfoxide, 0.22 g (0.39 mmol) of (S)-3-((4-(2-((S)-4-(4-(benzyloxy)benzyl)-2-oxooxazolidin-3-yl)ethyl)piperidin-1-yl)methyl)-2,3-dihydrobenzo[b][1,4]dioxine-6-carbonitrile are obtained in the form of a colorless oil, after purification on silica gel (eluent:gradient: dichloromethane=100% to ethyl acetate=100% over 30 min).

Yield: 41%

¹H NMR (CDCl₃) δ: 1.26 (m, 3H); 1.50 (m, 2H); 1.71 (m, 2H); 2.10 (m, 2H); 2.54 (dd, 1H, J=13.4 and 6 Hz); 2.63 (m, 2H); 2.87 (m, 1H); 2.94 (m, 1H); 3.07 (m, 2H); 3.57 (m, 1H); 4.01 (m, 3H); 4.13 (m, 1H); 4.28 (m, 1H); 4.36 (dd, 1H, J=11.2 and 2.4 Hz); 5.05 (s, 2H); 6.92 (m, 3H); 7.07 (d, 2H, J=8.4 Hz); 7.15 (m, 2H); 7.42 (m, 5H).

### Step 2: (S)-3-((4-(2-((S)-4-(4-hydroxybenzyl)-2-oxooxazolidin-3-yl)ethyl)piperidin-1-yl)methyl)-2,3-dihydrobenzo[b][1,4]dioxine-6-carbonitrile

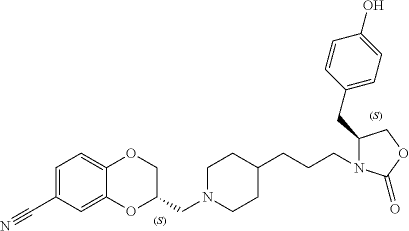

0.22 g (0.39 mmol) of the preceding compound (S)-3-((4-(2-((S)-4-(4-(benzyloxy)benzyl)-2-oxooxazolidin-3-yl)ethyl)piperidin-1-yl)methyl)-2,3-dihydrobenzo[b][1,4]dioxine-6-carbonitrile

are solubilized in 5 ml of methanol and 5 ml of dichloromethane. A catalytic quantity of 5% Pd on carbon is added, and hydrogenation is performed at RT for 9 hours. The medium is then filtered on Celite and concentrated. 0.12 g (0.25 mmol) of compound 40 are obtained in the form of a white foam after purification on silica gel (gradient: dichloromethane=100% to dichloromethane/methanol=95/5% over 30 min).

Yield: 64%

¹H NMR (CDCl₃) δ: 1.27 (m, 3H); 1.49 (m, 2H); 1.71 (m, 2H); 2.09 (m, 2H); 2.54 (dd, 1H, J=13.6 and 6 Hz); 2.65 (m, 2H); 2.89 (m, 1H); 2.98 (m, 2H); 3.09 (m, 1H); 3.56 (m, 1H); 4.00 (m, 3H); 4.18 (m, 1H); 4.31 (m, 1H); 4.36 (dd, 1H, J=11.4 and 2 Hz); 6.79 (d, 2H, J=6.4 Hz); 6.91 (m, 1H); 7.01 (d, 2H, J=6.4 Hz); 7.13 (m, 2H).

0.115 g (0.24 mmol) of compound 40 are dissolved in a 5 ml of methanol and 0.1 ml (0.5 mmol) of a 5 N solution of HCl in isopropanol are added. This is concentrated, a 1/2 dioxane/water mixture is added, and then it is lyophilized. 0.12 g (0.23 mmol) of compound 40 hydrochloride are obtained in the form of a white solid.

¹H NMR (DMSOd₆) δ: 1.48 (m, 5H); 1.91 (m, 2H); 2.56 (m, 3H); 2.92 (dd, 1H, J=13.8 and 4 Hz); 3.07 (m, 3H); 3.40 (m, 2H); 3.66 (m, 1H); 3.94 (dd, 1H, J=8 and 5.2 Hz); 4.02 (m, 1H); 4.16 (m, 2H); 4.42 (m, 1H); 4.87 (m, 1H); 6.70 (d, 2H, J=8.4 Hz); 7.05 (d, 2H, J=8.4 Hz); 7.12 (d, 1H, J=8.4 Hz); 7.39 (dd, 1H, J=8.4 and 1.6 Hz); 7.50 (d, 1H, J=1.6 Hz); 9.31 (s, 1H); 10.34 (ls, 1H).

MS m/z 478 (M+1).

HPLC (XBRIDGE C8, acetonitrile/water/KH₂PO₄ (30/70/6.8 g pH 4), 1 ml/min), retention time=6.95 min, chemical purity=97.79%

### EXAMPLE 41

### (S)-3-((4-(2-((S)-4-(4-fluorobenzyl)-2-oxooxazolidin-3-yl)ethyl)piperidin-1-yl)methyl)-2,3-dihydrobenzo[b][1,4]dioxine-6-carbonitrile

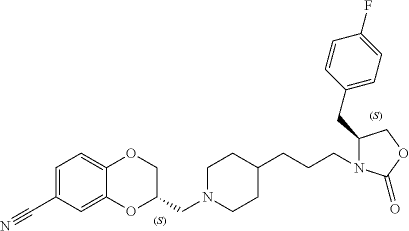

By proceeding as in Example 1 but replacing (R)-2-(bromomethyl)-2,3-dihydrobenzo[b][1,4]dioxine 2.1 by (S)-3-(bromomethyl)-2,3-dihydrobenzo[b][1,4]dioxine-6-carbonitrile 2.11, 3-(2-(piperidin-4-yl)ethyl)oxazolidin-2-one 3.1 by (S)-4-(4-fluorobenzyl)-3-(2-(piperidin-4-yl)ethyl)oxazolidin-2-one 3.12, the K₂CO₃/KI pair by DMAP (1 eq) and acetonitrile by dimethylsulfoxide, 0.26 g (0.54 mmol) of compound 41 are obtained in the form of a colorless oil, after purification on silica gel (gradient: =100% dichloromethane to dichloromethane/methanol=98/2% over 30 min).

Yield: 35%

¹H NMR (CDCl₃) δ: 1.26 (m, 3H); 1.49 (m, 2H); 1.71 (m, 2H); 2.10 (m, 2H); 2.54 (dd, 1H, J=13.4 and 6.4 Hz); 2.67 (m, 2H); 2.87 (m, 1H); 2.94 (m, 1H); 3.07 (m, 2H); 3.59 (m, 1H); 4.00 (m, 3H); 4.18 (m, 1H); 4.29 (m, m, 1H); 4.37 (dd, 1H, J=11.2 and 2.4 Hz); 6.91 (d, 1H, J=8.4 Hz); 7.03 (m, 2H); 7.14 (m, 4H).

0.26 g (0.54 mmol) of compound 41 are dissolved in a 5 ml of methanol and 0.12 ml (0.6 mmol) of a 5 N solution of HCl in isopropanol are added. This is concentrated, a 1/2 dioxane/water mixture is added, and then it is lyophilized. 0.27 g (0.52 mmol) of compound 41 hydrochloride are obtained in the form of a white solid.

¹H NMR (DMSOd₆) δ: 1.48 (m, 5H); 1.90 (m, 2H); 2.50 (m, 2H); 2.74 (m, 1H); 3.08 (m, 4H); 3.40 (m, 2H); 3.67 (m, 1H); 3.97 (dd, 1H, J=7.8 and 5.2 Hz); 4.13 (m, 3H); 4.46 (m, 1H); 4.98 (m, 1H); 7.14 (m, 3H); 7.33 (m, 2H); 7.38 (dd, 1H, J=8.4 and 2 Hz); 7.50 (d, 1H, J=2 Hz); 10.60 (is, 1H).

MS m/z 480 (M+1)

HPLC (XBRIDGE C8, acetonitrile/water/KH₂PO₄ (40/60/6.8 g pH 4), 1 ml/min), retention time=7.71 min, chemical purity=96.89%

### EXAMPLE 42

### (S)-4-benzyl-3-(3-(1-(((S)-2,3-dihydrobenzo[b][1,4]dioxin-2-yl)methyl)piperidin-4-yl)propyl)oxazolidin-2-one

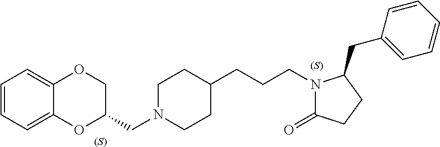

By proceeding as in Example 1 but replacing 3-(2-(piperidin-4-yl)ethyl)oxazolidin-2-one 3.1 by (S)-4-benzyl-3-(3-(piperidin-4-yl)propyl)oxazolidin-2-one 3.31 and acetonitrile by dimethylformamide, 0.6 g (1.33 mmol) of compound 42 are obtained in the form of a colorless oil, after purification by flash chromatography on silica gel (eluent: dichloromethane/methanol=95/5%).

Yield: 53%

0.6 g (1.33 mmol) of compound 42 are dissolved in a minimal amount of methanol and 0.108 g (1.20 mmol) of oxalic acid in solution in 5 ml of methanol are added. The medium is concentrated and triturated with ethyl ether. The precipitate, which tends to stick together, is filtered and left to dry in the vacuum chamber. 0.56 g (1.03 mmol) of compound 42 oxalate are recovered in the form of a white solid.

¹H NMR (DMSOd₆) δ: 1.17 (m, 2H); 1.42 (m, 5H); 1.76 (m, 2H); 2.71 (m, 3H); 3.04 (m, 4H); 3.31 (m, 3H); 3.98 (m, 2H); 4.09 (m, 1H); 4.17 (m, 1H); 4.29 (m, 1H); 4.62 (m, 1H); 6.88 (m, 4H); 7.29 (m, 5H).

MS m/z 451 (M+1).

HPLC (XBRIDGE C8, acetonitrile/water/KH₂PO₄ (40/60/6.8 g pH 4), 0.4 ml/min), retention time=5.00 min, chemical purity=98.40%

### EXAMPLE 43

### (S)-3-(2-(1-(((S)-2,3-dihydrobenzo[b][1,4]dioxin-2-yl)methyl)piperidin-4-yl)ethyl)-4-phenyloxazolidin-2-one

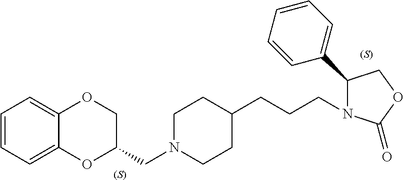

By proceeding as in Example 1 but replacing 3-(2-(piperidin-4-yl)ethyl)oxazolidin-2-one 3.1 by (S)-4-phenyl-3-(2-(piperidin-4-yl)ethyl)oxazolidin-2-one 3.6, K₂CO₃ by Cs₂CO₃ and acetonitrile by dimethylformamide, 0.30 g (0.71 mmol) of compound 43 are obtained in the form of a colorless oil, after purification on silica gel (gradient: dichloromethane=100% to dichloromethane/methanol=90/10% over 30 min).

Yield: 35%

¹H NMR (CDCl₃) δ: 1.20 (m, 3H); 1.36 (m, 2H); 1.59 (m, 2H); 2.04 (m, 2H); 2.51 (dd, 1H, J=13.2 and 6 Hz); 2.62 (dd, 1H, J=13.2 and 6 Hz); 2.85 (m, 3H); 3.48 (m, 1H); 3.95 (dd, 1H, 11.6 and 7.6 Hz); 4.13 (m, 1H); 4.27 (m, 2H); 4.61 (m, 1H); 4.76 (m, 1H); 6.84 (m, 4H); 7.29 (m, 2H); 7.40 (m, 3H).

0.30 g (0.71 mmol) of compound 43 are dissolved in a 5 ml of methanol and then 0.15 ml (0.75 mmol) of a 5 N solution of HCl in isopropanol are added. The medium is concentrated and triturated with ethyl ether. The solid is filtered and 0.30 g (0.65 mmol) of compound 43 hydrochloride are obtained in the form of a white solid.

F: 68° C.

¹H NMR (DMSOd₆) δ: 1.34 (m, 5H); 1.79 (m, 2H); 2.50 (m, 1H); 2.70 (m, 1H); 2.97 (m, 2H); 3.30 (m, 2H); 3.40 (m, 1H); 3.61 (m, 1H); 4.05 (m, 2H); 4.31 (m, 1H); 4.63 (m, 1H); 4.86 (m, 1H); 4.94 (m, 1H); 6.91 (m, 4H); 7.42 (m, 5H).

MS m/z 423 (M+1).

HPLC (XBRIDGE C8, acetonitrile/water/KH₂PO₄ (35/65/6.8 g pH 4), 0.4 ml/min), retention time=6.14 min, chemical purity=98.76%.

### EXAMPLE 44

### (R)-3-(2-(1-(((S)-2,3-dihydrobenzo[b][1,4]dioxin-2-yl)methyl)piperidin-4-yl)ethyl)-4-phenyloxazolidin-2-one

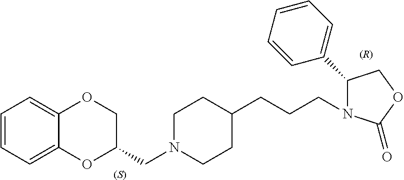

(s)

By proceeding as in Example 1 but replacing 3-(2-(piperidin-4-yl)ethyl)oxazolidin-2-one 3.1 by (R)-4-phenyl-3-(2-(piperidin-4-yl)ethyl)oxazolidin-2-one 3.5, 0.13 g (0.31 mmol) of compound 44 are obtained in the form of a colorless oil, after purification on silica gel (eluent: dichloromethane/ethyl acetate=70/30% then dichloromethane/methanol=95/5%).

Yield: 24%

0.13 g (0.31 mmol) of compound 44 are dissolved in 5 ml of methanol and 0.027 g (0.30 mmol) of oxalic acid in solution in 5 ml of methanol are added. A little ethyl ether is added; a white precipitate is formed. The salt is filtered and dried in the vacuum chamber. 0.054 g (0.11 mmol) of compound 44 oxalate are recovered in the form of a white solid.

F: 125° C.

¹H NMR (DMSOd₆) δ: 1.23 (m, 5H); 1.67 (m, 2H); 2.50 (m, 1H); 2.69 (m, 1H); 2.97-3.72 (m, 6H); 3.97 (m, 1H); 4.07 (m, 1H); 4.28 (m, 1H); 4.55 (m, 1H); 4.63 (m, 1H); 4.92 (m, 1H); 6.87 (m, 4H); 7.41 (m, 5H).

MS m/z 423 (M+1).

HPLC (XBRIDGE C8, acetonitrile/water/KH₂PO₄ (30/70/6.8 g pH 4), 1 ml/min), retention time=16.79 min, chemical purity=100%

### EXAMPLE 45

### 3-(2-(1-(((S)-2,3-dihydrobenzo[b][1,4]dioxin-2-yl)methyl)piperidin-4-yl)ethyl)-4-(2-methoxyphenyl)oxazolidin-2-one

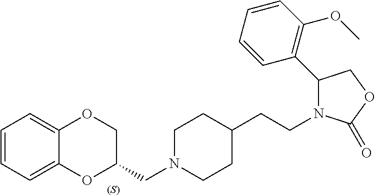

By proceeding as in Example 1 but replacing 3-(2-(piperidin-4-yl)ethyl)oxazolidin-2-one 3.1 by 4-(2-methoxyphenyl)-3-(2-(piperidin-4-yl)ethyl)oxazolidin-2-one 3.7, 2.5 g (5.52 mmol) of compound 45 are obtained in the form of a colorless oil, after purification on silica gel (eluent: dichloromethane=100% then dichloromethane/methanol=95/5%).

Yield: 54%

¹H NMR (CDCl₃) δ: 1.19 (m, 3H); 1.39 (m, 2H); 1.61 (m, 2H); 2.04 (m, 2H); 2.52 (m, 1H); 2.62 (m, 1H); 2.83 (m, 2H); 2.94 (m, 1H); 3.54 (m, 1H); 3.85 (s, 3H); 3.95 (dd, 1H, J=11.6 and 5.6 Hz); 4.10 (dd, 1H, J=8.4 and 5.6 Hz); 4.28 (m, 2H); 4.59 (m, 1H); 5.15 (m, 1H); 6.83 (m, 4H); 6.92 (d, 1H, J=8.2 Hz); 6.99 (m, 1H); 7.19 (dd, 1H, J=7.5 and 1.4 Hz); 7.31 (m, 1H).

0.90 g (1.99 mmol) of compound 45 are dissolved in 10 ml of methanol and then 0.17 g (1.99 mmol) of oxalic acid in solution in 10 ml of methanol are added. A little ethyl ether is added; a white precipitate is formed. The salt is filtered and dried in the vacuum chamber. 0.65 g (1.20 mmol) of compound 45 oxalate are recovered in the form of a white solid.

F: 145° C.

¹H NMR (DMSOd₆) δ: 1.27 (m, 5H); 1.70 (m, 2H); 2.67 (m, 2H); 3.05 (m, 2H); 3.30 (m, 4H); 3.80 (s, 3H); 3.98 (dd, 1H, J=11.4 and 6.6 Hz); 4.06 (dd, 1H, J=8.4 and 5.4 Hz); 4.28 (m, 1H); 4.58 (m, 2H); 5.12 (m, 1H); 6.87 (m, 4H); 7.00 (m, 1H); 7.09 (d, 1H, J=8.2 Hz); 7.21 (m, 1H); 7.36 (m, 1H).

MS m/z 453 (M+1).

HPLC (XBRIDGE C8, acetonitrile/water/KH₂PO₄ (35/65/6.8 g pH 4), 1 ml/min), retention time=9.57 min, chemical purity=99.11%

### EXAMPLE 46

### 3-(2-(1-(((S)-2,3-dihydrobenzo[b][1,4]dioxin-2-yl)methyl)piperidin-4-yl)ethyl)-4-(4-methoxyphenyl)oxazolidin-2-one

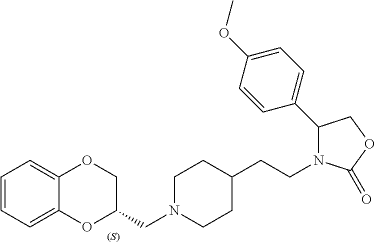

By proceeding as in Example 1 but replacing 3-(2-(piperidin-4-yl)ethyl)oxazolidin-2-one 3.1 by 4-(4-methoxyphenyl)-3-(2-(piperidin-4-yl)ethyl)oxazolidin-2-one 3.8, 1.7 g (3.76 mmol) of compound 46 are obtained in the form of a colorless oil, after purification on silica gel (eluent: dichloromethane=100% then dichloromethane/methanol=95/5%).

Yield: 59%

¹H NMR (CDCl₃) δ: 1.19 (m, 3H); 1.36 (m, 2H); 1.61 (m, 2H); 2.06 (m, 2H); 2.52 (dd, 1H, J=13.2 and 5.8 Hz); 2.62 (dd, 1H, J=13.2 and 5.3 Hz); 2.79 (m, 2H); 2.93 (m, 1H); 3.44 (m, 1H); 3.83 (s, 3H); 3.95 (m, 1H); 4.10 (m, 1H); 4.27 (m, 2H); 4.58 (m, 1H); 4.71 (m, 1H); 6.84 (m, 4H); 6.93 (d, 2H, J=8.1 Hz); 7.21 (d, 2H, J=8.1 Hz).

0.40 g (0.88 mmol) of compound 46 are dissolved in 5 ml of methanol and then 0.079 g (0.88 mmol) of oxalic acid in solution in 5 ml of methanol are added. A little ethyl ether is added; a white precipitate is formed. The salt is filtered and dried in the vacuum chamber. 0.40 g (0.73 mmol) of compound 46 oxalate are recovered in the form of a cream solid.

F: 105° C.

¹H NMR (DMSOd₆) δ: 1.29 (m, 5H); 1.68 (m, 2H); 2.67 (m, 2H); 3.07 (m, 2H); 3.32 (m, 4H); 3.76 (s, 3H); 4.01 (m, 2H); 4.29 (m, 1); 4.60 (m, 2H); 4.87 (m, 1H); 6.86 (m, 4H); 6.99 (d, 2H, J=7.5 Hz); 7.30 (d, 2H, J=7.5 Hz).

MS m/z 453 (M+1).

HPLC (XBRIDGE C8, acetonitrile/water/KH₂PO₄ (35/65/6.8 g pH 4), 1 ml/min), retention time=9.51 min, chemical purity=99.45%

### EXAMPLE 47

### 3-(2-(1-(((S)-2,3-dihydrobenzo[b][1,4]dioxin-2-yl)methyl)piperidin-4-yl)ethyl)-4-(4-hydroxyphenyl)oxazolidin-2-one

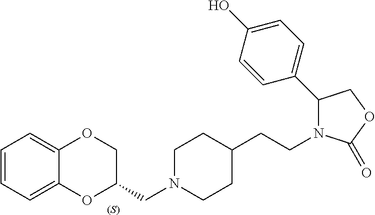

1.30 g (2.87 mmol) of the preceding 3-(2-(1-(((S)-2,3-dihydrobenzo[b][1,4]dioxin-2-yl)methyl)piperidin-4-yl)ethyl)-4-(4-methoxyphenyl)oxazolidin-2-one 46 are solubilized in 20 ml of dichloromethane. This is placed at −78° C. and 0.7 ml (7.18 mmol) of BBr₃ are added dropwise. It is allowed to return to room temperature. It is stirred for 1.5 hours. It is cooled to 0° C., 50 ml of water are added and it is extracted with dichloromethane The organic phase is dried on Na₂SO₄, filtered and evaporated, and 0.30 g (0.68 mmol) of compound 47 are obtained in the form of a white solid, after purification on silica gel (eluent: dichloromethane/methanol=95/5%).

Yield: 23%

¹H NMR (CDCl₃) δ: 1.23 (m, 3H); 1.35 (m, 2H); 1.61 (m, 2H); 2.06 (m, 2H); 2.54 (m, 1H); 2.64 (m, 1H); 2.81 (m, 2H); 2.97 (m, 1H); 3.42 (m, 1H); 3.95 (m, 1H); 4.10 (m, 1H); 4.27 (m, 2H); 4.58 (m, 1H); 4.69 (m, 1H); 6.85 (m, 6H); 7.16 (d, 2H, J=7.9 Hz).

0.08 g (0.18 mmol) of compound 47 are dissolved in 5 ml of methanol and then 0.016 g (0.18 mmol) of oxalic acid in solution in 5 ml of methanol are added. A little ethyl ether is added; a white precipitate is formed. The salt is filtered and dried in the vacuum chamber. 0.094 g (0.17 mmol) of compound 47 oxalate are recovered in the form of a white solid.

F: 113° C.

¹H NMR (DMSOd₆) δ: 1.27 (m, 5H); 1.68 (m, 2H); 2.67 (m, 2H); 3.01 (m, 2H); 3.21 (m, 4H); 4.00 (m, 2H); 4.28 (m, 1H); 4.57 (m, 2H); 4.79 (m, 1H); 6.79 (d, 2H, J=8 Hz); 6.87 (m, 4H); 7.17 (d, 2H, J=8 Hz).

MS m/z 439 (M+1).

HPLC (XBRIDGE C8, acetonitrile/water/KH₂PO₄ (30/70/6.8 g pH 4), 1 ml/min), retention time=7.48 min, chemical purity=89.56%

### EXAMPLE 48

### (S)-3-((4-(2-((R)-2-oxo-4-phenyloxazolidin-3-yl)ethyl)piperidin-1-yl)methyl)-2,3-dihydrobenzo[b][1,4]dioxine-6-carbonitrile

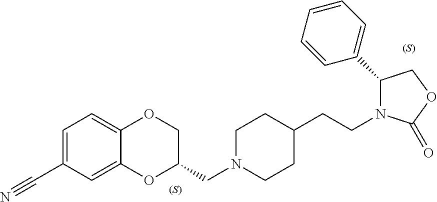

By proceeding as in Example 1 but replacing (R)-2-(bromomethyl)-2,3-dihydrobenzo[b][1,4]dioxine 2.1 by (S)-3-(bromomethyl)-2,3-dihydrobenzo[b][1,4]dioxine-6-carbonitrile 2.11, 3-(2-(piperidin-4-yl)ethyl)oxazolidin-2-one 3.1 by (R)-4-phenyl-3-(2-(piperidin-4-yl)ethyl)oxazolidin-2-one 3.5, the K₂CO₃/KI pair by DMAP (1 eq) and acetonitrile by dimethylsulfoxide, 0.2 g (0.45 mmol) of compound 48 are obtained in the form of a yellow oil, after purification by flash chromatography on silica gel (eluent; dichloromethane/ethyl acetate=70/30% then dichloromethane/methanol=95/5%).

Yield: 70%

¹H NMR (CDCl₃) δ: 1.18 (m, 3H); 1.36 (m, 2H); 1.62 (m, 2H); 2.04 (m, 2H); 2.51 (dd, 1H, J=13.4 and 6 Hz); 2.62 (dd, 1H, J=13.2 and 5.6 Hz); 2.79 (m, 2H); 2.91 (m, 1H); 3.48 (m, 1H); 3.99 (dd, 1H, J=11.4 and 7.2 Hz); 4.13 (dd, 1H, J=8.8 and 6.8 Hz); 4.26 (m, 1H); 4.35 (dd, 1H, J=11.4 and 2 Hz); 4.62 (m, 1H); 4.76 (dd, 1H, J=8.8 and 6.8 Hz); 6.90 (d, 1H, J=8.4 Hz); 7.14 (m, 2H); 7.29 (m, 2H); 7.41 (m, 3H).

0.2 g (0.45 mmol) of compound 48 are dissolved in 5 ml of methanol and then 0.040 g (0.45 mmol) of oxalic acid in solution in 5 ml of methanol are added. A little ethyl ether is added; a white precipitate is formed. The salt is filtered and dried in the vacuum chamber. 0.17 g (0.32 mmol) of compound 48 oxalate are recovered in the form of a white solid.

F: 200° C.

¹H NMR (DMSOd₆) δ: 1.24 (m, 5H); 1.68 (m, 2H); 2.63 (m, 3H); 3.04 (m, 2H); 3.17 (m, 1H); 3.29 (m, 2H); 4.09 (m, 2H); 4.39 (m, 1H); 4.63 (m, 2H); 4.91 (m, 1H); 7.08 (d, 1H, J=8.4 Hz); 7.40 (m, 7H).

MS m/z 448 (M+1).

HPLC (XBRIDGE C8, acetonitrile/water/KH₂PO₄ (35/65/6.8 g pH 4), 0.4 ml/min), retention time=6.33 min, chemical purity=99.01%

### EXAMPLE 49

### (R)-3-(2-(1-(((S)-2,3-dihydrobenzo[b][1,4]dioxin-2-yl)methyl)piperidin-4-yl)ethyl)-4-(hydroxymethyl)oxazolidin-2-one

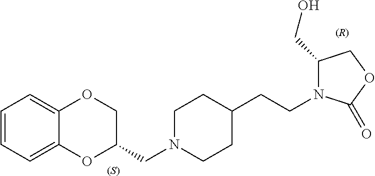

By proceeding as in Example 1 but replacing 3-(2-(piperidin-4-yl)ethyl)oxazolidin-2-one 3.1 by ((R)-4-(hydroxymethyl)-3-(2-(piperidin-4-yl)ethyl)oxazolidin-2-one 3.21 and K₂CO₃ by Cs₂CO₃, 0.4 g (1.06 mmol) of compound 49 are obtained in the form of a colorless oil, after purification on silica gel (gradient: dichloromethane=100% to dichloromethane/methanol=90/10% over 30 min).

Yield: 36%

¹H NMR (CDCl₃) δ: 1.29 (m, 3H); 1.51 (m, 2H); 1.72 (m, 2H); 2.09 (m, 2H); 2.54 (dd, 1H, J=13.4 and 6.4 Hz); 2.65 (dd, 1H, J=13.2 and 5.6 Hz); 2.88 (m, 1H); 2.99 (m, 1H); 3.15 (m, 1H); 3.52 (m, 1H); 3.70 (dd, 1H, J=11.4 and 4 Hz); 3.79 (dd, 1H, J=11.4 and 4 Hz); 3.96 (dd, 1H, J=11.6 and 7.6 Hz); 4.29 (m, 2H); 4.36 (m, 1H); 6.85 (m, 4H).

0.28 g (0.74 mmol) of compound 49 are dissolved in 5 ml of methanol and then 0.066 g (0.74 mmol) of oxalic acid in solution in 5 ml of methanol are added. A little ethyl ether is added; a white precipitate is formed. The salt is filtered and dried in the vacuum chamber. 0.29 g (0.62 mmol) of compound 49 oxalate are recovered in the form of a cream solid.

F: 76° C.

¹H NMR (DMSOd₆) δ: 1.40 (m, 5H); 1.83 (m, 2H); 2.74 (m, 2H); 3.11 (m, 3H); 3.38 (m, 4H); 3.56 (dd, 1H, J=11.6 and 3.6 Hz); 3.82 (m, 1H); 4.02 (m, 2H); 4.28 (m, 2H); 4.65 (m, 1H); 6.89 (m, 4H).

MS m/z 377 (M+1).

HPLC (XBRIDGE C8, acetonitrile/water/KH₂PO₄ (20/80/6.8 g pH 4), 0.4 ml/min), retention time=4.95 min, chemical purity=97.20%

### EXAMPLE 50

### (S)-3-(2-(1-(((S)-2,3-dihydrobenzo[b][1,4]dioxin-2-yl)methyl)piperidin-4-yl)ethyl)-4-(hydroxymethyl)oxazolidin-2-one

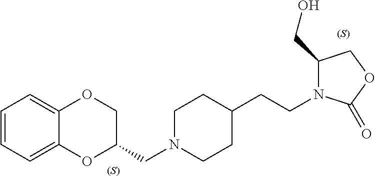

By proceeding as in Example 1 but replacing 3-(2-(piperidin-4-yl)ethyl)oxazolidin-2-one 3.1 by (S)-4-(hydroxymethyl)-3-(2-(piperidin-4-yl)ethyl)oxazolidin-2-one 3.22 and K₂CO₃ by Cs₂CO₃, 0.5 g (1.33 mmol) of compound 50 are obtained in the form of a colorless oil, after purification on silica gel (eluent: dichloromethane/methanol=90/10%).

Yield: 42%

¹H NMR (CDCl₃) δ: 1.28 (m, 3H); 1.51 (m, 2H); 1.68 (m, 1H); 1.75 (m, 1H); 1.86 (Is, 1H); 2.08 (m, 2H); 2.54 (dd, 1H, 13.4 and 5.6 Hz); 2.65 (dd, 1H, J=13.2 and 5.6 Hz); 2.89 (m, 1H); 2.99 (m, 1H); 3.15 (m, 1H); 3.53 (m, 1H); 3.69 (dd, 1H, J=11.4 and 3.6 Hz); 3.79 (dd, 1H, J=11.4 and 4 Hz); 3.88 (m, 1H); 3.96 (dd, 1H, J=11.6 and 8 Hz); 4.33 (m, 3H); 6.85 (m, 4H).

0.50 g (1.33 mmol) of compound 50 are dissolved in 10 ml of methanol and then 0.1 g (1.32 mmol) of oxalic acid in solution in 5 ml of methanol are added. This is concentrated to dryness and taken up with a little ethyl ether; a white precipitate is formed. The salt is filtered and dried in the vacuum chamber. 0.51 g (1.09 mmol) of compound 50 oxalate are recovered in the form of a white solid.

F: 60° C.

¹H NMR (DMSOd₆) δ: 1.39 (m, 5H); 1.80 (m, 2H); 2.66 (m, 2H); 3.08 (m, 3H); 3.23 (m, 1H); 3.34 (m, 2H); 3.42 (m, 1H); 3.56 (m, 1H); 3.81 (m, 1H); 4.01 (m, 2H); 4.28 (m, 2H); 4.59 (m, 1H); 6.87 (m, 4H).

MS m/z 377 (M+1).

HPLC (XBRIDGE C8, acetonitrile/water/KH₂PO₄ (20/80/6.8 g pH 4), 1 ml/min), retention time=7.60 min, chemical purity=98.19%

### EXAMPLE 51

### (S)-3-((4-(2-((S)-4-(hydroxymethyl)-2-oxooxazolidin-3-yl)ethyl)piperidin-1-yl)methyl)-2,3-dihydrobenzo[b][1,4]dioxine-6-carbonitrile

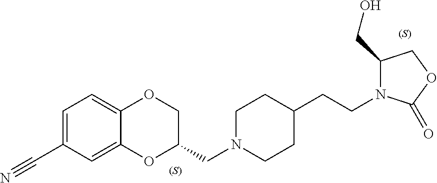

By proceeding as in Example 1 but replacing (R)-2-(bromomethyl)-2,3-dihydrobenzo[b][1,4]dioxine 2.1 by (R)-(7-cyano-2,3-dihydrobenzo[b][1,4]dioxin-2-yl)methyl 4-bromobenzenesulfonate 2.11, 3-(2-(piperidin-4-yl)ethyl)oxazolidin-2-one 3.1 by (S)-4-(hydroxymethyl)-3-(2-(piperidin-4-yl)ethyl)oxazolidin-2-one 3.22, the K₂CO₃/KI pair by diisopropylethylamine and acetonitrile by dimethylsulfoxide, 0.31 g (0.77 mmol) of compound 51 are obtained in the form of a colorless oil, after purification on silica gel (gradient; =100% dichloromethane to dichloromethane/(methanol:NH₄OH 9:1)=90/10% over 30 min).

Yield: 66%

¹H NMR (CDCl₃) δ: 1.29 (m, 3H); 1.51 (m, 2H); 1.72 (m, 2H); 1.90 (ls, 1H); 2.13 (m, 2H); 2.54 (dd, 1H, J=13.4 and 6.4 Hz); 2.66 (dd, 1H, 13.4 and 6 Hz); 2.87 (m, 1H); 2.95 (m, 1H); 3.15 (m, 1H); 3.53 (m, 1H); 3.68 (dd, 1H, J=11.6 and 3.6 Hz); 3.78 (dd, 1H, J=11.6 and 4 Hz); 3.88 (m, 1H); 4.02 (dd, 1H, J=11.4 and 7.2 Hz); 4.22 (dd, 1H, J=8.4 and 5.6 Hz); 4.30 (m, 1H); 4.36 (m, 2H); 6.73 (d, 1H, J=8.8 Hz); 7.15 (m, 2H).

0.31 g (0.77 mmol) of compound 51 are dissolved in a 20 ml of ethyl acetate, then 0.20 ml (1 mmol) of a 5 N solution of HCl in isopropanol are added. A white precipitate is formed. This precipitate is filtered and 0.31 g (0.71 mmol) of compound 51 hydrochloride are obtained in the form of a white solid.

F: 247° C.

¹H NMR (DMSOd₆) δ: 1.49 (m, 5H); 1.90 (m, 2H); 3.07 (m, 2H); 3.31 (m, 4H); 3.45 (m, 2H); 3.58 (m, 1H); 3.67 (m, 1H); 3.83 (m, 1H); 4.04 (m, 1H); 4.18 (dd, 1H, J=11.6 and 6.4 Hz); 4.27 (m, 1H); 4.45 (dd, 1H, J=11.6 and 2.4 Hz); 4.99 (m, 2H); 7.11 (d, 1H, J=8.6 Hz); 7.38 (dd, 1H, J=8.6 and 1.6 Hz); 7.51 (d, 1H, J=1.6 Hz); 10.70 (ls, 1H).

MS m/z 402 (M+1).

HPLC (XBRIDGE C8, acetonitrile/water/KH₂PO₄ (20/80/6.8 g pH 4), 1 ml/min), retention time=7.12 min, chemical purity=98.89%

HPLC (CHIRACEL OJ-H, heptane/ethanol/diethylamine (80/20/0.1), 1 ml/min); compound 51, retention time=24.92 min, compound 52, retention time=19.53 min, compound 53, retention time=15.39 min, compound 54, retention time=16.87 min, ratio of AUCs (51)/(52)/(53)/(54)=96.37/0.51/3.11/0.

### EXAMPLE 52

### (R)-3-((4-(2-((S)-4-(hydroxymethyl)-2-oxooxazolidin-3-yl)ethyl)piperidin-1-yl)methyl)-2,3-dihydrobenzo[b][1,4]dioxine-6-carbonitrile

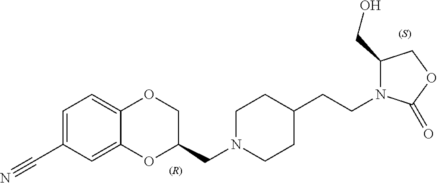

By proceeding as in Example 1 but replacing (R)-2-(bromomethyl)-2,3-dihydrobenzo[b][1,4]dioxine 2.1 by (S)-(7-cyano-2,3-dihydrobenzo[b][1,4]dioxin-2-yl)methyl 4-bromobenzenesulfonate 2.12, 3-(2-(piperidin-4-yl)ethyl)oxazolidin-2-one 3.1 by (S)-4-(hydroxymethyl)-3-(2-(piperidin-4-yl)ethyl)oxazolidin-2-one 3.22, the K₂CO₃/KI pair by diisopropylethylamine (1.4 eq) and acetonitrile by dimethylsulfoxide, 0.28 g (0.68 mmol) of compound 52 are obtained in the form of a pale yellow oil, after purification on silica gel (eluent:gradient: =100% dichloromethane to dichloromethane/(methanol:NH₄OH 9:1)=90/10% over 30 min).

Yield: 60%

¹H NMR (CDCl₃) δ: 1.28 (m, 3H); 1.52 (m, 2H); 1.73 (m, 2H); 1.98 (ls, 1H); 2.12 (m, 2H); 2.54 (dd, 1H, J=13.4 and 6 Hz); 2.65 (dd, 1H, J=13.6 and 8 Hz); 2.86 (m, 1H); 2.95 (m, 1H); 3.15 (m, 1H); 3.52 (m, 1H); 3.68 (m, 1H); 3.80 (m, 1H); 3.90 (m, 1H); 4.02 (dd, 1H, J=11.4 and 7.6 Hz); 4.22 (dd, 1H, J=8.8 and 5.6 Hz); 4.29 (m, 1H); 4.36 (m, 2H); 6.91 (d, 1H, J=8.4 Hz); 7.19 (m, 2H).

0.28 g (0.68 mmol) of compound 52 are dissolved in a 20 ml of ethyl acetate, then 0.20 ml (1 mmol) of a 5 N solution of HCl in isopropanol are added. A white precipitate is formed. This precipitate is filtered and 0.27 g (0.62 mmol) of compound 52 hydrochloride are obtained in the form of a white solid.

F: 220° C.

¹H NMR (DMSOd₆) δ: 1.53 (m, 5H); 1.89 (m, 2H); 3.06 (m, 2H); 3.35 (m, 4H); 3.45 (m, 2H); 3.57 (m, 1H); 3.67 (m, 1H); 3.83 (m, 1H); 4.04 (m, 1H); 4.18 (m, 1H); 4.27 (m, 1H); 4.44 (m, 1H); 5.00 (m, 2H); 7.11 (d, 1H, J=8.4 Hz); 7.38 (dd, 1H, J=8.4 and 2 Hz); 7.50 (d, 1H, J=2 Hz); 10.65 (ls, 1H).

MS m/z 402 (M+1).

HPLC (XBRIDGE C8, acetonitrile/water/KH₂PO₄ (20/80/6.8 g pH 4), 1 ml/min), retention time=7.13 min, chemical purity=99.64%

HPLC (CHIRACEL OJ-H, heptane/ethanol/diethylamine (80/20/0.1), 1 ml/min); ratio of AUCs (51)/(52)/(53)/(54)=0.74/96.13/0/3.13.

### EXAMPLE 53

### (S)-3-((4-(2-((R)-4-(hydroxymethyl)-2-oxooxazolidin-3-yl)ethyl)piperidin-1-yl)methyl)-2,3-dihydrobenzo[b][1,4]dioxine-6-carbonitrile

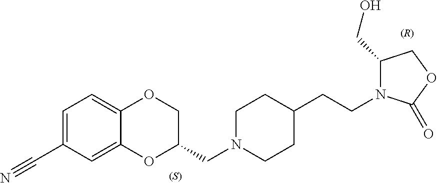

By proceeding as in Example 1 but replacing (R)-2-(bromomethyl)-2,3-dihydrobenzo[b][1,4]dioxine 2.1 by (R)-(7-cyano-2,3-dihydrobenzo[b][1,4]dioxin-2-yl)methyl 4-bromobenzenesulfonate 2.11, 3-(2-(piperidin-4-yl)ethyl)oxazolidin-2-one 3.1 by (R)-4-(hydroxymethyl)-3-(2-(piperidin-4-yl)ethyl)oxazolidin-2-one 3.21, the K₂CO₃/KI pair by diisopropylethylamine (1.4 eq) and acetonitrile by dimethylsulfoxide, 0.43 g (1.07 mmol) of compound 53 are obtained in the form of a pale yellow oil, after purification on silica gel (gradient: dichloromethane=100% to dichloromethane/methanol=95/5% over 40 min).

Yield: 46%

¹H NMR (CDCl₃) δ: 1.29 (m, 3H); 1.51 (m, 2H); 1.70 (m, 3H); 2.12 (m, 2H); 2.54 (dd, 1H, J=13.4 and 6.4 Hz); 2.65 (dd, 1H, J=13.2 and 5.6 Hz); 2.86 (m, 1H); 2.96 (m, 1H); 3.15 (m, 1H); 3.53 (m, 1H); 3.70 (dd, 1H, J=11.4 and 4 Hz); 3.79 (dd, 1H, J=11.4 and 4 Hz); 3.89 (in, 1H); 4.02 (dd, 1H, J=11.4 and 7.2 Hz); 4.21 (dd, 1H, J=8.8 and 6 Hz); 4.29 (m, 1H); 4.36 (m, 2H); 6.91 (d, 1H, J=8.4 Hz); 7.14 (dd, 1H, J=8.4 and 2 Hz); 7.16 (d, 1H, J=2 Hz).

0.43 g (1.07 mmol) of compound 53 are dissolved in 10 ml of methanol and then 0.096 ml (1.07 mmol) of oxalic acid in solution in 5 ml of methanol are added. The medium is concentrated and triturated with ether; a white precipitate is formed. This precipitate is filtered and 0.44 g (0.89 mmol) of compound 53 oxalate are obtained in the form of a white solid.

F: 170° C.

¹H NMR (DMSOd₆) δ: 1.35 (m, 5H); 1.78 (m, 2H); 2.57 (m, 2H); 3.05 (m, 3H); 3.27 (m, 3H); 3.43 (dd, 1H, J=11.8 and 3.2 Hz); 3.56 (dd, 1H, J=11.8 and 3.6 Hz); 3.81 (m, 1H); 4.04 (dd, 1H, J=8.4 and 5.6 Hz); 4.12 (dd, 1H, J=11.6 and 6.8 Hz); 4.27 (m, 1H); 4.40 (dd, 1H, J=11.6 and 2.4 Hz); 4.65 (m, 1H); 7.08 (d, 1H, J=8.4 Hz); 7.35 (dd, 1H, J=8.4 and 2 Hz); 7.44 (d, 1H, J=2 Hz).

MS m/z 402 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 15 to 40% in 5 min, 0.75 ml/min), retention time=1.47 min, chemical purity: 97.49%.

HPLC (CHIRACEL OJ-H, heptane/ethanol/diethylamine (80/20/0.1), 1 ml/min); ratio of AUCs (51)/(52)/(53)/(54)=2.69/0/96.87/0.44.

### EXAMPLE 54

### (R)-3-((4-(2-((R)-4-(hydroxymethyl)-2-oxooxazolidin-3-yl)ethyl)piperidin-1-yl)methyl)-2,3-dihydrobenzo[b][1,4]dioxine-6-carbonitrile

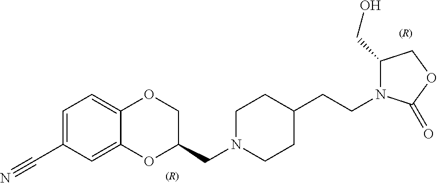

By proceeding as in Example 1 but replacing (R)-2-(bromomethyl)-2,3-dihydrobenzo[b][1,4]dioxine 2.1 by (S)-(7-cyano-2,3-dihydrobenzo[b][1,4]dioxin-2-yl)methyl 4-bromobenzenesulfonate 2.12, 3-(2-(piperidin-4-yl)ethyl)oxazolidin-2-one 3.1 by (R)-4-(hydroxymethyl)-3-(2-(piperidin-4-yl)ethyl)oxazolidin-2-one 3.21, the K₂CO₃/KI pair by diisopropylethylamine (1.4 eq) and acetonitrile by dimethylsulfoxide, 0.7 g (1.7 mmol) of compound 54 are obtained in the form of a pale yellow oil, after purification on silica gel (gradient: dichloromethane=100% to dichloromethane/(methanol:NH₄OH 9:1)=95/5% over 30 min).

Yield: 72%

¹H NMR (CDCl₃) δ: 1.29 (m, 3H); 1.51 (m, 2H); 1.73 (m, 2H); 2.10 (m, 3H); 2.54 (dd, 1H, J=13.4 and 6 Hz); 2.66 (dd, 1H, J=13.6 and 5.6 Hz); 2.87 (m, 1H); 2.95 (m, 1H); 3.16 (m, 1H); 3.53 (m, 1H); 3.69 (dd, 1H, J=11.2 and 3.6 Hz); 3.79 (dd, 1H, J=11.6 and 4.4 Hz); 3.89 (m, 1H); 4.02 (dd, 1H, J=11.4 and 7.2 Hz); 4.22 (dd, 1H, J=8.8 and 5.6 Hz); 4.29 (m, 1H); 4.36 (m, 2H); 6.91 (d, 1H, J=8.4 Hz); 7.14 (dd, 1H, J=8.4 and 2 Hz); 7.17 (d, 1H, J=2 Hz).

0.7 g (1.7 mmol) of compound 54 are dissolved in a 20 ml of ethyl acetate, then 0.40 ml (2 mmol) of a 5 N solution of HCl in isopropanol are added. A white precipitate is formed. This precipitate is filtered and 0.69 g (1.6 mmol) of compound 54 hydrochloride are obtained in the form of a white solid.

F: 247° C.

¹H NMR (DMSOd₆) δ: 1.50 (m, 5H); 1.91 (m, 2H); 3.04 (m, 2H); 3.39 (m, 6H); 3.57 (m, 1H); 3.68 (m, 1H); 3.83 (m, 1H); 4.04 (m, 1H); 4.18 (m, 1H); 4.27 (m, 1H); 4.45 (m, 1H); 5.00 (m, 2H); 7.11 (d, 1H, J=8.4 Hz); 7.38 (d, 1H, J=8.4 Hz); 7.51 (s, 1H).

MS m/z 402 (M+1).

HPLC (XBRIDGE C8, acetonitrile/water/KH₂PO₄ (20/80/6.8 g pH 4), 1 ml/min), retention time=7.13 min, chemical purity=98.81%

HPLC (CHIRACEL OJ-H, heptane/ethanol/diethylamine (80/20/0.1), 1 ml/min); ratio of AUCs (51)/(52)/(53)/(54)=0/2.40/0.46/0/97.14.

### EXAMPLE 55

### (S)-2-((4-(2-((R)-4-(hydroxymethyl)-2-oxooxazolidin-3-yl)ethyl)piperidin-1-yl)methyl)-2,3-dihydrobenzo[b][1,4]dioxine-5-carbonitrile

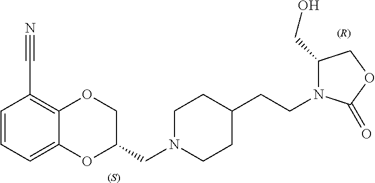

By proceeding as in Example 1 but replacing (R)-2-(bromomethyl)-2,3-dihydrobenzo[b][1,4]dioxine 2.1 by (R)-(5-cyano-2,3-dihydrobenzo[b][1,4]dioxin-2-yl)methyl 4-bromobenzenesulfonate 2.14, 3-(2-(piperidin-4-yl)ethyl)oxazolidin-2-one 3.1 by (R)-4-(hydroxymethyl)-3-(2-(piperidin-4-yl)ethyl)oxazolidin-2-one 3.21, the K₂CO₃/KI pair by diisopropylethylamine (1.4 eq), 0.072 g (0.18 mmol) of compound 55 are obtained in the form of a colorless oil, after purification on silica gel (eluent; dichloromethane/methanol=90/10%).

Yield: 37%

¹H NMR (DMSOd₆) δ: 1.17 (m, 3H); 1.41 (m, 2H); 1.64 (m, 2H); 2.00 (m, 2H); 2.55 (m, 1H); 2.88 (m, 2H); 3.05 (m, 1H); 3.17 (m, 1H); 3.30 (m, 1H); 3.44 (m, 1H); 3.55 (m, 1H); 3.81 (m, 1H); 4.03 (m, 1H); 4.12 (m, 1H); 4.27 (m, 1H); 4.47 (m, 2H); 4.98 (m, 1H); 6.97 (m, 1H); 7.22 (d, 1H, J=8 Hz); 7.28 (d, 1H, J=7.6 Hz).

0.072 g (0.18 mmol) of compound 55 are dissolved in 5 ml of acetonitrile and then 0.016 g (0.18 mmol) of oxalic acid and 1 ml of water are added. After lyophilization, 0.081 g (0.16 mmol) of compound 55 oxalate are recovered in the form of a white solid.

¹H NMR (DMSOd₆); 1.42 (m, 5H); 1.80 (m, 2H); 2.67 (m, 2H); 3.09 (m, 3H); 3.30 (m, 3H); 3.43 (dd, 1H, J=11.6 and 7.2 Hz); 3.56 (dd, 1H, J=11.8 and 3.6 Hz); 3.82 (m, 1H); 4.04 (dd, 1H, J=8.4 and 5.6 Hz); 4.19 (dd, 1H, J=11.6 and 6.8 Hz); 4.27 (m, 1H); 4.49 (dd, 1H, J=11.6 and 2.4 Hz); 4.74 (m, 1H); 7.02 (m, 1H); 7.27 (d, 1H, J=8 Hz); 7.34 (dd, 1H, J=7.8 and 1.6 Hz).

HPLC (X-BRIDGE C18, Phase A: 100% H₂O. 0.1% TFA, Phase B: 100% acetonitrile, gradient B: 0 to 100% in 6 min, 1.8 ml/min), retention time=4.11 min, chemical purity: 99.4%.

### EXAMPLE 56

### (R)-3-(2-(1-(((S)-7-chloro-2,3-dihydrobenzo[b][1,4]dioxin-2-yl)methyl)piperidin-4-yl)ethyl)-4-(hydroxymethyl)oxazolidin-2-one

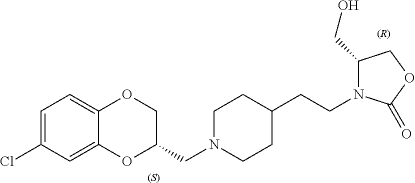

By proceeding as in Example 1 but replacing (R)-2-(bromomethyl)-2,3-dihydrobenzo[b][1,4]dioxine 2.1 by (R)-2-(bromomethyl)-7-chloro-2,3-dihydrobenzo[b][1,4]dioxine 2.7, 3-(2-(piperidin-4-yl)ethyl)oxazolidin-2-one 3.1 by (R)-4-(hydroxymethyl)-3-(2-(piperidin-4-yl)ethyl)oxazolidin-2-one 3.21, K₂CO₃ by Cs₂CO₃ and acetonitrile by dimethylformamide, 0.9 g (2.19 mmol) of compound 56 are obtained in the form of a colorless oil, after purification on silica gel (eluent: dichloromethane/methanol=90/10%).

Yield: 60%

¹H NMR (CDCl₃) δ: 1.29 (m, 3H); 1.50 (m, 2H); 1.72 (m, 3H); 2.08 (m, 2H); 2.53 (dd, 1H, J=13.4 and 6 Hz); 2.63 (dd, 1H, J=13.4 and 5.6 Hz); 2.87 (m, 1H); 2.97 (m, 1H); 3.15 (m, 1H); 3.53 (m, 1H); 3.69 (dd, 1H. J=11.4 and 3.6 Hz); 3.79 (dd, 1H, J=11.4 and 4 Hz); 3.89 (m, 1H); 3.94 (dd, 1H, J=12 and 7.6 Hz); 4.22 (dd, 1H, J=8.8 and 5.6 Hz); 4.27 (m, 2H); 4.36 (m, 1H); 6.78 (m, 2H); 6.88 (m, 1H).

0.9 g (2.19 mmol) of the compound are dissolved in 20 ml of methanol and then 0.19 g (2.11 mmol) of oxalic acid in solution in 10 ml of methanol are added. Several ml of ethyl ether are added and after trituration, 0.81 g (1.62 mmol) of compound 56 oxalate are obtained in the form of a white solid.

¹H NMR (DMSOd₆) δ: 1.4 (m, 5H); 1.82 (m, 2H); 2.70 (m, 2H); 3.09 (m, 3H); 3.33 (m, 3H); 3.43 (dd, 1H, J=11.6 and 3.6 Hz); 3.56 (dd, 1H, J=11.6 and 3.6 Hz); 3.82 (m, 1H); 4.04 (m, 2H); 4.28 (m, 2H); 4.67 (lm, 1H); 6.92 (m, 2H); 7.02 (d, 1H, J=1.6 Hz).

MS m/z 411 (M+1).

HPLC (XBRIDGE C8, acetonitrile/water/KH₂PO₄ (50/50/6.8 g pH 4), 0.4 ml/min), retention time=6.63 min, chemical purity=99.75%

### EXAMPLE 57

### (R)-3-(2-(1-(((S)-7-fluoro-2,3-dihydrobenzo[b][1,4]dioxin-2-yl)methyl)piperidin-4-yl)ethyl)-4-(hydroxymethyl)oxazolidin-2-one

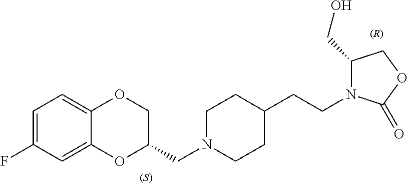

By proceeding as in Example 1 but replacing (R)-2-(bromomethyl)-2,3-dihydrobenzo[b][1,4]dioxine 2.1 by (R)-(7-fluoro-2,3-dihydrobenzo[b][1,4]dioxin-2-yl)methyl 4-bromobenzenesulfonate 2.17, 3-(2-(piperidin-4-yl)ethyl)oxazolidin-2-one 3.1 by (R)-4-(hydroxymethyl)-3-(2-(piperidin-4-yl)ethyl)oxazolidin-2-one 3.21, and K₂CO₃/KI pair by diisopropylethylamine (1.4 eq), 0.097 g (0.25 mmol) of compound 57 are obtained in the form of a colorless oil, after purification on silica gel (eluent: dichloromethane/methanol=90/10%).

Yield: 49%

¹H NMR (DMSOd₆) δ: 1.16 (m, 3H); 1.41 (m, 2H); 1.63 (m, 2H); 1.99 (m, 2H); 2.50 (m, 2H); 2.83 (m, 1H); 2.91 (m, 1H); 3.05 (m, 1H); 3.33 (m, 1H); 3.43 (m, 1H); 3.54 (m, 1H); 3.81 (m, 1H); 3.93 (dd, 1H, J=11.4 and 7.2 Hz); 4.03 (dd, 1H, J=8.4 and 5.6 Hz); 4.27 (m, 2H); 4.35 (m, 1H); 4.98 (t, 1H, J=5.2 Hz); 6.65 (m, 1H); 6.76 (m, 1H); 6.87 (m, 1H).

0.097 g (0.24 mmol) of compound 57 are dissolved in 5 ml of acetonitrile and then 0.022 g (0.24 mmol) of oxalic acid and 1 ml of water are added. After lyophilization, 0.112 g (0.23 mmol) of compound 57 oxalate are recovered in the form of a white solid.

¹H NMR (DMSOd₆); 1.40 (m, 5H); 1.80 (m, 2H); 2.67 (m, 2H); 3.08 (m, 3H); 3.30 (m, 3H); 3.43 (dd, 1H, J=11.8 and 3.6 Hz); 3.56 (dd, 1H, J=11.8 and 3.6 Hz); 3.82 (m, 1H); 4.02 (m, 2H); 4.26 (m, 2H); 4.65 (m, 1H); 6.71 (m, 1H); 6.82 (m, 1H); 6.92 (m, 1H).

MS m/z 395 (M+1).

HPLC (X-BRIDGE C18, Phase A: 100% H₂O. 0.1% TFA, Phase B: 100% acetonitrile, gradient B: 0 to 100% in 6 min then 2 min acetonitrile, 1.8 ml/min) retention time=4.24 min, chemical purity: 100%.

### EXAMPLE 58

### (R)-3-(2-(1-(((S)-6-fluoro-2,3-dihydrobenzo[b][1,4]dioxin-2-yl)methyl)piperidin-4-yl)ethyl)-4-(hydroxymethyl)oxazolidin-2-one

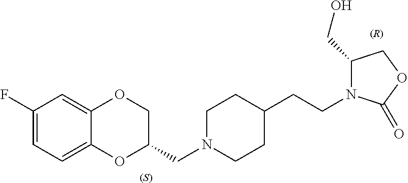

By proceeding as in Example 1 but replacing (R)-2-(bromomethyl)-2,3-dihydrobenzo[b][1,4]dioxine 2.1 by (R)-(6-fluoro-2,3-dihydrobenzo[b][1,4]dioxin-2-yl)methyl 4-bromobenzenesulfonate 2.18, 3-(2-(piperidin-4-yl)ethyl)oxazolidin-2-one 3.1 by (R)-4-(hydroxymethyl)-3-(2-(piperidin-4-yl)ethyl)oxazolidin-2-one 3.21, and the K₂CO₃/KI pair by diisopropylethylamine (1.4 eq), 0.089 g (0.22 mmol) of compound 58 are obtained in the form of a white solid, after purification on silica gel (eluent: dichloromethane/methanol=90/10%).

Yield: 44%

¹H NMR (DMSOd₆) δ: 1.17 (m, 3H); 1.41 (m, 2H); 1.63 (m, 2H); 1.99 (m, 2H); 2.50 (m, 2H); 2.83 (m, 1H); 2.91 (m, 1H); 3.04 (m, 1H); 3.33 (m, 1H); 3.42 (m, 1H); 3.54 (m, 1H); 3.80 (m, 1H); 3.95 (dd, 1H, J=11.8 and 7.6 Hz); 4.03 (dd, 1H, J=8.4 and 5.6 Hz); 4.28 (m, 3H); 4.98 (t, 1H, J=5.2 Hz); 6.65 (m, 1H); 6.77 (m, 1H); 6.86 (m, 1H).

0.089 g (0.22 mmol) of compound 58 are dissolved in 5 ml of acetonitrile and then 0.020 g (0.22 mmol) of oxalic acid and 1 ml of water are added. After lyophilization, 0.103 g (0.21 mmol) of compound 58 oxalate are recovered in the form of a white solid.

¹H NMR (DMSOd₆); 1.31 (m, 3H); 1.43 (m, 2H); 1.77 (m, 2H); 2.52 (m, 2H); 2.95 (m, 2H); 3.07 (m, 1H); 3.16 (m, 1H); 3.24 (m, 1H); 3.33 (m, 1H); 3.42 (dd, 1H, J=12 and 2.8 Hz); 3.56 (dd, 1H, J=11.6 and 3.6 Hz); 3.82 (m, 1H); 4.02 (m, 2H); 4.29 (m, 2H); 4.52 (m, 1H); 6.70 (m, 1H); 6.81 (m, 1H); 6.92 (m, 1H).

MS m/z 395 (M+1).

HPLC (X-BRIDGE C18, Phase A: 100% H₂O. 0.1% TFA, Phase B: 100% acetonitrile, gradient B: 0 to 100% in 6 min then 2 min acetonitrile 1.8 ml/min), retention time=4.31 min, chemical purity: 100%.

### EXAMPLE 59

### (R)-3-(2-(1-(((S)-8-fluoro-2,3-dihydrobenzo[b][1,4]dioxin-2-yl)methyl)piperidin-4-yl)ethyl)-4-(hydroxymethyl)oxazolidin-2-one

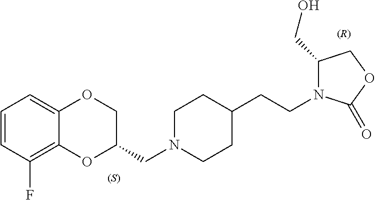

By proceeding as in Example 1 but replacing (R)-2-(bromomethyl)-2,3-dihydrobenzo[b][1,4]dioxine 2.1 by (R)-(8-fluoro-2,3-dihydrobenzo[b][1,4]dioxin-2-yl)methyl 4-bromobenzenesulfonate 2.16, 3-(2-(piperidin-4-yl)ethyl)oxazolidin-2-one 3.1 by (R)-4-(hydroxymethyl)-3-(2-(piperidin-4-yl)ethyl)oxazolidin-2-one 3.21, and K₂CO₃/KI pair by diisopropylethylamine (1.4 eq), 0.079 g (0.20 mmol) of compound 59 are obtained in the form of a colorless oil, after purification on silica gel (eluent: dichloromethane/methanol=90/10%).

Yield: 40%

¹H NMR (DMSOd₆) δ: 1.17 (m, 3H); 1.41 (m, 2H); 1.64 (m, 2H); 2.01 (m, 2H); 2.56 (m, 2H); 2.83 (m, 1H); 2.94 (m, 1H); 3.04 (m, 1H); 3.33 (m 1H); 3.42 (m, 1H); 3.55 (m, 1H); 3.81 (m, 1H); 4.01 (m, 2H); 4.32 (m, 3H); 4.98 (t, 1H, J=5.2 Hz); 6.71 (m, 1H); 6.78 (m, 2H).

0.079 g (0.20 mmol) of compound 59 are dissolved in 5 ml of acetonitrile and then 0.018 g (0.20 mmol) of oxalic acid and 1 ml of water are added. After lyophilization, 0.090 g (0.19 mmol) of compound 59 oxalate are recovered in the form of a white solid.

¹H NMR (DMSOd₆); 1.30 (m, 3H); 1.44 (m, 2H); 1.78 (m, 2H); 2.57 (m, 2H); 3.05 (m, 3H); 3.17 (m, 1H); 3.31 (m, 2H); 3.42 (dd, 1H, J=11.8 and 3.6 Hz); 3.56 (dd, 1H, J=11.8 and 3.6); 3.81 (m, 1H); 4.05 (m, 2H); 4.27 (m, 1H); 4.35 (m, 1H); 4.62 (m, 1H); 6.74 (m, 1H); 6.83 (m, 2H).

MS m/z 395 (M+1).

HPLC (X-BRIDGE C18, Phase A: 100% H₂O. 0.1% TFA, Phase B: 100% acetonitrile, gradient B: 0 to 100% in 6 min then 2 min acetonitrile 1.8 ml/min, retention time=4.23 min, chemical purity: 100%.

### EXAMPLE 60

### (R)-3-(2-(1-(((S)-5,7-difluoro-2,3-dihydrobenzo[b][1,4]dioxin-2-yl)methyl)piperidin-4-yl)ethyl)-4-(hydroxymethyl)oxazolidin-2-one

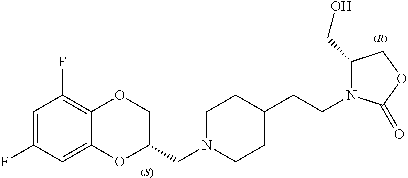

By proceeding as in Example 1 but replacing (R)-2-(bromomethyl)-2,3-dihydrobenzo[b][1,4]dioxine 2.1 by (R)-(5,7-difluoro-2,3-dihydrobenzo[b][1,4]dioxin-2-yl)methyl 4-bromobenzenesulfonate 2.19, 3-(2-(piperidin-4-yl)ethyl)oxazolidin-2-one 3.1 by (R)-4-(hydroxymethyl)-3-(2-(piperidin-4-yl)ethyl)oxazolidin-2-one 3.21, and K₂CO₃/KI pair by diisopropylethylamine (1.4 eq), 0.099 g (0.24 mmol) of compound 60 are obtained in the form of a colorless oil, after purification on silica gel (eluent: dichloromethane/methanol=90/10%).

Yield: 49%

¹H NMR (DMSOd₆) δ: 117 (m, 3H); 1.41 (m, 2H); 1.63 (m, 2H); 1.99 (m, 2H); 2.54 (m, 2H); 2.84 (m, 1H); 2.91 (m, 1H); 3.06 (m, 1H); 3.34 (m, 1H); 3.43 (m, 1H); 3.54 (m, 1H); 3.81 (m, 1H); 4.00 (m, 2H); 4.27 (m, 1H); 4.35 (m, 1H); 4.43 (m, 1H); 4.98 (t, 1H, J=5.2 Hz); 6.69 (m, 1H); 6.84 (m, 1H).

0.099 g (0.24 mmol) of compound 60 are dissolved in 5 ml of acetonitrile and then 0.022 g (0.24 mmol) of oxalic acid and 1 ml of water are added. After lyophilization, 0.115 g (0.23 mmol) of compound 60 oxalate are recovered in the form of a white solid.

¹H NMR (DMSOd₆): 1.33 (m, 3H); 1.44 (m, 2H); 1.77 (m, 2H); 2.52 (m, 2H); 2.97 (m, 2H); 3.07 (m, 1H); 3.19 (m, 2H); 3.34 (m, 1H); 3.43 (dd, 1H, J=11.8 and 3.6 Hz); 3.56 (dd, 1H, J=11.8 and 3.6 Hz); 3.81 (m, 1H); 4.05 (m, 2H); 4.27 (m, 1H); 4.36 (m, 1H); 4.66 (m, 1H); 6.73 (m, 1H); 6.90 (m, 1H).

MS m/z 413 (M+1).

HPLC (X-BRIDGE C18, Phase A: 100% H₂O. 0.1% TFA, Phase B: 100% acetonitrile, gradient B: 0 to 100% in 6 min then 2 min acetonitrile 1.8 ml/min), retention time=4.36 min, chemical purity: 100%.

### EXAMPLE 61

### (R)-3-(2-(1-(((S)-6.7-difluoro-2,3-dihydrobenzo[b][1,4]dioxin-2-yl)methyl)piperidin-4-yl)ethyl)-4-(hydroxymethyl)oxazolidin-2-one

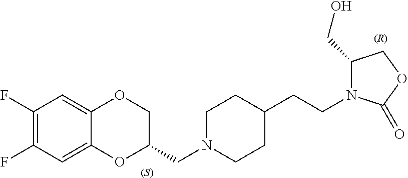

By proceeding as in Example 1 but replacing (R)-2-(bromomethyl)-2,3-dihydrobenzo[b][1,4]dioxine 2.1 by (R)-(6,7-difluoro-2,3-dihydrobenzo[b][1,4]dioxin-2-yl)methyl 4-bromobenzenesulfonate 2.22, 3-(2-(piperidin-4-yl)ethyl)oxazolidin-2-one 3.1 by (R)-4-(hydroxymethyl)-3-(2-(piperidin-4-yl)ethyl)oxazolidin-2-one 3.21, and the K₂CO₃/KI pair by diisopropylethylamine (1.4 eq), 0.066 g (0.16 mmol) of compound 61 are obtained in the form of a colorless oil, after purification on silica gel (eluent: dichloromethane/methanol=90/10%).

Yield: 33%

¹H NMR (DMSOd₆) δ: 1.15 (m, 3H); 1.40 (m, 2H); 1.64 (m, 2H); 1.98 (m, 2H); 2.50 (m, 2H); 2.83 (m, 1H); 2.90 (m, 1H); 3.04 (m, 1H); 3.32 (m, 1H); 3.42 (m, 1H); 3.55 (m, 1H); 3.80 (m, 1H); 3.95 (dd, 1H. J=11.2 and 7.2 Hz); 4.03 (dd, 1H, J=8.4 and 5.6 Hz); 4.29 (m, 3H); 4.98 (t, 1H, J=5.6 Hz); 7.04 (m, 2H).

0.066 g (0.16 mmol) of compound 61 are dissolved in 5 ml of acetonitrile and then 0.014 g (0.16 mmol) of oxalic acid and 1 ml of water are added. After lyophilization, 0.072 g (0.14 mmol) of compound 61 oxalate are recovered in the form of a white solid.

¹H NMR (DMSOd₆): 1.37 (m, 5H); 1.79 (m, 2H); 2.63 (m, 2H); 3.07 (m, 3H); 3.23 (m, 1H); 3.33 (m, 2H); 3.43 (dd, 1H, J=11.8 and 3.6 Hz); 3.56 (dd, 1H, J=11.6 and 4 Hz); 3.82 (m, 1H); 4.02 (m, 2H); 4.26 (m, 2H); 4.63 (m, 1H); 7.09 (m, 2H).

MS m/z 413 (M+1).

HPLC (X-BRIDGE C18, Phase A: 100% H₂O. 0.1% TFA, Phase B: 100% acetonitrile, gradient B: 0 to 100% in 6 min then 2 min acetonitrile 1.8 ml/min), retention time=4.40 min, chemical purity: 98.1%.

### EXAMPLE 62

### (R)-3-(2-(1-(((S)-7-chloro-5-fluoro-2,3-dihydrobenzo[b][1,4]dioxin-2-yl)methyl)piperidin-4-yl)ethyl)-4-(hydroxymethyl)oxazolidin-2-one

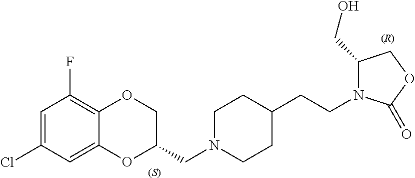

By proceeding as in Example 1 but replacing (R)-2-(bromomethyl)-2,3-dihydrobenzo[b][1,4]dioxine 2.1 by (R)-(7-chloro-5-fluoro-2,3-dihydrobenzo[b][1,4]dioxin-2-yl)methyl 4-bromobenzenesulfonate 2.20, 3-(2-(piperidin-4-yl)ethyl)oxazolidin-2-one 3.1 by (R)-4-(hydroxymethyl)-3-(2-(piperidin-4-yl)ethyl)oxazolidin-2-one 3.21, and the K₂CO₃/KI pair by diisopropylethylamine (1.4 eq), 0.084 g (0.19 mmol) of compound 62 are obtained in the form of a colorless oil, after purification on silica gel (eluent: dichloromethane/methanol=90/10%).

Yield: 42%

¹H NMR (DMSOd₆) δ: 1.15 (m, 3H); 1.41 (m, 2H); 1.64 (m, 2H); 1.99 (m, 2H); 2.54 (m, 2H); 2.84 (m, 1H); 2.91 (m, 1H); 3.05 (m, 1H); 3.33 (m, 1H); 3.42 (m, 1H); 3.55 (m, 1H); 3.81 (m, 1H); 4.02 (m, 2H); 4.27 (m, 1H); 4.37 (m, 1H); 4.44 (m, 1H); 4.98 (t, 1H, J=5.6 Hz); 6.87 (s, 1H); 7.00 (m, 1H).

0.084 g (0.19 mmol) of compound 62 are dissolved in 5 ml of acetonitrile and then 0.017 g (0.19 mmol) of oxalic acid and 1 ml of water are added. After lyophilization, 0.097 g (0.18 mmol) of compound 62 oxalate are recovered in the form of a white solid.

¹H NMR (DMSOd₆): 1.35 (m, 3H); 1.44 (m, 2H); 1.80 (m, 2H); 2.63 (m, 2H); 3.07 (m, 3H); 3.22 (m, 1H); 3.33 (m, 2H); 3.43 (dd, 1H, J=11.8 and 3.6 Hz); 3.56 (dd, 1H, J=11.8 and 3.6 Hz); 3.82 (m, 1H); 4.04 (dd, 1H, J=8.4 and 5.6 Hz); 4.10 (dd, 1H, J=11.6 and 6.4 Hz); 4.25 (m, 1H); 4.38 (m, 1H); 4.72 (m, 1H); 6.92 (m, 1H); 7.07 (m, 1H).

MS m/z 429 (M+1).

HPLC (X-BRIDGE C18, Phase A: 100% H₂O. 0.1% TFA, Phase B: 100% acetonitrile, gradient B: 0 to 100% in 6 min then 2 min acetonitrile, 1.8 ml/min), retention time=4.58 min, chemical purity: 99.5%.

### EXAMPLE 63

### (R)-3-(2-(1-(((S)-5-chloro-7-fluoro-2,3-dihydrobenzo[b][1,4]dioxin-2-yl)methyl)piperidin-4-yl)ethyl)-4-(hydroxymethyl)oxazolidin-2-one

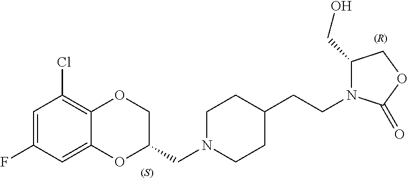

By proceeding as in Example 1 but replacing (R)-2-(bromomethyl)-2,3-dihydrobenzo[b][1,4]dioxine 2.1 by (R)-(5-chloro-7-fluoro-2,3-dihydrobenzo[b][1,4]dioxin-2-yl)methyl 4-bromobenzenesulfonate 2.21, 3-(2-(piperidin-4-yl)ethyl)oxazolidin-2-one 3.1 by (R)-4-(hydroxymethyl)-3-(2-(piperidin-4-yl)ethyl)oxazolidin-2-one 3.21, and the K₂CO₃/KI pair by diisopropylethylamine (1.4 eq), 0.067 g (0.15 mmol) of compound 63 are obtained in the form of a colorless oil, after purification on silica gel (eluent: dichloromethane/methanol=90/10%).

Yield: 34%

¹H NMR (DMSOd₆) δ: 1.17 (m, 3H); 1.41 (m, 2H); 1.63 (m, 2H); 1.99 (m, 2H); 2.53 (m, 2H); 2.84 (m, 1H); 2.91 (m, 1H); 3.05 (m, 1H); 3.33 (m, 1H); 3.43 (m, 1H); 3.53 (m, 1H); 3.80 (m, 1H); 4.02 (m, 2H); 4.27 (m, 1H); 4.39 (m, 2H); 4.98 (t, 1H, J=5.6 Hz); 6.84 (dd, 1H, J=9.6 and 2.8 Hz); 6.97 (dd, 1H, J=8.4 and 2.8 Hz).

0.067 g (0.15 mmol) of compound 63 are dissolved in 5 ml of acetonitrile and then 0.013 g (0.15 mmol) of oxalic acid and 1 ml of water are added. After lyophilization, 0.072 g (0.14 mmol) of compound 63 oxalate are recovered in the form of a white solid.

¹H NMR (DMSOd₆): 1.33 (m, 3H); 1.44 (m, 2H); 1.79 (m, 2H); 2.60 (m, 2H); 3.07 (m, 3H); 3.22 (m, 1H); 3.33 (m, 2H); 3.43 (dd, 1H, J=11.6 and 3.6 Hz); 3.56 (dd, 1H, J=11.6 and 3.6 Hz); 3.81 (m, 1H); 4.04 (dd, 1H, J=8.4 and 5.6 Hz); 4.10 (dd, 1H, J=11.8 and 6.4 Hz); 4.27 (m, 1H); 4.40 (m, 1H); 4.69 (m, 1H); 6.89 (dd, 1H, J=9.6 and 2.8 Hz); 7.04 (dd, 1H, J=8.6 and 2.8 Hz).

MS m/z 429 (M+1).

HPLC (X-BRIDGE C18, Phase A: 100% H₂O. 0.1% TFA, Phase B: 100% acetonitrile, gradient B: 0 to 100% in 6 min then 2 min acetonitrile 1.8 ml/min), retention time=4.54 min, chemical purity: 100%.

### EXAMPLE 64

### (R)-4-(hydroxymethyl)-3-(2-(1-(((S)-7-(methylsulfonyl)-2,3-dihydrobenzo[b][1,4]dioxin-2-yl)methyl)piperidin-4-yl)ethyl)oxazolidin-2-one

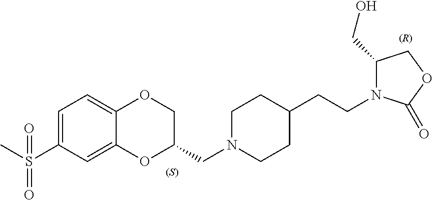

By proceeding as in Example 1 but replacing (R)-2-(bromomethyl)-2,3-dihydrobenzo[b][1,4]dioxine 2.1 by (R)-(7-(methylsulfonyl)-2,3-dihydrobenzo[b][1,4]dioxin-2-yl)methyl 4-bromobenzenesulfonate 2.23, 3-(2-(piperidin-4-yl)ethyl)oxazolidin-2-one 3.1 by (R)-4-(hydroxymethyl)-3-(2-(piperidin-4-yl)ethyl)oxazolidin-2-one 3.21, and the K₂CO₃/KI pair by diisopropylethylamine (1.4 eq), 0.068 g (0.15 mmol) of compound 64 are obtained in the form of a white foam, after purification on silica gel (eluent: dichloromethane/methanol=90/10%).

Yield: 52%

¹H NMR (DMSOd₆) δ: 1.17 (m, 3H); 1.41 (m, 2H); 1.65 (m, 2H); 2.00 (m, 2H); 2.55 (m, 2H); 2.85 (m, 1H); 2.93 (m, 1H); 3.04 (m, 1H); 3.16 (s, 3H); 3.33 (m, 1H); 3.43 (m, 1H); 3.54 (m, 1H); 3.81 (m, 1H); 4.05 (m, 2H); 4.27 (m, 1H); 4.40 (m, 2H); 4.98 (t, 1H, J=5.2 Hz); 7.10 (d, 1H, J=8.4 Hz); 7.37 (m, 2H).

0.068 g (0.15 mmol) of compound 64 are dissolved in 5 ml of acetonitrile and then 0.013 g (0.15 mmol) of oxalic acid and 1 ml of water are added. After lyophilization, 0.075 g (0.14 mmol) of compound 64 oxalate are recovered in the form of a white solid.

¹H NMR (DMSOd₆): 1.35 (m, 3H); 1.45 (m, 2H); 1.78 (m, 2H); 2.62 (m, 2H); 3.08 (m, 3H); 3.17 (s, 3H); 3.23 (m, 1H); 3.34 (m, 2H); 3.43 (dd, 1H, J=11.8 and 3.2 Hz); 3.56 (dd, 1H, J=11.8 and 3.2 Hz); 3.82 (m, 1H); 4.04 (dd, 1H, J=8.4 and 5.6 Hz); 4.12 (dd, 1H, J=11.6 and 6.8 Hz); 4.27 (m, 1H); 4.41 (m, 1H); 4.69 (m, 1H); 7.15 (d, 1H, J=8.4 Hz); 7.43 (m, 2H).

MS m/z 455 (M+1).

HPLC (X-BRIDGE C18, Phase A: 100% H₂O. 0.1% TFA, Phase B: 100% acetonitrile, gradient B: 0 to 100% in 6 min then 2 min acetonitrile, 1.8 ml/min), retention time=3.82 min, chemical purity: 100%.

### EXAMPLE 65

### (S)-methyl 3-((4-(2-((R)-4-(hydroxymethyl)-2-oxooxazolidin-3-yl)ethyl)piperidin-1-yl)methyl)-2,3-dihydrobenzo[b][1,4]dioxine-6-carboxylate

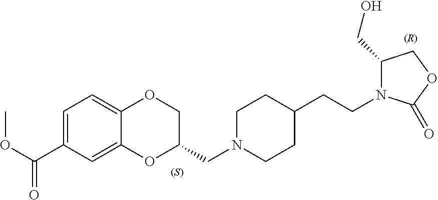

By proceeding as in Example 1 but replacing (R)-2-(bromomethyl)-2,3-dihydrobenzo[b][1,4]dioxine 2.1 by (R)-methyl 3-((((4-bromophenyl)sulfonyl)oxy)methyl)-2,3-dihydrobenzo[b][1,4]dioxine-6-carboxylate 2.24, 3-(2-(piperidin-4-yl)ethyl)oxazolidin-2-one 3.1 by (R)-4-(hydroxymethyl)-3-(2-(piperidin-4-yl)ethyl)oxazolidin-2-one 3.21, and the K₂CO₃/KI pair by diisopropylethylamine (1.4 eq), 0.046 g (0.10 mmol) of compound 65 are obtained in the form of a white solid, after purification on silica gel (eluent: dichloromethane/methanol=90/10%).

Yield: 23%

¹H NMR 1.16 (m, 3H); 1.40 (m, 2H); 1.65 (m, 2H); 2.00 (m, 2H); 2.54 (m, 2H); 2.83 (m, 1H); 2.93 (m, 1H); 3.05 (m, 1H); 3.35 (m, 1H); 3.42 (m, 1H); 3.55 (m, 1H); 3.80 (m, 4H); 4.04 (m, 2H); 4.27 (m, 1H); 4.37 (m, 2H); 4.98 (t, 1H, J=5.2 Hz); 6.98 (d, 1H, J=8.4 Hz); 7.39 (d, 1H, J=2 Hz); 7.46 (dd, 1H, J=8.4 and 2 Hz).

0.046 g (0.10 mmol) of compound 65 are dissolved in 5 ml of acetonitrile and then 0.009 g (0.10 mmol) of oxalic acid and 1 ml of water are added. After lyophilization, 0.047 g (0.09 mmol) of compound 65 oxalate are recovered in the form of a white solid.

¹H NMR (DMSOd₆): 1.40 (m, 5H); 1.82 (m, 2H); 2.67 (m, 2H); 3.08 (m, 3H); 3.33 (m, 3H); 3.43 (dd, 1H, J=11.8 and 3.6 Hz); 3.56 (dd, 1H, J=11.8 and 3.6 Hz); 3.81 (m, 4H); 4.04 (dd, 1H, J=8.4 and 5.6 Hz); 4.10 (dd, 1H, J=11.6 and 6.8 Hz); 4.27 (m, 1H); 4.39 (m, 1H); 4.67 (m, 1H); 7.02 (m, 1H); 7.50 (m, 2H).

MS m/z 435 (M+1).

HPLC (X-BRIDGE C18, Phase A: 100% H₂O. 0.1% TFA, Phase B: 100% acetonitrile, gradient B: 0 to 100% in 6 min then 2 min acetonitrile 1.8 ml/min), retention time=4.31 min, chemical purity: 98.5%.

### EXAMPLE 66

### (R)-3-(2-(1-(((S)-2,3-dihydrobenzo[b][1,4]dioxin-2-yl)methyl)piperidin-4-yl)ethyl)-4-(phenoxymethyl)oxazolidin-2-one

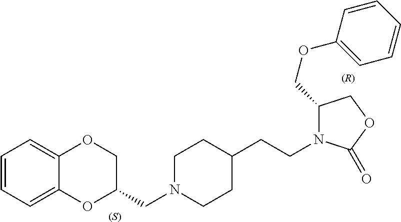

By proceeding as in Example 1 but replacing 3-(2-(piperidin-4-yl)ethyl)oxazolidin-2-one 3.1 by (R)-4-(phenoxymethyl)-3-(2-(piperidin-4-yl)ethyl)oxazolidin-2-one 3.13, 1.6 g (3.53 mmol) of compound 66 are obtained in the form of a colorless oil, after purification on silica gel (eluent: dichloromethane=100% then dichloromethane/methanol=95/5%).

Yield: 67%

¹H NMR (CDCl₃) δ: 1.28 (m, 3H); 1.54 (m, 2H); 1.69 (m, 2H); 2.07 (m, 2H); 2.53 (dd, 1H, J=13.2 and 6 Hz); 2.64 (dd, 1H, J=13.2 and 5.6 Hz); 2.86 (m, 1H); 2.98 (m, 1H); 3.21 (m, 1H); 3.54 (m, 1H); 3.96 (dd, 1H, J=11.7 and 7.7 Hz); 4.04 (m, 2H); 4.19 (m, 2H); 4.35 (m, 2H); 4.45 (m, 1H); 6.85 (m, 6H); 7.01 (m, 1H); 7.31 (m, 2H).

1.6 g (3.53 mmol) of compound 66 are dissolved in 10 ml of methanol and then 0.31 g (3.53 mmol) of oxalic acid in solution in methanol are added. This precipitate is filtered and 1.6 g (2.95 mmol) of compound 66 hydrochloride are obtained in the form of a white solid.

F: 164° C.

¹H NMR (DMSOd₆) δ: 1.38 (m, 5H); 1.77 (m, 2H); 2.67 (m, 2H); 3.13 (m, 3H); 3.24 (m, 1H); 3.35 (m, 2H); 3.99 (dd, 1H, J=11.5 and 6.7 Hz); 4.08 (m, 1H); 4.15 (m, 3H); 4.28 (m, 1H); 4.41 (m, 1H); 4.62 (m, 1H); 6.87 (m, 4H); 6.95 (m, 3H); 7.31 (m, 2H).

MS m/z 453 (M+1).

HPLC (XBRIDGE C8, acetonitrile/water/KH₂PO₄ (35/65/6.8 g pH 4), 1 ml/min), retention time=11.35 min, chemical purity=99.41%

### EXAMPLE 67

### (S)-3-(2-(1-(((S)-2,3-dihydrobenzo[b][1,4]dioxin-2-yl)methyl)piperidin-4-yl)ethyl)-4-(phenoxymethyl)oxazolidin-2-one

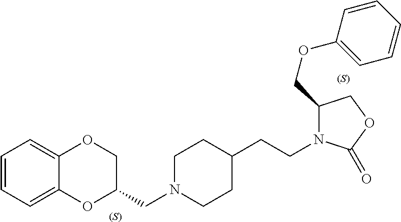

By proceeding as in Example 1 but replacing 3-(2-(piperidin-4-yl)ethyl)oxazolidin-2-one 3.1 by (S)-4-(phenoxymethyl)-3-(2-(piperidin-4-yl)ethyl)oxazolidin-2-one 3.14, 0.5 g (1.10 mmol) of compound 67 are obtained in the form of a colorless oil, after purification on silica gel (eluent: dichloromethane=100% then dichloromethane/methanol=95/5%).

Yield: 45%

¹H NMR (CDCl₃) δ: 1.27 (m, 3H); 1.51 (m, 2H); 1.69 (m, 2H); 2.03 (m, 1H); 2.11 (m, 1H); 2.55 (m, 1H); 2.65 (m, 1H); 2.87 (m, 1H); 2.97 (m, 1H); 3.21 (m, 1H); 3.56 (m, 1H); 3.96 (m, 1H); 4.04 (m, 2H); 4.19 (m, 2H); 4.29 (m, 2H); 4.45 (m, 1H); 6.85 (m, 6H); 7.01 (m, 1H); 7.31 (m, 2H).

0.5 g (1.10 mmol) of compound 67 are dissolved in 10 ml of methanol and then 0.099 g (1.10 mmol) of oxalic acid in solution in methanol are added. Ethyl ether is added and after trituration, 0.52 g (0.96 mmol) of compound 67 oxalate are obtained in the form of a white solid.

F: 172° C.

¹H NMR (DMSOd₆) δ: 1.38 (m, 5H); 1.77 (m, 2H); 2.65 (m, 2H); 3.22 (m, 6H); 3.99 (dd, 1H, J=11.6 and 3.2 Hz); 4.06 (dd, 1H, J=10 and 6.8 Hz); 4.17 (m, 3H); 4.28 (m, 1H); 4.41 (m, 1H); 4.60 (m, 1H); 6.87 (m, 4H); 6.97 (m, 3H); 7.31 (m, 2H).

MS m/z 453 (M+1).

HPLC (XBRIDGE C8, acetonitrile/water/KH₂PO₄ (35/65/6.8 g pH 4), 1 ml/min), retention time=11.31 min, chemical purity=99.40%

### EXAMPLE 68

### S)-3-((4-(2-((R)-2-oxo-4-(phenoxymethyl)oxazolidin-3-yl)ethyl)piperidin-1-yl)methyl)-2,3-dihydrobenzo[b][1,4]dioxine-6-carbonitrile

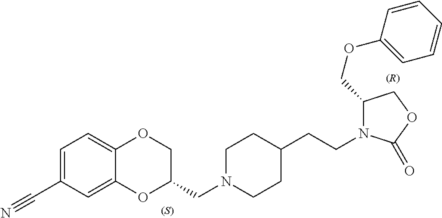

By proceeding as in Example 1 but replacing (R)-2-(bromomethyl)-2,3-dihydrobenzo[b][1,4]dioxine 2.1 by (R)-3-(bromomethyl)-2,3-dihydrobenzo[b][1,4]dioxine-6-carbonitrile 2.11, 3-(2-(piperidin-4-yl)ethyl)oxazolidin-2-one 3.1 by (R)-4-(phenoxymethyl)-3-(2-(piperidin-4-yl)ethyl)oxazolidin-2-one 3.13, K₂CO₃ by Cs₂CO₃ and acetonitrile by dimethylformamide, 0.19 g (0.40 mmol) of compound 68 are obtained in the form of a pale yellow oil, after purification on silica gel (eluent: n-heptane=100% then dichloromethane/methanol=95/5%).

Yield: 42%

¹H NMR (CDCl₃) δ: 1.26 (m, 3H); 1.55 (m, 2H); 1.71 (m, 2H); 2.09 (m, 2H); 2.53 (dd, 1H, J=13.2 and 6.4 Hz); 2.64 (dd, 1H, J=13.4 and 5.6 Hz); 2.84 (m, 1H); 2.94 (m, 1H); 3.22 (m, 1H); 3.56 (m, 1H); 4.02 (m, 3H); 4.20 (m, 3H); 4.36 (m, 1H); 4.56 (m, 1H); 6.90 (m, 3H); 7.00 (m, 1H); 7.14 (m, 1H); 7.31 (m, 2H); 8.00 (m, 1H).

0.19 g (0.40 mmol) of compound 68 are dissolved in 5 ml of methanol and then 0.036 g (0.40 mmol) of oxalic acid in solution in 5 ml of methanol are added. A little ethyl ether is added, the medium is triturated and 0.13 g (0.23 mmol) of compound 68 oxalate are obtained in the form of a white solid.

F=139° C.

¹H NMR (DMSOd₆) δ: 1.36 (5H); 1.75 (m, 2H); 2.57 (m, 2H); 3.18 (m, 6H); 4.12 (m, 5H); 4.41 (m, 2H); 4.65 (m, 1H); 6.97 (m, 3H); 7.08 (m, 1H); 7.33 (m, 3H); 7.44 (s, 1H).

MS m/z 478 (M+1).

HPLC (XBRIDGE C8, acetonitrile/water/KH₂PO₄ (35/65/6.8 g pH 4), 0.4 ml/min), retention time=6.36 min, chemical purity=99.12%

### EXAMPLE 69

### (R)-3-(2-(1-(((S)-7-bromo-2,3-dihydrobenzo[b][1,4]dioxin-2-yl)methyl)piperidin-4-yl)ethyl)-4-(phenoxymethyl)oxazolidin-2-one

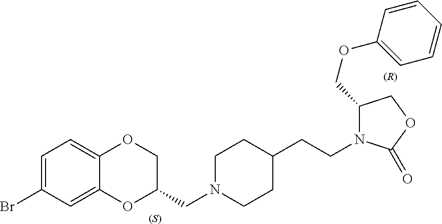

By proceeding as in Example 1 but replacing (R)-2-(bromomethyl)-2,3-dihydrobenzo[b][1,4]dioxine 2.1 by (R)-7-bromo-2-(bromomethyl)-2,3-dihydrobenzo[b][1,4]dioxine 2.5 and 3-(2-(piperidin-4-yl)ethyl)oxazolidin-2-one 3.1 by (R)-4-(phenoxymethyl)-3-(2-(piperidin-4-yl)ethyl)oxazolidin-2-one 3.13, K₂CO₃ by Cs₂CO₃ and acetonitrile by dimethylformamide, 0.40 g (0.75 mmol) of compound 69 are obtained in the form of a pale yellow oil, after purification by flash chromatography on silica gel (eluent: n-heptane=100% then dichloromethane/methanol=95/5%).

Yield: 51%

¹H NMR (CDCl₃) δ: 1.27 (m, 3H); 1.54 (m, 2H); 1.70 (m, 2H); 2.08 (m, 2H); 2.52 (dd, 1H, J=13.4 and 6 Hz); 2.62 (dd, 1H, J=13.2 and 5.6 Hz); 2.84 (m, 1H); 2.94 (m, 1H); 3.21 (m, 1H); 3.55 (m, 1H); 3.94 (dd, 1H, J=11.6 and 7.6 Hz); 4.04 (m, 2H); 4.22 (m, 4H); 4.45 (m, 1H); 6.72 (m, 1H); 6.92 (m, 3H); 7.02 (m, 2H); 7.31 (m, 2H).

0.40 g (0.75 mmol) of compound 69 are dissolved in 5 ml of methanol and then 0.068 g (0.75 mmol) of oxalic acid in solution in 5 ml of methanol are added. This is concentrated and then a little acetone is added, the medium is triturated and 0.31 g (0.50 mmol) of compound 69 oxalate are obtained in the form of a white solid.

F=115° C.

¹H NMR (DMSOd₆) δ: 1.38 (m, 5H); 1.77 (m, 2H); 2.67 (m, 2H); 3.15 (m, 4H); 3.37 (m, 2H); 4.04 (m, 2H); 4.17 (m, 3H); 4.29 (m, 1H); 4.41 (m, 1H); 4.65 (m, 1H); 6.87 (d, 1H, J=8.8 Hz); 6.97 (m, 3H); 7.03 (dd, 1H, J=8.6 and 2.4 Hz); 7.13 (d, 1H, J=2.4 Hz); 7.31 (m, 2H).

MS m/z 531-533 (M+1)

HPLC (XBRIDGE C8, acetonitrile/water/KH₂PO₄ (40/60/6.8 g pH 4), 0.4 ml/min), retention time=7.13 min, chemical purity=98.17%

### EXAMPLE 70

### (R)-3-(2-(1-(((S)-7-fluoro-2,3-dihydrobenzo[b][1,4]dioxin-2-yl)methyl)piperidin-4-yl)ethyl)-4-(phenoxymethyl)oxazolidin-2-one

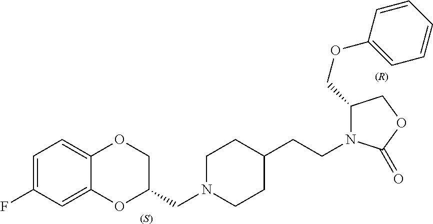

By proceeding as in Example 1 but replacing (R)-2-(bromomethyl)-2,3-dihydrobenzo[b][1,4]dioxine 2.1 by (R)-7-fluoro-2-(bromomethyl)-2,3-dihydrobenzo[b][1,4]dioxine 2.17 and 3-(2-(piperidin-4-yl)ethyl)oxazolidin-2-one 3.1 by (R)-4-(phenoxymethyl)-3-(2-(piperidin-4-yl)ethyl)oxazolidin-2-one 3.13, K₂CO₃ by Cs₂CO₃ and acetonitrile by dimethylformamide, 0.50 g (1.06 mmol) of compound 70 are obtained in the form of a pale yellow oil, after purification by flash chromatography on silica gel (eluent: n-heptane=100% then dichloromethane/methanol=95/5%).

Yield: 32%

¹H NMR (CDCl₃) δ: 1.27 (m, 3H); 1.55 (m, 2H); 1.70 (m, 2H); 2.09 (m, 2H); 2.52 (dd, 1H, J=13.4 and 6 Hz); 2.62 (dd, 1H, J=13.2 and 5.6 Hz); 2.85 (m, 1H); 2.96 (m, 1H); 3.22 (m, 1H); 3.56 (m, 1H); 3.92 (dd, 1H, J=11.4 and 7.2 Hz); 4.04 (m, 2H); 4.20 (m, 4H); 4.45 (m, 1H); 6.53 (m, 1H); 6.61 (m, 1H); 6.78 (m, 1H); 6.89 (m, 2H); 7.01 (m, 1H); 7.29 (m, 2H).

0.25 g (0.53 mmol) of compound 70 are dissolved in 5 ml of methanol and then 0.048 g (0.53 mmol) of oxalic acid in solution in 5 ml of methanol are added. This is concentrated and then a little ether and acetone are added, the medium is triturated and 0.22 g (0.39 mmol) of compound 70 oxalate are obtained in the form of a white solid.

F=103° C.

¹H NMR (DMSOd₆) δ: 1.29 (m, 2H); 1.44 (m, 3H); 1.75 (m, 2H); 2.60 (m, 2H); 3.02 (m, 2H); 3.18 (m, 2H); 3.33 (m, 2H); 3.99 (m, 1H); 4.06 (m, 1H); 4.17 (m, 3H); 4.27 (m, 1H); 4.41 (m, 1H); 4.61 (m, 1H); 6.71 (m, 1H); 6.82 (m, 1H); 6.94 (m, 4H); 7.31 (m, 2H).

MS m/z 471 (M+1).

HPLC (XBRIDGE C8, acetonitrile/water/KH₂PO₄ (40/60/6.8 g pH 4), 0.4 ml/min), retention time=5.20 min, chemical purity=92.96%

### EXAMPLE 71

### (R)-3-(2-(1-(((S)-6-fluoro-2,3-dihydrobenzo[b][1,4]dioxin-2-yl)methyl)piperidin-4-yl)ethyl)-4-(phenoxymethyl)oxazolidin-2-one

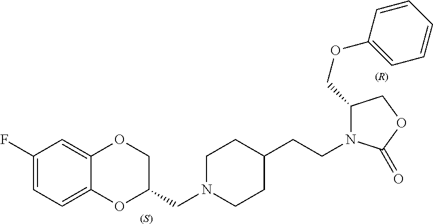

By proceeding as in Example 1 but replacing (R)-2-(bromomethyl)-2,3-dihydrobenzo[b][1,4]dioxine 2.1 by (R)-6-fluoro-2-(bromomethyl)-2,3-dihydrobenzo[b][1,4]dioxine 2.18, 3-(2-(piperidin-4-yl)ethyl)oxazolidin-2-one 3.1 by (R)-4-(phenoxymethyl)-3-(2-(piperidin-4-yl)ethyl)oxazolidin-2-one 3.13, K₂CO₃ by Cs₂CO₃ and acetonitrile by dimethylformamide, 0.40 g (0.85 mmol) of compound 71 are obtained in the form of a pale yellow oil, after purification by flash chromatography on silica gel (eluent: n-heptane=100% then dichloromethane/methanol=95/5%).

Yield: 46%

¹H NMR (CDCl₃) δ: 1.28 (m, 3H); 1.54 (m, 2H); 1.70 (m, 2H); 2.07 (m, 2H); 2.51 (dd, 1H, J=13.4 and 6 Hz); 2.63 (dd, 1H, J=13.4 and 6 Hz); 2.85 (m, 1H); 2.96 (m, 1H); 3.21 (m, 1H); 3.56 (m, 1H); 3.94 (dd, 1H, J=11.2 and 7.2 Hz); 4.04 (m, 2H); 4.19 (m, 3H); 4.29 (m, 1H); 4.45 (m, 1H); 6.55 (m, 1H); 6.61 (m, 1H); 6.79 (m, 1H); 6.89 (m, 2H); 7.01 (m, 1H); 7.31 (m, 2H).

0.40 g (0.85 mmol) of compound 71 are dissolved in 5 ml of methanol and then 0.077 g (0.85 mmol) of oxalic acid in solution in 5 ml of methanol are added. This is concentrated and then a little ether and acetone are added, the medium is triturated and 0.31 g (0.55 mmol) of compound 71 oxalate are obtained in the form of a white solid.

F=115° C.

¹H NMR (DMSOd₆) δ: 1.38 (m, 5H); 1.77 (m, 2H); 2.67 (m, 2H); 3.06 (m, 2H); 3.20 (m, 2H); 3.36 (m, 2H); 4.03 (m, 2H); 4.17 (m, 3H); 4.31 (m, 1H); 4.41 (m, 1H); 4.59 (m, 1H); 6.70 (m, 1H); 6.82 (m, 1H); 6.95 (m, 4H); 7.31 (m, 2H).

MS m/z 471 (M+1).

HPLC (XBRIDGE C8, acetonitrile/water/KH₂PO₄ (40/60/6.8 g pH 4), 0.4 ml/min), retention time=5.26 min, chemical purity=98.86%

### EXAMPLE 72

### (R)-3-(2-(1-(((S)-6-hydroxy-2,3-dihydrobenzo[b][1,4]dioxin-2-yl)methyl)piperidin-4-yl)ethyl)-4-(phenoxymethyl)oxazolidin-2-one

### Step 1: (R)-3-(2-(1-(((S)-6-(benzyloxy)-2,3-dihydrobenzo[b][1,4]dioxin-2-yl)methyl)piperidin-4-yl)ethyl)-4-(phenoxymethyl)oxazolidin-2-one

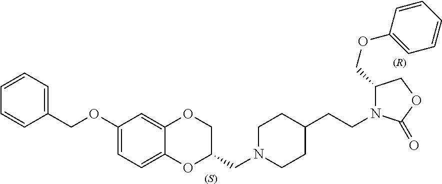

By proceeding as in Example 1 but replacing (R)-2-(bromomethyl)-2,3-dihydrobenzo[b][1,4]dioxine 2.1 by (R)-(6-(benzyloxy)-7-bromo-2,3-dihydrobenzo[b][1,4]dioxin-2-yl)methyl methanesulfonate 2.26 and 3-(2-(piperidin-4-yl)ethyl)oxazolidin-2-one 3.1 by (R)-4-(phenoxymethyl)-3-(2-(piperidin-4-yl)ethyl)oxazolidin-2-one 3.13, 0.80 g (1.25 mmol) of compound (R)-3-(2-(1-(((S)-6-(benzyloxy)-2,3-dihydrobenzo[b][1,4]dioxin-2-yl)methyl)piperidin-4-yl)ethyl)-4-(phenoxymethyl)oxazolidin-2-one are obtained in the form of a clear oil, after purification by flash chromatography on silica gel (eluent; dichloromethane/methanol=95/5%).

Yield: 38%

¹H NMR (CDCl₃) δ: 1.28 (m, 3H); 1.55 (m, 2H); 1.70 (m, 2H); 2.06 (m, 2H); 2.50 (m, 1H); 2.60 (m, 1H); 2.84 (m, 1H); 2.95 (m, 1H); 3.21 (m, 1H); 3.55 (m, 1H); 3.92 (m, 1H); 4.04 (m, 2H); 4.19 (m, 4H); 4.46 (m, 1H); 5.04 (s, 2H); 6.51 (s, 1H); 6.88 (m, 2H); 7.01 (m, 1H); 7.09 (s, 1H); 7.31 (m, 3H); 7.38 (m, 2H); 7.46 (m, 2H).

### Step 2: (R)-3-(2-(1-(((S)-6-hydroxy-2,3-dihydrobenzo[b][1,4]dioxin-2-yl)methyl)piperidin-4-yl)ethyl)-4-(phenoxymethyl)oxazolidin-2-one

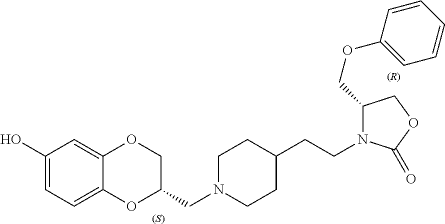

1 g (1.57 mmol) of the preceding compound (R)-3-(2-(1-(((S)-6-(benzyloxy)-7-bromo-2,3-dihydrobenzo[b][1,4]dioxin-2-yl)methyl)piperidin-4-yl)ethyl)-4-(phenoxymethyl)oxazolidin-2-one is solubilized in a mixture of methanol/dichloromethane=5/5 ml. A catalytic quantity of 5% Pd on carbon is added, and hydrogenation is performed at RT for 2 hours. The medium is then filtered on Celite and concentrated. 0.4 g (0.85 mmol) of product 72 are obtained in the form of a white solid, after purification on silica gel (eluent: dichloromethane/methanol=95/5%).

Yield: 54%

¹H NMR (CDCl₃) δ: 1.49 (m, 5H); 1.78 (m, 2H); 2.30 (m, 2H); 2.73 (ls, 2H); 3.03 (m, 1H); 3.27 (m, 2H); 3.52 (m, 1H); 3.95 (m, 1H); 4.04 (m, 2H); 4.20 (m, 3H); 4.46 (m, 2H); 6.35 (m, 1H); 6.40 (s, 1H); 6.69 (m, 1H); 6.88 (m, 2H); 7.01 (m, 1H); 7.31 (m, 2H).

0.30 g (0.64 mmol) of compound 72 are dissolved in 5 ml of methanol and then 0.058 g (0.64 mmol) of oxalic acid in solution in 5 ml of methanol are added. This is concentrated and then a little ether and acetone are added, the medium is triturated and 0.20 g (0.36 mmol) of compound 72 oxalate are obtained in the form of a white solid.

F=131° C.

¹H NMR (DMSOd₆) δ: 1.40 (m, 5H); 1.79 (m, 2H); 2.76 (m, 2H); 3.16 (m, 2H); 3.37 (m, 4H); 3.94 (m, 1H); 4.07 (m, 1H); 4.18 (m, 4H); 4.41 (m, 1H); 4.54 (m, 1H); 6.28 (m, 2H); 6.71 (m, 1H); 6.97 (m, 3H); 7.31 (m, 2H).

MS m/z 469 (M+1).

HPLC (XBRIDGE C8, acetonitrile/water/KH₂PO₄ (35/65/6.8 g pH 4), 0.4 ml/min), retention time=4.01 min, chemical purity=98.69%

### EXAMPLE 73

### (R)-3-(2-(1-(((S)-2,3-dihydrobenzo[b][1,4]dioxin-2-yl)methyl)piperidin-4-yl)ethyl)-4-((4-fluorophenoxy)methyl)oxazolidin-2-one

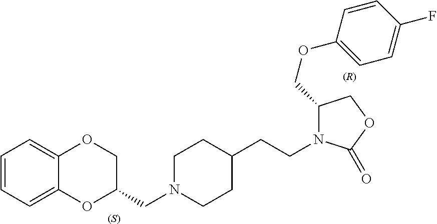

By proceeding as in Example 1 but replacing 3-(2-(piperidin-4-yl)ethyl)oxazolidin-2-one 3.1 by (R)-4-((4-fluorophenoxy)methyl)oxazolidin-2-one 3.15, K₂CO₃ by Cs₂CO₃ and acetonitrile by dimethylformamide, 0.24 g (0.51 mmol) of compound 73 are obtained in the form of a pale yellow oil, after purification by flash chromatography on silica gel (eluent: dichloromethane/methanol=95/5%).

Yield: 51%

¹H NMR (CDCl₃) δ: 1.29 (m, 3H); 1.53 (m, 2H); 1.70 (m, 2H); 2.09 (m, 2H); 2.54 (dd, 1H, J=13.4 and 6 Hz); 2.64 (dd, 1H, J=13.2 and 5.6 Hz); 2.87 (m, 1H); 2.98 (m, 1H); 3.19 (m, 1H); 3.55 (m, 1H); 3.97 (m, 3H); 4.18 (m, 2H); 4.29 (m, 2H); 4.45 (m, 1H); 6.85 (m, 6H); 7.00 (m, 2H).

0.24 g (0.51 mmol) of compound 73 are dissolved in 5 ml of methanol and then 0.046 g (0.51 mmol) of oxalic acid in solution in 5 ml of methanol are added. This is concentrated and then a little acetone is added, the medium is triturated and 0.15 g (0.27 mmol) of compound 73 oxalate are obtained in the form of a white solid.

F=114° C.

¹H NMR (DMSOd₆) δ: 1.38 (m, 5H); 1.77 (m, 2H); 2.65 (m, 2H); 3.12 (m, 3H); 3.24 (m, 1H); 3.33 (m, 2H); 4.02 (m, 2H); 4.16 (m, 3H); 4.29 (m, 1H); 4.40 (m, 1H); 4.60 (m, 1H); 6.87 (m, 4H); 6.97 (m, 2H); 7.14 (m, 2H).

MS m/z 471 (M+1).

HPLC (XBRIDGE C8, acetonitrile/water/KH₂PO₄ (40/60/6.8 g pH 4), 0.4 ml/min), retention time=5.30 min, chemical purity=96.29%

### EXAMPLE 74

### (R)-3-(2-(1-(((S)-2,3-dihydrobenzo[b][1,4]dioxin-2-yl)methyl)piperidin-4-yl)ethyl)-4-((3-fluorophenoxy)methyl)oxazolidin-2-one

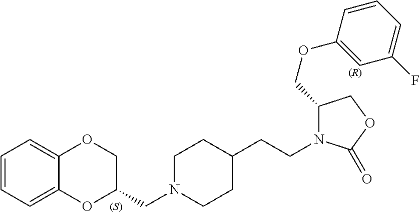

By proceeding as in Example 1 but replacing 3-(2-(piperidin-4-yl)ethyl)oxazolidin-2-one 3.1 by (R)-4-((3-fluorophenoxy)methyl)oxazolidin-2-one 3.16, K₂CO₃ by Cs₂CO₃ and acetonitrile by dimethylformamide, 0.6 g (1.27 mmol) of compound 74 are obtained in the form of a pale yellow oil, after purification by flash chromatography on silica gel (eluent: n-heptane=100% then dichloromethane/methanol=95/5%).

Yield: 43%

¹H NMR (CDCl₃) δ: 1.29 (m, 3H); 1.53 (m, 2H); 1.69 (m, 2H); 2.08 (m, 2H); 2.54 (dd, 1H, J=13.2 and 6 Hz); 2.64 (dd, 1H, J=13.4 and 6 Hz); 2.87 (m, 1H); 2.98 (m, 1H); 3.20 (m, 1H); 3.56 (m, 1H); 3.96 (dd, 1H, J=11.6 and 8 Hz); 4.02 (m, 2H); 4.18 (m, 2H); 4.29 (m, 2H); 4.45 (m, 1H); 6.60 (m, 1H); 6.64 (m, 1H); 6.71 (m, 1H); 6.85 (m, 4H); 7.26 (m, 1H).

0.59 g (1.25 mmol) of compound 74 are dissolved in 5 ml of methanol and then 0.11 g (1.25 mmol) of oxalic acid in solution in 5 ml of methanol are added. This is concentrated and a little ethyl ether is added, the medium is triturated and 0.47 g (0.84 mmol) of compound 74 oxalate are obtained in the form of a white solid.

F=136° C.

¹H NMR (DMSOd₆) δ: 1.36 (m, 5H); 1.77 (m, 2H); 2.67 (m, 2H); 3.12 (m, 3H); 3.23 (m, 1H); 3.35 (m, 2H); 3.99 (dd, 1H, J=11.6 and 6.8 Hz); 4.11 (m, 2H); 4.20 (m, 2H); 4.28 (m, 1H); 4.42 (m, 1H); 4.61 (m, 1H); 6.85 (m, 7H); 7.33 (m, 1H).

MS m/z 471 (M+1).

HPLC (XBRIDGE C8, acetonitrile/water/KH₂PO₄ (35/65/6.8 g pH 4), 0.4 ml/min), retention time=10.35 min, chemical purity=98.56%

### EXAMPLE 75

### (R)-3-(2-(1-(((S)-2,3-dihydrobenzo[b][1,4]dioxin-2-yl)methyl)piperidin-4-yl)ethyl)-4-((3,4-dimethoxyphenoxy)methyl)oxazolidin-2-one

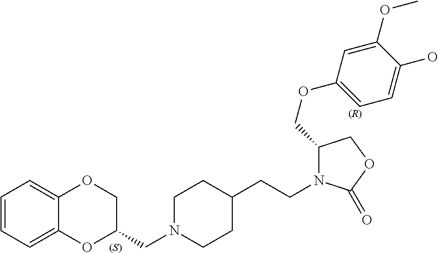

By proceeding as in Example 1 but replacing 3-(2-(piperidin-4-yl)ethyl)oxazolidin-2-one 3.1 by (R)-4-((3,4-dimethoxyphenoxy)methyl)oxazolidin-2-one 3.17, K₂CO₃ by Cs₂CO₃ and acetonitrile by dimethylformamide, 0.6 g (1.17 mmol) of compound 75 are obtained in the form of a pale yellow oil, after purification by flash chromatography on silica gel (eluent: dichloromethane/ethyl acetate=70/30% then dichloromethane/mnethanol=95/5%).

Yield: 43%

¹H NMR (CDCl₃) δ: 1.27 (m, 3H); 1.52 (m, 2H); 1.71 (m, 2H); 2.08 (m, 2H); 2.53 (dd, 1H, J=13.4 and 6.4 Hz); 2.64 (dd, 1H, J=13.6 and 5.6 Hz); 2.87 (m, 1H); 2.97 (m, 1H); 3.19 (m, 1H); 3.55 (m, 1H); 3.84 (s, 3H); 3.86 (s, 3H); 3.97 (m, 3H); 4.13 (m, 1H); 4.26 (m, 3H); 4.44 (m, 1H); 6.36 (dd, 1H, J=8.8 and 2.8 Hz); 6.50 (d, 1H, J=2.8 Hz); 6.83 (m, 5H).

0.40 g (0.78 mmol) of compound 75 are dissolved in 5 ml of methanol and then 0.07 g (0.78 mmol) of oxalic acid in solution in 5 ml of methanol are added. This is concentrated and a little ethyl ether is added, the medium is triturated and 0.38 g (0.63 mmol) of compound 75 oxalate are obtained in the form of a white solid.

F=147° C.

¹H NMR (DMSOd₆) δ: 1.38 (m, 5H); 1.78 (m, 2H); 2.67 (m, 2H); 3.12 (m, 3H); 3.23 (m, 1H); 3.34 (m, 2H); 3.68 (s, 3H); 3.74 (s, 3H); 3.99 (m, 2H); 4.14 (m, 3H); 4.28 (m, 1H); 4.40 (m, 1H); 4.60 (m, 1H); 6.46 (dd, 1H, J=8.8 and 2.8 Hz); 6.56 (d, 1H, J=2.8 Hz); 6.88 (m, 5H).

MS m/z 513 (M+1).

HPLC (XBRIDGE C8, acetonitrile/water/KH₂PO₄ (35/65/6.8 g pH 4), 0.4 ml/min), retention time=5.12 min, chemical purity=95.93%

### EXAMPLE 76

### (R)-3-(2-(1-(((S)-2,3-dihydrobenzo[b][1,4]dioxin-2-yl)methyl)piperidin-4-yl)ethyl)-4-((3-hydroxyphenoxy)methyl)oxazolidin-2-one

### Step 1: (R)-4-((3-(benzyloxy)phenoxy)methyl)-3-(2-(1-(((S)-2,3-dihydrobenzo[b][1,4]dioxin-2-yl)methyl)piperidin-4-yl)ethyl)oxazolidin-2-one

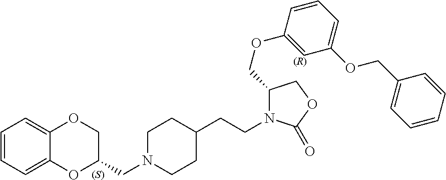

By proceeding as in Example 1 but replacing 3-(2-(piperidin-4-yl)ethyl)oxazolidin-2-one 3.1 by (R)-4-((3-(benzyloxy)phenoxy)methyl)oxazolidin-2-one 3.18, K₂CO₃ by Cs₂CO₃ and acetonitrile by dimethylformamide, 0.85 g (1.52 mmol) of compound (R)-4-((3-(benzyloxy)phenoxy)methyl)-3-(2-(1-(((S)-2,3-dihydrobenzo[b][1,4]dioxin-2-yl)methyl)piperidin-4-yl)ethyl)oxazolidin-2-one are obtained in the form of a pale yellow oil, after purification by flash chromatography on silica gel (eluent: dichloromethane/ethyl acetate=70/30% then dichloromethane/methanol=95/5%).

Yield: 48%

¹H NMR (CDCl₃) δ: 1.29 (m, 3H); 1.52 (m, 2H); 1.69 (m, 2H); 2.07 (m, 2H); 2.53 (dd, 1H, J=13.2 and 6 Hz); 2.64 (dd, 1H, J=13.2 and 5.6 Hz); 2.87 (m, 1H); 2.97 (m, 1H); 3.19 (m, 1H); 3.54 (m, 1H); 3.95 (dd, 1H, J=11.6 and 8 Hz); 4.01 (m, 1H); 4.15 (m, 3H); 4.28 (m, 2H); 4.44 (m, 1H); 5.04 (s, 2H); 6.51 (m, 2H); 6.63 (m, 1H); 6.85 (m, 4H); 7.20 (m, 1H); 7.37 (m, 5H).

### Step 2: (R)-3-(2-(1-(((S)-2,3-dihydrobenzo[b][1,4]dioxin-2-yl)methyl)piperidin-4-yl)ethyl)-4-((3-hydroxyphenoxy)methyl)oxazolidin-2-one

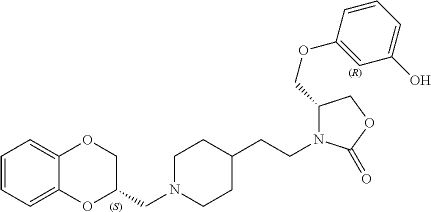

0.85 g (1.52 mmol) of the preceding compound (R)-4-((3-(benzyloxy)phenoxy)methyl)-3-(2-(1-(((S)-2,3-dihydrobenzo[b][1,4]dioxin-2-yl)methyl)piperidin-4-yl)ethyl)oxazolidin-2-one are solubilized in 10 ml of ethanol. A catalytic quantity of 5% Pd on carbon is added, and hydrogenation is performed at RT for 3.5 hours. The medium is then filtered on Celite and concentrated. 0.40 g (0.85 mmol) of product 76 are obtained in the form of a white solid, after purification on silica gel (eluent: dichloromethane/methanol=95/5%).

Yield: 56%

¹H NMR (CDCl₃) δ: 1.27 (m, 3H); 1.52 (m, 2H); 1.69 (m, 2H); 2.07 (m, 2H); 2.53 (dd, 1H, J=13.2 and 5.6 Hz); 2.66 (dd, 1H, J=13.4 and 6 Hz); 2.90 (m, 1H); 3.01 (m, 1H); 3.20 (m, 1H); 3.51 (m, 1H); 3.95 (dd, 1H, J=11.2 and 7.6 Hz); 4.00 (m, 2H); 4.16 (m, 2H); 4.28 (m, 2H); 4.44 (m, 1H); 6.42 (m, 2H); 6.49 (m, 1H); 6.84 (m, 4H); 7.13 (m, 1H).

0.40 g (0.85 mmol) of compound 76 are dissolved in 5 ml of methanol and then 0.077 g (0.85 mmol) of oxalic acid in solution in 5 ml of methanol are added. This is concentrated and a little ethyl ether is added, the medium is triturated and 0.35 g (0.63 mmol) of compound 76 oxalate are obtained in the form of a white solid.

F=123° C.

¹H NMR (DMSOd₆) δ: 1.37 (m, 5H); 1.76 (m, 2H); 2.58 (m, 2H); 3.00 (m, 2H); 3.17 (m, 2H); 3.35 (m, 2H); 3.98 (m, 2H); 4.11 (m, 2H); 4.18 (m, 1H); 4.28 (m, 1H); 4.39 (m, 1H); 4.56 (m, 1H); 6.36 (m, 3H); 6.87 (m, 4H); 7.06 (m, 1H).

MS m/z 469 (M+1).

HPLC (XBRIDGE C8, acetonitrile/water/KH₂PO₄ (30/70/6.8 g pH 4), 0.4 ml/min), retention time=7.13 min, chemical purity=98.95%

### EXAMPLE 77

### (R)-3-(2-(1-(((S)-2,3-dihydrobenzo[b][1,4]dioxin-2-yl)methyl)piperidin-4-yl)ethyl)-4-((4-hydroxyphenoxy)methyl)oxazolidin-2-one

### Step 1: (R)-4-((4-(benzyloxy)phenoxy)methyl)-3-(2-(1-(((S)-2,3-dihydrobenzo[b][1,4]dioxin-2-yl)methyl)piperidin-4-yl)ethyl)oxazolidin-2-one

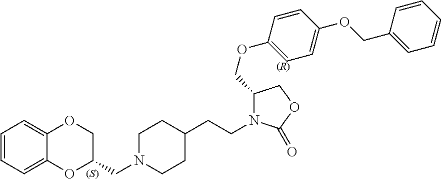

By proceeding as in Example 1 but replacing 3-(2-(piperidin-4-yl)ethyl)oxazolidin-2-one 3.1 by (R)-4-((4-(benzyloxy)phenoxy)methyl)oxazolidin-2-one 3.19, K₂CO₃ by Cs₂CO₃ and acetonitrile by dimethylformamide, 0.80 g (1.43 mmol) of compound 77 are obtained in the form of a pale yellow oil, after purification by flash chromatography on silica gel (eluent: dichloromethane/ethyl acetate=70/30% then dichloromethane/methanol=95/5%).

Yield: 51%

¹H NMR (CDCl₃) δ: 1.29 (m, 3H); 1.53 (m, 2H); 1.69 (m, 2H); 2.09 (m, 2H); 2.53 (dd, 1H, J=13.2 and 6 Hz); 2.64 (dd, 1H, J=13.2 and 5.6 Hz); 2.87 (m, 1H); 2.97 (min, 1H); 3.20 (m, 1H); 3.54 (m, 1H); 3.96 (m, 3H); 4.16 (m, 2H); 4.29 (m, 2H); 4.44 (m, 1H); 5.02 (s, 2H); 6.86 (m, 8H); 7.37 (m, 5H).

### Step 2: (R)-3-(2-(1-(((S)-2,3-dihydrobenzo[b][1,4]dioxin-2-yl)methyl)piperidin-4-yl)ethyl)-4-((4-hydroxyphenoxy)methyl)oxazolidin-2-one

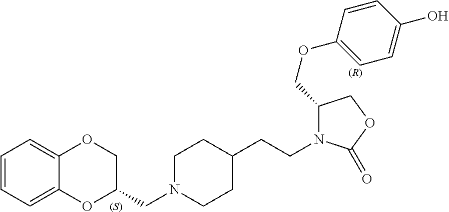

0.80 g (1.43 mmol) of the preceding compound ((R)-4-((4-(benzyloxy)phenoxy)methyl)-3-(2-(1-(((S)-2,3-dihydrobenzo[b][1,4]dioxin-2-yl)methyl)piperidin-4-yl)ethyl)oxazolidin-2-one are solubilized in a 10/5 ml ethanol/dichloromethane mixture. A catalytic quantity of 5% Pd on carbon is added, and hydrogenation is performed at RT for 3.5 hours. The medium is then filtered on Celite and concentrated. 0.65 g (1.39 mmol) of compound 77 are obtained in the form of a clear oil.

Yield: 97%

¹H NMR (CDCl₃) δ: 1.33 (m, 3H); 1.52 (m, 2H); 1.71 (m, 2H); 2.13 (m, 2H); 2.65 (m, 2H); 2.94 (m, 1H); 3.09 (m, 1H); 3.21 (m, 1H); 3.52 (m, 1H); 3.97 (m, 3H); 4.12 (m, 1H); 4.20 (m, 1H); 4.27 (m, 1H); 4.42 (m, 2H); 6.76 (m, 4H); 6.83 (m, 4H).

0.65 g (1.39 mmol) of compound 77 are dissolved in 5 ml of methanol and then 0.125 g (1.39 mmol) of oxalic acid in solution in 5 ml of methanol are added. This is concentrated and a little ethyl ether is added, the medium is triturated and 0.55 g (0.98 mmol) of compound 77 oxalate are obtained in the form of a white solid.

F=93° C.

¹H NMR (DMSOd₆) δ: 1.38 (m, 5H); 1.78 (m, 2H); 2.67 (m, 2H); 3.12 (m, 3H); 3.24 (m, 1H); 3.34 (m, 2H); 4.00 (m, 3H); 4.13 (m, 2H); 4.29 (m, 1H); 4.39 (m, 1H); 4.62 (m, 1H); 6.68 (d, 2H, J=8.4 Hz); 6.77 (d, 2H, J=8.4 Hz); 6.88 (m, 4H).

MS m/z 469 (M+1).

HPLC (XBRIDGE C8, acetonitrile/water/KH₂PO₄ (25/75/6.8 g pH 4), 0.4 ml/min), retention time=9.87 min, chemical purity=98.56%

### EXAMPLE 78

### (R)-3-(2-(1-(((S)-2,3-dihydrobenzo[b][1,4]dioxin-2-yl)methyl)piperidin-4-yl)ethyl)-4-((2,6-dimethylphenoxy)methyl)oxazolidin-2-one

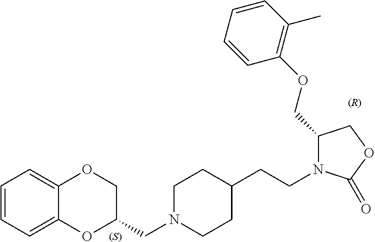

By proceeding as in Example 1 but replacing 3-(2-(piperidin-4-yl)ethyl)oxazolidin-2-one 3.1 by (R)-4-((2,6-dimethylphenoxy)methyl)-3-(2-(piperidin-4-yl)ethyl)oxazolidin-2-one 3.20, K₂CO₃ by Cs₂CO₃ and acetonitrile by dimethylformamide, 0.6 g (1.25 mmol) of compound 78 are obtained in the form of a pale yellow oil, after purification by flash chromatography on silica gel (eluent: dichloromethane/ethyl acetate=70/30% then dichloromethane/methanol=95/5%).

Yield: 46%

¹H NMR (CDCl₃) δ: 1.30 (m, 3H); 1.55 (m, 2H); 1.72 (m, 2H); 2.08 (m, 2H); 2.54 (dd, 1H, J=13.2 and 6 Hz); 2.64 (dd, 1H, J=13.4 and 5.6 Hz); 2.87 (m, 1H); 2.99 (m, 1H); 3.25 (m, 1H); 3.63 (m, 1H); 3.80 (m, 1H); 3.95 (m, 2H); 4.17 (m, 1H); 4.30 (m, 3H); 4.48 (m, 1H); 6.85 (m, 4H); 6.96 (m, 1H); 7.03 (m, 2H).

0.60 g (1.25 mmol) of compound 78 are dissolved in 5 ml of methanol and then 0.112 g (1.25 mmol) of oxalic acid in solution in 5 ml of methanol are added. This is concentrated and a little ethyl ether is added, the medium is triturated and 0.50 g (0.88 mmol) of compound 78 oxalate are obtained in the form of a white solid.

F=187° C.

¹H NMR (DMSOd₆) δ: 1.44 (m, 5H); 1.84 (m, 2H); 2.22 (s, 6H); 2.74 (m, 2H); 3.17 (m, 3H); 3.31 (m, 1H); 3.40 (m, 1H); 3.47 (m, 1H); 3.89 (m, 2H); 4.00 (dd, 1H, J=11.6 and 6.8 Hz); 4.19 (m, 1H); 4.30 (m, 2H); 4.42 (m, 1H); 4.65 (m, 1H); 6.90 (m, 5H); 7.03 (m, 2H).

MS m/z 481 (M+1).

HPLC (XBRIDGE C8, acetonitrile/water/KH₂PO₄ (40/60/6.8 g pH 4), 0.4 ml/min, retention time=7.72 min, chemical purity=97.97%

### EXAMPLE 79

### (S)-3-(2-(1-(((S)-2,3-dihydrobenzo[b][1,4]dioxin-2-yl)methyl)piperidin-4-yl)ethyl)-4-(2-hydroxypropan-2-yl)oxazolidin-2-one

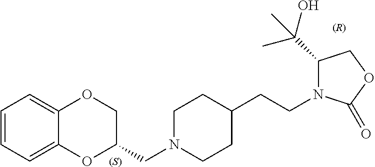

By proceeding as in Example 1 but replacing 3-(2-(piperidin-4-yl)ethyl)oxazolidin-2-one 3.1 by (S)-4-(2-hydroxypropan-2-yl)-3-(2-(piperidin-4-yl)ethyl)oxazolidin-2-one 3.24 and acetonitrile by dimethylformamide, 0.63 g (1.56 mmol) of compound 79 are obtained in the form of a colorless oil, after purification by flash chromatography on silica gel (gradient: dichloromethane/methanol=100/0% to 95/5%).

Yield: 61%

¹H NMR (CDCl₃) δ: 1.22 (s, 3H); 1.25 (s, 3H); 1.28 (m, 3H); 1.57 (m, 2H); 1.69 (m, 2H); 2.07 (m, 2H); 2.54 (dd, 1H, J=13.2 and 6.8 Hz); 2.64 (dd, 1H, J=13.8 and 6 Hz); 2.87 (m, 1H); 2.98 (m, 1H); 3.38 (m, 1H); 3.65 (m, 1H); 3.73 (dd, 1H, J=9.2 and 5.2 Hz); 3.99 (m, 2H); 4.28 (m, 3H); 6.85 (m, 4H).

0.63 g (1.56 mmol) of compound 79 are dissolved in 5 ml of methanol and then 0.126 g (1.40 mmol) of oxalic acid in solution in 5 ml of methanol are added. This is concentrated and then a little ethyl ether is added, the medium is triturated and 0.64 g (1.29 mmol) of compound 79 oxalate are obtained in the form of a white solid.

¹H NMR (DMSOd₆) δ: 1.08 (s, 6H); 1.37 (m, 3H); 1.51 (m, 2H); 1.80 (m, 2H); 2.70 (m, 2H); 3.07 (m, 2H); 3.27 (m, 1H); 3.40 (m, 3H); 3.68 (dd, 1H, J=9.2 and 5.2 Hz); 3.99 (m, 2H); 4.21 (m, 1H); 4.38 (m, 1H); 4.63 (m, 1H); 6.88 (m, 4H).

MS m/z 405 (M+1).

HPLC (XBRIDGE C8, acetonitrile/water/KH₂PO₄ (25/75/6.8 g pH 4), 0.4 ml/min, retention time=4.83 min, chemical purity=96.98%

### EXAMPLE 80

### (S)-3-((4-(2-((S)-4-(2-hydroxypropan-2-yl)-2-oxooxazolidin-3-yl)ethyl)piperidin-1-yl)methyl)-2,3-dihydrobenzo[b][1,4]dioxine-6-carbonitrile

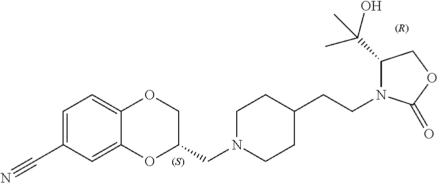

By proceeding as in Example 1 but replacing (R)-2-(bromomethyl)-2,3-dihydrobenzo[b][1,4]dioxine 2.1 by (R)-(7-cyano-2,3-dihydrobenzo[b][1,4]dioxin-2-yl)methyl 4-bromobenzenesulfonate 2.11, 3-(2-(piperidin-4-yl)ethyl)oxazolidin-2-one 3.1 by (S)-4-(2-hydroxypropan-2-yl)-3-(2-(piperidin-4-yl)ethyl)oxazolidin-2-one 3.24, the K₂CO₃/KI pair by diisopropylethylamine (1.4 eq) and acetonitrile by dimethylsulfoxide, 0.65 g (1.51 mmol) of compound 80 are obtained in the form of a pale yellow solid, after purification by flash chromatography on silica gel (gradient=100% dichloromethane to dichloromethane/(methanol:NH₄OH 9:1)=90/10% over 30 min).

Yield: 63%

¹H NMR (CDCl₃) δ: 1.22 (s, 3H); 1.25 (s, 3H); 1.27 (m, 3H); 1.57 (m, 2H); 1.71 (m, 2H); 2.08 (m, 2H); 2.53 (dd, 1H, J=13.2 and 6.4 Hz); 2.64 (dd, 1H, J=14.4 and 5.6 Hz); 2.85 (m, 1H); 2.95 (m, 1H); 3.39 (m, 1H); 3.65 (m, 1H); 3.73 (dd, 1H, J=9.2 and 5.2 Hz); 4.01 (m, 2H); 4.27 (m, 2H); 4.37 (m, 1H); 6.91 (d, 1H, J=8.4 Hz); 7.14 (dd, 1H, J=8.4 and 1.6 Hz); 7.16 (d, 1H, J=1.6 Hz).

0.65 g (1.51 mmol) of compound 80 are dissolved in a 20 ml of acetone, then 0.6 ml of a 5 N solution of HCl in isopropanol are added. This is concentrated and then a little ethyl acetate is added, the medium is triturated and 0.59 g (1.27 mmol) of compound 80 hydrochloride are obtained in the form of a white solid.

F=187° C.

¹H NMR (DMSOd₆) δ: 1.55 (m, 5H); 1.87 (m, 2H); 3.01 (m, 2H); 3.37 (m, 5H); 3.69 (m, 2H); 3.99 (m, 1H); 4.18 (m, 2H); 4.46 (m, 1H); 4.85 (m, 1H); 7.11 (d, 1H, J=8.4 Hz); 7.39 (dd, 1H, J=8.4 and 2 Hz); 7.50 (d, 1H, J=2 Hz); 10.79 (ls, 1H).

MS m/z 430 (M+1)

HPLC (XBRIDGE C8, acetonitrile/water/KH₂PO₄ (20/80/3.4 g pH 4), 0.4 ml/min, retention time=9.03 min, chemical purity=97.77%

### EXAMPLE 81

### (S)-3-((4-(2-((R)-4-(hydroxymethyl)-5,5-dimethyl-2-oxooxazolidin-3-yl)ethyl)piperidin-1-yl)methyl)-2,3-dihydrobenzo[b][1,4]dioxine-6-carbonitrile

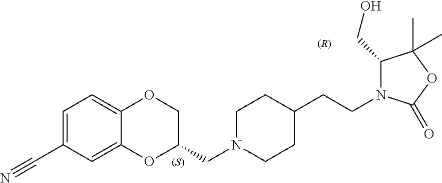

By proceeding as in Example 1 but replacing (R)-2-(bromomethyl)-2,3-dihydrobenzo[b][1,4]dioxine 2.1 by (R)-(7-cyano-2,3-dihydrobenzo[b][1,4]dioxin-2-yl)methyl 4-bromobenzenesulfonate 2.11, 3-(2-(piperidin-4-yl)ethyl)oxazolidin-2-one 3.1 by (R)-4-(hydroxymethyl)-5,5-dimethyl-3-(2-(piperidin-4-yl)ethyl)oxazolidin-2-one 3.23, the K₂CO₃/KI pair by diisopropylethylamine (1.4 eq) and acetonitrile by dimethylsulfoxide, 0.80 g (1.85 mmol) of compound 81 are obtained in the form of a colorless oil, after purification by flash chromatography on silica gel (gradient=100% dichloromethane to dichloromethane/(methanol:NH₄OH 9:1)=90/10% over 30 min).

Yield: 85%

¹H NMR (CDCl₃) δ: 1.28 (m, 3H); 1.44 (s, 3H); 1.45 (s, 3H); 1.51 (m, 2H); 1.72 (m, 2H); 2.09 (m, 2H); 2.54 (dd, 1H, J=13.4 and 6 Hz); 2.65 (dd, 1H, J=13.4 and 6 Hz); 2.86 (m, 1H); 2.95 (m, 1H); 3.13 (m, 1H); 3.43 (t, 1H, J=4.8 Hz); 3.55 (m, 1H); 3.81 (d, 2H, J=4.8 Hz); 4.02 (dd, 1H, J=11.4 and 7.2 Hz); 4.28 (m, 1H); 4.37 (m, 1H); 6.91 (d, 1H, J=8 Hz); 7.14 (dd, 1H, J=8 and 2 Hz); 7.16 (d, 1H, J=2 Hz).

HPLC (XBRIDGE C8, acetonitrile/water/5 mM HCl (23/77), 1 ml/min, retention time=7.23 min, chemical purity=98.41%

0.80 g (1.85 mmol) of compound 81 are dissolved in a 15 ml of acetone, then 0.37 ml of a 5 N solution of HCl in isopropanol are added. 0.71 g (1.52 mmol) of compound 81 hydrochloride are obtained in the form of a white solid.

F=208° C.

¹H NMR (DMSOd₆) δ: 1.34 (s, 3H); 1.35 (s, 3H); 1.53 (m, 5H); 1.89 (m, 2H); 3.06 (m, 2H); 3.34 (m, 4H); 3.46 (m, 2H); 3.60 (m, 2H); 3.68 (m, 1H); 4.19 (dd, 1H, J=11.6 and 6.4 Hz); 4.45 (m, 1H); 4.89 (m, 1H); 7.12 (d, 1H, J=8.4 Hz); 7.39 (dd, 1H, J=8.4 and 2 Hz); 7.51 (d, 1H, J=2 Hz); 10.84 (ls, 1H).

MS m/z 430 (M+1).

### EXAMPLE 82

### (3S)-3-((4-(2-((4R)-4-(hydroxymethyl)-2-oxo-5-phenyloxazolidin-3-yl)ethyl)piperidin-1-yl)methyl)-2,3-dihydrobenzo[b][1,4]dioxine-6-carbonitrile

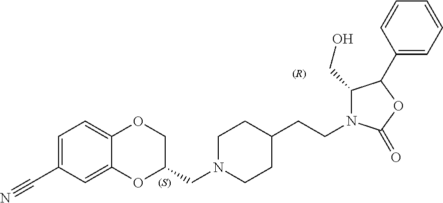

By proceeding as in Example 1 but replacing (R)-2-(bromomethyl)-2,3-dihydrobenzo[b][1,4]dioxine 2.1 by (R)-(7-cyano-2,3-dihydrobenzo[b][1,4]dioxin-2-yl)methyl 4-bromobenzenesulfonate 2.11, 3-(2-(piperidin-4-yl)ethyl)oxazolidin-2-one 3.1 by (4R,5R)-4-(hydroxymethyl)-5-phenyl-3-(2-(piperidin-4-yl)ethyl)oxazolidin-2-one 3.25, the K₂CO₃/KI pair by diisopropylethylamine (2.2 eq) and acetonitrile by dimethylsulfoxide, 1.27 g (2.66 mmol) of compound 82 are obtained in the form of a white solid, after purification by flash chromatography on silica gel (gradient=100% dichloromethane to dichloromethane/(methanol:NH₄OH 9:1)=95/5% over 30 min).

Yield: 84%

¹H NMR (CDCl₃) δ: 1.23 (m, 3H); 1.48 (m, 2H); 1.61 (m, 1H); 1.73 (m, 1H); 2.04 (m, 3H); 2.53 (dd, 1H, J=13.6 and 6.4 Hz); 2.64 (dd, 1H, J=13.6 and 5.6 Hz); 2.85 (m, 1H); 2.92 (m, 1H); 3.17 (m, 1H); 3.59 (m, 1H); 3.66 (m, 1H); 3.79 (m, 1H); 3.89 (m, 1H); 4.00 (dd, 1H, J=11.2 and 7.6 Hz); 4.27 (m, 1H); 4.36 (m, 1H); 5.35 (d, 1H, J=5.6 Hz); 6.91 (d, 1H, J=8.4 Hz); 7.14 (m, 2H); 7.38 (m, 5H).

1.27 g (2.66 mmol) of compound 82 are dissolved in a 30 ml of ethyl acetate, then 1 ml of a 5 N solution of HCl in isopropanol is added. This is concentrated and a little isopropyl ether is added, the medium is triturated and 1.35 g (2.62 mmol) of compound 82 hydrochloride are obtained in the form of a white solid.

F=130° C.

¹H NMR (DMSOd₆) δ: 1.53 (m, 5H); 1.82 (m, 1H); 1.92 (m, 1H); 2.48 (m, 2H); 3.16 (m, 1H); 3.38 (m, 5H); 3.57 (m, 1H); 3.66 (m, 2H); 4.18 (dd, 1H, J=11.6 and 6.4 Hz); 4.45 (m, 1H); 5.01 (m, 1H); 5.30 (d, 1H, J=5.6 Hz); 7.11 (d, 1H, J=8.4 Hz); 7.44 (m, 7H).

MS m/z 478 (M+1).

HPLC (XBRIDGE C8, acetonitrile/water/KH₂PO₄ (33/67/6.8 g pH 4), 1 ml/min, retention time=7.07 min, chemical purity=98.69%

### EXAMPLE 83

### (S)-3-((4-(2-((S)-4-((R or S)-hydroxy(phenyl)methyl)-2-oxooxazolidin-3-yl)ethyl)piperidin-1-yl)methyl)-2,3-dihydrobenzo[b][1,4]dioxine-6-carbonitrile

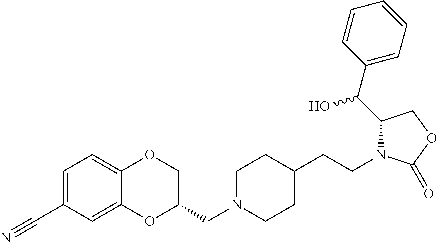 [SMILES: N#Cc1ccc2c(c1)O[C@@H](CN1CCC(CCN3C(=O)OC[C@H]3C(O)c3ccccc3)CC1)CO2]

By proceeding as in Example 1 but replacing (R)-2-(bromomethyl)-2,3-dihydrobenzo[b][1,4]dioxine 2.1 by (R)-(7-cyano-2,3-dihydrobenzo[b][1,4]dioxin-2-yl)methyl 4-bromobenzenesulfonate 2.11, 3-(2-(piperidin-4-yl)ethyl)oxazolidin-2-one 3.1 by (S)-4-((S or R)-hydroxy(phenyl)methyl)-3-(2-(piperidin-4-yl)ethyl)oxazolidin-2-one 3.26, the K₂CO₃/KI pair by diisopropylethylamine (2.2 eq) 1 g (2.09 mmol) of compound 83 is obtained in the form of a beige foam, after purification by flash chromatography on silica gel (gradient=100% dichloromethane to dichloromethane/(methanol:NH₄OH 9:1)=95/5% over 30 min).

Yield: 72%

¹H NMR (CDCl₃) δ: 1.26 (m, 3H); 1.58 (m, 2H); 1.70 (m, 2H); 2.08 (m, 2H); 2.24 (Is, 1H); 2.53 (dd, 1H, J=13.4 and 6 Hz); 2.64 (dd, 1H, J=13.4 and 6 Hz); 2.84 (m, 1H); 2.95 (m, 1H); 3.40 (m, 1H); 3.61 (m, 1H); 3.87 (dd, 1H, J=9.2 and 6 Hz); 4.02 (m, 3H); 4.28 (m, 1H); 4.36 (m, 1H); 4.75 (m, 1H); 6.91 (d, 1H, J=8.4 Hz); 7.14 (m, 2H); 7.38 (m, 5H).

1 g (2.09 mmol) of compound 83 is dissolved in a 30 ml of ethyl acetate, then 0.8 ml of a 5 N solution of HCl in isopropanol are added. This is concentrated and a little ethyl acetate is added, the medium is triturated and 0.91 g (1.77 mmol) of compound 83 hydrochloride are obtained in the form of a white solid.

F=120° C.

¹H NMR (DMSOd₆) δ: 1.41 (m, 3H); 1.53 (m, 2H); 1.81 (m, 2H); 2.99 (m, 2H); 3.32 (m, 5H); 3.65 (m, 1H); 3.95 (m, 1H); 4.03 (m, 1H); 4.15 (m, 2H); 4.45 (m, 1H); 4.72 (m, 1H); 4.99 (m, 1H); 5.86 (m, 1H); 7.11 (d, 1H, J=8.4 Hz); 7.29 (m, 1H); 7.37 (m, 5H); 7.49 (m, 1H).

MS m/z 478 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 25 to 60% in 5 min, 0.75 ml/min), retention time=1.62 min, chemical purity: 99.73%.

### EXAMPLE 84

### (S)-3-((4-(2-((S)-4-((S or R)-hydroxy(phenyl)methyl)-2-oxooxazolidin-3-yl)ethyl)piperidin-1-yl)methyl)-2,3-dihydrobenzo[b][1,4]dioxine-6-carbonitrile

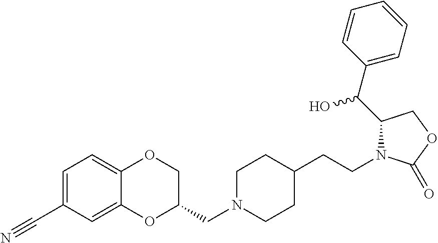 [SMILES: N#Cc1ccc2c(c1)O[C@@H](CN1CCC(CCN3C(=O)OC[C@H]3C(O)c3ccccc3)CC1)CO2]

By proceeding as in Example 1 but replacing (R)-2-(bromomethyl)-2,3-dihydrobenzo[b][1,4]dioxine 2.1 by (R)-(7-cyano-2,3-dihydrobenzo[b][1,4]dioxin-2-yl)methyl 4-bromobenzenesulfonate 2.11, 3-(2-(piperidin-4-yl)ethyl)oxazolidin-2-one 3.1 by (S)-4-((S or R)-hydroxy(phenyl)methyl)-3-(2-(piperidin-4-yl)ethyl)oxazolidin-2-one 3.27 and the K₂CO₃/KI pair by diisopropylethylamine (2.2 eq), 0.5 g (1.05 mmol) of compound 84 are obtained in the form of a white foam, after purification by flash chromatography on silica gel (gradient: dichloromethane=100% to dichloromethane/(methanol:NH₄OH 9:1)=95/5% over 30 min).

Yield: 64%

¹H NMR (CDCl₃) δ: 1.25 (m, 3H); 1.49 (m, 2H); 1.67 (m, 2H); 2.08 (m, 2H); 2.53 (dd, 1H, J=13.4 and 6 Hz); 2.64 (dd, 1H, J=13.4 and 6 Hz); 2.85 (m, 1H); 2.96 (m, 2H); 3.55 (m, 1H); 4.04 (m, 3H); 4.28 (m, 1H); 4.37 (m, 2H); 4.94 (m, 1H); 6.91 (d, 1H, J=8.4 Hz); 7.14 (m, 2H); 7.37 (m, 5H).

0.5 g (1.05 mmol) of compound 84 are dissolved in a 30 ml of ethyl acetate, then 0.4 ml of a 5 N solution of HCl in isopropanol are added. This is concentrated and then a little ethyl acetate is added, the medium is triturated and 0.47 g (0.91 mmol) of compound 84 hydrochloride are obtained in the form of a white solid.

F=160° C.

¹H NMR (DMSOd₆) δ: 1.58 (m, 5H); 1.90 (m, 2H); 3.06 (m, 3H); 3.40 (m, 4H); 3.68 (m, 1H); 3.94 (m, 1H); 4.04 (m, 1H); 4.10 (m, 1H); 4.17 (m, 1H); 4.45 (m, 1H); 4.92 (ls, 1H); 5.00 (m, 1H); 5.84 (ls, 1H); 7.11 (d, 1H, J=8.4 Hz); 7.28 (m, 1H); 7.39 (m, 5H); 7.49 (m, 1H).

MS m/z 478 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 25 to 60% in 5 min, 0.75 ml/min), retention time=1.83 min, chemical purity: 97.94%.

### EXAMPLE 85

### (S)-3-((4-(2-(3-oxomorpholino)ethyl)piperidin-1-yl)methyl)-2,3-dihydrobenzo[b][1,4]dioxine-6-carbonitrile

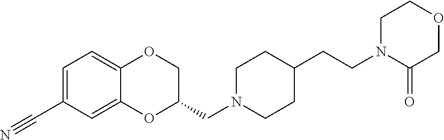 [SMILES: N#Cc1ccc2c(c1)O[C@@H](CN1CCC(CCN3CCOCC3=O)CC1)CO2]

By proceeding as in Example 1 but replacing (R)-2-(bromomethyl)-2,3-dihydrobenzo[b][1,4]dioxine 2.1 by (R)-(7-cyano-2,3-dihydrobenzo[b][1,4]dioxin-2-yl)methyl 4-bromobenzenesulfonate 2.11, 3-(2-(piperidin-4-yl)ethyl)oxazolidin-2-one 3.1 by 4-(2-(piperidin-4-yl)ethyl)morpholin-3-one 3.28, the K₂CO₃/KI pair by DMAP (1 eq) and acetonitrile by dimethylsulfoxide, 0.68 g (1.76 mmol) of compound 85 are obtained in the form of a clear oil, after purification by flash chromatography on silica gel (eluent: dichloromethane/methanol=95/5%).

Yield: 62%

¹H NMR (CDCl₃) δ: 1.28 (m, 3H); 1.51 (m, 2H); 1.73 (m, 2H); 2.08 (m, 2H); 2.54 (dd, 1H, J=13.6 and 6.4 Hz); 2.65 (dd, 1H, J=13.2 and 5.6 Hz); 2.86 (m, 1H); 2.95 (m, 1H); 3.35 (m, 2H); 3.45 (t, 2H, J=7.6 Hz); 3.88 (m, 2H); 4.02 (dd, 1H, J=11.4 and 7.2 Hz); 4.16 (s, 2H); 4.29 (m, 1H); 4.37 (dd, 1H, J=11.4 and 2.4); 6.91 (d, 1H, J=8.2 Hz); 7.14 (dd, 1H, J=8.2 and 2 Hz); 7.16 (d, 1H, J=2 Hz).

0.63 g (1.63 mmol) of compound 85 are dissolved in 10 ml of methanol and 0.147 g (1.63 mmol) of oxalic acid in solution in 5 ml of methanol are added. This is concentrated and then a little ethyl ether is added, the medium is triturated and 0.64 g (1.34 mmol) of compound 85 oxalate are obtained in the form of a white solid.

F=168° C.

¹H NMR (DMSOd₆) δ: 1.36 (m, 3H); 1.44 (m, 2H); 1.81 (m, 2H); 2.65 (m, 2H); 3.07 (m, 2H); 3.23 (m, 1H); 3.35 (m, 5H); 3.81 (m, 2H); 4.00 (s, 2H); 4.12 (dd, 1H, J=11.6 and 6.4 Hz); 4.40 (dd, 1H, J=11.6 and 2.4 Hz); 4.69 (m, 1H); 7.08 (d, 1H, J=8.4 Hz); 7.35 (dd, 1H, J=8.4 and 2 Hz); 7.45 (d, 1H, =2 Hz).

MS m/z 386 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 10 to 40% in 5 min, 0.75 ml/min), retention time=2.33 min, chemical purity: 98.80%.

### EXAMPLE 86

### (S)-2-((4-(2-(3-oxomorpholino)ethyl)piperidin-1-yl)methyl)-2,3-dihydrobenzo[b][1,4]dioxine-5-carbonitrile

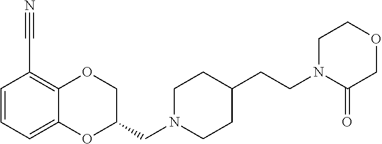 [SMILES: N#Cc1cccc2c1OC[C@H](CN1CCC(CCN3CCOCC3=O)CC1)O2]

By proceeding as in Example 1 but replacing (R)-2-(bromomethyl)-2,3-dihydrobenzo[b][1,4]dioxine 2.1 by (R)-(5-cyano-2,3-dihydrobenzo[b][1,4]dioxin-2-yl)methyl 4-bromobenzenesulfonate 2.14, 3-(2-(piperidin-4-yl)ethyl)oxazolidin-2-one 3.1 by 4-(2-(piperidin-4-yl)ethyl)morpholin-3-one 3.28 and the K₂CO₃/KI pair by diisopropylethylamine (1.4 eq), 0.18 g (0.47 mmol) of compound 86 are obtained in the form of a clear oil, after purification by flash chromatography on silica gel (gradient: dichloromethane=100% to dichloromethane/methanol=96/4% over 30 min).

Yield: 63%

¹H NMR (DMSOd₆) δ: 1.16 (m, 3H); 1.40 (m, 2H); 1.65 (m, 2H); 1.99 (m, 2H); 2.53 (m, 2H); 2.88 (m, 2H); 3.32 (m, 4H); 3.80 (m, 2H); 3.99 (s, 2H); 4.12 (dd, 1H, J=11.2 and 7.2 Hz); 4.46 (m, 2H); 6.97 (m, 1H); 7.21 (dd, 1H, J=8.2 and 1.6 Hz); 7.28 (dd, 1H, J=7.8 and 1.6 Hz).

0.18 g (0.47 mmol) of compound 86 are dissolved in 0.5 ml of acetonitrile and then 0.042 g (0.47 mmol) of oxalic acid and 2 ml of water are added. This lyophilized and 0.22 g (0.46 mmol) of compound 86 oxalate are obtained in the form of a white solid.

¹H NMR (DMSOd₆) δ: 1.37 (m, 3H); 1.44 (m, 2H); 1.82 (m, 2H); 2.68 (m, 2H); 3.11 (m, 2H); 3.31 (m, 6H); 3.81 (m, 2H); 4.00 (s, 2H); 4.20 (dd, 1H, J=11.6 and 6.8 Hz); 4.49 (dd, 1H, J=11.6 and 2.4 Hz); 4.75 (m, 1H); 7.02 (m, 1H); 7.27 (dd, 1H, J=8 and 1.2 Hz); 7.34 (dd, 1H, J=7.6 and 1.2 Hz).

MS m/z 386 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.23 min, chemical purity: 98.58%.

### EXAMPLE 87

### (S)-4-(2-(1-((7-chloro-2,3-dihydrobenzo[b][1,4]dioxin-2-yl)methyl)piperidin-4-yl)ethyl)morpholin-3-one

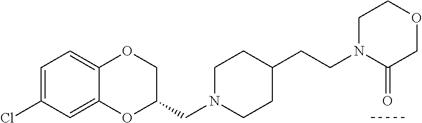

By proceeding as in Example 1 but replacing (R)-2-(bromomethyl)-2,3-dihydrobenzo[b][1,4]dioxine 2.1 by (R)-(7-chloro-2,3-dihydrobenzo[b][1,4]dioxin-2-yl)methyl 4-bromobenzenesulfonate 2.7, 3-(2-(piperidin-4-yl)ethyl)oxazolidin-2-one 3.1 by 4-(2-(piperidin-4-yl)ethyl)morpholin-3-one 3.28 and the K₂CO₃/KI pair by diisopropylethylamine (1.4 eq), 0.21 g (0.53 mmol) of compound 87 are obtained in the form of a clear oil, after purification by flash chromatography on silica gel (gradient: dichloromethane=100% to dichloromethane/methanol=90/10% over 30 min).

Yield: 73%

¹H NMR (DMSOd₆) δ: 1.15 (m, 3H); 1.41 (m, 2H); 1.65 (m, 2H); 1.97 (m, 2H); 2.50 (m, 2H); 2.82 (m, 1H); 2.91 (m, 1H); 3.32 (m, 4H); 3.80 (m, 2H); 3.96 (dd, 1H, J=11.6 and 7.2 Hz); 3.99 (s, 2H); 4.29 (dd, 1H, J=11.6 and 2 Hz); 4.35 (m, 1H); 6.87 (m, 2H); 6.95 (d, 1H, J=2.4 Hz).

0.21 g (0.53 mmol) of compound 87 are dissolved in 0.5 ml of acetonitrile and then 0.043 g (0.48 mmol) of oxalic acid and 2 ml of water are added. This lyophilized and 0.25 g (0.51 mmol) of compound 87 oxalate are obtained in the form of a white solid.

¹H NMR (DMSOd₆) δ: 1.38 (m, 3H); 1.45 (m, 2H); 1.83 (m, 2H); 2.72 (m, 2H); 3.11 (m, 2H); 3.33 (m, 6H); 3.81 (m, 2H); 4.00 (s, 2H); 4.04 (dd, 1H, J=11.6 and 6.4 Hz); 4.30 (dd, 1H, J=11.6 and 2 Hz); 4.87 (m, 1H); 6.92 (m, 2H); 7.02 (d, 1H, J=2 Hz).

MS m/z 395 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.59 min, chemical purity: 100%.

### EXAMPLE 88

### (S)-4-(2-(1-((7-fluoro-2,3-dihydrobenzo[b][1,4]dioxin-2-yl)methyl)piperidin-4-yl)ethyl)morpholin-3-one

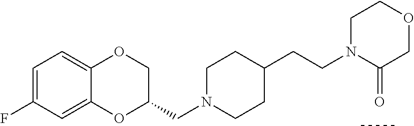

By proceeding as in Example 1 but replacing (R)-2-(bromomethyl)-2,3-dihydrobenzo[b][1,4]dioxine 2.1 by (R)-(7-fluoro-2,3-dihydrobenzo[b][1,4]dioxin-2-yl)methyl 4-bromobenzenesulfonate 2.17, 3-(2-(piperidin-4-yl)ethyl)oxazolidin-2-one 3.1 by 4-(2-(piperidin-4-yl)ethyl)morpholin-3-one 3.28 and the K₂CO₃/KI pair by diisopropylethylamine (1.4 eq), 0.17 g (0.45 mmol) of compound 88 are obtained in the form of a clear oil, after purification by flash chromatography on silica gel (gradient: dichloromethane=100% to dichloromethane/methanol=96/4% over 30 min).

Yield: 59%

¹H NMR (DMSOd₆) δ: 1.14 (m, 3H); 1.41 (m, 2H); 1.65 (m, 2H); 2.48 (m, 2H); 2.51 (m, 2H); 2.83 (m, 1H); 2.91 (m, 1H); 3.32 (m, 4H); 3.80 (m, 2H); 3.93 (dd, 1H, J=11.6 and 7.2 Hz); 3.99 (s, 2H); 4.26 (dd, 1H, J=11.6 and 2.4 Hz); 4.35 (m, 1H); 6.65 (m, 1H); 6.76 (m, 1H); 6.87 (m, 1H).

0.17 g (0.45 mmol) of compound 88 are dissolved in 0.5 ml of acetonitrile and then 0.041 g (0.45 mmol) of oxalic acid and 2 ml of water are added. This lyophilized and 0.21 g (0.45 mmol) of compound 88 oxalate are obtained in the form of a white solid.

¹H NMR (DMSOd₆) δ: 1.38 (m, 3H); 1.44 (m, 2H); 1.82 (m, 2H); 2.70 (m, 2H); 3.10 (m, 2H); 3.35 (m, 6H); 3.81 (m, 2H); 4.00 (m, 3H); 4.27 (dd, 1H, J=11.4 and 2 Hz); 4.68 (m, 1H); 6.71 (m, 1H); 6.83 (m, 1H); 6.92 (m, 1H).

MS m/z 379 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.35 min, chemical purity: 99.30%.

### EXAMPLE 89

### (S)-4-(2-(1-((8-fluoro-2,3-dihydrobenzo[b][1,4]dioxin-2-yl)methyl)piperidin-4-yl)ethyl)morpholin-3-one

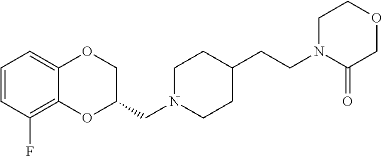 [SMILES: O=C1COCCN1CCC1CCN(C[C@H]2COc3cccc(F)c3O2)CC1]

By proceeding as in Example 1 but replacing (R)-2-(bromomethyl)-2,3-dihydrobenzo[b][1,4]dioxine 2.1 by (R)-(8-fluoro-2,3-dihydrobenzo[b][1,4]dioxin-2-yl)methyl 4-bromobenzenesulfonate 2.16, 3-(2-(piperidin-4-yl)ethyl)oxazolidin-2-one 3.1 by 4-(2-(piperidin-4-yl)ethyl)morpholin-3-one 3.28 and the K₂CO₃/KI pair by diisopropylethylamine (1.4 eq), 0.16 g (0.42 mmol) of compound 89 are obtained in the form of a clear oil, after purification by flash chromatography on silica gel (gradient: dichloromethane=100% to dichloromethane/methanol=96/4% over 30 min).

Yield: 56%

¹H NMR (DMSOd₆) δ: 1.16 (m, 3H); 1.42 (m, 2H); 1.65 (m, 2H); 1.99 (m, 2H); 2.56 (m, 2H); 2.83 (m, 1H); 2.94 (m, 1H); 3.32 (m, 4H); 3.80 (m, 2H); 3.99 (s, 2H); 4.01 (m, 1H); 4.36 (m, 2H); 6.71 (m, 1H); 6.79 (m, 2H).

0.16 g (0.42 mmol) of compound 89 are dissolved in 0.5 ml of acetonitrile and then 0.039 g (0.42 mmol) of oxalic acid and 2 ml of water are added. This lyophilized and 0.18 g (0.38 mmol) of compound 89 oxalate are obtained in the form of a white solid.

¹H NMR (DMSOd₆) δ: 1.38 (m, 3H); 1.45 (m, 2H); 1.84 (m, 2H); 2.76 (m, 2H); 3.16 (m, 2H); 3.34 (m, 6H); 3.81 (m, 2H); 4.00 (s, 2H); 4.08 (dd, 1H, J=11.6 and 6.8 Hz); 4.35 (dd, 1H, J=11.6 and 2 Hz); 4.73 (m, 1H); 6.76 (m, 1H); 6.84 (m, 2H).

MS m/z 379 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.32 min, chemical purity: 99.21%.

### EXAMPLE 90

### (S)-4-(2-(1-((6-fluoro-2,3-dihydrobenzo[b][1,4]dioxin-2-yl)methyl)piperidin-4-yl)ethyl)morpholin-3-one

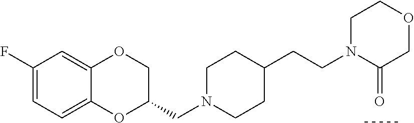

By proceeding as in Example 1 but replacing (R)-2-(bromomethyl)-2,3-dihydrobenzo[b][1,4]dioxine 2.1 by (R)-(6-fluoro-2,3-dihydrobenzo[b][1,4]dioxin-2-yl)methyl 4-bromobenzenesulfonate 2.18, 3-(2-(piperidin-4-yl)ethyl)oxazolidin-2-one 3.1 by 4-(2-(piperidin-4-yl)ethyl)morpholin-3-one 3.28 and the K₂CO₃/KI pair by diisopropylethylamine (1.4 eq), 0.18 g (0.47 mmol) of compound 90 are obtained in the form of a clear oil, after purification by flash chromatography on silica gel (gradient: dichloromethane=100% to dichloromethane/methanol=96/4% over 30 min).

Yield: 59%

¹H NMR (DMSOd₆) δ: 1.15 (m, 3H); 1.40 (m, 2H); 1.65 (m, 2H); 2.48 (m, 2H); 2.51 (m, 2H); 2.82 (m, 1H); 2.91 (m, 1H); 3.32 (m, 4H); 3.80 (m, 2H); 3.95 (dd, 1H, J=11.6 and 7.6 Hz); 3.99 (s, 2H); 4.29 (m, 2H); 6.65 (m, 1H); 6.77 (m, 1H); 6.87 (m, 1H).

0.18 g (0.47 mmol) of compound 90 are dissolved in 0.5 ml of acetonitrile and then 0.043 g (0.47 mmol) of oxalic acid and 2 ml of water are added. This lyophilized and 0.21 g (0.45 mmol) of compound 90 oxalate are obtained in the form of a white solid.

¹H NMR (DMSOd₆) δ: 1.42 (m, 5H); 1.83 (m, 2H); 2.74 (m, 2H); 3.12 (m, 2H); 3.34 (m, 6H); 3.81 (m, 2H); 4.00 (s, 2H); 4.02 (m, 1H); 4.32 (dd, 1H, J=11.6 and 2.4 Hz); 4.63 (m, 1H); 6.71 (m, 1H); 6.82 (m, 1H); 6.94 (m, 1H).

MS m/z 379 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.38 min, chemical purity: 99.33%.

### EXAMPLE 91

### (S)-4-(2-(1-((5,7-difluoro-2,3-dihydrobenzo[b][1,4]dioxin-2-yl)methyl)piperidin-4-yl)ethyl)morpholin-3-one

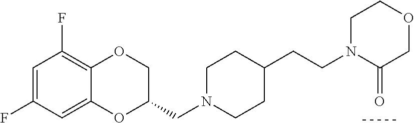

By proceeding as in Example 1 but replacing (R)-2-(bromomethyl)-2,3-dihydrobenzo[b][1,4]dioxine 2.1 (R)-(5,7-difluoro-2,3-dihydrobenzo[b][1,4]dioxin-2-yl)methyl 4-bromobenzenesulfonate 2.19, 3-(2-(piperidin-4-yl)ethyl)oxazolidin-2-one 3.1 by 4-(2-(piperidin-4-yl)ethyl)morpholin-3-one 3.28 and the K₂CO₃/KI pair by diisopropylethylamine (1.4 eq), 0.15 g (0.38 mmol) of compound 91 are obtained in the form of a clear oil, after purification by flash chromatography on silica gel (gradient: dichloromethane=100% to dichloromethane/methanol=96/4% over 30 min).

Yield: 54%

¹H NMR (DMSOd₆) δ: 1.14 (m, 3H); 1.41 (m, 2H); 1.65 (m, 2H); 1.99 (m, 2H); 2.54 (m, 2H); 2.84 (m, 1H); 2.90 (m, 1H); 3.32 (m, 4H); 3.80 (m, 2H); 3.98 (m, 1H); 3.99 (s, 2H); 4.34 (dd, 1H, J=11.6 and 2.4 Hz); 4.43 (m, 1H); 6.69 (m, 1H); 6.84 (m, 1H).

0.15 g (0.38 mmol) of compound 91 are dissolved in 0.5 ml of acetonitrile and then 0.034 g (0.38 mmol) of oxalic acid and 2 ml of water are added. This is lyophilized and 0.17 g (0.35 mmol) of compound 91 oxalate are obtained in the form of a white solid.

¹H NMR (DMSOd₆) δ: 1.41 (m, 5H); 1.82 (m, 2H); 2.69 (m, 2H); 3.10 (m, 2H); 3.31 (m, 6H); 3.81 (m, 2H); 4.00 (s, 2H); 4.07 (dd, 1H, J=11.6 and 6.4 Hz); 4.35 (dd, 1H, J=11.6 and 2.4 Hz); 4.76 (m, 1H); 6.75 (m, 1H); 6.91 (m, 1H).

MS m/z 397 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.45 min, chemical purity: 100%.

### EXAMPLE 92

### (S)-4-(2-(1-((6.7-difluoro-2,3-dihydrobenzo[b][1,4]dioxin-2-yl)methyl)piperidin-4-yl)ethyl)morpholin-3-one

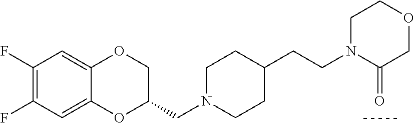

By proceeding as in Example 1 but replacing (R)-2-(bromomethyl)-2,3-dihydrobenzo[b][1,4]dioxine 2.1 by (R)-(6,7-difluoro-2,3-dihydrobenzo[b][1,4]dioxin-2-yl)methyl 4-bromobenzenesulfonate 2.22, 3-(2-(piperidin-4-yl)ethyl)oxazolidin-2-one 3.1 by 4-(2-(piperidin-4-yl)ethyl)morpholin-3-one 3.28 and the K₂CO₃/KI pair by diisopropylethylamine (1.4 eq), 0.13 g (0.33 mmol) of compound 92 are obtained in the form of a clear oil, after purification by flash chromatography on silica gel (gradient: dichloromethane=100% to dichloromethane/methanol=96/4% over 30 min).

Yield: 46%

¹H NMR (DMSOd₆) δ: 1.14 (m, 3H); 1.41 (m, 2H); 1.65 (m, 2H); 1.98 (m, 2H); 2.51 (m, 2H); 2.82 (m, 1H); 2.90 (m, 1H); 3.32 (m, 4H); 3.80 (m, 2H); 3.94 (dd, 1H, J=12 and 7.2 Hz); 3.99 (s, 2H); 4.31 (m, 2H); 7.04 (m, 2H).

0.13 g (0.33 mmol) of compound 92 are dissolved in 0.5 ml of acetonitrile and then 0.030 g (0.38 mmol) of oxalic acid and 2 ml of water are added. This is lyophilized and 0.16 g (0.33 mmol) of compound 92 oxalate are obtained in the form of a white solid.

¹H NMR (DMSOd₆) δ: 1.41 (m, 5H); 1.82 (m, 2H); 2.70 (m, 2H); 3.10 (m, 2H); 3.31 (m, 6H); 3.81 (m, 2H); 4.00 (s, 2H); 4.02 (m, 1H); 4.30 (dd, 1H, J=11.6 and 2 Hz); 4.67 (m, 1H); 7.10 (m, 2H).

MS m/z 397 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.45 min, chemical purity: 98.74%.

### EXAMPLE 93

### (S)-4-(2-(1-((5-chloro-7-fluoro-2,3-dihydrobenzo[b][1,4]dioxin-2-yl)methyl)piperidin-4-yl)ethyl)morpholin-3-one

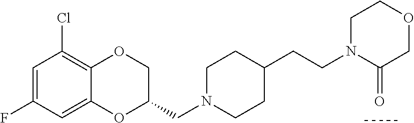

By proceeding as in Example 1 but replacing (R)-2-(bromomethyl)-2,3-dihydrobenzo[b][1,4]dioxine 2.1 by (R)-(5-chloro-7-fluoro-2,3-dihydrobenzo[b][1,4]dioxin-2-yl)methyl 4-bromobenzenesulfonate 2.21, 3-(2-(piperidin-4-yl)ethyl)oxazolidin-2-one 3.1 by 4-(2-(piperidin-4-yl)ethyl)morpholin-3-one 3.28 and the K₂CO₃/KI pair by diisopropylethylamine (1.4 eq), 0.6 g (1.45 mmol) of compound 93 are obtained in the form of a clear oil, after purification by flash chromatography on silica gel (eluent: dichloromethane/methanol=95/5%).

Yield: 58%

¹H NMR (CDCl₃) δ: 1.28 (m, 3H); 1.50 (m, 2H); 1.72 (m, 2H); 2.08 (m, 2H); 2.55 (dd, 1H, J=13.4 and 6 Hz); 2.65 (dd, 1H, J=13.4 and 5.6 Hz); 2.87 (m, 1H); 2.96 (m, 1H); 3.35 (t, 2H, J=5.2 Hz); 3.44 (t, 2H, J=7.6 Hz); 3.88 (t, 2H, J=5.2 Hz); 4.00 (dd, 1H, J=11.4 and 7.6 Hz); 4.16 (s, 2H); 4.30 (m, 1H); 4.39 (dd, 1H, J=11.4 and 2.4 Hz); 6.56 (1H, J=9.2 and 2.8 Hz); 6.69 (dd, 1H, J=8.2 and 2.8 Hz).

0.6 g (1.45 mmol) of compound 93 are dissolved in a 10 ml of ethanol and 0.58 ml (2.90 mmol) of a 5 N solution of HCl in isopropanol are added. This is concentrated and then a little acetone and ethyl acetate are added, the medium is triturated and 0.45 g (1 mmol) of compound 93 hydrochloride are obtained in the form of a white solid.

F=180° C.

¹H NMR (DMSOd₆) δ: 1.45 (m, 3H); 1.54 (m, 2H); 1.90 (m, 2H); 3.00 (m, 2H); 3.32 (m, 6H); 3.46 (m, 1H); 3.66 (m, 1H); 3.82 (m, 2H); 4.01 (s, 2H); 4.18 (dd, 1H, J=11.6 and 6 Hz); 4.44 (dd, 1H, J=11.6 and 2.4 Hz); 5.04 (m, 1H); 6.94 (dd, 1H, J=9.6 and 2.8 Hz); 7.08 (dd, 1H, J=8.4 and 3.2 Hz).

MS m/z 413 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 25 to 60% in 5 min, 0.75 ml/min), retention time=1.51 min, chemical purity: 98.94%.

### EXAMPLE 94

### (S)-4-(2-(1-((7-chloro-5-fluoro-2,3-dihydrobenzo[b][1,4]dioxin-2-yl)methyl)piperidin-4-yl)ethyl)morpholin-3-one

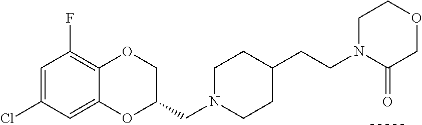

By proceeding as in Example 1 but replacing (R)-2-(bromomethyl)-2,3-dihydrobenzo[b][1,4]dioxine 2.1 by (R)-(5-fluoro-7-chloro-2,3-dihydrobenzo[b][1,4]dioxin-2-yl)methyl 4-bromobenzenesulfonate 2.20, 3-(2-(piperidin-4-yl)ethyl)oxazolidin-2-one 3.1 by 4-(2-(piperidin-4-yl)ethyl)morpholin-3-one 3.28 and the K₂CO₃/KI pair by diisopropylethylamine (1.4 eq), 0.17 g (0.41 mmol) of compound 94 are obtained in the form of a clear oil, after purification by flash chromatography on silica gel (gradient: dichloromethane=100% to dichloromethane/methanol=96/4% over 30 min).

Yield: 59%

¹H NMR (DMSOd₆) δ: 1.14 (m, 3H); 1.41 (m, 2H); 1.65 (m, 2H); 1.99 (m, 2H); 2.54 (m, 2H); 2.83 (m, 1H); 2.90 (m, 1H); 3.32 (m, 4H); 3.80 (m, 2H); 3.99 (s, 2H); 4.02 (dd, 1H, J=11.6 and 7.2 Hz); 4.37 (dd, 1H, J=11.6 and 2 Hz); 4.44 (m, 1H); 6.87 (m, 1H); 7.01 (dd, 1H, J=10.4 and 2.4 Hz).

0.17 g (0.41 mmol) of compound 94 are dissolved in 0.5 ml of acetonitrile and then 0.037 g (0.41 mmol) of oxalic acid and 2 ml of water are added. This is lyophilized and 0.20 g (0.40 mmol) of compound 94 oxalate are obtained in the form of a white solid.

¹H NMR (DMSOd₆) δ: 1.37 (m, 3H); 1.44 (m, 2H); 1.82 (m, 2H); 2.68 (m, 2H); 3.11 (m, 2H); 3.31 (m, 6H); 3.81 (m, 2H); 4.00 (s, 2H); 4.11 (dd, 1H, J=11.6 and 6.4 Hz); 4.38 (dd, 1H, J=11.6 and 2 Hz); 4.75 (m, 1H); 6.93 (m, 1H); 7.07 (dd, 1H, J=10.4 and 2.4 Hz).

MS m/z 413 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.67 min, chemical purity: 99.52%.

### EXAMPLE 95

### (S)-methyl 3-((4-(2-(3-oxomorpholino)ethyl)piperidin-1-yl)methyl)-2,3-dihydrobenzo[b][1,4]dioxine-6-carboxylate

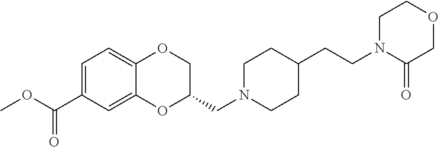 [SMILES: COC(=O)c1ccc2c(c1)O[C@@H](CN1CCC(CCN3CCOCC3=O)CC1)CO2]

By proceeding as in Example 1 but replacing (R)-2-(bromomethyl)-2,3-dihydrobenzo[b][1,4]dioxine 2.1 by (R)-methyl 3-((((4-bromophenyl)sulfonyl)oxy)methyl)-2,3-dihydrobenzo[b][1,4]dioxine-6-carboxylate 2.24, 3-(2-(piperidin-4-yl)ethyl)oxazolidin-2-one 3.1 by 4-(2-(piperidin-4-yl)ethyl)morpholin-3-one 3.28 and the K₂CO₃/KI pair by diisopropylethylamine (1.4 eq), 0.13 g (0.31 mmol) of compound 95 are obtained in the form of a clear oil, after purification by flash chromatography on silica gel (gradient: dichloromethane=100% to dichloromethane/methanol=96/4% over 30 min).

Yield: 47%

¹H NMR (DMSOd₆) δ: 1.14 (m, 3H); 1.41 (m, 2H); 1.65 (m, 2H); 2.00 (m, 2H); 2.54 (m, 2H); 2.83 (m, 1H); 2.92 (m, 1H); 3.32 (m, 4H); 3.80 (m, 2H); 3.99 (s, 2H); 4.04 (dd, 1H, J=11.8 and 7.2 Hz); 4.37 (m, 2H); 6.98 (d, 1H, J=8.4 Hz); 7.39 (d, 1H, J=2 Hz); 7.46 (dd, 1H, J=8.4 and 2 Hz).

0.13 g (0.31 mmol) of compound 95 are dissolved in 0.5 ml of acetonitrile and then 0.028 g (0.31 mmol) of oxalic acid and 2 ml of water are added. This is lyophilized and 0.15 g (0.29 mmol) of compound 95 oxalate are obtained in the form of a white solid.

¹H NMR (DMSOd₆) δ: 1.42 (m, 5H); 1.84 (m, 2H); 2.74 (m, 2H); 3.16 (m, 2H); 3.35 (m, 6H); 3.81 (m, 2H); 4.00 (s, 2H); 4.11 (dd, 1H, J=11.6 and 6.8 Hz); 4.39 (dd, 1H, J=11.6 and 2 Hz); 4.71 (m, 1H); 7.02 (m, 1H); 7.50 (m, 1H).

MS m/z 419 (M+1).

HPLC (X-BRIDGE C18, Phase A: 100% H₂O. 0.1% TFA, Phase B: 100% acetonitrile, gradient B: 0 to 100% in 6 min, 1.8 ml/min), retention time=4.35 min, chemical purity: 99.00%.

### EXAMPLE 96

### (R)-4-(2-(1-(((S)-2,3-dihydrobenzo[b][1,4]dioxin-2-yl)methyl)piperidin-4-yl)ethyl)-6-phenylmorpholin-3-one

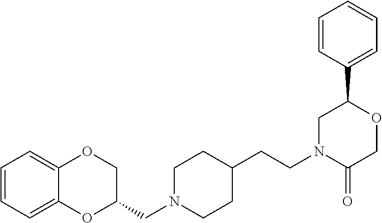 [SMILES: O=C1CO[C@H](c2ccccc2)CN1CCC1CCN(C[C@H]2COc3ccccc3O2)CC1]

By proceeding as in Example 1 but replacing 3-(2-(piperidin-4-yl)ethyl)oxazolidin-2-one 3.1 by (4-(2-(piperidin-4-yl)ethyl)morpholin-3-one 3.29, 1 g (2.29 mmol) of compound 96 is obtained in the form of a clear oil, after purification by flash chromatography on silica gel (eluent: dichloromethane/methanol=95/5%).

Yield: 48%

¹H NMR (CDCl₃) δ: 1.26 (m, 3H); 1.51 (m, 2H); 1.71 (m, 2H); 2.10 (m, 2H); 2.54 (m, 1H); 2.65 (m, 1H); 2.88 (m, 1H); 2.98 (m, 1H); 3.34 (m, 2H); 3.52 (m, 2H); 3.96 (dd, 1H, J=11.6 and 7.8 Hz); 4.36 (m, 4H); 4.79 (dd, 1H, J=10.4 and 2.8 Hz); 6.85 (m, 4H); 7.40 (m, 5H).

1 g (2.29 mmol) of compound 96 is dissolved in 10 ml of methanol and 0.18 g (2.06 mmol) of oxalic acid in solution in 5 ml of methanol are added. This is concentrated and then a little ethyl ether is added, the medium is triturated and 1 g (1.90 mmol) of compound 96 oxalate is obtained in the form of a white solid.

F=133° C.

¹H NMR (DMSOd₆) δ: 1.35 (m, 3H); 1.49 (m, 2H); 1.84 (m, 2H); 2.68 (m, 2H); 3.10 (m, 2H); 3.38 (m, 6H); 3.99 (dd, 1H, J=11.6 and 6.8 Hz); 4.24 (s, 2H); 4.29 (dd, 1H, J=11.4 and 2.4 Hz); 4.62 (m, 1H); 4.89 (dd, 1H, J=10.4 and 3.2 Hz); 6.88 (m, 4H); 7.39 (m, 5H).

HPLC (XBRIDGE C8, acetonitrile/water/KH₂PO₄ (40/60/6.8 g pH 4), 1 ml/min, retention time=6.53 min, chemical purity=90.33%

### EXAMPLE 97

### (S)-3-((4-(2-((R)-5-oxo-2-phenylmorpholino)ethyl)piperidin-1-yl)methyl)-2,3-dihydrobenzo[b][1,4]dioxine-6-carbonitrile

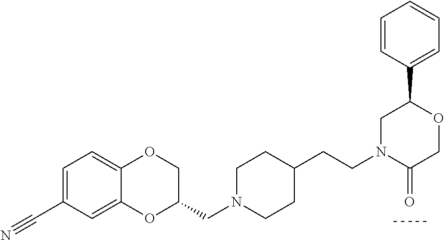

By proceeding as in Example 1 but replacing (R)-2-(bromomethyl)-2,3-dihydrobenzo[b][1,4]dioxine 2.1 by (R)-(7-cyano-2,3-dihydrobenzo[b][1,4]dioxin-2-yl)methyl 4-bromobenzenesulfonate 2.11, 3-(2-(piperidin-4-yl)ethyl)oxazolidin-2-one 3.1 by 4-(2-(piperidin-4-yl)ethyl)morpholin-3-one 3.29, the K₂CO₃/KI pair by DMAP (1.4 eq) and acetonitrile by dimethylsulfoxide, 1.13 g (2.45 mmol) of compound 97 are obtained in the form of a clear oil, after purification by flash chromatography on silica gel (eluent: dichloromethane/methanol=95/5%).

Yield: 53%

¹H NMR (CDCl₃) δ: 1.27 (m, 2H); 1.55 (m, 3H); 1.73 (m, 2H); 2.09 (m, 2H); 2.54 (dd, 1H, J=13.4 and 6.4 Hz); 2.65 (dd, 1H, J=13.2 and 5.6 Hz); 2.86 (m, 1H); 2.94 (m, 1H); 3.35 (m, 2H); 3.55 (m, 2H); 4.01 (dd, 1H, J=11.2 and 7.6 Hz); 4.29 (m, 1H); 4.39 (m, 3H); 4.79 (dd, 1H, J=10.4 and 3.2 Hz); 6.90 (m, 1H); 7.14 (m, 2H); 7.37 (m, 5H).

1.13 g (2.45 mmol) of compound 97 are dissolved in 10 ml of methanol and then 0.23 g (2.45 mmol) of oxalic acid in solution in 5 ml of methanol are added. This is concentrated and then a little ether is added, the medium is triturated and 1.1 g (1.99 mmol) of compound 97 oxalate are obtained in the form of a white solid.

F=184° C.

¹H NMR (DMSOd₆) δ: 1.37 (m, 3H); 1.48 (m, 2H); 1.84 (m, 2H); 2.67 (m, 2H); 3.09 (m, 2H); 3.38 (m, 6H); 4.12 (m, 1H); 4.25 (s, 2H); 4.40 (m, 1H); 4.70 (m, 1H); 4.89 (dd, 1H, J=10.4 and 3.2 Hz); 7.09 (m, 1H); 7.38 (m, 7H).

MS m/z 462 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=1.30 min, chemical purity: 98.78%.

### EXAMPLE 98

### (S)-3-{4-[4-(2-oxo-oxazolidin-3-yl)-butyl]-piperidin-1-ylmethyl}-2,3-dihydrobenzo[b][1,4]dioxine-6-carbonitrile

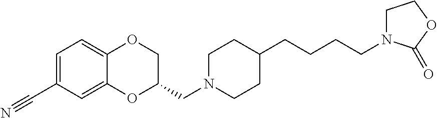 [SMILES: N#Cc1ccc2c(c1)O[C@@H](CN1CCC(CCCCN3CCOC3=O)CC1)CO2]

By proceeding as in Example 1 but replacing (R) 2-(bromomethyl)-2,3-dihydrobenzo[b][1,4]dioxine 2.1 by (R)-(7-cyano-2,3-dihydrobenzo[b][1,4]dioxin-2-yl)methyl 4-bromobenzenesulfonate 2.11, 3-(2-(piperidin-4-yl)ethyl)oxazolidin-2-one 3.1 by 3-(4-piperidin-4-yl-butyl)-oxazolidin-2-one 3.34, the K₂CO₃/KI pair by N,N′-diisopropylethylamine (1.4 eq), compound 98 is obtained in the form of a clear oil, after purification by flash chromatography on silica gel (eluent: dichloromethane/methanol=95/5%).

Yield: 75%

¹H NMR (CDCl₃) δ: 1.27 (m, 4H); 1.50-1.66 (m, 7H); 2.65 (m, 2H,); 2.86 (m, 2H); 2.95 (dd, 2H, J=8 and 6 Hz); 3.25 (t, 2H, J=7 Hz); 3.50 (m, 2H); 4.02 (dd, 1H, J=10 and 7 Hz); 4.35 (m, 4H); 6.89 (d, 1H, J=8 Hz); 7.15 (dd, 1H, J=8 and 2 Hz); 7.16 (d, 1H, J=2 Hz).

0.55 g (1.38 mmol) of compound 98 are dissolved in 10 ml of methanol and 0.128 g (1.38 mmol) of oxalic acid in solution in 5 ml of methanol are added. This is concentrated and then a little ether is added, the medium is triturated and 0.4 g (0.77 mmol) of compound 97 oxalate are obtained in the form of a white solid.

F=182° C.

¹H NMR (DMSOd₆) δ: 1.17 (m, 4H); 1.49 (m, 5H); 1.82 (m, 2H); 3.03 (m, 2H); 3.13 (m, 2H); 3.35 (m, 5H); 3.67 (m, 1H); 4.2 (m, 3H); 4.46 (dd, 1H, J=12 and 2.4 Hz); 5 (m, 1H); 7.1 (d, 1H, J=7.9 Hz); 7.39 (dd, 1H, J=7.9 and 2 Hz); 7.52 (d, 1H, J=2 Hz).

MS m/z 400 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=1.18 min, chemical purity: 99.7%.

### EXAMPLE 99

### (S)-3-{4-[3-((R)-2-oxo-4-phenyl-oxazolidin-3-yl)propyl]-piperidin-1-ylmethyl}-2,3-dihydrobenzo[b][1,4]dioxine-6-carbonitrile

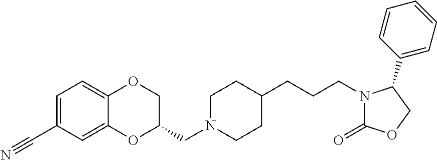 [SMILES: N#Cc1ccc2c(c1)O[C@@H](CN1CCC(CCCN3C(=O)OC[C@H]3c3ccccc3)CC1)CO2]

By proceeding as in Example 1 but replacing (R)-2-(bromomethyl)-2,3-dihydrobenzo[b][1,4]dioxine 2.1 by (R)-(7-cyano-2,3-dihydrobenzo[b][1,4]dioxin-2-yl)methyl 4-bromobenzenesulfonate 2.11, 3-(2-(piperidin-4-yl)ethyl)oxazolidin-2-one 3.1 by (R)-4-phenyl-3-(3-(piperidin-4-yl)propyl)-oxazolidin-2-one 3.32, the K₂CO₃/KI pair by N,N′-diisopropylethylamine (1.4 eq), compound 99 is obtained in the form of a clear oil, after purification by flash chromatography on silica gel (eluent: dichloromethane/methanol=95/5%).

Yield: 53%

¹H NMR (CDCl₃) δ: 1.16 (m, 5H); 1.43 (m, 2H); 1.58 (m, 2H); 2.05 (m, 2H,); 2.55 (dd, 1H); 2.65 (dd, 1H); 2.7-3 (m, 3H); 3.4 (m, 1H,); 4-4.15 (m, 2H); 4.3 (m, 1 Hz); 4.35 (dd, 1H, J=4 and 7 Hz); 4.55 (dd, 1H, J=4 and 7 Hz); 4.7 (t, 1H); 6.90 (d, 1H, J=7 Hz); 7.15 (dd, 1H, J=7 and 2 Hz); 7.16 (d, 1H, J=2 Hz); 7.41 (m, 5H).

0.17 g (0.37 mmol) of compound 99 are dissolved in 10 ml of ethanol and then 0.033 g (0.37 mmol) of oxalic acid in solution in 5 ml of ethanol are added. This is concentrated and then a little acetone is added, the medium is triturated and 0.17 g (0.31 mmol) of compound 99 oxalate are obtained in the form of a white solid.

F=196° C.

¹H NMR (DMSOd₆) δ: 1.33 (m, 5H); 1.71 (m, 4H); 2.60 (m, 3H); 3.10 (m, 2H); 3.12 (m, 1H); 3.32 (m, 2H); 4.10 (m, 2H); 4.39 (m, 1H); 4.63 (m, 2H); 4.91 (m, 1H); 7.20 (2 d, 2H); 7.40 (m, 5H).

MS m/z 462 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=1.39 min, chemical purity: 99.7%.

### EXAMPLE 100

### 3-(3-{1-[(S)-1-(2,3-dihydrobenzo[b][1,4]dioxin-2-yl)methyl]-piperidin-4-yl}-propyl)-oxazolidin-2-one

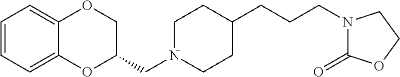 [SMILES: O=C1OCCN1CCCC1CCN(C[C@H]2COc3ccccc3O2)CC1]

By proceeding as in Example 1 but replacing 3-(2-(piperidin-4-yl)ethyl)oxazolidin-2-one 3.1 by 3-(3-(piperidin-4-yl)propyl)oxazolidin-2-one 3.30 and the K₂CO₃/KI pair by N,N′-diisopropylethylamine (1.4 eq), compound 100 is obtained in the form of a clear oil, after purification by flash chromatography on silica gel (eluent: dichloromethane/methanol=95/5%).

Yield: 51%

¹H NMR (CDCl₃) δ: 1.27 (s, 4H); 1.48-1.57 (m, 5H); 2.1 (m, 2H); 2.65 (m, 2H); 2.89 (d, 1H, J=5 Hz); 2.99 (m, 1H, J=5 Hz); 3.25 (t, 2H, J=7.5 Hz); 3.55 (t, 2H, J=8 Hz); 3.97 (dd, 1H, J=7 and 8 Hz); 4.35 (m, 4H); 6.83 (m, 4H).

0.200 g (0.55 mmol) of compound 100 are dissolved in 5 ml of ethanol and then 0.051 g (0.55 mmol) of oxalic acid in solution in 5 ml of ethanol are added. This is concentrated and then a little ether is added, the medium is triturated and 0.127 g (0.28 mmol) of compound 100 oxalate are obtained in the form of a white solid.

F=129° C.

¹H NMR (DMSOd₆) δ: 1.45-1.55 (m, 5H); 1.9 (m, 4H); 3.11 (m, 2H); 3.22 (t, 2H, J=7.8 Hz); 3.31 (m, 1H); 3.49 (m, 2H); 3.50 (t, 2H, J=8 Hz); 3.7 (m, 1H); 4.05 (m, 1H); 4.22 (t, 2H, J=8 Hz); 4.32 (m, 1H); 4.86 (m, 1H); 6.91 (m, 4H); 10.3 (s, 2H).

MS m/z 361 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=1.01 min, chemical purity: 99.43%.

### EXAMPLE 101

### 4-(3-{1-[(S)-1-(2,3-dihydrobenzo[b][1,4]dioxin-2-yl)methyl]-piperidin-4-yl}-propyl)-morpholin-3-one

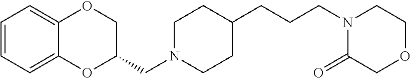 [SMILES: O=C1COCCN1CCCC1CCN(C[C@H]2COc3ccccc3O2)CC1]

By proceeding as in Example 1 but replacing 3-(2-(piperidin-4-yl)ethyl)oxazolidin-2-one 3.1 by 4-(3-piperidin-4-yl-propyl)-morpholin-3-one 3.33 and the K₂CO₃/KI pair by N,N′-diisopropylethylamine (1.4 eq), compound 101 is obtained in the form of a clear oil, after purification by flash chromatography on silica gel (eluent: dichloromethane/methanol=95/5%).

Yield: 40%

¹H NMR (CDCl₃) δ: 1.25 (s, 4H); 1.50-1.60 (m, 5H); 2.1 (m, 2H); 2.5-2.65 (m, 2H); 2.90 (d, 1H, J=6 Hz); 2.99 (m, 1H, J=6 Hz); 3.45 (m, 4H); 3.9 (t, 2H, J=7.6 Hz); 4 (m, 1H); 4.16 (s, 2H); 4.35 (dd, 2H, J=10 and 4); 6.84 (m, 4H)

0.160 g (427 mmol) of compound 101 are dissolved in 5 ml of ethanol and then 0.038 g (0.427 mmol) of oxalic acid in solution in 5 ml of ethanol are added. This is concentrated and then a little ether is added, the medium is triturated and 0.1 g (0.21 mmol) of compound 101 oxalate is obtained in the form of a white solid.

F=139° C.

¹H NMR (DMSOd₆) δ: 1.36-1.44 (m, 5H); 1.85 (m, 4H); 2.63 (m, 2H); 3.10 (m, 2H); 3.20 (m, 1H); 3.33 (m, 5H); 3.81 (m, 2H); 4.10 (s, 2H); 4.12 (dd, 1H. J=12 and 6 Hz); 4.40 (dd, 1H, J=12 and 6 Hz); 4.69 (m, 1H); 7.08 (d, 1H, J=7.5 Hz); 7.35-7.48 (m, 4H); 10.2 (s, 2H).

MS m/z 375 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=0.97 min, chemical purity: 99.6%.

### EXAMPLE 102

### (S)-3-{4-[3-(3-oxo-morpholin-4-yl)-propyl]-piperidin-1-ylmethyl}-2,3-dihydrobenzo[b][1,4]dioxine-6-carbonitrile

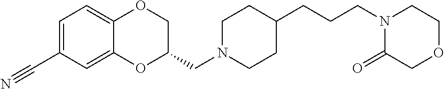 [SMILES: N#Cc1ccc2c(c1)O[C@@H](CN1CCC(CCCN3CCOCC3=O)CC1)CO2]

By proceeding as in Example 1 but replacing (R)-2-(bromomethyl)-2,3-dihydrobenzo[b][1,4]dioxine 2.1 by (R)-(7-cyano-2,3-dihydrobenzo[b][1,4]dioxin-2-yl)methyl 4-bromobenzenesulfonate 2.11, 3-(2-(piperidin-4-yl)ethyl)oxazolidin-2-one 3.1 by 4-(3-piperidin-4-yl-propyl)-morpholin-3-one 3.33 and the K₂CO₃/KI pair by N,N′-diisopropylethylamine (1.4 eq), compound 102 is obtained in the form of a clear oil, after purification by flash chromatography on silica gel (eluent: dichloromethane/methanol=95/5%).

Yield: 66%

¹H NMR (CDCl₃) δ: 1.25 (s, 4H); 1.55-1.60 (m, 5H); 2.1 (m, 2H); 2.5-2.65 (m, 2H); 2.85 (m, 2H); 3.40 (m, 4H); 3.9 (t, 2H, J=7.6 Hz); 4 (m, 1H); 4.16 (s, 2H); 4.35 (dd, 2H, J=10 and 4); 6.90 (d, 1H, J=7 Hz); 7.15 (dd, 1H, J=7 and 2 Hz); 7.16 (d, 1H, J=2 Hz).

0.27 g (0.67 mmol) of compound 102 are dissolved in 5 ml of ethanol and then 0.061 g (0.67 mmol) of oxalic acid in solution in 5 ml of ethanol are added. This is concentrated and then a little ether is added, the medium is triturated and 0.148 g (0.302 mmol) of compound 102 oxalate are obtained in the form of a white solid.

F=168° C.

¹H NMR (DMSOd₆) δ: 1.34-1.44 (m, 5H); 1.77 (m, 4H); 2.63 (m, 2H); 3.10 (m, 2H); 3.20 (m, 1H); 3.33 (m, 5H); 3.81 (m, 2H); 4.10 (s, 2H); 4.12 (dd, 1H, J=12 and 6 Hz); 4.40 (dd, 1H, J=12 and 2 Hz); 4.69 (m, 1H); 7.08 (d, 1H, J=7.5 Hz); 7.35 (dd, 1H. J=7.5 and 2 Hz); 7.44 (d, 1H, =2 Hz); 10.5 (s, 2H).

MS m/z 400 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=1.98 min, chemical purity: 99.93%.

### EXAMPLE 103

### 3-{3-[1-((S)-7-chloro-2,3-dihydrobenzo[b][1,4]dioxin-2-ylmethyl)-piperidin-4-yl]-propyl}-oxazolidin-2-one

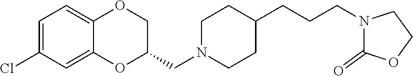 [SMILES: O=C1OCCN1CCCC1CCN(C[C@H]2COc3ccc(Cl)cc3O2)CC1]

By proceeding as in Example 1 but replacing (R)-2-(bromomethyl)-2,3-dihydrobenzo[b][1,4]dioxine 2.1 by (R)-2-(bromomethyl)-7-chloro-2,3-dihydrobenzo[b][1,4]dioxine 2.7, 3-(2-(piperidin-4-yl)ethyl)oxazolidin-2-one 3.1 by 3-(3-(piperidin-4-yl)propyl)oxazolidin-2-one 3.30, the K₂CO₃/KI pair by N,N′-diisopropylethylamine (1.4 eq), compound 103 is obtained in the form of a clear oil, after purification by flash chromatography on silica gel (eluent: dichloromethane/methanol=95/5%).

Yield: 60%

¹H NMR (CDCl₃) δ: 1.25 (s, 4H); 1.50-1.60 (m, 5H); 2.1 (m, 2H); 2.65 (m, 2H); 2.88 (d, 1H, J=5 Hz); 3 (d, 1H, J=5 Hz); 3.24 (t, 2H, J=7.5 Hz); 3.55 (t, 2H, J=8 Hz); 3.97 (dd, 1H, J=7 and 8 Hz); 4.35 (m, 4H); 6.79 (2d, 2H); 6.88 (s, 1H).

0.17 g (0.43 mmol) of compound 103 are dissolved in 5 ml of ethanol and then 0.04 g (0.43 mmol) of oxalic acid in solution in 2 ml of ethanol are added. This is concentrated and then a little ether is added, the medium is triturated and 0.158 g (0.32 mmol) of compound 103 oxalate are obtained in the form of a white solid.

F=149° C.

¹H NMR (DMSOd₆) δ: 1.44 (m, 5H); 1.89 (m, 4H); 3.02 (m, 2H); 3.20 (t, 2H); 3.38 (m, 3H); 3.52 (t, 2H, J=6.8 Hz); 3.63 (m, 1H); 4.08 (dd, 1H, J=12 and 6 Hz); 4.21 (t, 2H, J=7 Hz); 4.30 (m, 1H); 4.87 (m, 1H); 6.92 (ls, 2H); 7.1 (ls, 1H); 10.2 (Is, 1H).

MS m/z 395 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=0.89 min, chemical purity: 99.9%.

### EXAMPLE 104

### 3-{3-[1-((S)-7-chloro-5-fluoro-2,3-dihydrobenzo[b][1,4]dioxin-2-ylmethyl)-piperidin-4-yl]-propyl}-oxazolidin-2-one

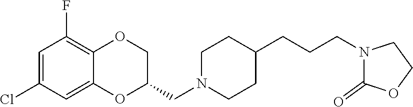 [SMILES: O=C1OCCN1CCCC1CCN(C[C@H]2COc3c(F)cc(Cl)cc3O2)CC1]

By proceeding as in Example 1 but replacing (R)-2-(bromomethyl)-2,3-dihydrobenzo[b][1,4]dioxine 2.1 (R)-(7-chloro-5-fluoro-2,3-dihydrobenzo[b][1,4]dioxin-2-yl)methyl 4-bromobenzenesulfonate 2.20, 3-(2-(piperidin-4-yl)ethyl)oxazolidin-2-one 3.1 by 3-(3-(piperidin-4-yl)propyl)oxazolidin-2-one 3.30, the K₂CO₃/KI pair by N,N′-diisopropylethylamine (1.4 eq), compound 104 is obtained in the form of a clear oil, after purification by flash chromatography on silica gel (eluent: dichloromethane/methanol=95/5%).

Yield: 67%

¹H NMR (CDCl₃) δ: 1.26 (s, 4H); 1.56-1.65 (m, 5H); 2.1 (m, 2H); 2.65 (m, 2H); 2.75 (m, 1H); 2.9 (dd, 2H, J=8 and 5 Hz); 3.25 (t, 2H, J=7 Hz); 3.55 (t, 2H, J=7 Hz); 3.97 (m, 1H); 4.35 (m, 4H); 6.70 (2d, 2H).

0.190 g (0.46 mmol) of compound 104 are dissolved in 5 ml of ethanol and then 0.041 g (0.46 mmol) of oxalic acid in solution in 5 ml of ethanol are added. This is concentrated and then a little ether is added, the medium is triturated and 0.2 g (0.394 mmol) of compound 104 oxalate are obtained in the form of a white solid.

F=160° C.

¹H NMR (DMSOd₆) δ: 1.30-1.45 (m, 5H); 1.92 (m, 4H); 3. (m, 2H); 3.17 (t, 2H, J=6 Hz); 3.42 (m, 3H); 3.49 (t, 2H, J=8 Hz); 3.64 (m, 1H); 4.16 (dd, 1H); 4.25 (t, 2H, J=8 Hz); 4.40 (m, 1H); 4.97 (m, 1H); 6.97 (m, 1H); 7.12 (m, 1H).

MS m/z 413 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=1.03 min, chemical purity: 99.8%.

### EXAMPLE 105

### 3-{3-[1-((S)-5,7difluoro-2,3-dihydro-benzo[b][1,4]dioxin-2-ylmethyl)-piperidin-4-yl]-propyl}-oxazolidin-2-one

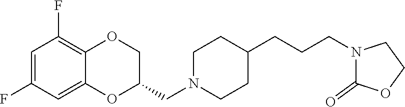 [SMILES: O=C1OCCN1CCCC1CCN(C[C@H]2COc3c(F)cc(F)cc3O2)CC1]

By proceeding as in Example 1 but replacing (R)-2-(bromomethyl)-2,3-dihydrobenzo[b][1,4]dioxine 2.1 by (R)-(5,7-difluoro-2,3-dihydrobenzo[b][1,4]dioxin-2-yl)methyl 4-bromobenzenesulfonate 2.19, 3-(2-(piperidin-4-yl)ethyl)oxazolidin-2-one 3.1 by 3-(3-(piperidin-4-yl)propyl)oxazolidin-2-one 3.30 and the K₂CO₃/KI pair by N,N′-diisopropylethylamine (1.4 eq), compound 105 is obtained in the form of a clear oil, after purification by flash chromatography on silica gel (eluent: dichloromethane/methanol=95/5%).

Yield: 96%

¹H NMR (CDCl₃) δ: 1.25 (s, 4H); 1.55-1.60 (m, 5H); 2.1 (m, 2H); 2.65 (m, 2H); 2.80 (d, 1H, J=8 Hz); 2.95 (d, 1H, J=8 Hz); 3.30 (t, 2H, J=7 Hz); 3.55 (t, 2H, J=7 Hz); 4 (m, 1H) 4.33 (m, 4H); 6.45 (2d, 2H).

0.270 g (0.68 mmol) of compound 105 are dissolved in 5 ml of ethanol and then 0.61 g (0.68 mmol) of oxalic acid in solution in 5 ml of ethanol are added. This is concentrated and then a little ether is added, the medium is triturated and 0.19 g (0.39 mmol) of compound 105 oxalate are obtained in the form of a white solid.

F=184° C.

¹H NMR (DMSOd₆) δ: 1.35-1.47 (m, 5H); 1.92 (m, 4H); 3.1 (m, 2H); 3.20 (m, 2H); 3.40 (m, 3H); 3.552 (t, 2H J=8 Hz); 3.65 (m, 1H); 4.13 (dd, 1H, J=10 and 5 Hz); 4.25 (t, 2H, J=8 Hz); 4.38 (m, 1H); 4.97 (m, 1H); 6.79 (m, 1H); 6.95 (m, 1H); 10 (s, 1H).

MS m/z 397 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=1.49 min, chemical purity: 99.6%.

### EXAMPLE 106

### 3-{3-[1-((S)-7-fluoro-2,3-dihydro-benzo[b][1,4]dioxin-2-ylmethyl)-piperidin-4-yl]-propyl}-oxazolidin-2-one

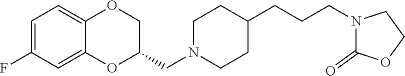 [SMILES: O=C1OCCN1CCCC1CCN(C[C@H]2COc3ccc(F)cc3O2)CC1]

By proceeding as in Example 1 but replacing (R)-2-(bromomethyl)-2,3-dihydrobenzo[b][1,4]dioxine 2.1 by (R)-(7-fluoro-2,3-dihydrobenzo[b][1,4]dioxin-2-yl)methyl 4-bromobenzenesulfonate 2.17, 3-(2-(piperidin-4-yl)ethyl)oxazolidin-2-one 3.1 by 3-(3-(piperidin-4-yl)propyl)oxazolidin-2-one 3.30 and the K₂CO₃/KI pair by N,N′-diisopropylethylamine (1.4 eq), compound 106 is obtained in the form of a clear oil, after purification by flash chromatography on silica gel (eluent: dichloromethane/methanol=95/5%).

Yield: 60%

¹H NMR (CDCl₃) δ: 1.24 (s, 4H); 1.52-1.60 (m, 5H); 2.05 (m, 2H); 2.65 (m, 2H); 2.88 (d, 1H, J=5 Hz); 3 (d, 1H, J=5 Hz); 3.24 (t, 2H, J=7.5 Hz); 3.55 (t, 2H, J=8 Hz); 3.97 (m, 1H); 4.33 (m, 4H); 6.55 (1d, 1H, J=6 Hz); 6.77 (1d, 1H, J=6 Hz); 6.80 (s, 1H).

0.17 g (449 mmol) of compound 106 are dissolved in 5 ml of ethanol and then 0.04 g (0.449 mmol) of oxalic acid in solution in 5 ml of ethanol are added. This is concentrated and then a little ether is added, the medium is triturated and 0.17 g (0.36 mmol) of compound 106 oxalate are obtained in the form of a white solid.

F=136° C.

¹H NMR (DMSOd₆) δ: 1.45 (m, 5H); 1.92 (m, 4H); 3.01 (m, 2H); 3.20 (m, 2H); 3.35 (m, 3H); 3.52 (t, 2H,); 3.65 (m, 1H); 4.05 (dd, 1H, J=10 and 7 Hz); 4.23 (t, 2H, J=8 Hz); 4.25 (m, 1H); 4.67 (m, 1H); 6.72 (m, 1H); 6.85 (m, 1H); 6.93 (m, 1H); 10.2 (Is, 1H).

MS m/z 379 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=1.28 min, chemical purity: 99.4%.

### EXAMPLE 107

### (S)-3-{4-[3-((S)-4-benzyl-2-oxo-oxazolidin-3-yl)-propyl]-piperidin-1-ylmethyl}-2,3-dihydrobenzo[b][1,4]dioxine-6-carbonitrile

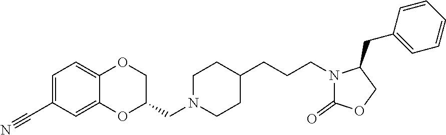 [SMILES: N#Cc1ccc2c(c1)O[C@@H](CN1CCC(CCCN3C(=O)OC[C@@H]3Cc3ccccc3)CC1)CO2]

By proceeding as in Example 1 but replacing (R)-2-(bromomethyl)-2,3-dihydrobenzo[b][1,4]dioxine 2.1 by (R)-(7-cyano-2,3-dihydrobenzo[b][1,4]dioxin-2-yl)methyl 4-bromobenzenesulfonate 2.11, 3-(2-(piperidin-4-yl)ethyl)oxazolidin-2-one 3.1 by (S)-4-benzyl-3-(3-(piperidin-4-yl)propyl)oxazolidin-2-one 3.31 and the K₂CO₃/KI pair by N,N′-diisopropylethylamine (1.4 eq), compound 107 is obtained in the form of a clear oil, after purification by flash chromatography on silica gel (eluent: dichloromethane/methanol=95/5%).

Yield: 43%

¹H NMR (CDCl₃) δ: 1.25 (m, 5H); 1.50 (m, 4H); 2.1 (m, 2H,); 2.55 (dd, 1H); 2.65 (dd, 1H); 2.7-3 (m, 3H); 3.4 (m, 1H); 3.47 (m, 2H); 4-4.15 (m, 2H); 4.3 (m, 1 Hz); 4.35 (dd, 1H, J=4 and 7 Hz); 4.55 (dd, 1H, J=4 and 7 Hz); 4.7 (t, 1H); 6.91 (d, 1H, J=7 Hz); 7.15 (m, 4H); 7.3 (m, 3H); 7.41 (m, 5H).

0.2 g (0.42 mmol) of compound 107 are dissolved in 5 ml of ethanol and then 0.038 g (0.42 mmol) of oxalic acid in solution in 5 ml of ethanol are added. This is concentrated and then a little acetone is added, the medium is triturated and 0.196 g (0.35 mmol) of compound 107 oxalate are obtained in the form of a white solid.

F=160° C.

¹H NMR (DMSOd₆) δ: 1.44 (m, 5H); 1.92 (m, 4H); 2.51 (m, 2H); 2.72 (m, 1H); 3.08 (m, 4H); 3.47 (m, 2H); 3.66 (m, 1H); 3.97 (m, 1H); 4.14 (m, 3H); 4.44 (m, 1H); 4.97 (m, 1H); 7.08 (d, 1H, J=8 Hz); 7.28-7.36 (m, 5H); 7.47 (d, 1H, J=2 Hz); 10.2 (Is, 1H).

MS m/z 476 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=1.72 min, chemical purity: 99.94%.

### EXAMPLE 108

### 3-{3-[1-((S)-5-chloro-7-fluoro-2,3-dihydro-benzo[b][1,4]dioxin-2-ylmethyl)-piperidin-4-yl]-propyl}-oxazolidin-2-one

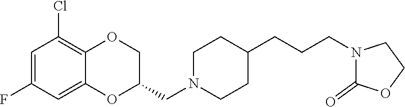 [SMILES: O=C1OCCN1CCCC1CCN(C[C@H]2COc3c(Cl)cc(F)cc3O2)CC1]

By proceeding as in Example 1 but replacing (R)-2-(bromomethyl)-2,3-dihydrobenzo[b][1,4]dioxine 2.1 by (R)-(5-chloro-7-fluoro-2,3-dihydrobenzo[b][1,4]dioxin-2-yl)methyl 4-bromobenzenesulfonate 2.21, 3-(2-(piperidin-4-yl)ethyl)oxazolidin-2-one 3.1 by 3-(3-(piperidin-4-yl)propyl)oxazolidin-2-one 3.30 and the K₂CO₃/KI pair by N,N′-diisopropylethylamine (1.4 eq), compound 108 is obtained in the form of a clear oil, after purification by flash chromatography on silica gel (eluent: dichloromethane/methanol=95/5%).

Yield: 38%

¹H NMR (CDCl₃) δ: 1.24 (m, 4H); 1.56-1.67 (m, 5H); 2.05 (m, 2H); 2.60 (m, 2H); 2.75 (m, 1H); 2.9 (dd, 2H, J=8 and 5 Hz); 3.25 (t, 2H, J=7 Hz); 3.56 (t, 2H, J=7 Hz); 3.97 (m, 1H); 4.35 (m, 4H); 6.56 (d, 1H, J=6 Hz); 6.7 (d, 1H, J=6 Hz).

0.12 g (0.29 mmol) of compound 108 are dissolved in 5 ml of ethanol and then 0.026 g (0.29 mmol) of oxalic acid in solution in 5 ml of ethanol are added. This is concentrated and then a little ether is added, the medium is triturated and 0.12 g (0.24 mmol) of compound 108 oxalate are obtained in the form of a white solid.

F=108° C.

¹H NMR (DMSOd₆) δ: 1.35-1.50 (m, 5H); 1.91 (m, 4H); 3 (m, 2H); 3.20 (t, 2H); 3.40 (m, 3H); 3.50 (t, 2H, J=8 Hz); 3.60 (m, 1H); 4.20 (dd, 1H, J=12 and 6 Hz); 4.25 (t, 2H, J=8 Hz); 4.41 (m, 1H); 4.92 (m, 1H); 6.88 (d, 1H, J=5 Hz); 7.04 (d, 1H, J=5 Hz).

MS m/z 413 (M+1).

HPLC (XBridge BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=1.08 min, chemical purity: 98.99%.

### EXAMPLE 109

### 3-{3-[1-((S)-7-methanesulfonyl-2,3-dihydro-benzo[b][1,4]dioxin-2-ylmethyl)-piperidin-4-yl]-propyl}-oxazolidin-2-one

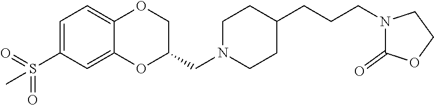 [SMILES: CS(=O)(=O)c1ccc2c(c1)O[C@@H](CN1CCC(CCCN3CCOC3=O)CC1)CO2]

By proceeding as in Example 1 but replacing (R)-2-(bromomethyl)-2,3-dihydrobenzo[b][1,4]dioxine 2.1 by (R)-(7-(methylsulfonyl)-2,3-dihydrobenzo[b][1,4]dioxin-2-yl)methyl 4-bromobenzenesulfonate 2.23, 3-(2-(piperidin-4-yl)ethyl)oxazolidin-2-one 3.1 by 3-(3-(piperidin-4-yl)propyl)oxazolidin-2-one 3.30 and the K₂CO₃/KI pair by N,N′-diisopropylethylamine (1.4 eq), compound 109 is obtained in the form of a clear oil, after purification by flash chromatography on silica gel (eluent: dichloromethane/methanol=95/5%).

Yield: 58%

¹H NMR (CDCl₃) δ: 1.26 (s, 4H); 1.52-1.60 (m, 5H); 2.05 (m, 2H); 2.65 (m, 2H); 2.88 (d, 1H, J=5 Hz); 3 (d, 1H, J=5 Hz); 3.24 (t, 2H, J=7.5 Hz); 3.55 (t, 2H, J=8 Hz); 3.97 (m, 1H); 4.33 (m, 4H); 6.54 (1d, 1H, J=6 Hz); 6.75 (1d, 1H, J=6 Hz); 6.77 (s, 1H).

0.16 g (0.365 mmol) of compound 109 are dissolved in 5 ml of ethanol and then 0.033 g (0.365 mmol) of oxalic acid in solution in 5 ml of ethanol are added. This is concentrated and then a little acetone is added, the medium is triturated and 0.125 g (1.99 mmol) of compound 109 oxalate are obtained in the form of a white solid.

F=127° C.

¹H NMR (DMSOd₆) δ: 1.27-1.40 (m, 5H); 1.85 (m, 2H); 2.70 (m, 2H); 3.06 (ls, 2H); 3.20 (t, 2H, J=7.5 Hz); 3.30 (m, 2H); 3.51 (t, 2H, J=7 Hz); 4.12 (m, 1H); 4.22 (m, 2H); 4.48 (m, 1H); 4.74 (ls, 1H); 7.14 (d, 1H, J=2 Hz); 7.45 (m, 2H).

MS m/z 439 (M+1).

HPLC (XBridge BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=1.44 min, chemical purity: 99.26%.

### EXAMPLE 110

### (S)-8-fluoro-3-{4-[3-(2-oxo-oxazolidin-3-yl)-propyl]-piperidin-1-ylmethyl}-2,3-dihydrobenzo[b][1,4]dioxine-6-carbonitrile

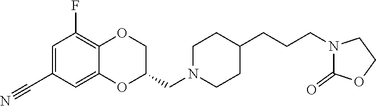 [SMILES: N#Cc1cc(F)c2c(c1)O[C@@H](CN1CCC(CCCN3CCOC3=O)CC1)CO2]

By proceeding as in Example 1 but replacing (R)-2-(bromomethyl)-2,3-dihydrobenzo[b][1,4]dioxine 2.1 by (R)-(7-cyano-5-fluoro-2,3-dihydrobenzo[b][1,4]dioxin-2-yl)methyl-4-bromobenzene sulfonate 2.26, 3-(2-(piperidin-4-yl)ethyl)oxazolidin-2-one 3.1 by 3-(3-(piperidin-4-yl)propyl)oxazolidin-2-one 3.30 and the K₂CO₃/KI pair by N,N′-diisopropylethylamine (1.4 eq), compound 110 is obtained in the form of a clear oil, after purification by flash chromatography on silica gel (eluent: dichloromethane/methanol=95/5%).

Yield: 47%

¹H NMR (CDCl₃) δ: 1.25 (s, 4H); 1.55-1.65 (m, 5H); 2.05 (m, 2H); 2.60 (m, 2H); 2.80 (m, 1H); 2.9 (dd, 2H, J=7 and 5 Hz); 3.25 (t, 2H, J=7 Hz); 3.55 (t, 2H, J=7 Hz); 4 (m, 1H); 4.30 (m, 4H); 6.98 (2d, 2H).

0.110 g (0.273 mmol) of compound 110 are dissolved in 5 ml of ethanol and then 0.025 g (0.273 mmol) of oxalic acid in solution in 5 ml of ethanol are added. This is concentrated and then a little ether is added, the medium is triturated and 0.16 g (0.235 mmol) of compound 110 oxalate are obtained in the form of a white solid.

F=171° C.

¹H NMR (DMSOd₆) δ: 1.20-1.40 (m, 5H); 1.80 (m, 2H); 2.60 (m, 2H); 3.06 (ls, 2H); 3.20 (t, 2H, J=6.8 Hz); 3.30 (m, 2H); 3.51 (t, 2H, J=7 Hz); 4.12 (m, 1H); 4.22 (m, 2H); 4.48 (m, 1H); 4.75 (ls, 1H); 7.37 (d, 1H, J=2 Hz); 7.51 (d, 1H, J=2 Hz).

MS m/z 404 (M+1).

HPLC (Acquity BEH C8, Phase A: 100% H₂O. 0.01 M pH 4 HCOONH₄, Phase B: 90% acetonitrile 10% H₂O. 0.01 M pH 4 HCOONH₄, gradient B: 35 to 60% in 5 min, 0.75 ml/min), retention time=1.12 min, chemical purity: 97%.

### “Benzodioxane” Part

Structures of the (2,3-dihydrobenzo[b][1,4]dioxin-2-yl)methanol type, optionally substituted on the aromatic ring and optically pure are prepared according to two methods, depending on whether 2-hydroxybenzaldehydes (J. Med. Chem.; 1997; 40; 4235-4256; method A) or 2-bromophenols (J. Am. Chem. Soc.; 2001; 123; 12202-12206; method B) are used as raw materials. Methods A or B are chosen according to the commercial availability or synthetic accessibility of the raw materials.

### Method A:

J. Med. Chem.; 1997; 40; 4235-4256

### Intermediate 4.1: (S)-(2,3-dihydrobenzo[b][1,4]dioxin-2-yl)methanol

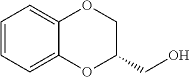 [SMILES: OC[C@H]1COc2ccccc2O1]

¹H NMR (CDCl₃) δ: 1.94 (t, 1H, J=6 Hz); 3.88 (m, 2H); 4.11 (m, 1H); 4.30 (m, 2H); 6.86 (m, 4H).

### Intermediate 4.2: (R)-(2,3-dihydrobenzo[b][1,4]dioxin-2-yl)methanol

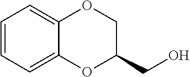 [SMILES: OC[C@@H]1COc2ccccc2O1]

¹H NMR (CDCl₃) δ: see NMR intermediate 4.1

### Intermediate 4.3: (S)-(7-methyl-2,3-dihydrobenzo[b][1,4]dioxin-2-yl)methanol

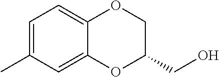

¹H NMR (CDCl₃) δ: 1.94 (t, 1H, J=6.4 Hz); 2.25 (s, 3H); 3.86 (m, 2H); 4.10 (m, 1H); 4.23 (m, 2H); 6.65 (dd, 1H, J=8 and 1.6 Hz); 6.71 (d, 1H, J=1.6 Hz); 6.77 (d, 1H, J=8 Hz).

### Intermediate 4.4: (S)-(7-(trifluoromethyl)-2,3-dihydrobenzo[b][1,4]dioxin-2-yl)methanol

2-hydroxy-5-(trifluoromethyl)benzaldehyde is not available commercially, it is synthesized according to the method reported in Bioorg. & Med. Chem. Lett.; 2006; Vol 16; 14; 3657-3662.

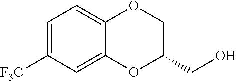 [SMILES: OC[C@H]1COc2ccc(C(F)(F)F)cc2O1]

¹H NMR (CDCl₃) δ: 1.88 (t, 1H, J=6.4 Hz); 3.86 (m, 1H); 3.94 (m, 1H); 4.16 (m, 1H); 4.26 (m, 1H); 4.36 (m, 1H); 6.96 (d, 1H, J=8.8 Hz); 7.12 (dd, 1H, J=8.8 and 2 Hz); 7.17 (d, 1H, J=2 Hz).

### Intermediate 4.5: (S)-(7-chloro-2,3-dihydrobenzo[b][1,4]dioxin-2-yl)methanol

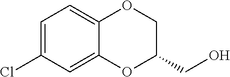 [SMILES: OC[C@H]1COc2ccc(Cl)cc2O1]

¹H NMR (CDCl₃) δ: 1.94 (t, 1H, J=6.4 Hz); 3.87 (m, 2H); 4.11 (m, 1H); 4.24 (m, 2H); 6.81 (m, 2H); 6.90 (m, 1H).

### Intermediate 4.6: (S)-(7-fluoro-2,3-dihydrobenzo[b][1,4]dioxin-2-yl)methanol

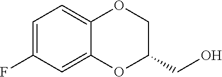 [SMILES: OC[C@H]1COc2ccc(F)cc2O1]

¹H NMR (CDCl₃) δ: 1.93 (t, 1H, J=6.4 Hz); 3.87 (m, 2H); 4.08 (dd, 1H, J=11.8 and 8 Hz); 4.27 (m, 2H); 6.56 (m, 1H); 6.64 (m, 1H); 6.81 (m, 1H).

### Intermediate 4.7: (S)-(8-bromo-2,3-dihydrobenzo[b][1,4]dioxin-2-yl)methanol

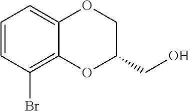 [SMILES: OC[C@H]1COc2cccc(Br)c2O1]

¹H NMR (CDCl₃) δ: 2.14 (t, 1H, J=6.4 Hz); 3.88 (m, 1H); 3.97 (m, 1H); 4.13 (m, 1H); 4.32 (m, 2H); 6.73 (m, 1H); 6.85 (dd, 1H, J=8.2 and 1.6 Hz); 7.11 (dd, 1H, J=8 and 1.6 Hz).

### Intermediate 4.8: (S)-(7-bromo-2,3-dihydrobenzo[b][1,4]dioxin-2-yl)methanol

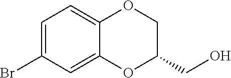 [SMILES: OC[C@H]1COc2ccc(Br)cc2O1]

¹H NMR (CDCl₃) δ: 1.93 (t, 1H, J=6.4 Hz); 3.87 (m, 2H); 4.09 (dd, 1H, J=11 and 8 Hz); 4.27 (m, 2H); 6.75 (d, 1H, J=8.4 Hz); 6.95 (d, 1H, J=8.4 Hz); 7.05 (s, 1H).

### Intermediate 4.9: (S)-(6-bromo-2,3-dihydrobenzo[b][1,4]dioxin-2-yl)methanol

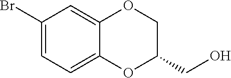 [SMILES: OC[C@H]1COc2cc(Br)ccc2O1]

¹H NMR (CDCl₃) δ: 1.91 (t, 1H, J=6.4 Hz); 3.87 (m, 2H); 4.10 (dd, 1H, J=11.2 and 7.6 Hz); 4.23 (m, 1H); 4.29 (m, 1H); 6.77 (d, 1H, J=8.4 Hz); 6.96 (dd, 1H, J=8.4 and 2.4 Hz); 7.03 (d, 1H, J=2.4 Hz).

### Intermediate 4.10: (S)-3-(hydroxymethyl)-2,3-dihydrobenzo[b][1,4]dioxine-6-carbonitrile

Product prepared from intermediate 4.7, (S)-(7-bromo-2,3-dihydrobenzo[b][1,4]dioxin-2-yl)methanol, previously described, according to the method reported in Tetrahedron; 2008; 64; 10802-10809.

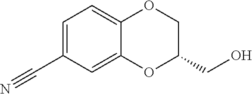 [SMILES: N#Cc1ccc2c(c1)O[C@@H](CO)CO2]

¹H NMR (CDCl₃) δ: 1.88 (t, 1H, J=6 Hz); 3.91 (m, 2H); 4.18 (dd, 1H, J=11.4 and 7.6 Hz); 4.26 (m, 1H); 4.39 (dd, 1H, J=11.4 and 2.4 Hz); 6.94 (d, 1H, J=8.4 Hz); 7.17 (dd, 1H, J=8.4 and 2 Hz); 7.19 (d, 1H, J=2 Hz).

### Intermediate 4.11: (S)-3-(hydroxymethyl)-2,3-dihydrobenzo[b][1,4]dioxine-5-carbonitrile

Product prepared from intermediate 4.6, (S)-(8-bromo-2,3-dihydrobenzo[b][1,4]dioxin-2-yl)methanol, previously described, according to the method reported in Tetrahedron, 2008 64; 10802-10809.

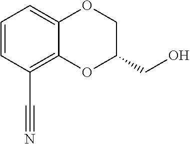 [SMILES: N#Cc1cccc2c1O[C@@H](CO)CO2]

¹H NMR (CDCl₃) δ: 2.25 (t, 1H, J=6.4 Hz); 3.91 (m, 1H); 4.00 (m, 1H); 4.18 (dd, 1H, J=12 and 8.4 Hz); 4.36 (m, 2H); 6.90 (m, 1H); 7.10 (dd, 1H, J=8.4 and 1.6 Hz); 7.15 (dd, 1H, J=7.8 and 1.6 Hz).

### Intermediate 4.12: (S)-(7-(benzyloxy)-2,3-dihydrobenzo[b][1,4]dioxin-2-yl)methanol

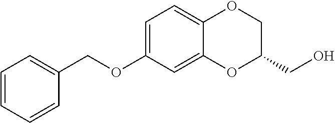 [SMILES: OC[C@H]1COc2ccc(OCc3ccccc3)cc2O1]

¹H NMR (CDCl₃) δ: 1.89 (t, 1H, J=6.4 Hz); 3.86 (m, 2H); 4.06 (m, 1H); 4.25 (m, 2H); 4.98 (s, 2H); 6.50 (dd, 1H, J=8.8 and 2.8 Hz); 6.56 (d, 1H, J=2.8 Hz); 6.79 (d, 1H, J=8.8 Hz); 7.36 (m, 5H).

### Intermediate 4.13: (S)-(6-(benzyloxy)-2,3-dihydrobenzo[b][1,4]dioxin-2-yl)methanol

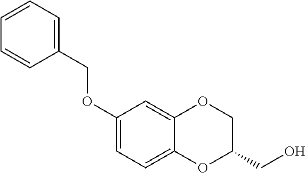 [SMILES: OC[C@H]1COc2cc(OCc3ccccc3)ccc2O1]

¹H NMR (CDCl₃) δ: 1.90 (t, 1H, J=6.4 Hz); 3.85 (m, 2H); 4.09 (m, 11H); 4.20 (m, 1H); 4.29 (m, 1H); 4.98 (s, 2H); 6.52 (m, 2H); 6.80 (d, 1H, J=8.8 Hz); 7.36 (m, 5H).

### Intermediate 4.14: (S)-(7-(trifluoromethoxy)-2,3-dihydrobenzo[b][1,4]dioxin-2-yl)methanol

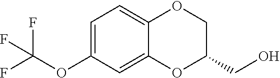 [SMILES: OC[C@H]1COc2ccc(OC(F)(F)F)cc2O1]

¹H NMR (CDCl₃) δ: 1.88 (t, 1H, J=6.4 Hz); 3.89 (m, 2H); 4.11 (dd, 1H, J=11 and 7.2 Hz); 4.26 (m, 1H); 4.31 (dd, 1H, J=11 and 2.4 Hz); 6.73 (m, 1H); 6.81 (d, 1H, J=2 Hz); 6.87 (d, 1H, J=8.8 Hz).

### Intermediate 4.15: (S)-(6-bromo-7-methoxy-2,3-dihydrobenzo[b][1,4]dioxin-2-yl)methanol

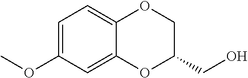

¹H NMR (CDCl₃) δ: 1.93 (t, 1H, J=6.4 Hz); 3.74 (s, 3H); 3.86 (m, 2H); 4.06 (dd, 1H, J=11.8 and 8 Hz); 4.27 (m, 2H); 6.43 (dd, 1H, J=8.8 and 2.8 Hz); 6.49 (d, 1H, J=2.8 Hz); 6.80 (d, 1H, J=8.8 Hz).

### Method B;

J. Am. Chem. Soc.; 2001; 123; 12202-12206

### Intermediate 4.16: (S)-(5-bromo-2,3-dihydrobenzo[b][1,4]dioxin-2-yl)methanol

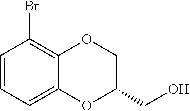 [SMILES: OC[C@H]1COc2c(Br)cccc2O1]

¹H NMR (CDCl₃) δ: 1.87 (t, 1H, J=6.4 Hz); 3.85 (m, 1H); 3.94 (m, 1H); 4.19 (dd, 1H, J=11.2 and 8 Hz); 4.27 (m, 1H); 4.43 (dd, 1H, J=11.2 and 2 Hz); 6.74 (m, 1H); 6.87 (dd, 1H, J=8.4 and 1.6 Hz); 7.11 (dd, 1H, J=8 and 2 Hz).

### Intermediate 4.17: (S)-(8-fluoro-2,3-dihydrobenzo[b][1,4]dioxin-2-yl)methanol

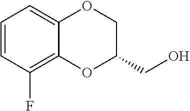 [SMILES: OC[C@H]1COc2cccc(F)c2O1]

¹H NMR (DMSOd₆) δ: 3.66 (m, 2H); 4.05 (dd, 1H, J=11.6 and 7.2 Hz); 4.20 (m, 1H); 4.38 (m, 1H); 5.12 (t, 1H, J=6 Hz); 6.72 (m, 1H); 6.79 (m, 2H).

### Intermediate 4.18: (S)-(6-fluoro-2,3-dihydrobenzo[b][1,4]dioxin-2-yl)methanol

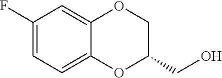 [SMILES: OC[C@H]1COc2cc(F)ccc2O1]

¹H NMR (DMSOd₆) δ: 3.61 (m, 2H); 4.00 (dd, 1H, J=11.2 and 7.6 Hz); 4.11 (m, 1H); 4.32 (m, 1H); 5.07 (t, 1H, J=6 Hz); 6.67 (m, 1H); 6.77 (m, 1H); 6.87 (m, 1H).

### Intermediate 4.19: (S)-(5,7-difluoro-2,3-dihydrobenzo[b][1,4]dioxin-2-yl)methanol

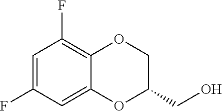 [SMILES: OC[C@H]1COc2c(F)cc(F)cc2O1]

¹H NMR (DMSOd₆) δ: 3.64 (m, 2H); 4.03 (dd, 1H, J=11.4 and 7.2 Hz); 4.25 (m, 1H); 4.39 (dd, 1H, J=11.4 and 2.4 Hz); 5.13 (t, 1H, J=5.6 Hz); 6.69 (m, 1H); 6.85 (m, 1H).

### Intermediate 4.20: (S)-(7-chloro-5-fluoro-2,3-dihydrobenzo[b][1,4]dioxin-2-yl)methanol

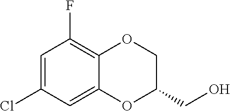 [SMILES: OC[C@H]1COc2c(F)cc(Cl)cc2O1]

¹H NMR (DMSOd₆) δ: 3.64 (m, 2H); 4.07 (dd, 1H, J=11.6 and 7.2 Hz); 4.25 (m, 1H); 4.42 (dd, 1H, J=11.6 and 2.4 Hz); 5.14 (t, 1H, J=5.6 Hz); 6.86 (m, 1H); 7.01 (m, 1H).

### Intermediate 4.21: (S)-(5-chloro-7-fluoro-2,3-dihydrobenzo[b][1,4]dioxin-2-yl)methanol

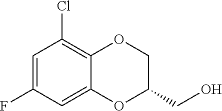 [SMILES: OC[C@H]1COc2c(Cl)cc(F)cc2O1]

¹H NMR (DMSOd₆) δ: 3.63 (m, 2H); 4.07 (dd, 1H, J=11.4 and 7.6 Hz); 2.23 (m, 1H); 4.43 (dd, 1H, J=11.4 and 2.4 Hz); 5.14 (t, 1H, J=5.6 Hz); 6.84 (m, 1H); 6.98 (m, 1H).

### Intermediate 4.22: (S)-(6,7-difluoro-2,3-dihydrobenzo[b][1,4]dioxin-2-yl)methanol

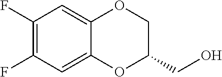 [SMILES: OC[C@H]1COc2cc(F)c(F)cc2O1]

¹H NMR (DMSOd₆) δ: 3.61 (m, 2H); 3.49 (dd, 1H, J=11.4 and 7.2 Hz); 4.15 (m, 1H); 4.33 (dd, 1H, J=11.4 and 2.4 Hz); 5.09 (t, 1H, J=5.6 Hz); 7.04 (m, 2H).

### Intermediate 4.23: (S)-(7-(methylsulfonyl)-2,3-dihydrobenzo[b][1,4]dioxin-2-yl)methanol

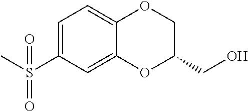 [SMILES: CS(=O)(=O)c1ccc2c(c1)O[C@@H](CO)CO2]

¹H NMR (DMSOd₆) δ: 3.16 (m, 2H); 4.12 (dd, 1H, J=11.4 and 7.6 Hz); 4.24 (m, 1H); 4.43 (dd, 1H, J=11.4 and 2 Hz); 5.13 (t, 1H, J=5.6 Hz); 7.11 (m, 1H); 7.37 (m, 2H).

### Intermediate 4.24: (S)-methyl 3-(hydroxymethyl)-2,3-dihydrobenzo[b][1,4]dioxine-6-carboxylate

Commercial 3-bromo-4-hydroxybenzoique acid is used, from which Weinreb amine is prepared. This amine is transformed into ester at the deprotection of (R)-3-bromo-4-((2,2-dimethyl-1,3-dioxolan-4-yl)methoxy)-N-methoxy-N-methylbenzamide (see method B) by a treatment in acid medium.

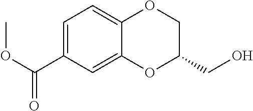 [SMILES: COC(=O)c1ccc2c(c1)O[C@@H](CO)CO2]

¹H NMR (DMSOd₆) δ: 3.64 (m, 2H); 3.80 (s, 3H); 4.09 (dd, 1H, J=11.4 and 7.2 Hz); 4.20 (m, 1H); 4.41 (dd, 1H, J=11.4 and 2 Hz); 5.10 (t, 1H, J=5.6 Hz); 6.98 (d, 1H, J=8.4 Hz); 7.40 (d, 1H, J=2 Hz); 7.46 (dd, 1H, J=8.4 and 2 Hz).

### Intermediate 4.25: (S)-2-(hydroxymethyl)-2,3-dihydrobenzo[b][1,4]dioxine-5-carbonitrile

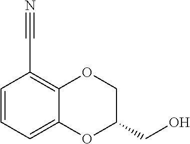 [SMILES: N#Cc1cccc2c1OC[C@H](CO)O2]

¹H NMR (DMSOd₆) δ: 3.65 (m, 2H); 4.17 (dd, 1H, J=11.4 and 7.6 Hz); 4.25 (m, 1H); 4.52 (dd, 1H, J=11.4 and 2 Hz); 5.15 (t, 1H, J=5.6 Hz); 6.98 (m, 1H); 7.22 (dd, 1H, J=8 and 1.6 Hz); 7.28 (dd, 1H, J=8 and 1.6 Hz).

### Intermediate 4.26: (R)-3-(hydroxymethyl)-2,3-dihydrobenzo[b][1,4]dioxine-6-carbonitrile

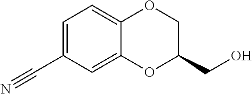 [SMILES: N#Cc1ccc2c(c1)O[C@H](CO)CO2]

¹H NMR (CDCl₃) δ: see NMR of intermediate 4.10

### Intermediate 4.27: (S)-8-fluoro-3-hydroxymethyl-2,3-dihydrobenzo[b][1,4]dioxine-6-carbonitrile

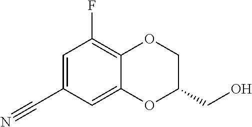 [SMILES: N#Cc1cc(F)c2c(c1)O[C@@H](CO)CO2]

¹H NMR (CDCl₃) δ: 1.89 (s, 1H, OH); 3.96 (m, 2H); 4.18 (m, 1H); 4.28 (m, 1H); 4.47 (m, 1H); 7.02 (m, 2H)

Type 2 halogen, mesylate or brosylate intermediates are prepared from (2,3-dihydrobenzo[b][1,4]dioxin-2-yl)methanol 1 intermediates previously described, according to diagram 2.

Bromine derivatives: prepared in the conventional NBS/PPh₃/DMF manner (Eur. J. Med. Chem., 2000; 35; 663-676)

### Intermediate 2.1: (R)-2-(bromomethyl)-2,3-dihydrobenzo[b][1,4]dioxine

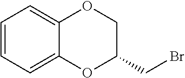 [SMILES: BrC[C@H]1COc2ccccc2O1]

¹H NMR (DMSOd₆) δ: 3.70 (dd, 1H, J=11 and 6 Hz); 3.79 (dd, 1H, J=11 and 4.4 Hz); 4.03 (dd, 1H, J=11.2 and 6.4 Hz); 4.37 (dd, 1H, J=11.2 and 2.4 Hz); 4.42 (m, 1H); 6.87 (m, 4H).

### Intermediate 2.2: (S)-2-(bromomethyl)-2,3-dihydrobenzo[b][1,4]dioxine

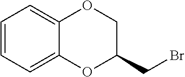 [SMILES: BrC[C@@H]1COc2ccccc2O1]

¹H NMR (CDCl₃) δ: see NMR intermediate 2.1

### Intermediate 2.3: (R)-2-(bromomethyl)-7-methyl-2,3-dihydrobenzo[b][1,4]dioxine

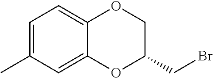

¹H NMR (CDCl₃) δ: 2.25 (s, 3H); 3.52 (m, 2H); 4.16 (dd, 1H, J=11.4 and 5.6 Hz); 4.31 (dd, 1H, J=11.4 and 2.4 Hz); 4.38 (m, 1H); 6.65 (d, 1H, J=8 Hz); 6.72 (s, 1H); 6.77 (d, 1H, J=8 Hz).

### Intermediate 2.4: (R)-8-bromo-2-(bromomethyl)-2,3-dihydrobenzo[b][1,4]dioxine

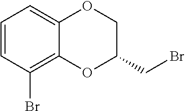 [SMILES: BrC[C@H]1COc2cccc(Br)c2O1]

¹H NMR (CDCl₃) δ: 3.52 (m, 1H); 3.63 (dd, 1H, J=10 and 4 Hz); 4.23 (dd, 1H, J=11.8 and 5.2 Hz); 4.36 (m, 1H); 4.53 (m, 1H); 6.74 (m, 1H); 6.86 (d, 1H, J=8 Hz); 7.12 (d, 1H, J=8 Hz).

### Intermediate 2.5: (R)-7-bromo-2-(bromomethyl)-2,3-dihydrobenzo[b][1,4]dioxine

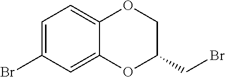 [SMILES: BrC[C@H]1COc2ccc(Br)cc2O1]

¹H NMR (CDCl₃) δ: 3.52 (m, 2H); 4.17 (dd, 1H, J=11.4 and 6 Hz); 4.33 (dd, 1H, J=11.4 and 2.4 Hz); 4.39 (m, 1H) 6.77 (d, 1H, J=8.6 Hz); 6.96 (dd, 1H, J=8.6 and 2.4 Hz); 7.06 (d, 1H, J=2.4 Hz).

### Intermediate 2.6: (R)-6-bromo-2-(bromomethyl)-2,3-dihydrobenzo[b][1,4]dioxine

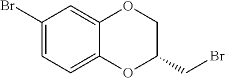 [SMILES: BrC[C@H]1COc2cc(Br)ccc2O1]

¹H NMR (CDCl₃) δ: 3.52 (m, 2H); 4.16 (dd, 1H, J=11.6 and 6 Hz); 4.34 (dd, 1H, J=11.6 and 2.4 Hz); 4.38 (m, 1H); 6.77 (d, 1H, J=8.4 Hz); 6.97 (dd, 1H, J=8.4 and 2.4 Hz); 7.04 (d, 1H, J=2.4 Hz).

### Intermediate 2.7: (R)-2-(bromomethyl)-7-chloro-2,3-dihydrobenzo[b][1,4]dioxine

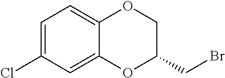 [SMILES: Clc1ccc2c(c1)O[C@@H](CBr)CO2]

¹H NMR (CDCl₃) δ: 3.52 (m, 2H); 4.17 (dd, 1H, J=11.4 and 6 Hz); 4.33 (dd, 1H, J=11.4 and 2.4 Hz); 4.39 (m, 1H); 6.81 (s, 2H); 6.91 (m, 1H).

### Intermediate 2.8: (R)-2-(bromomethyl)-7-(trifluoromethoxy)-2,3-dihydrobenzo[b][1,4]dioxine

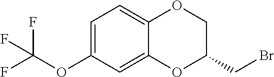 [SMILES: FC(F)(F)Oc1ccc2c(c1)O[C@@H](CBr)CO2]

¹H NMR (CDCl₃) δ: ¹H NMR (CDCl₃) δ: 3.53 (m, 2H); 4.18 (dd, 1H, J=11.6 and 6 Hz); 4.35 (dd, 1H, J=11.6 and 2 Hz); 4.41 (m, 1H); 6.73 (dd, 1H, J=9 and 2 Hz); 6.81 (d, 1H, J=2 Hz); 6.88 (d, 1H, J=9 Hz).

### Intermediate 2.9: (R)-6-bromo-2-(bromomethyl)-7-methoxy-2,3-dihydrobenzo[b][1,4]dioxine

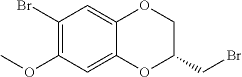

The conditions for bromination of (S)-(6-bromo-7-methoxy-2,3-dihydrobenzo[b][1,4]dioxin-2-yl)methanol also lead to bromination in position 6 of the aromatic ring.

¹H NMR (CDCl₃) δ: 3.53 (m, 2H); 3.81 (s, 3H); 4.12 (dd, 1H, J=11.6 and 6 Hz); 4.29 (dd, 1H, J=11.6 and 2 Hz); 4.38 (m, 1H); 6.51 (s, 1H); 7.09 (s, 1H).

Brosylate derivatives: preparation according to the conventional manner with 4-bromobenzene-1-sulfonyl chloride/Et₃N/DMAP/toluene (WO2004/24731A1, page 32).

### Intermediate 2.10: (R)-(7-(trifluoromethyl)-2,3-dihydrobenzo[b][1,4]dioxin-2-yl)methyl 4-bromobenzenesulfonate

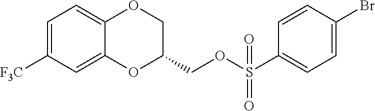 [SMILES: O=S(=O)(OC[C@H]1COc2ccc(C(F)(F)F)cc2O1)c1ccc(Br)cc1]

¹H NMR (CDCl₃) δ: 4.10 (dd, 1H, J=11.8 and 6.8 Hz); 4.28 (m, 3H); 4.42 (m, 1H); 6.93 (d, 1H, J=8.4 Hz); 7.03 (d, 1H, J=2 Hz); 7.11 (dd, 1H, J=8.4 and 2 Hz); 7.75 (m, 4H).

### Intermediate 2.11: (R)-(7-cyano-2,3-dihydrobenzo[b][1,4]dioxin-2-yl)methyl 4-bromobenzenesulfonate

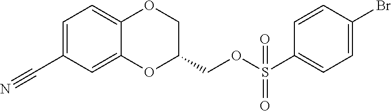 [SMILES: N#Cc1ccc2c(c1)O[C@@H](COS(=O)(=O)c1ccc(Br)cc1)CO2]

¹H NMR (CDCl₃) δ: 3.50 (dd, 1H, J=10.8 and 7.2 Hz); 3.57 (dd, 1H, J=10.8 and 4.8 Hz); 4.22 (m, 1H); 4.37 (m, 2H); 6.95 (d, 1H, J=8.4 Hz); 7.18 (dd, 1H, J=8.4 and 2 Hz); 7.21 (d, 1H, J=2 Hz).

### Intermediate 2.12: (S)-(7-cyano-2,3-dihydrobenzo[b][1,4]dioxin-2-yl)methyl 4-bromobenzenesulfonate

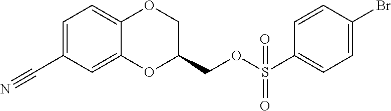 [SMILES: N#Cc1ccc2c(c1)O[C@H](COS(=O)(=O)c1ccc(Br)cc1)CO2]

¹H NMR (CDCl₃) δ: same as intermediate 2.11

### Intermediate 2.13: (R)-(8-cyano-2,3-dihydrobenzo[b][1,4]dioxin-2-yl)methyl 4-bromobenzenesulfonate

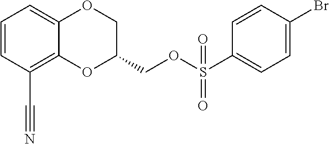 [SMILES: N#Cc1cccc2c1O[C@@H](COS(=O)(=O)c1ccc(Br)cc1)CO2]

¹H NMR (CDCl₃) δ: 4.14 (dd, 1H, J=12 and 6.8 Hz); 4.32 (m, 3H); 4.44 (m, 1H); 6.91 (m, 1H); 7.09 (dd, 1H, J=8.2 and 1.6 Hz); 7.15 (dd, 1H, J=7.6 and 1.6 Hz); 7.78 (m, 4H).

### Intermediate 2.14: (R)-(5-cyano-2,3-dihydrobenzo[b][1,4]dioxin-2-yl)methyl 4-bromobenzenesulfonate

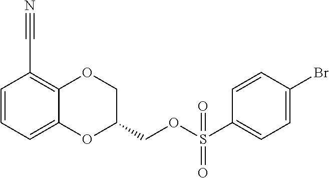 [SMILES: N#Cc1cccc2c1OC[C@H](COS(=O)(=O)c1ccc(Br)cc1)O2]

¹H NMR (DMSOd₆) δ: 4.15 (dd, 1H, J=11.4 and 7.2 Hz); 4.31 (dd, 1H, J=11.6 and 6 Hz); 4.47 (m, 2H); 4.55 (m, 1H); 6.98 (m, 1H); 7.11 (dd, 1H, J=8.2 and 1.6 Hz); 7.30 (dd, 1H, J=7.6 and 1.6 Hz); 7.87 (m, 4H).

### Intermediate 2.15: (R)-(5-bromo-2,3-dihydrobenzo[b][1,4]dioxin-2-yl)methyl 4-bromobenzenesulfonate

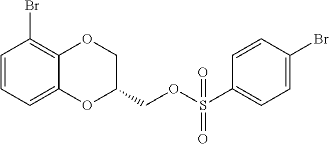 [SMILES: O=S(=O)(OC[C@H]1COc2c(Br)cccc2O1)c1ccc(Br)cc1]

¹H NMR (CDCl₃) δ: 4.13 (dd, 1H, J=11.6 and 2.4 Hz); 4.28 (m, 2H); 4.35 (m, 1H); 4.42 (m, 1H); 6.72 (m, 2H); 7.09 (m, 1H); 7.71 (m, 2H); 7.78 (m, 2H).

### Intermediate 2.16: (R)-(8-fluoro-2,3-dihydrobenzo[b][1,4]dioxin-2-yl)methyl 4-bromobenzenesulfonate

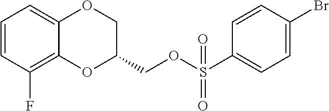 [SMILES: O=S(=O)(OC[C@H]1COc2cccc(F)c2O1)c1ccc(Br)cc1]

¹H NMR (DMSOd₆) δ: 4.04 (dd, 1H, J=11.6 and 6.8 Hz); 4.29 (dd, 1H, J=11.6 and 6.4 Hz); 4.34 (m, 1H); 4.45 (dd, 1H, J=11.2 and 2.8 Hz); 4.55 (m, 1H); 6.70 (m; 1H); 6.79 (m, 2H); 7.86 (m, 4H).

### Intermediate 2.17: (R)-(7-fluoro-2,3-dihydrobenzo[b][1,4]dioxin-2-yl)methyl 4-bromobenzenesulfonate

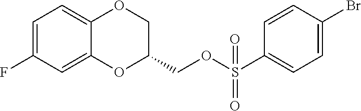 [SMILES: O=S(=O)(OC[C@H]1COc2ccc(F)cc2O1)c1ccc(Br)cc1]

¹H NMR (CDCl₃) δ: 4.03 (dd, 1H, J=11.6 and 6 Hz); 4.21 (dd, 1H, J=9 and 2.4 Hz); 4.24 (m, 2H); 4.41 (m, 1H); 6.49 (m, 1H); 6.55 (m, 1H); 6.79 (m, 1H); 7.71 (m, 2H); 7.77 (m, 2H).

### Intermediate 2.18: (R)-(6-fluoro-2,3-dihydrobenzo[b][1,4]dioxin-2-yl)methyl 4-bromobenzenesulfonate

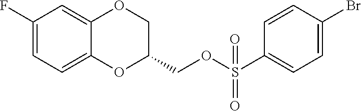 [SMILES: O=S(=O)(OC[C@H]1COc2cc(F)ccc2O1)c1ccc(Br)cc1]

¹H NMR (DMSOd₆) δ: 3.98 (dd, 1H, J=11.4 and 6.8 Hz); 4.26 (dd, 1H, J=11.4 and 6.4 Hz); 4.29 (dd, 1H, J=11.6 and 1.6 Hz); 4.42 (m, 2H); 6.67 (m, 1H); 6.77 (m, 2H); 7.85 (m, 2H); 7.89 (m, 2H).

### Intermediate 2.19: (R)-(5,7-difluoro-2,3-dihydrobenzo[b][1,4]dioxin-2-yl)methyl 4-bromobenzenesulfonate

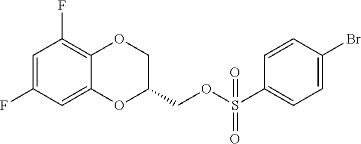 [SMILES: O=S(=O)(OC[C@H]1COc2c(F)cc(F)cc2O1)c1ccc(Br)cc1]

¹H NMR (DMSOd₆) δ: 4.02 (dd, 1H, J=11.6 and 6.8 Hz); 4.29 (dd, 1H, J=11.6 and 6.4 Hz); 4.35 (dd, 1H, J=11.6 and 2.4 Hz); 4.46 (dd, 1H, J=11.6 and 3.2 Hz); 4.58 (m, 1H); 6.57 (m, 1H); 6.87 (m, 1H); 7.88 (m, 4H).

### Intermediate 2.20: (R)-(7-chloro-5-fluoro-2,3-dihydrobenzo[b][1,4]dioxin-2-yl)methyl 4-bromobenzenesulfonate

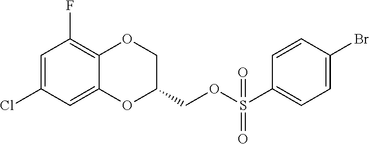 [SMILES: O=S(=O)(OC[C@H]1COc2c(F)cc(Cl)cc2O1)c1ccc(Br)cc1]

¹H NMR (DMSOd₆) δ: 4.05 (dd, 1H, J=11.6 and 6.8 Hz); 4.29 (dd, 1H, J=11.4 and 6 Hz); 4.38 (dd, 1H, J=11.6 and 2.4 Hz); 4.46 (dd, 1H, J=11.4 and 2.4 Hz); 4.58 (m, 1H); 6.72 (m, 1H); 7.02 (dd, 1H, J=10.4 and 2.4 Hz); 7.88 (m, 4H).

### Intermediate 2.21: (R)-(5-chloro-7-fluoro-2,3-dihydrobenzo[b][1,4]dioxin-2-yl)methyl 4-bromobenzenesulfonate

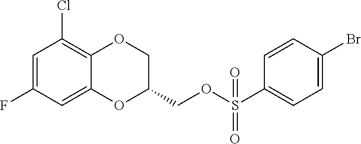 [SMILES: O=S(=O)(OC[C@H]1COc2c(Cl)cc(F)cc2O1)c1ccc(Br)cc1]

¹H NMR (DMSOd₆) δ: 4.05 (dd, 1H, J=11.6 and 7.2 Hz); 4.28 (dd, 1H, J=11.6 and 6 Hz); 4.39 (dd, 1H, J=11.6 and 2.8 Hz); 4.45 (dd, 1H, J=11.6 and 2.8 Hz); 4.57 (m, 1H); 6.73 (dd, 1H, J=9.6 and 2.8 Hz); 7.00 (dd, 1H, J=8.4 and 2.8 Hz); 7.89 (m, 4H).

### Intermediate 2.22: (R)-(6,7-difluoro-2,3-dihydrobenzo[b][1,4]dioxin-2-yl)methyl 4-bromobenzenesulfonate

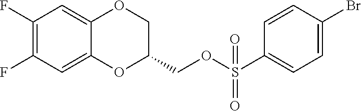 [SMILES: O=S(=O)(OC[C@H]1COc2cc(F)c(F)cc2O1)c1ccc(Br)cc1]

¹H NMR (DMSOd₆) δ: 3.98 (dd, 1H, J=11.4 and 6.8 Hz); 4.25 (dd, 1H, J=11.4 and 6.4 Hz); 4.29 (dd, 1H, J=11.4 and 2 Hz); 4.43 (dd, 1H, J=11.4 and 2.8 Hz); 4.51 (m, 1H); 6.93 (dd, 1H, J=11.2 and 8 Hz); 7.04 (dd, 1H, J=11.2 and 8 Hz); 7.87 (m, 4H).

### Intermediate 2.23: (R)-(7-(methylsulfonyl)-2,3-dihydrobenzo[b][1,4]dioxin-2-yl)methyl 4-bromobenzenesulfonate

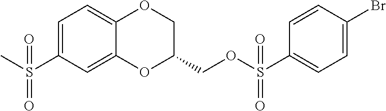 [SMILES: CS(=O)(=O)c1ccc2c(c1)O[C@@H](COS(=O)(=O)c1ccc(Br)cc1)CO2]

¹H NMR (DMSOd₆) δ: 3.17 (s, 3H); 4.07 (dd, 1H, J=11.6 and 7.2 Hz); 4.32 (dd, 1H, J=11.2 and 6 Hz); 4.42 (dd, 1H, J=11.6 and 2.4 Hz); 4.46 (dd, 1H, J=11.2 and 2.8 Hz); 4.53 (m, 1H); 7.10 (d, 1H, J=8.4 Hz); 7.33 (d, 1H, J=2.4 Hz); 7.39 (dd, 1H, J=8.4 and 2.4 Hz); 7.89 (m, 4H).

### Intermediate 2.24: (R)-methyl 3-((((4-bromophenyl)sulfonyl)oxy)methyl)-2,3-dihydrobenzo[b][1,4]dioxine-6-carboxylate

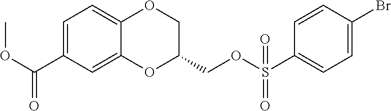 [SMILES: COC(=O)c1ccc2c(c1)O[C@@H](COS(=O)(=O)c1ccc(Br)cc1)CO2]

### Intermediate 2.25: (R)-(7-(benzyloxy)-2,3-dihydrobenzo[b][1,4]dioxin-2-yl)methyl 4-bromobenzenesulfonate

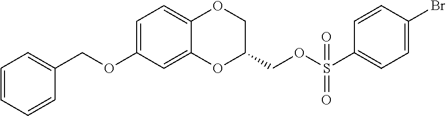 [SMILES: O=S(=O)(OC[C@H]1COc2ccc(OCc3ccccc3)cc2O1)c1ccc(Br)cc1]

¹H NMR (DMSOd₆) δ: 4.01 (dd, 1H, J=11.6 and 6 Hz); 4.18 (dd, 1H, J=11.6 and 2 Hz); 4.24 (m, 2H); 4.40 (m, 1H); 4.97 (s, 2H); 6.40 (d, 1H, J=2.8 Hz); 6.49 (dd, 1H, J=8.8 and 2.8 Hz); 6.75 (d, 1H, J=8.8 Hz); 7.36 (m, 5H); 7.70 (m, 2H); 7.77 (m, 2H).

### Intermediate 2.26: (R)-(7-cyano-5-fluoro-2,3-dihydrobenzo[b][1,4]dioxin-2-yl)methyl-4-bromobenzene sulfonate

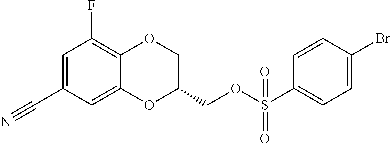 [SMILES: N#Cc1cc(F)c2c(c1)O[C@@H](COS(=O)(=O)c1ccc(Br)cc1)CO2]

¹H NMR (CDCl3) δ: 4.14 (m, 1H); 4.32 (m, 2H); 4.46 (m, 1H); 4.53 (m, 1H); 6.92 (d, 1H, J=5 Hz); 7 (d, 1H, J=6 Hz); 7.75 (m, 4H).

Mesylate derivatives: preparation in the conventional manner with methanesulfonyl chloride/Et₃N/THF (J. Med. Chem.; 1995; 38; 1119-1131)

### Intermediate 2.27: (R)-(6-(benzyloxy)-2,3-dihydrobenzo[b][1,4]dioxin-2-yl)methyl methanesulfonate

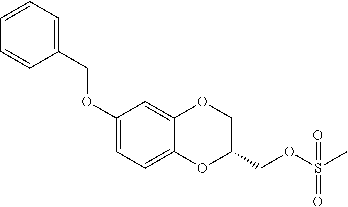 [SMILES: CS(=O)(=O)OC[C@H]1COc2cc(OCc3ccccc3)ccc2O1]

¹H NMR (CDCl₃) δ: 3.07 (s, 3H); 4.10 (m, 1H); 4.27 (m, 1H); 4.38 (m, 3H); 5.05 (s, 2H); 6.45 (m, 2H); 6.80 (m, 1H); 7.38 (m, 5H).

### “Piperidine Part”

### Intermediate 3.1 3-(2-(piperidin-4-yl)ethyl)oxazolidin-2-one

### Step 1: Preparation of tert-butyl 4-(2-iodoethyl)piperidine-1-carboxylate

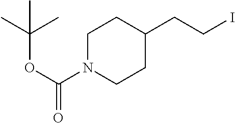 [SMILES: CC(C)(C)OC(=O)N1CCC(CCI)CC1]

In a 1 L single-neck flask, 250 ml of dichloromethane, 18.53 g (71 mmol of triphenylphosphine and 4.83 g (71 mmol) of imidazole are put into solution. 19.82 g (78 mmol) of iodide are carefully added and the mixture is stirred for 15 min at room temperature. Then 16.20 g (71 mmol) of commercial tert-butyl 4-(2-hydroxyethyl)piperidine-1-carboxylate in solution in 120 ml of dichloromethane are added dropwise. The mixture is stirred at room temperature overnight. The medium is then evaporated in the presence of silica. The solid obtained is deposited on a silica frit and purified by simple filtration using an n-heptane/EtOAc=90/10 mixture as an eluent. 20.91 g (62 mmol) tert-butyl 4-(2-iodoethyl)piperidine-1-carboxylate are recovered in the form of a very light yellow oil.

Yield: 87%

¹H NMR (CDCl₃) δ: 1.10 (m, 2H); 1.45 (s, 9H); 1.60 (m, 3H); 1.78 (m, 2H); 2.70 (m, 2H); 3.22 (t, 2H, J=7.2 Hz); 4.09 (m, 2H).

### Step 2: Preparation of tert-butyl 4-(2-(2-oxooxazolidin-3-yl)ethyl)piperidine-1-carboxylate

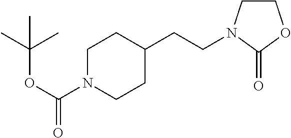 [SMILES: CC(C)(C)OC(=O)N1CCC(CCN2CCOC2=O)CC1]

1.5 g (37.5 mmol) of NaH and 50 ml of dimethylformamide are introduced into a 500-ml triple-neck flask. 3.2 g (37 mmol) of commercial 2-oxazolidinone in solution in 30 ml of dimethylformamide are added dropwise. As soon as gas release is no longer observed, 12.5 g (37 mmol) of tert-butyl 4-(2-iodoethyl)piperidine-1-carboxylate in solution in 50 ml of dimethylformamide are added. The medium is then heated at 70° C. overnight. The solvent is then concentrated with a vane pump and two water/dichloromethane extractions are performed; the organic phase is dried on MgSO4, filtered, evaporated and purified by flash chromatography on silica gel (eluent: n-heptane/acetone=60/40). 8.6 g (29 mmol) of tert-butyl 4-(2-(2-oxooxazolidin-3-yl)ethyl)piperidine-1-carboxylate are recovered in the form of a white solid.

Yield: 78%

¹H NMR (CDCl₃) δ: 1.12 (m, 2H); 1.45 (s, 9H); 1.49 (m, 3H); 1.69 (m, 2H); 2.68 (m, 2H); 3.32 (t, 2H, J=7.2 Hz); 3.55 (t, 2H, J=8 Hz); 4.08 (m, 2H); 4.33 (m, 2H).

### Step 3: Preparation of 3-(2-(piperidin-4-yl)ethyl)oxazolidin-2-one

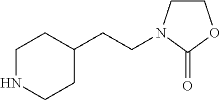 [SMILES: O=C1OCCN1CCC1CCNCC1]

In a 150-ml single-neck flask, 8.6 g (29 mmol) of tert-butyl 4-(2-(2-oxooxazolidin-3-yl)ethyl)piperidine-1-carboxylate solubilized in 50 ml of ethanol are introduced. 20 ml of a 5 N HCl solution in isopropanol (100 mmol) are added. The medium is brought to 70° C. for 4 hours. The medium is then concentrated and 50 ml of water are added, and then it is basified with aq NaOH and extracted with 2×50 ml of dichloromethane. The organic phase is dried on MgSO₄, filtered, evaporated and purified by flash chromatography on silica gel (eluent: dichloromethane/methanol/NH₄OH=90/9/1%). 5 g (25 mmol) of intermediate 3.1 are recovered in the form of a brittle white solid.

Yield: 87%

¹H NMR (CDCl₃) δ: 1.27 (m, 2H); 1.47 (m, 3H); 1.78 (m, 2H); 2.65 (m, 2H); 3.16 (m, 2H); 3.31 (t, 2H, J=3.2 Hz); 3.50 (ls, 1H); 3.55 (t, 2H, J=8 Hz); 4.33 (t, 2H, J=8 Hz).

### Intermediate 3.2 (S)-4-isopropyl-3-(2-(piperidin-4-yl)ethyl)oxazolidin-2-one

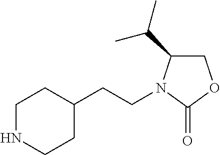 [SMILES: CC(C)[C@H]1COC(=O)N1CCC1CCNCC1]

By proceeding as in Example 3.1 (steps 2 and 3), but by replacing 2-oxazolidinone by commercial (S)-4-isopropyloxazolidin-2-one, intermediate 3.2 is obtained in the form of a pale yellow oil, after purification by flash chromatography on silica gel (eluent: dichloromethane/methanol=90/10%).

Yield: 97%

¹H NMR (CDCl₃) δ: 0.87 (d, 3H, J=6.8 Hz); 0.91 (d, 3H, J=6.8 Hz); 1.16 (m, 2H); 1.40 (m, 1H); 1.48 (m, 2H); 1.68 (m, 1H); 1.76 (m, 1H); 1.95 (m, 1H); 2.05 (m, 1H); 2.59 (m, 2H); 2.98 (m, 1H); 3.06 (m, 2H); 3.58 (m, 1H); 3.74 (m, 1H); 4.07 (dd, 1H, J=9 and 1.6 Hz); 4.21 (m, 1H).

### Intermediate 3.3 6-methoxy-3-(2-(piperidin-4-yl)ethyl)benzo[d]oxazol-2(3H)-one

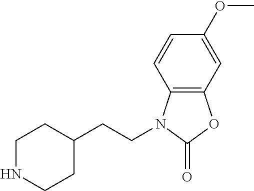 [SMILES: COc1ccc2c(c1)oc(=O)n2CCC1CCNCC1]

By proceeding as in Example 3.1 (steps 2 and 3), but by replacing 2-oxazolidinone by commercial 6-methoxybenzo[d]oxazol-2(3H)-one, intermediate 3.3 is obtained in the form of a cream solid without purification.

Yield: 96%

¹H NMR (CDCl₃) δ: 1.27 (m, 2H); 1.46 (m, 1H); 1.71 (m, 2H); 1.81 (m, 2H); 2.34 (is, 1H) 2.62 (m, 2H); 3.14 (m, 2H); 3.81 (s, 3H); 3.84 (m, 2H); 6.73 (dd, 1H, J=8.8 and 2.4 Hz); 6.84 (m, 2H).

### Intermediate 3.4 (S)-4-benzyl-3-(2-(piperidin-4-yl)ethyl)oxazolidin-2-one

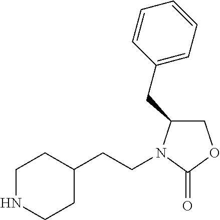 [SMILES: O=C1OC[C@H](Cc2ccccc2)N1CCC1CCNCC1]

By proceeding as in Example 3.1 (steps 2 and 3), but by replacing 2-oxazolidinone by commercial (S)-4-benzyloxazolidin-2-one, intermediate 3.4 is obtained in the form of a pale yellow oil, after purification by flash chromatography on silica gel (eluent: dichloromethane/methanol=90/10%).

Yield: 79%

¹H NMR (CDCl₃) δ: 1.22 (m, 2H); 1.40 (m, 1H); 1.50 (m, 2H); 1.73 (m, 2H); 2.64 (m, 4H); 3.10 (m, 3H); 3.57 (m, 1H); 4.01 (m, 2H); 4.15 (m, 1H); 7.16 (m, 1H); 7.32 (m, 2H).

### Intermediate 3.5 (R)-4-phenyl-3-(2-(piperidin-4-yl)ethyl)oxazolidin-2-one

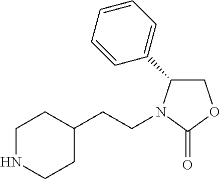 [SMILES: O=C1OC[C@@H](c2ccccc2)N1CCC1CCNCC1]

By proceeding as in Example 3.1 (steps 2 and 3), but by replacing 2-oxazolidinone by commercial by (R)-4-phenyloxazolidin-2-one, intermediate 3.5 is obtained in the form of a pale yellow oil, after purification by flash chromatography on silica gel (eluent: dichloromethane/methanol=95/5%).

Yield: 64%

¹H NMR (CDCl₃) δ: 1.05 (m, 2H); 1.35 (m, 3H); 1.61 (m, 2H); 2.53 (m, 2H); 2.78 (m, 1H); 3.02 (m, 2H); 3.47 (m, 1H); 4.13 (dd, 1H, J=8.6 and 6.4 Hz); 4.62 (m, 1H); 4.76 (dd, 1H, J=8.8 and 6.8 Hz); 7.31 (m, 2H); 7.40 (m, 3H).

### Intermediate 3.6 (S)-4-phenyl-3-(2-(piperidin-4-yl)ethyl)oxazolidin-2-one

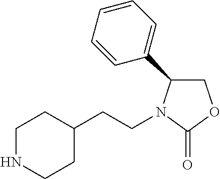 [SMILES: O=C1OC[C@H](c2ccccc2)N1CCC1CCNCC1]

By proceeding as in Example 3.1 (steps 2 and 3), but by replacing 2-oxazolidinone by commercial (S)-4-phenyloxazolidin-2-one, intermediate 3.6 is obtained in the form of a pale yellow oil, after purification by flash chromatography on silica gel (eluent: dichloromethane/methanol=95/5%).

Yield: 60%

¹H NMR (CDCl₃) δ: same as intermediate 3.5.

### Intermediate 3.7 4-(2-methoxyphenyl)-3-(2-(piperidin-4-yl)ethyl)oxazolidin-2-one (product prepared according to Synthetic communication; 30; vol 6; p-1095-1102; 2000)

### Step 1: Preparation of 2-(benzylamino)-2-(2-methoxyphenyl)acetonitrile

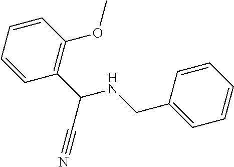 [SMILES: COc1ccccc1C(C#N)NCc1ccccc1]

In a 500-ml triple-neck flask, 12.1 g (63 mmol) of Na₂S₂O₅, 110 ml of water and 10 ml of methanol are introduced. 17.2 g (126 mmol) of o-anisaldehyde are added and 6.9 g (139 mmol) NaCN are added all at once. The mixture is stirred at 28° C. for 15 min, and then it is placed at 0° C. and 15 ml (126 mmol) of benzaldehyde in solution in 30 ml of methanol are added. The medium is stirred at 35° C. for 8 hours. After return to room temperature, 50 ml of water are added and it is extracted twice with 100 ml of dichloromethane. The organic phases are dried on MgSO₄, filtered, evaporated and purified by flash chromatography on silica gel (eluent: n-heptane/ethyl acetate=70/30%). 30 g (119 mmol) of 2-(benzylamino)-2-(2-methoxyphenyl)acetonitrile are recovered in the form of a clear oil.

Yield: 94%

¹H NMR (CDCl₃) δ: 2.25 (ls, 1H); 3.85 (s, 3H); 3.92 (d, 1H, J=13 Hz); 4.06 (d, 1H, J=13 Hz); 4.80 (s, 1H); 6.95 (m, 2H); 7.33 (m, 7H).

### Step 2: Preparation of 2-(benzylamino)-2-(2-methoxyphenyl)acetamide

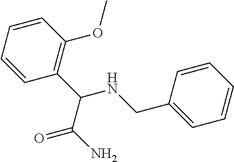 [SMILES: COc1ccccc1C(NCc1ccccc1)C(N)=O]

In a 500-ml single-neck flask, 30 g (119 mmol) of 2-(benzylamino)-2-(2-methoxyphenyl)acetonitrile obtained previously are solubilized in 160 ml of dimethylsulfoxide. 8.2 g (59.5 mmol) of K₂CO₃ are added and the medium is heated to 35° C. Then 19.4 ml (190 mmol) of 30% H₂O₂ are added. The mixture is stirred at 35° C. for 7 hours. After return to room temperature, 100 ml of ice water are added and it is extracted three times with 100 ml of ethyl acetate. The organic phases are collected, washed twice with 150 ml of ice water, dried on MgSO₄, filtered, evaporated and purified by flash chromatography on silica gel (eluent: n-heptane/ethyl acetate=70/30%). 23.4 g (86.6 mmol) of 2-(benzylamino)-2-(2-methoxyphenyl)acetamide are recovered in the form of a white solid.

Yield: 73%

### Step 3: Preparation of 2-(benzylamino)-2-(2-methoxyphenyl)acetic acid

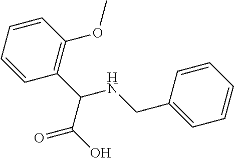 [SMILES: COc1ccccc1C(NCc1ccccc1)C(=O)O]

In a 500-ml single-neck flask, 23.4 g (86.6 mmol) of 2-(benzylamino)-2-(2-methoxyphenyl)acetamide obtained previously are solubilized in 156 ml of IMS (140 ml of ethanol/16 ml of methanol) and 35 ml of water. 5.1 g (129.8 mmol) of sodium hydroxide pellets are added and the medium is brought to 80° C. for 30 hours. The medium is concentrated and 50 ml of water are added and it is washed twice with 100 ml of dichloromethane. The aqueous phase is acidified to pH 3 with 10 N HCl, concentrated and the oily residue is taken up in ether. After trituration, filtration and drying, 17.2 g (63.4 mmol) of 2-(benzylamino)-2-(2-methoxyphenyl)acetic acid are obtained in the form of a cream solid.

Yield: 73%

¹H NMR (DMSOd₆) δ: 3.76 (s, 3H); 4.04 (s, 2H); 5.11 (s, 1H); 7.03 (m, 1H); 7.10 (m, 1H); 7.43 (m, 7H).

### Step 4: Preparation of 2-(benzylamino)-2-(2-methoxyphenyl)ethanol

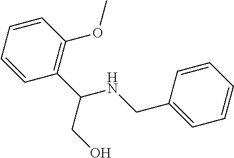 [SMILES: COc1ccccc1C(CO)NCc1ccccc1]

In a 500-ml triple-neck flask, 3.6 g (95 mmol) of LiAlH₄ in 200 ml of tetrahydrofuran are introduced. 17.2 g (63.4 mmol) of 2-(benzylamino)-2-(2-methoxyphenyl)acetic acid obtained previously are added at 0° C. by portions. The medium is then stirred at 50° C. for 20 hours. After return to room temperature, the medium is cooled to 0° C. and 18 ml of 10% NaOH are added dropwise. The aluminates are filtered. The filtrate is then washed by 100 ml of water and extracted twice with 100 ml of dichloromethane. The organic phases are then dried on Na₂SO₄, filtered, evaporated and purified by flash chromatography on silica gel (eluent: dichloromethane/methanol=90/10%). 8.3 g (32.3 mmol) of 2-(benzylamino)-2-(2-methoxyphenyl)ethanol are recovered in the form of a clear oil.

Yield: 51%

¹H NMR (CDCl₃) δ: 1.99 (ls, 1H); 2.97 (ls, 1H); 3.68 (m, 4H); 3.82 (s, 3H); 4.10 (m, 1H); 6.90 (m, 1H); 6.97 (m, 1H); 7.31 (m, 7H).

### Step 5: Preparation of 2-amino-2-(2-methoxyphenyl)ethanol

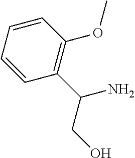

In a 250-ml triple-neck flask, 7 g (27.2 mmol) of 2-(benzylamino)-2-(2-methoxyphenyl)ethanol obtained previously are solubilized in 100 ml of methanol. 0.7 g of Pd(OH)₂/C and 8.6 g (136 mmol) of ammonium formiate are added, and then the medium is brought to 70° C. for 2.5 h. After return to room temperature, the palladium is filtered on Celite, rinsed with 20 methanol and the filtrate is concentrated and purified by flash chromatography on silica gel (eluent: dichloromethane/methanol/NH₄OH=90/9/1%). 4.3 g (25.7 mmol) of 2-amino-2-(2-methoxyphenyl)ethanol are recovered in the form of a clear oil.

Yield: 95%

¹H NMR (CDCl₃) δ: 2.02 (ls, 3H); 3.61 (m, 1H); 3.74 (dd, 1H, J=10.3 and 2.4 Hz); 3.84 (s, 3H); 4.26 (m, 1H); 6.88 (m, 1H); 6.95 (m, 1H); 7.26 (m, 2H).

### Step 6: Preparation of 4-(2-methoxyphenyl)oxazolidin-2-one

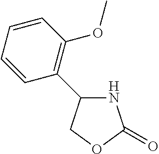

In a 250-ml triple-neck flask, 4.2 g (25 mmol) of 2-amino-2-(2-methoxyphenyl)ethanol obtained previously and 2.5 g (25 mmol) of KHCO₃ are solubilized in 40 ml of water. After 10 min of stirring, it cooled to 0° C. and 3.4 g (25 mmol) of K₂CO₃, are added and then 17 ml (32.6 mmol) of 20% phosgene in toluene are added dropwise. The medium is stirred at 0° C. for 6 h. The formation of a white product is observed, which is filtered and dried. 3.6 g (18.6 mmol) of 4-(2-methoxyphenyl)oxazolidin-2-one are recovered in the form of a white solid.

Yield: 75%

¹H NMR (CDCl₃) δ: 3.85 (s, 3H); 4.18 (dd, 1H, J=8.8 and 6.4 Hz); 4.80 (m, 1H); 5.25 (dd, 1H, J=8.6 and 6.4 Hz); 5.46 (is, 1H); 6.90 (m, 1H); 7.01 (m, 1H); 7.32 (m, 2H).

### Step 7: Preparation of tert-butyl 4-(2-(4-(2-methoxyphenyl)-2-oxooxazolidin-3-yl)ethyl)piperidine-1-carboxylate

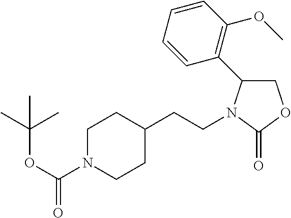 [SMILES: COc1ccccc1C1COC(=O)N1CCC1CCN(C(=O)OC(C)(C)C)CC1]

By proceeding as in Example 3.1 (step 2), but by replacing 2-oxazolidinone by 4-(2-methoxyphenyl)oxazolidin-2-one obtained previously, tert-butyl 4-(2-(4-(2-methoxyphenyl)-2-oxooxazolidin-3-yl)ethyl)piperidine-1-carboxylate is obtained in the form of a pale yellow oil, after purification by flash chromatography on silica gel (eluent: n-heptane/ethyl acetate=70/30%).

Yield: 57%

¹H NMR (CDCl₃) δ: 1.03 (m, 2H); 1.40 (m, 3H); 1.44 (s, 9H); 1.61 (m, 2H); 2.63 (m, 2H); 2.79 (m, 1H); 3.54 (m, 1H); 3.85 (s, 3H); 4.02 (m, 2H); 4.11 (dd, 1H, J=8.4 and 5.6 Hz); 4.59 (m, 1H); 5.15 (dd, 1H, J=9 and 5.6 Hz); 6.93 (m, 1H); 6.99 (m, 1H); 7.18 (m, 1H); 7.34 (m, 1H).

### Step 8: Preparation of 4-(2-methoxyphenyl)-3-(2-(piperidin-4-yl)ethyl)oxazolidin-2-one

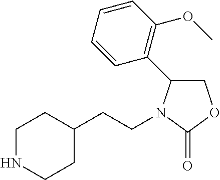 [SMILES: COc1ccccc1C1COC(=O)N1CCC1CCNCC1]

By proceeding as in Example 3.1 (step 3), but by replacing tert-butyl 4-(2-(2-oxooxazolidin-3-yl)ethyl)piperidine-1-carboxylate by tert-butyl 4-(2-(4-(2-methoxyphenyl)-2-oxooxazolidin-3-yl)ethyl)piperidine-1-carboxylate, intermediate 3.7 is obtained in the form of a pale yellow oil, after purification by flash chromatography on silica gel (eluent: dichloromethane/methanol=90/10%).

Yield: 96%

¹H NMR (CDCl₃) δ: 1.18 (m, 2H); 1.39 (m, 2H); 1.69 (m, 2H); 2.63 (m, 4H); 3.13 (m, 2H); 3.53 (m, 1H); 3.84 (s, 3H); 4.11 (dd, 1H, J=8.4 and 5.6 Hz); 4.59 (m, 1H); 5.15 (dd, 1H, J=9 and 5.6 Hz); 6.93 (m, 1H); 6.99 (m, 1H); 7.18 (m, 1H); 7.33 (m, 1H).

### Intermediate 3.8 4-(4-methoxyphenyl)-3-(2-(piperidin-4-yl)ethyl)oxazolidin-2-one

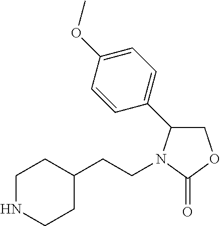 [SMILES: COc1ccc(C2COC(=O)N2CCC2CCNCC2)cc1]

By proceeding as in Example 3.7, but by replacing o-anisaldehyde by commercial p-anisaldehyde, intermediate 3.8 is obtained in the form of a pale yellow oil, after purification by flash chromatography on silica gel (eluent: dichloromethane/methanol=90/10%).

Yield: 80%

¹H NMR (CDCl₃) δ: 1.15 (m, 2H); 1.38 (m, 2H); 1.61 (m, 2H); 2.57 (m, 2H); 2.82 (m, 2H); 3.07 (m, 2H); 3.44 (m, 1H); 3.83 (s, 3H); 4.10 (m, 1H); 4.59 (m, 1H); 4.71 (m, 1H); 6.93 (d, 2H, J=8.1 Hz); 7.21 (d, 2H, J=8.1 Hz).

### Intermediate 3.9 (S)-4-(4-methoxybenzyl)-3-(2-(piperidin-4-yl)ethyl) oxazolidin-2-one

### Step 1: Preparation of (S)-2-amino-3-(4-methoxyphenyl)propan-1-ol

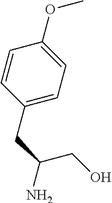

In a 100-ml triple-neck flask, 0.5 g (2.6 mmol) of (S)-2-amino-3-(4-methoxyphenyl)propanoic acid are solubilized in 5 ml of tetrahydrofuran. The mixture is cooled to 0° C. and 12.8 ml (12.8 mmol) of a 1 M solution of BH₃ in tetrahydrofuran are added dropwise. The medium is then heated at 70° C. for 4 hours. The mixture is cooled again to 0° C. and 30 ml of a 1 N HCl solution are added. The reaction medium is washed with 30 ml of dichloromethane. The aqueous phase is then basified by 30 ml of a 1 N NaOH solution and extracted twice with 100 ml of dichloromethane. These organic phases are then dried on Na₂SO₄, filtered and evaporated. 0.3 g (1.7 mmol) of (S)-2-amino-3-(4-methoxyphenyl)propan-1-ol are recovered in the form of a white solid.

Yield: 65%

¹H NMR (CDCl₃) δ: 1.59 (ls, 3H); 2.47 (dd, 1H, J=13.6 and 8.4 Hz); 2.73 (dd, 1H, J=13.6 and 5.2 Hz); 3.07 (m, 1H); 3.36 (dd, 1H, J=10.4 and 7.2 Hz); 3.63 (dd, 1H, J=10.4 and 7.6 Hz); 3.80 (s, 3H); 6.85 (d, 2H, J=8.8 Hz); 7.11 (d, 2H, J=8.8 Hz).

### Step 2: Preparation of (S)-4-(4-methoxybenzyl)oxazolidin-2-one

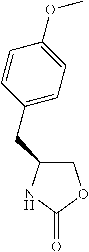

By proceeding as in Example 3.7 (step 6), but by replacing (S)2-amino-2-(2-methoxyphenyl)ethanol by (S)-2-amino-3-(4-methoxyphenyl)propan-1-ol, (S)-4-(4-methoxybenzyl)oxazolidin-2-one is obtained in the form of a pale yellow oil after purification by flash chromatography on silica gel (eluent: dichloromethane/methanol=95/5%).

Yield: 87%

¹H NMR (CDCl₃) δ: 2.81 (m, 2H); 3.80 (s, 3H); 4.04 (m, 1H); 4.14 (dd, 1H, J=8.8 and 5.6 Hz); 4.45 (m, 1H); 6.87 (d, 2H, J=8.8 Hz); 7.09 (d, 2H, J=8.8 Hz).

### Step 3: Preparation of (S)-tert-butyl 4-(2-(4-(4-methoxybenzyl)-2-oxooxazolidin-3-yl)ethyl)piperidine-1-carboxylate

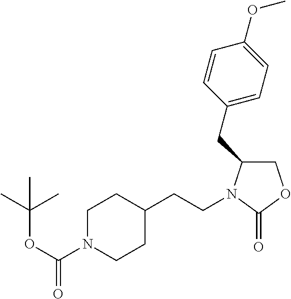 [SMILES: COc1ccc(C[C@H]2COC(=O)N2CCC2CCN(C(=O)OC(C)(C)C)CC2)cc1]

By proceeding as in Example 3.1 (step 2), but by replacing 2-oxazolidinone by (S)-4-(4-methoxybenzyl)oxazolidin-2-one, (S)-tert-butyl 4-(2-(4-(4-methoxybenzyl)-2-oxooxazolidin-3-yl)ethyl)piperidine-1-carboxylate is obtained in the form of a pale yellow oil after purification by flash chromatography on silica gel (eluent: n-heptane/ethyl acetate=70/30%).

Yield: 71%

¹H NMR (CDCl₃) δ: 1.11 (m, 2H); 1.45 (s, 9H); 1.46 (m, 3H); 1.68 (m, 2H); 2.63 (m, 3H); 3.07 (m, 2H); 3.56 (m, 1H); 3.80 (s, 3H); 3.98 (m, 2H); 4.14 (m, 3H); 6.86 (d, 2H, J=8.4 Hz); 7.07 (d, 2H, J=8.4 Hz).

### Step 4: Preparation of (S)-4-(4-methoxybenzyl)-3-(2-(piperidin-4-yl)ethyl)oxazolidin-2-one

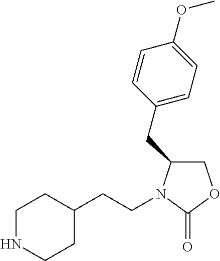 [SMILES: COc1ccc(C[C@H]2COC(=O)N2CCC2CCNCC2)cc1]

By proceeding as in Example 3.1 (step 3), but by replacing 4-(2-(4-(4-methoxybenzyl)-2-oxooxazolidin-3-yl)ethyl)piperidine-1-carboxylate, by (S)-tert-butyl 4-(2-(4-(4-methoxybenzyl)-2-oxooxazolidin-3-yl)ethyl)piperidine-1-carboxylate, intermediate 3.9 is obtained in the form of a pale yellow oil without purification.

Yield: 84%

¹H NMR (CDCl₃) δ: 1.27 (m, 2H); 1.42 (m, 1H); 1.51 (m, 2H); 1.76 (m, 2H); 2.63 (m, 4H); 3.10 (m, 3H); 3.56 (m, 1H); 3.80 (s, 3H); 3.98 (m, 2H); 4.18 (m, 1H); 6.86 (d, 2H, J=8.4 Hz); 7.07 (d, 2H, J=8.4 Hz).

### Intermediate 3.10 (S)-4-(4-(benzyloxy)benzyl)-3-(2-(piperidin-4-yl)ethyl) oxazolidin-2-one

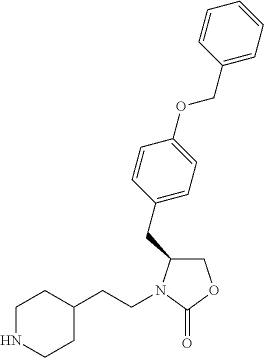 [SMILES: O=C1OC[C@H](Cc2ccc(OCc3ccccc3)cc2)N1CCC1CCNCC1]

By proceeding as in Example 3.9, but replacing (S)-2-amino-3-(4-methoxyphenyl)propanoic acid by (S)-2-amino-3-(4-(benzyloxy)phenyl)propanoic acid, intermediate 3.10 is obtained in the form of a pale yellow oil without purification.

Yield: 81%

¹H NMR (CDCl₃) δ: 1.25 (m, 2H); 1.41 (m, 1H); 1.49 (m, 2H); 1.75 (m, 2H); 2.62 (m, 4H); 3.08 (m, 3H); 3.56 (m, 1H); 3.98 (m, 2H); 2.67 (m, 1H); 5.05 (s, 2H); 6.94 (d, 2H, J=8.4 Hz); 7.07 (d, 2H, J=8.4 Hz); 7.38 (m, 5H).

### Intermediate 3.11 (S)-4-(3,4-dimethoxybenzyl)-3-(2-(piperidin-4-yl)ethyl) oxazolidin-2-one

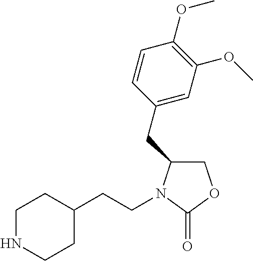 [SMILES: COc1ccc(C[C@H]2COC(=O)N2CCC2CCNCC2)cc1OC]

By proceeding as in Example 3.9, but replacing (S)-2-amino-3-(4-methoxyphenyl)propanoic acid by (S)-2-amino-3-(3,4-dimethoxyphenyl)propanoic acid, intermediate 3.11 is obtained in the form of a pale yellow oil without purification.

Yield: 94%

¹H NMR (CDCl₃) δ: 1.22 (m, 2H); 1.42 (m, 1H); 1.52 (m, 2H); 1.73 (m, 2H); 2.36 (is, 1H); 2.62 (m, 3H); 3.07 (m, 4H); 3.57 (m, 1H); 3.87 (s, 3H); 3.88 (s, 3H); 3.99 (m, 2H); 4.17 (m, 1H); 6.64 (d, 1H, J=2 Hz); 6.70 (dd, 1H, J=8 and 2 Hz); 6.82 (d, 1H, J=8 Hz).

### Intermediate 3.12 (S)-4-(4-fluorobenzyl)-3-(2-(piperidin-4-yl)ethyl)oxazolidin-2-one

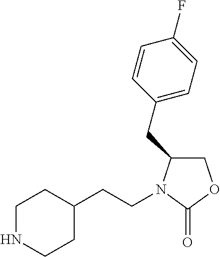 [SMILES: O=C1OC[C@H](Cc2ccc(F)cc2)N1CCC1CCNCC1]

By proceeding as in Example 3.9, but replacing (S)-2-amino-3-(4-methoxyphenyl)propanoic acid by (S)-2-amino-3-(4-fluorophenyl)propanoic acid, intermediate 3.12 is obtained in the form of a colorless oil after purification by flash chromatography on silica gel (eluent: dichloromethane/methanol/NH₄OH=90/9/1%).

Yield: 80%

¹H NMR (CDCl₃) δ: 1.20 (m, 2H); 1.39 (m, 1H); 1.50 (m, 2H); 1.72 (m, 2H); 1.92 (ls, 1H); 2.63 (m, 3H); 3.08 (m, 4H); 3.58 (m, 1H); 3.98 (m, 2H); 4.17 (m, 1H); 7.03 (m, 2H); 7.13 (m, 2H).

### Intermediate 3.13 (R)-4-(phenoxymethyl)-3-(2-(piperidin-4-yl)ethyl) oxazolidin-2-one

### Step 1: Preparation of (S)-methyl 2-oxooxazolidine-4-carboxylate

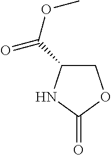

By proceeding as in Example 3.7 (step 6), but by replacing 2-amino-2-(2-methoxyphenyl)ethanol by (S)-methyl 2-amino-3-hydroxypropanoate hydrochloride, (S)-methyl 2-oxooxazolidine-4-carboxylate is obtained in the form of a colorless oil after purification by flash chromatography on silica gel (eluent: dichloromethane/methanol/NH₄OH=90/9/1%).

Yield: 88%

¹H NMR (DMSOd₆) δ: 3.71 (s, 3H); 4.34 (m, 1H); 4.48 (m, 2H); 8.24 (s, 1H).

### Step 2: Preparation of (R)-4-(hydroxymethyl)oxazolidin-2-one

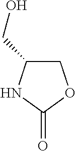

In a 250-ml triple-neck flask, 2.2 g (59.95 mmol) of NaBH₄ in 20 ml of ethanol are introduced. 5.8 g (39.96 mmol) of (S)-methyl 2-oxooxazolidine-4-carboxylate obtained previously in solution in 80 ml of methanol are added dropwise at 0° C. After 3 h of stirring at 0° C., the mixture is brought back to room temperature and then treated with 15 ml of a saturated solution of NH₄Cl. The white precipitate that forms is filtered. The filtrate is concentrated to give 4.5 g (38.43 mmol) of (R)-4-(hydroxymethyl)oxazolidin-2-one in the form of a white solid.

Yield: 96%

¹H NMR (D₂O) δ: 3.60 (dd, 1H, J=12 and 4 Hz); 3.68 (dd, 1H, J=12 and 3.6 Hz); 4.07 (m, 1H); 4.30 (m, 1H); 4.57 (m, 1H).

### Step 3: Preparation of (S)-(2-oxooxazolidin-4-yl)methyl 4-methylbenzenesulfonate

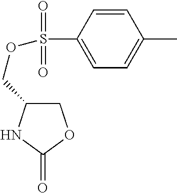 [SMILES: Cc1ccc(S(=O)(=O)OC[C@@H]2COC(=O)N2)cc1]

4 g (34.16 mmol) of (R)-4-(hydroxymethyl)oxazolidin-2-one previously obtained are solubilized in 40 ml of dichloromethane. The mixture is cooled to 0° C. and 7.1 g (37.57 mmol) of tosyl chloride and 5.2 ml (37.57 mmol) of triethylamine are added. The mixture is stirred at room temperature for 20 h, and then it is concentrated to dryness. Two extractions with water/ethyl acetate are then performed. The organic phases are then dried on MgSO₄, filtered and concentrated, and 4.3 g (15.85 mmol) of (S)-(2-oxooxazolidin-4-yl)methyl 4-methylbenzenesulfonate are obtained in the form of a white solid after purification by flash chromatography on silica gel (eluent: ethyl acetate=100%).

Yield: 47%

¹H NMR (CDCl₃) δ: 2.47 (s, 3H); 4.07 (m, 4H); 4.48 (m, 1H); 5.51 (s, 1H); 7.39 (d, 2H, J=8 Hz); 7.80 (d, 2H, J=8 Hz).

### Step 4: Preparation of (R)-4-(phenoxymethyl)oxazolidin-2-one

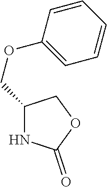 [SMILES: O=C1N[C@H](COc2ccccc2)CO1]

4.3 g (15.85 mmol) of (S)-(2-oxooxazolidin-4-yl)methyl 4-methylbenzenesulfonate described previously are solubilized in 40 ml acetonitrile. Then 7.9 g (24.32 mmol) of Cs₂CO₃ and 1.6 g (17.84 mmol) of phenol are added, and then the medium is heated at around 80° C. for 2 h. After return to room temperature, the Cs₂CO₃ is filtered and the medium is concentrated. Two extractions with water/dichloromethane are performed. The organic phases are dried on MgSO₄, filtered and evaporated and after trituration of the residue in diethyl ether, 1.9 g (9.83 mmol) of (R)-4-(phenoxymethyl)oxazolidin-2-one are obtained in the form of a white solid.

Yield: 65%

¹H NMR (CDCl₃) δ: 4.04 (m, 2H); 4.28 (m, 2H); 4.59 (m, 1H); 5.65 (s, 1H); 6.88 (m, 2H); 7.00 (m, 1H); 7.30 (m, 2H).

### Step 5: Preparation of (R)-tert-butyl 4-(2-(2-oxo-4-(phenoxymethyl)oxazolidin-3-yl)ethyl)piperidine-1-carboxylate

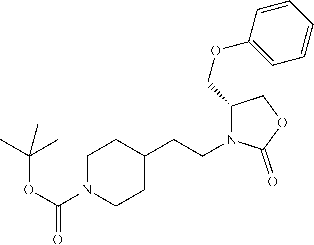 [SMILES: CC(C)(C)OC(=O)N1CCC(CCN2C(=O)OC[C@H]2COc2ccccc2)CC1]

By proceeding as in Example 3.1 (step 2), but by replacing commercial 2-oxazolidinone by (R)-4-(phenoxymethyl)oxazolidin-2-one previously described, (R)-tert-butyl 4-(2-(2-oxo-4-(phenoxymethyl)oxazolidin-3-yl)ethyl)piperidine-1-carboxylate is obtained in the form of a white solid after purification by flash chromatography on silica gel (eluent: dichloromethane/ethyl acetate=70/30%).

Yield: 65%

¹H NMR (CDCl₃) δ: 1.17 (m, 2H); 1.45 (s, 9H); 1.53 (m, 3H); 1.68 (m, 2H); 2.67 (m, 2H); 3.24 (m, 1H); 3.55 (m, 1H); 4.13 (m, 6H); 4.46 (m, 1H); 6.88 (m, 2H); 7.01 (m, 1H); 7.31 (m, 2H).

### Step 6: Preparation of (R)-4-(phenoxymethyl)-3-(2-(piperidin-4-yl)ethyl)oxazolidin-2-one

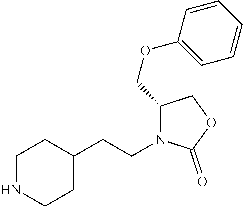 [SMILES: O=C1OC[C@@H](COc2ccccc2)N1CCC1CCNCC1]

By proceeding as in Example 3.1 (step 3), but by replacing tert-butyl 4-(2-(2-oxooxazolidin-3-yl)ethyl)piperidine-1-carboxylate by (R)-tert-butyl4-(2-(2-oxo-4-(phenoxymethyl)oxazolidin-3-yl)ethyl)piperidine-1-carboxylate previously obtained, (R)-4-(phenoxymethyl)-3-(2-(piperidin-4-yl)ethyl)oxazolidin-2-one is obtained in the form of a white solid after treatment and evaporation.

Yield: 89%

¹H NMR (CDCl₃) δ: 1.13 (m, 2H); 1.40 (m, 1H); 1.53 (m, 2H); 1.68 (m, 2H); 1.87 (is, 1H); 2.57 (m, 2H); 3.06 (m, 2H); 3.21 (m, 1H); 3.55 (m, 1H); 4.04 (m, 2H); 4.19 (m, 2H); 4.45 (m, 1H); 6.89 (m, 2H); 7.01 (m, 1H); 7.31 (m, 2H).

### Intermediate 3.14 (S)-4-(phenoxymethyl)-3-(2-(piperidin-4-yl)ethyl)oxazolidin-2-one

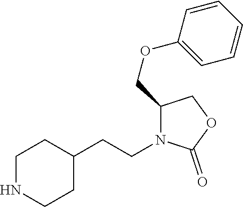 [SMILES: O=C1OC[C@H](COc2ccccc2)N1CCC1CCNCC1]

By proceeding as in Example 3.13, but replacing (S)-methyl 2-amino-3-hydroxypropanoate by (R)-methyl 2-amino-3-hydroxypropanoate, intermediate 3.14 is obtained in the form of a white solid.

Yield: 82%

¹H NMR (CDCl₃) δ: same as intermediate 3.13

### Intermediate 3.15 (R)-4-((4-fluorophenoxy)methyl)-3-(2-(piperidin-4-yl)ethyl)oxazolidin-2-one

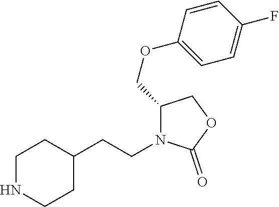 [SMILES: O=C1OC[C@@H](COc2ccc(F)cc2)N1CCC1CCNCC1]

By proceeding as in Example 3.13 (step 4), but replacing phenol by 4-fluoro phenol, intermediate 3.15 is obtained in the form of a clear oil.

Yield: 79%

¹H NMR (CDCl₃) δ: 1.17 (m, 2H); 1.41 (m, 1H); 1.50 (m, 2H); 1.71 (m, 2H); 1.87 (ls, 1H); 2.59 (m, 2H); 3.07 (m, 2H); 3.20 (m, 1H); 3.55 (m, 1H); 4.00 (d, 2H, J=4 Hz); 4.18 (m, 2H); 4.45 (m, 1H); 6.83 (m, 2H); 7.00 (m, 2H).

### Intermediate 3.16 (R)-4-((3-fluorophenoxy)methyl)-3-(2-(piperidin-4-yl)ethyl) oxazidin-2-one

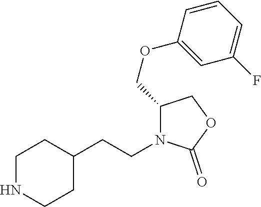 [SMILES: O=C1OC[C@@H](COc2cccc(F)c2)N1CCC1CCNCC1]

By proceeding as in Example 3.13 (step 4), but replacing phenol by 3-fluoro phenol, intermediate 3.16 is obtained in the form of a white solid.

Yield: 100%

¹H NMR (CDCl₃) δ: 1.20 (m, 2H); 1.43 (m, 1H); 1.52 (m, 2H); 1.71 (m, 2H); 2.38 (ls, 1H); 2.59 (m, 2H); 3.08 (m, 2H); 3.20 (m, 1H); 3.56 (m, 1H); 4.03 (d, 2H, J=4.4 Hz); 4.19 (m, 2H); 4.46 (m, 1H); 6.61 (m, 1H); 6.71 (m, 2H); 7.26 (m, 1H).

### Intermediate 3.17 (R)-4-((3,4-dimethoxyphenoxy)methyl)-3-(2-(piperidin-4-yl)ethyl) oxazolidin-2-one

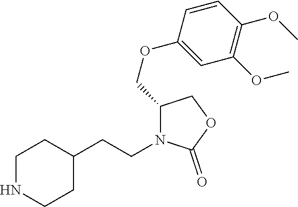 [SMILES: COc1ccc(OC[C@@H]2COC(=O)N2CCC2CCNCC2)cc1OC]

By proceeding as in Example 3.13 (step 4), but replacing phenol by 3,4-dimethoxy phenol, intermediate 3.17 is obtained in the form of a clear oil.

Yield: 100%

¹H NMR (CDCl₃) δ: 1.24-1.92 (m, 7H); 2.68 (m, 4H); 3.22 (m, 2H); 3.55 (m, 1H); 3.84 (s, 3H); 3.86 (s, 3H); 3.99 (m, 2H); 4.13 (m, 1H); 4.23 (m, 1H); 4.45 (m, 1H); 6.36 (dd, 1H, J=8.8 and 2.8 Hz); 6.49 (d, 1H, J=2.8 Hz); 6.78 (d, 1H, J=8.8 Hz).

### Intermediate 3.18 (R)-4-((3-(benzyloxy)phenoxy)methyl)-3-(2-(piperidin-4-yl)ethyl) oxazolidin-2-one

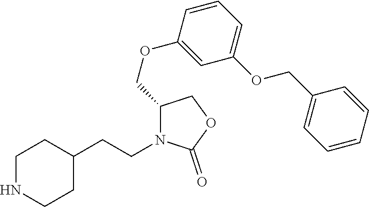 [SMILES: O=C1OC[C@@H](COc2cccc(OCc3ccccc3)c2)N1CCC1CCNCC1]

By proceeding as in Example 3.13 (step 4), but replacing phenol by 3-(benzyloxy)phenol, itself prepared from resorcinol according to the procedure described in J.O.C. vol 62, No. 10, 1997, p 3062-75, intermediate 3.18 is obtained in the form of a cream solid.

Yield: 45%

¹H NMR (CDCl₃) δ: 1.15 (m, 2H); 1.40 (m, 1H); 1.49 (m, 2H); 1.70 (m, 2H); 1.91 (ls, 1H); 2.58 (m, 2H); 3.06 (m, 2H); 3.19 (m, 1H); 3.54 (m, 1H); 3.98 (m, 2H); 4.13 (m, 1H); 4.20 (m, 1H); 4.43 (m, 1H); 5.02 (s, 2H); 6.81 (m, 2H); 6.91 (m, 2H); 7.37 (m, 5H).

### Intermediate 3.19 (R)-4-((4-(benzyloxy)phenoxy)methyl)-3-(2-(piperidin-4 yl)ethyl)oxazolidin-2-one

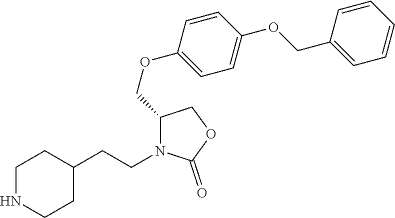 [SMILES: O=C1OC[C@@H](COc2ccc(OCc3ccccc3)cc2)N1CCC1CCNCC1]

By proceeding as in Example 3.13 (step 4), but replacing phenol by 4-(benzyloxy)phenol, intermediate 3.19 is obtained in the form of a cream solid.

Yield: 60%

¹H NMR (CDCl₃) δ: 1.20 (m, 2H); 1.43 (m, 1H); 1.50 (in, 2H); 1.73 (m, 2H); 2.43 (ls, 1H); 2.60 (m, 2H); 3.10 (m, 2H); 3.19 (m, 1H); 3.54 (m, 1H); 4.01 (d, 2H, J=4.4 Hz); 4.17 (m, 2H); 4.44 (m, 1H); 5.04 (s, 2H); 6.50 (m, 2H); 6.64 (m, 1H); 7.20 (m, 1H); 7.38 (m, 5H);

### Intermediate 3.20 (R)-4-((2,6-dimethylphenoxy)methyl)-3-(2-(piperidin-4-yl)ethyl) oxazolidin-2-one

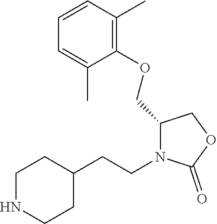 [SMILES: Cc1cccc(C)c1OC[C@@H]1COC(=O)N1CCC1CCNCC1]

By proceeding as in Example 3.13 (step 4), but replacing phenol by 2,6-dimethylphenol, intermediate 3.20 is obtained in the form of a clear oil.

Yield: 98%

¹H NMR (CDCl₃) δ: 1.22 (m, 2H); 1.44 (m, 1H); 1.55 (m, 2H); 1.75 (m, 2H); 2.11 (ls, 1H); 2.27 (s, 6H); 2.62 (m, 2H); 3.12 (m, 2H); 3.26 (m, 1H); 3.64 (m, 1H); 3.80 (m, 1H); 3.93 (m, 1H); 4.18 (m, 1H); 4.31 (m, 1H); 4.50 (m, 1H); 6.96 (m, 1H); 7.02 (m, 2H).

### Intermediate 3.21 (R)-4-(hydroxymethyl)-3-(2-(piperidin-4-yl)ethyl)oxazolidin-2-one

### Step 1: Preparation of (S)-tert-butyl 4-(2-((3-hydroxy-1-methoxy-1-oxopropan-2-yl)amino)ethyl)piperidine-1-carboxylate

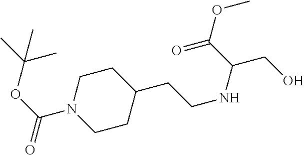 [SMILES: COC(=O)C(CO)NCCC1CCN(C(=O)OC(C)(C)C)CC1]

5.33 g (34.3 mmol) of (S)-methyl 2-amino-3-hydroxypropanoate hydrochloride, 100 ml of dichloromethane and 4.78 ml (34.3 mmol) of triethylamine are introduced into a 250-ml flask. The mixture is stirred for 15 min, and then 7.79 g (34.3 mmol) of tert-butyl 4-(2-oxoethyl)piperidine-1-carboxylate are added (prepared according to the method reported in Journal of Medicinal Chemistry, 2005, Vol. 48, No. 6, p 2100 2107) in solution in 10 ml of dichloromethane and 1.25 g of anhydrous MgSO₄. After one night at room temperature, the medium is filtered and concentrated and then 70 ml of methanol and 1.85 g (34.3 mmol) of KBH₄ are added at 0° C. At the end of 4 h, 100 ml of ethyl acetate are added and this is washed with 100 ml of water. After three extractions with ethyl acetate, the organic phases are collected, dried on Na₂SO₄, filtered and evaporated. 7.38 g (22.33 mmol) of (S)-tert-butyl 4-(2-((3-hydroxy-1-methoxy-1-oxopropan-2-yl)amino)ethyl)piperidine-1-carboxylate are obtained in the form of an orange oil, after purification by flash chromatography on silica gel (gradient; dichloromethane=100% to dichloromethane/(methanol:NH₄OH 9:1)=95/5% over 30 min).

Yield: 67%

¹H NMR (CDCl₃) δ: 1.11 (m, 2H); 1.45 (s, 9H); 1.48 (m, 3H); 1.63 (m, 2H); 2.00 (lm, 2H); 2.53 (m, 1H); 2.71 (m, 3H); 3.36 (dd, 1H, J=6.6 and 4.4 Hz); 3.57 (dd, 1H, J=10.6 and 6.8 Hz); 3.76 (s, 3H); 3.9 (m, 1H); 4.07 (m, 2H).

### Step 2: Preparation of (S)-methyl 3-(2-(1-(tert-butoxycarbonyl)piperidin-4-yl)ethyl)-2-oxooxazolidine-4-carboxylate

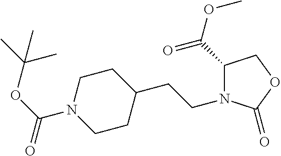 [SMILES: COC(=O)[C@@H]1COC(=O)N1CCC1CCN(C(=O)OC(C)(C)C)CC1]

In a 250-ml flask, 7.38 g (22.33 mmol) of (S)-tert-butyl 4-(2-((3-hydroxy-1-methoxy-1-oxopropan-2-yl)amino)ethyl)piperidine-1-carboxylate previously described are solubilized in 80 ml of tetrahydrofuran. 7.24 g (44.7 mmol) of 1,1′-carbonyldiimidazole are added and the medium is brought to 70° C. for 8 hours. Then 100 ml of water are added and the mixture is extracted twice with 100 ml of diethyl ether. The organic phases are collected, dried on Na₂SO₄, filtered and evaporated and 7 g (19.64 mmol) of (S)-methyl 3-(2-(1-(tert-butoxycarbonyl)piperidin-4-yl)ethyl)-2-oxooxazolidine-4-carboxylate are obtained in the form of a yellow oil, after purification on silica gel (gradient: dichloromethane=100% to dichloromethane/ethyl acetate=60/40% over 30 min).

Yield: 88%

¹H NMR (CDCl₃) δ: 1.12 (m, 2H); 1.45 (s, 9H); 1.46 (m, 3H); 1.69 (m, 2H); 2.67 (m, 2H); 3.18 (m, 1H); 3.62 (m, 1H); 3.82 (s, 3H); 4.09 (m, 2H); 4.34 (m, 2H); 4.47 (m, 1H).

### Step 3: Preparation of (R)-tert-butyl 4-(2-(4-(hydroxymethyl)-2-oxooxazolidin-3-yl)ethyl)piperidine-1-carboxylate

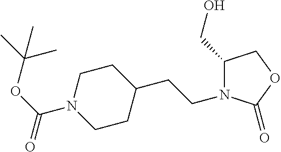 [SMILES: CC(C)(C)OC(=O)N1CCC(CCN2C(=O)OC[C@H]2CO)CC1]

1 g (2.81 mmol) of (S)-methyl 3-(2-(1-(tert-butoxycarbonyl)piperidin-4-yl)ethyl)-2-oxooxazolidine-4-carboxylate described previously are solubilized in 10 ml of ethanol. The mixture is placed at 0° C. and 0.16 g (4.21 mmol) of NaBH₄ are gently added by portions, taking care that this temperature does not exceed 5° C. The mixture is left for 1 h at 0° C. and then it is gently brought back to room temperature. It is then brought to 0° C. to add a saturated aqueous solution of NH₄Cl, and then water is added and it is extracted three times with ethyl acetate. The organic phases are collected, dried on Na₂SO₄, and 0.9 g (2.74 mmol) of (R)-tert-butyl 4-(2-(4-(hydroxymethyl)-2-oxooxazolidin-3-yl)ethyl)piperidine-1-carboxylate are obtained, after purification on silica gel (gradient: n-heptane=100% to ethyl acetate=100% over 30 min).

Yield: 98%

¹H NMR (CDCl₃) δ: 1.12 (m, 2H); 1.44 (m, 1H); 1.45 (s, 9H); 1.52 (m, 2H); 1.68 (m, 2H); 2.07 (m, 1H); 2.68 (m, 2H); 3.17 (m, 1H); 3.52 (m, 1H); 3.70 (m, 1H); 3.79 (m, 1H); 3.88 (m, 1H); 4.08 (m, 2H); 4.20 (m, 1H); 4.36 (m, 1H).

### Step 4: Preparation of (R)-4-(hydroxymethyl)-3-(2-(piperidin-4-yl)ethyl)oxazolidin-2-one

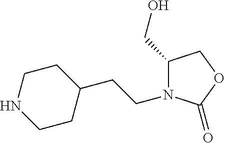 [SMILES: O=C1OC[C@@H](CO)N1CCC1CCNCC1]

0.75 g (2.30 mmol) are solubilized in 10 ml of ethanol and 2 ml (10 mmol) of a 5 N HCl solution in isopropanol are added. The medium is heated to 70° C. for 5 h, and then the medium is concentrated, basified and extracted with dichloromethane. The organic phase is dried on Na₂SO₄, filtered, evaporated and 0.37 g (1.61 mmol) of a clear oil are recovered after purification on silica gel (eluent: dichloromethane/methanol/NH₄OH=90/9/1%).

Yield: 70%

¹H NMR (CD₃OD) δ: 1.40 (m, 2H); 1.63 (m, 3H); 2.02 (m, 2H); 2.97 (m, 2H); 3.25 (m, 1H); 3.37 (m, 2H); 3.50 (m, 1H); 3.57 (m, 1H); 3.75 (m, 1H); 3.94 (m, 1H); 4.19 (m, 1H); 4.39 (m, 1H).

### Intermediate 3.22 (S)-4-(hydroxymethyl)-3-(2-(piperidin-4-yl)ethyl)oxazolidin-2-one

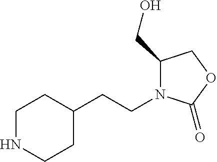 [SMILES: O=C1OC[C@H](CO)N1CCC1CCNCC1]

By proceeding as in Example 3.21, but replacing (S)-methyl 2-amino-3-hydroxypropanoate hydrochloride by (R)-methyl 2-amino-3-hydroxypropanoate hydrochloride, intermediate 3.22 is obtained in the form of a white solid.

Yield: 90%

¹H NMR (CDCl₃) δ: same as intermediate 3.21

### Intermediate 3.23 (R)-4-(hydroxymethyl)-5,5-dimethyl-3-(2-(piperidin-4-yl)ethyl) oxazolidin-2-one

### Step 1: Preparation of (R)-methyl 2-((tert-butoxycarbonyl)amino)-3-((tert-butyldimethylsilyl)oxy)propanoate

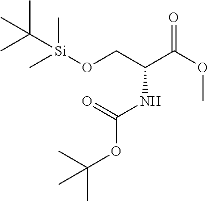 [SMILES: COC(=O)[C@@H](CO[Si](C)(C)C(C)(C)C)NC(=O)OC(C)(C)C]

In a 250-ml flask, 10 g (45.6 mmol) of (R)-methyl 2-((tert-butoxycarbonyl)amino)-3-hydroxypropanoate are solubilized in 50 ml of dimethylformamide. The medium is cooled to 0° C. and then 4.66 g (68.4 mmol) of imidazole and 7.56 g (50.2 mmol) of tert-butylchlorodimethylsilane are added. After return to room temperature, the mixture is stirred for 3 h. The medium is then concentrated and two water/ethyl acetate extractions are performed. The organic phases are collected, dried on Na₂SO₄, and 14.5 g (43.48 mmol) of (R)-methyl 2-((tert-butoxycarbonyl)amino)-3-((tert-butyldimethylsilyl)oxy)propanoate are obtained in the form of a colorless oil, after purification by flash chromatography on silica gel (gradient: n-heptane=100% to n-heptane/ethyl acetate=50/50% over 30 min).

Yield: 95%

¹H NMR (CDCl₃) δ: 0.02 (s, 3H); 0.04 (s, 3H); 0.86 (s, 9H); 1.46 (s, 9H); 3.74 (s, 3H); 3.82 (dd, 1H, J=10 and 3.2 Hz); 4.04 (dd, 1H, J=10 and 2.4 Hz); 4.35 (m, 1H); 5.33 (m, 1H).

### Step 2: Preparation of (R)-tert-butyl(1-((tert-butyldimethylsilyl)oxy)-3-hydroxy-3-methylbutan-2-yl)carbamate

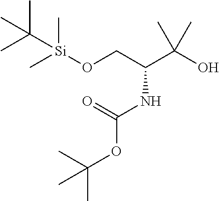 [SMILES: CC(C)(C)OC(=O)N[C@H](CO[Si](C)(C)C(C)(C)C)C(C)(C)O]

In a 500-ml flask swept with nitrogen, 7 g (20.99 mmol) of R)-methyl 2-((tert-butoxycarbonyl)amino)-3-((tert-butyldimethylsilyl)oxy)propanoate previously described are solubilized in 200 ml of tetrahydrofuran. 25 ml (75 mmol) of a 3M solution of methyl magnesium bromide in ether are added dropwise at 0° C. The solution is stirred for 24 h at room temperature. A saturated solution of NH₄Cl is added at 0° C. and the mixture is extracted three times with ethyl acetate. The organic phases are collected, dried on Na₂SO₄, filtered and evaporated, and 4.5 g (13.49 mmol) of (R)-tert-butyl(1-((tert-butyldimethylsilyl)oxy)-3-hydroxy-3-methylbutan-2-yl)carbamate are obtained in the form of a pale yellow oil, after purification by flash chromatography on silica gel (gradient: n-heptane=100% to n-heptane/ethyl acetate=50/50% over 30 min).

Yield: 64%

¹H NMR (CDCl₃) δ: 0.08 (s, 6H); 0.90 (s, 9H); 1.19 (s, 3H); 1.34 (s, 3H); 1.45 (s, 9H); 3.39 (m, 1H); 3.60 (s, 1H); 3.85 (dd, 1H, J=10.6 and 2 Hz); 4.06 (dd, 1H, J=10.6 and 2.4 Hz); 5.36 (m, 1H).

### Step 3: Preparation of (R)-4-(((tert-butyldimethylsilyl)oxy)methyl)-5,5-dimethyloxazolidin-2-one

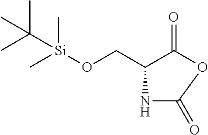 [SMILES: CC(C)(C)[Si](C)(C)OC[C@H]1NC(=O)OC1=O]

In a 500-ml flask containing 50 ml of tetrahydrofuran, 0.65 g (16.19 mmol) of 60% NaH in oil are introduced; then 4.5 g (13.49 mmol) of (R)-tert-butyl(1-((tert-butyldimethylsilyl)oxy)-3-hydroxy-3-methylbutan-2-yl)carbamate, previously prepared, in solution in 100 ml of tetrahydrofuran are added dropwise. The mixture is refluxed for 3 h. After return to room temperature, the medium is poured into a saturated NH₄Cl solution and extracted three times with ethyl acetate. The organic phases are collected, dried on Na₂SO₄, filtered and evaporated, and 2.9 g (11.18 mmol) of (R)-4-(((tert-butyldimethylsilyl)oxy)methyl)-5,5-dimethyloxazolidin-2-one are obtained in the form of a white solid, after purification by flash chromatography on silica gel (gradient: n-heptane=100% to n-heptane/ethyl acetate=70/30% over 30 min).

Yield: 83%

¹H NMR (CDCl₃) δ: 0.07 (s, 6H); 0.88 (s, 9H); 1.38 (s, 3H); 1.49 (s, 3H); 3.60 (m, 3H); 5.41 (s, 1H).

### Step 4: Preparation of (R)-tert-butyl 4-(2-(4-(((tert-butyldimethylsilyl)oxy)methyl)-5,5-dimethyl-2-oxooxazolidin-3-yl)ethyl)piperidine-1-carboxylate

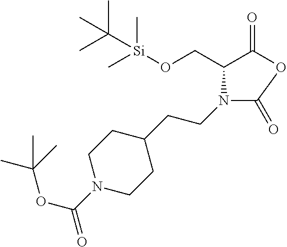 [SMILES: CC(C)(C)OC(=O)N1CCC(CCN2C(=O)OC(=O)[C@H]2CO[Si](C)(C)C(C)(C)C)CC1]

By proceeding as in Example 3.1 (step 2), but by replacing commercial 2-oxazolidinone by (R)-4-(((tert-butyldimethylsilyl)oxy)methyl)-5,5-dimethyloxazolidin-2-one prepared previously, (R)-tert-butyl 4-(2-(4-(((tert-butyldimethylsilyl)oxy)methyl)-5,5-dimethyl-2-oxooxazolidin-3-yl)ethyl)piperidine-1-carboxylate is obtained in the form of a pale yellow oil.

Yield: 92%

¹H NMR (CDCl₃) δ: 0.07 (s, 3H); 0.08 (s, 3H); 0.89 (s, 9H); 1.11 (m, 2H); 1.40 (s, 3H); 1.43 (s, 3H); 1.47 (m, 3H); 1.68 (m, 2H); 2.67 (m, 2H); 3.07 (m, 1H); 3.37 (m, 1H); 3.54 (m, 1H); 3.70 (m, 2H); 4.07 (m, 2H).

### Step 5: Preparation of (R)-4-(hydroxymethyl)-5,5-dimethyl-3-(2-(piperidin-4-yl)ethyl)oxazolidin-2-one

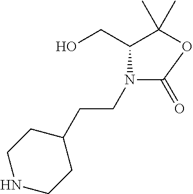 [SMILES: CC1(C)OC(=O)N(CCC2CCNCC2)[C@@H]1CO]

By proceeding as in Example 3.1 (step 3), but by replacing tert-butyl 4-(2-(2-oxooxazolidin-3-yl)ethyl)piperidine-1-carboxylate by (R)-tert-butyl 4-(2-(4-(((tert-butyldimethylsilyl)oxy)methyl)-5,5-dimethyl-2-oxooxazolidin-3-yl)ethyl)piperidine-1-carboxylate previously prepared, (R)-tert-butyl 4-(2-(4-(((tert-butyldimethylsilyl)oxy)methyl)-5,5-dimethyl-2-oxooxazolidin-3-yl)ethyl)piperidine-1-carboxylate is obtained in the form of a pale yellow oil.

Yield: 49%

¹H NMR (CDCl₃) δ: 1.17 (m, 2H); 1.43 (s, 3H); 1.45 (s, 3H); 1.50 (m, 3H); 1.73 (m, 2H); 2.33 (ls, 2H); 2.59 (m, 2H); 3.06 (m, 2H); 3.15 (m, 1H); 3.44 (m, 1H); 3.53 (m, 1H); 3.78 (d, 2H, J=5.2 Hz).

### Intermediate 3.24 (S)-4-(2-hydroxypropan-2-yl)-3-(2-(piperidin-4-yl)ethyl) oxazolidin-2-one

### Step 1: Preparation of (S)-tert-butyl 4-(2-(4-(2-hydroxypropan-2-yl)-2-oxooxazolidin-3-yl)ethyl)piperidine-1-carboxylate

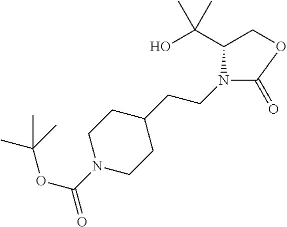 [SMILES: CC(C)(C)OC(=O)N1CCC(CCN2C(=O)OC[C@H]2C(C)(C)O)CC1]

1.5 g (4.21 mmol) of (S)-methyl 3-(2-(1-(tert-butoxycarbonyl)piperidin-4-yl)ethyl)-2-oxooxazolidine-4-carboxylate (described for intermediate 3.21, step 2) are solubilized in 40 ml of tetrahydrofuran. The medium is cooled to 0° C. and 3.4 ml (10.10 mmol) of a 3M solution of methyl magnesium bromide in ether are added dropwise. The mixture is allowed to gently return to room temperature, and then it is stirred for 5 h at this temperature. A saturated solution of NH₄Cl is added and it is extracted three times with ethyl acetate. The organic phases are collected, then dried on Na₂SO₄, filtered and evaporated. 1.1 g (3.08 mmol) of (S)-tert-butyl 4-(2-(4-(2-hydroxypropan-2-yl)-2-oxooxazolidin-3-yl)ethyl)piperidine-1-carboxylate are obtained in the form of a colorless oil, after purification by flash chromatography on silica gel (gradient: n-heptane=100% to n-heptane/ethyl acetate=80/20% over 30 min).

Yield: 73%

¹H NMR (CDCl₃) δ: 1.14 (m, 2H); 1.18 (s, 3H); 1.22 (s, 3-1); 1.38 (m, 1H); 1.45 (s, 9H); 1.66 (m, 4H); 2.59 (m, 1H); 2.68 (m, 2H); 3.43 (m, 1H); 3.64 (m, 1H); 3.73 (m, 1H); 4.05 (m, 3H); 4.25 (m, 1H).

### Step 2: Preparation of (S)-4-(2-hydroxypropan-2-yl)-3-(2-(piperidin-4-yl)ethyl)oxazolidin-2-one

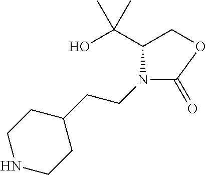 [SMILES: CC(C)(O)[C@@H]1COC(=O)N1CCC1CCNCC1]

By proceeding as in Example 3.1 (step 3), but by replacing tert-butyl 4-(2-(2-oxooxazolidin-3-yl)ethyl)piperidine-1-carboxylate by (S)-tert-butyl 4-(2-(4-(2-hydroxypropan-2-yl)-2-oxooxazolidin-3-yl)ethyl)piperidine-1-carboxylate previously prepared, (S)-4-(2-hydroxypropan-2-yl)-3-(2-(piperidin-4-yl)ethyl)oxazolidin-2-one is obtained in the form of a yellow solid.

Yield: 94%

¹H NMR (CDCl₃) δ: 1.22 (s, 3H); 1.24 (m, 2H); 1.25 (s, 3H); 1.41 (m, 1H); 1.57 (m, 2H); 1.76 (m, 2H); 1.88 (ls, 2H); 2.62 (m, 2H); 3.11 (m, 2H); 3.39 (m, 1H); 3.65 (m, 1H); 3.74 (m, 1H); 4.00 (m, 1H); 4.27 (m, 1H).

### Intermediate 3.25 (4R)-4-(((tert-butyldimethylsilyl)oxy)methyl)-5-phenyl-3-(2-(piperidin-4-yl)ethyl)oxazolidin-2-one

### Step 1: Preparation of tert-butyl 4-(2-((4R)-4-(((tert-butyldimethylsilyl)oxy)methyl)-2-oxo-5-phenyloxazolidin-3-yl)ethyl)piperidine-1-carboxylate

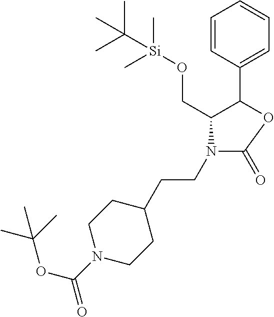

By proceeding as in Example 3.1 (step 2), but by replacing commercial 2-oxazolidinone by (4R)-4-(((tert-butyldimethylsilyl)oxy)methyl)-5-phenyloxazolidin-2-one, synthesized according to J. Org. Chem., 63, No. 24, 1998, p 8837-8842, tert-butyl 4-(2-((4R)-4-(((tert-butyldimethylsilyl)oxy)methyl)-2-oxo-5-phenyloxazolidin-3-yl)ethyl)piperidine-1-carboxylate is obtained in the form of a colorless oil. The predominant isomer is tert-butyl 4-(2-((4R,5R)-4-(((tert-butyldimethylsilyl)oxy)methyl)-2-oxo-5-phenyloxazolidin-3yl)ethyl)piperidine-1-carboxylate if one refers to the synthesized used to prepare oxazolidinone; however, this product should also contain a little of the other isomer, tert-butyl 4-(2-((4R,5S)-4-(((tert-butyldimethylsilyl)oxy)methyl)-2-oxo-5-phenyloxazolidin-3-yl)ethyl)piperidine-1-carboxylate.

Yield: 78%

¹H NMR (CDCl₃) δ: 0.10 (s, 3H); 0.11 (s, 3H); 0.92 (s, 9H); 1.06 (m, 2H); 1.30 (m, 1H); 1.44 (s, 9H); 1.46 (m, 2H); 1.54 (m, 1H); 1.70 (m, 1H); 2.62 (m, 2H); 3.13 (m, 1H); 3.58 (m, 2H); 3.77 (d, 2H, J=4 Hz); 4.05 (lm, 2H); 5.24 (d, 1H, J=4.8 Hz); 7.37 (m, 5H).

### Step 2: Preparation of tert-butyl 4-(2-((4R)-4-(hydroxymethyl)-2-oxo-5-phenyloxazolidin-3-yl)ethyl)piperidine-1-carboxylate

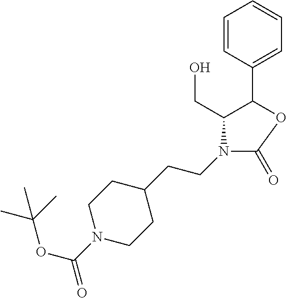

2.4 g (4.63 mmol) of tert-butyl 4-(2-((4R)-4-(((tert-butyldimethylsilyl)oxy)methyl)-2-oxo-5-phenyloxazolidin-3-yl)ethyl)piperidine-1-carboxylate previously prepared are solubilized in 25 ml of tetrahydrofuran. 6.9 ml (6.9 mmol) of a 1 M solution of tetrabutyl ammonium fluoride in tetrahydrofuran are added dropwise and the mixture is stirred at room temperature for 3 h. The medium is concentrated, and then it is washed with a 10% NaHCO₃ aqueous solution and extracted three times with ethyl acetate. The organic phases are dried on Na₂SO₄, filtered and evaporated, and 1.7 g (4.2 mmol) of tert-butyl 4-(2-((4R)-4-(hydroxymethyl)-2-oxo-5-phenyloxazolidin-3-yl)ethyl)piperidine-1-carboxylate are obtained in the form of a colorless oil after purification by flash chromatography on silica gel (gradient: n-heptane=100% to n-heptane/ethyl acetate=80/20% over 30 min).

Yield: 91%

¹H NMR (CDCl₃) δ: 1.08 (m, 2H); 1.35 (m, 1H); 1.44 (s, 9H); 1.48 (m, 2H); 1.58 (m, 1H); 1.59 (ls, 2H); 1.71 (m, 1H); 2.63 (m, 2H); 3.18 (m, 1H); 3.58 (m, 1H); 3.65 (m, 1H); 3.79 (m, 1H); 3.88 (m, 1H); 4.05 (m, 2H); 5.34 (d, 1H, J=5.6 Hz); 7.38 (m, 5H).

### Step 3: Preparation of (4R)-4-(hydroxymethyl)-5-phenyl-3-(2-(piperidin-4-yl)ethyl)oxazolidin-2-one

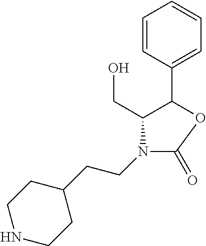

By proceeding as in Example 3.1 (step 3), but by replacing tert-butyl 4-(2-(2-oxooxazolidin-3-yl)ethyl)piperidine-1-carboxylate by tert-butyl 4-(2-((4R)-4-(hydroxymethyl)-2-oxo-5-phenyloxazolidin-3-yl)ethyl)piperidine-1-carboxylate previously prepared, (4R)-4-(hydroxymethyl)-5-phenyl-3-(2-(piperidin-4-yl)ethyl)oxazolidin-2-one is obtained in the form of a pale yellow solid.

Yield: 91%

¹H NMR (CDCl₃) δ: 1.20 (m, 2H); 1.37 (m, 1H); 1.49 (m, 2H); 1.64 (m, 1H); 1.77 (m, 1H); 2.10 (lm, 2H); 2.58 (m, 2H); 3.09 (m, 2H); 3.19 (m, 1H); 3.57 (m, 1H); 3.65 (m, 1H); 3.79 (dd, 1H, J=11.6 and 7.6 Hz); 3.88 (dd, 1H, J=11.6 and 4 Hz); 5.34 (d, 1H, J=5.6 Hz); 7.34 (m, 5H).

### Intermediate 3.26 (S)-4-((R or S)-hydroxy(phenyl)methyl)-3-(2-(piperidin-4-yl)ethyl)oxazolidin-2-one

### Step 1: Preparation of (S)-tert-butyl 4-(2-(4-formyl-2-oxooxazolidin-3-yl)ethyl)piperidine-1-carboxylate

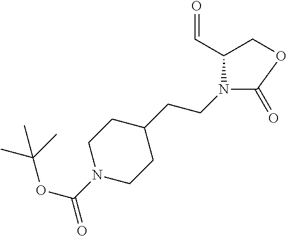 [SMILES: CC(C)(C)OC(=O)N1CCC(CCN2C(=O)OC[C@H]2C=O)CC1]

In a 250-ml flask swept with nitrogen, 2.5 g of (S)-methyl 3-(2-(1-(tert-butoxycarbonyl)piperidin-4-yl)ethyl)-2-oxooxazolidine-4-carboxylate (intermediate 3.21, step 2) are solubilized in 100 ml of dichloromethane. The mixture is cooled to −78° C. and 17.54 ml (17.54 mmol) of a 1 M solution of DIBAL in dichloromethane are added dropwise. After 3 h at −78° C., methanol and a saturated solution of potassium sodium tartrate are added. The mixture is removed from the cold bath and stirred for 1 h before extracting 3 times with dichloromethane. The organic phases are then dried on Na₂SO₄, filtered and concentrated. The anhydride thus obtained is used in the crude form for the next step.

### Step 2: Preparation of tert-butyl 4-(2-((S)-4-((R or S)-hydroxy(phenyl)methyl)-2-oxooxazolidin-3-yl)ethyl)piperidine-1-carboxylate

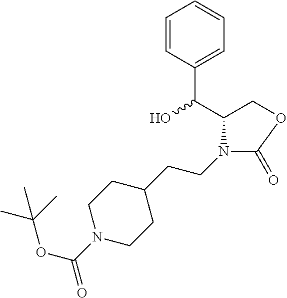 [SMILES: CC(C)(C)OC(=O)N1CCC(CCN2C(=O)OC[C@H]2C(O)c2ccccc2)CC1]

In a 150-ml flask swept with nitrogen, 2.28 g (crude product) of S)-tert-butyl 4-(2-(4-formyl-2-oxooxazolidin-3-yl)ethyl)piperidine-1-carboxylate, previously prepared, are solubilized in 25 ml of tetrahydrofuran. The mixture is cooled to 0° C. and 5.12 ml (15.37 mmol) of a 3M phenyl magnesium bromide solution in diethyl ether are added dropwise. The mixture is stirred for 1.5 h at 0° C. and then it is gently brought back to room temperature overnight. A saturated solution of NH₄Cl is added at 0° C. and extracted three times with ethyl acetate. The organic phases are then dried on Na₂SO₄, filtered and evaporated, and 1.8 g (4.45 mmol) of tert-butyl-(2-((4S)-4-(hydroxy(phenyl)methyl)-2-oxooxazolidin-3-yl)ethyl)piperidine-1-carboxylate are obtained in the form of a pale yellow oil after purification by flash chromatography on silica gel (gradient: n-heptane=100% to ethyl acetate=100% over 30 min).

Yield: 64%

The two isomers formed can be separated in reverse phase; HPLC (XBRIDGE C8, acetonitrile/water/KH₂PO₄ (60/40/6.8 g pH 4), 1 ml/min), retention time intermediate 3.26 (step 2)=5.34 min, retention time intermediate 3.27 (step 2)=5.73 min.

¹H NMR (CDCl₃) δ: 1.12 (m, 2H); 1.39 (m, 1H); 1.44 (s, 9H); 1.63 (m, 4H); 2.47 (ls, 1H); 2.66 (m, 2H); 3.41 (m, 1H); 3.61 (m, 1H); 3.86 (m, 1H); 3.96 (m, 1H); 4.06 (m, 3H); 4.74 (d, 1H, J=6.8 Hz); 7.37 (m, 5H).

### Step 3: Preparation of (S)-4-((R or S)-hydroxy(phenyl)methyl)-3-(2-(piperidin-4-yl)ethyl)oxazolidin-2-one

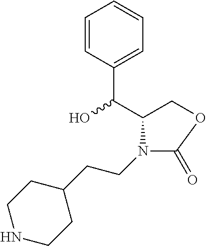 [SMILES: O=C1OC[C@@H](C(O)c2ccccc2)N1CCC1CCNCC1]

By proceeding as in Example 3.1 (step 3), but by replacing tert-butyl 4-(2-(2-oxooxazolidin-3-yl)ethyl)piperidine-1-carboxylate by tert-butyl 4-(2-((S)-4-((R or S)-hydroxy(phenyl)methyl)-2-oxooxazolidin-3-yl)ethyl)piperidine-1-carboxylate previously prepared, (S)-4-((R or S)-hydroxy(phenyl)methyl)-3-(2-(piperidin-4-yl)ethyl)oxazolidin-2-one is obtained in the form of a pale yellow oil.

Yield: 90%

¹H NMR (CDCl₃) δ: 1.17 (m, 2H); 1.35 (m, 1H); 1.57 (m, 1H); 1.66 (m, 3H); 2.51 (m, 2H); 2.93 (m, 2H); 3.42 (m, 1H); 3.59 (m, 1H); 3.84 (m, 1H); 3.93 (m, 1H); 4.05 (m, 1H); 4.69 (d, 1H, J=7.2 Hz); 7.36 (m, 5H).

### Intermediate 3.27 (S)-4-((S or R)-hydroxy(phenyl)methyl)-3-(2-(piperidin-4yl)ethyl)oxazolin-2-one

### Step 2: Preparation of tert-butyl 4-(2-((S)-4-((S or R)-hydroxy(phenyl)methyl)-2-oxooxazolidin-3-yl)ethyl)piperidine-1-carboxylate

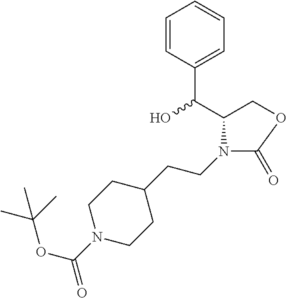 [SMILES: CC(C)(C)OC(=O)N1CCC(CCN2C(=O)OC[C@H]2C(O)c2ccccc2)CC1]

This product is obtained by separation in reverse phase (intermediate 3.26, step 2)

¹H NMR (CDCl₃) δ: 1.09 (m, 2H); 1.39 (m, 1H); 1.45 (s, 9H); 1.47 (m, 2H); 1.64 (m, 2H); 2.63 (m, 3H); 2.95 (m, 1H); 3.54 (m, 1H); 3.98 (m, 1H); 4.09 (m, 3H); 4.38 (m, 1H); 4.93 (d, 1H, J=3.2 Hz); 7.37 (m, 5H). 1.08 (m, 2H); 1.33 (m, 1H);

### Step 3: Preparation of (S)-4-((S or R)-hydroxy(phenyl)methyl)-3-(2-(piperidin-4yl)ethyl)oxazolin-2-one

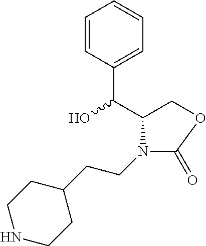 [SMILES: O=C1OC[C@@H](C(O)c2ccccc2)N1CCC1CCNCC1]

By proceeding as in Example 3.1 (step 3), but by replacing tert-butyl 4-(2-(2-oxooxazolidin-3-yl)ethyl)piperidine-1-carboxylate by tert-butyl 4-(2-((S)-4-((S or R)-hydroxy(phenyl)methyl)-2-oxooxazolidin-3-yl)ethyl)piperidine-1-carboxylate previously prepared, (S)-4-((S or R)-hydroxy(phenyl)methyl)-3-(2-(piperidin-4-yl)ethyl)oxazolidin-2-one is obtained in the form of a pale yellow oil.

Yield: 90%

¹H NMR (CDCl₃) δ: 1.08 (m, 2H); 1.33 (m, 1H); 1.47 (m, 2H); 1.64 (m, 2H); 2.51 (m, 3H); 2.95 (m, 3H); 3.54 (m, 1H); 3.96 (m, 1H); 4.03 (m, 1H); 4.37 (m, 1H); 4.92 (d, 1H, J=3.6 Hz); 7.35 (m, 5H).

### Intermediate 3.28 4-(2-(piperidin-4-yl)ethyl)morpholin-3-one

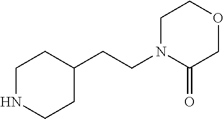 [SMILES: O=C1COCCN1CCC1CCNCC1]

By proceeding as in Example 3.1 (steps 2 and 3), but replacing commercial 2-oxazolidinone by morpholin-3-one, 4-(2-(piperidin-4-yl)ethyl)morpholin-3-one is obtained in the form of a clear oil.

¹H NMR (CDCl₃) δ: 1.18 (m, 2H); 1.40 (m, 1H); 1.49 (m, 2H); 1.73 (m, 2H); 2.21 (s, 1H); 2.59 (m, 2H); 3.08 (m, 2H); 3.35 (m, 2H); 3.45 (m, 2H); 3.88 (m, 2H); 4.16 (s, 2H).

### Intermediate 3.29 (R)-6-phenyl-4-(2-(piperidin-4-yl)ethyl)morpholin-3-one

### Step 1: Preparation of tert-butyl 4-(2-(1,3-dioxoisoindolin-2-yl)ethyl)piperidine-1-carboxylate

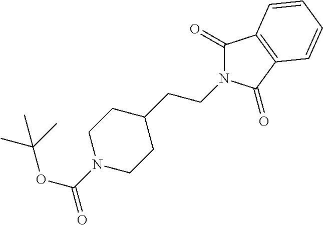 [SMILES: CC(C)(C)OC(=O)N1CCC(CCN2C(=O)c3ccccc3C2=O)CC1]

In a 250-ml flask, 5 g (21.80 mmol) of tert-butyl 4-(2-hydroxyethyl)piperidine-1-carboxylate are solubilized in 80 ml of tetrahydrofuran. 5.75 g (21.92 mmol) of Ph₃P and 3.25 g (22.08 mmol) of phthalimide are added. The medium is cooled to 0° C. and 4.30 ml (21.84 mmol) of DIAD are added dropwise. The mixture is then returned to room temperature, and then it is stirred for 24 h. The medium is concentrated, and 5.5 g (15.34 mmol) tert-butyl 4-(2-(1,3-dioxoisoindolin-2-yl)ethyl)piperidine-1-carboxylate are obtained in the form of a white solid after purification by flash chromatography on silica gel (eluent: n-heptane/acetone=80/20%)

Yield: 70%

¹H NMR (DMSOd₆) δ: 0.98 (m, 2H); 1.38 (s, 9H); 1.40 (is, 1H); 1.52 (m, 2H); 1.70 (m, 2H); 2.65 (m, 2H); 3.60 (m, 2H); 3.90 (m, 2H); 7.85 (m, 4H).

### Step 2: Preparation of tert-butyl 4-(2-aminoethyl)piperidine-1-carboxylate

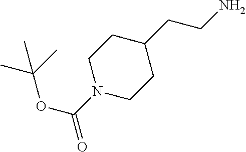 [SMILES: CC(C)(C)OC(=O)N1CCC(CCN)CC1]

5.5 g (15.34 mmol) of tert-butyl 4-(2-(1,3-dioxoisoindolin-2-yl)ethyl)piperidine-1-carboxylate prepared previously are solubilized in 40 ml of ethanol amine and the mixture is stirred at room temperature for 5.5 h. Then it is poured into 150 ml of water and extracted twice with 200 ml of dichloromethane. The organic phases are dried on MgSO₄, filtered and evaporated, and 2.74 g (11.95 mmol) of tert-butyl 4-(2-aminoethyl)piperidine-1-carboxylate are obtained in the form of a clear oil after purification by flash chromatography on silica gel (eluent: dichloromethane/methanol/NH₄OH=90/9/1%) Yield: 78%

¹H NMR (CDCl₃) δ: 1.10 (m, 2H); 1.40 (m, 2H); 1.45 (s, 9H); 1.49 (Is; 3H); 1.64 (m, 2H); 3.58 (m, 4H); 4.07 (m, 2H).

### Step 3: Preparation of (R)-tert-butyl 4-(2-((2-hydroxy-2-phenylethyl)amino)ethyl)piperidine-1-carboxylate

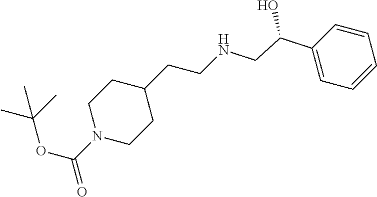 [SMILES: CC(C)(C)OC(=O)N1CCC(CCNC[C@H](O)c2ccccc2)CC1]

In a 100-ml flask, 2.74 g (11.95 mmol) of tert-butyl 4-(2-aminoethyl)piperidine-1-carboxylate previously prepared are introduced and 0.68 ml (5.94 mmol) of (R)-2-phenyloxirane and 5 ml of tetrahydrofuran are added. The mixture is heated at 70° C. for 40 hours. Then the medium is taken up with 50 ml of water and extracted twice with 100 ml of dichloromethane. The organic phases are dried on MgSO₄ and filtered and 1.17 g (3.36 mmol) of (R)-tert-butyl 4-(2-((2-hydroxy-2-phenylethyl)amino)ethyl)piperidine-1-carboxylate are obtained in the form of a cream oil that tends to solidify after purification by flash chromatography on silica gel (eluent: dichloromethane/methanol=90/10%)

Yield: 57%

¹H NMR (CDCl₃) δ: 1.10 (m, 2H); 1.43 (m, 3H); 1.45 (s, 9H); 1.63 (m, 2H); 2.68 (m, 5H); 2.89 (m, 1H); 4.06 (m, 2H); 4.71 (dd, 1H, J=8.9 and 3.4 Hz); 7.28 (m, 1H); 7.35 (m, 4H).

### Step 4: Preparation of (R)-tert-butyl 4-(2-(5-oxo-2-phenylmorpholino)ethyl)piperidine-1-carboxylate

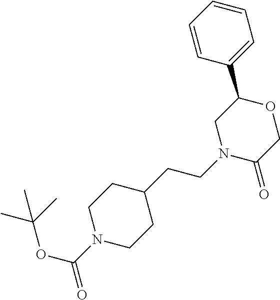 [SMILES: CC(C)(C)OC(=O)N1CCC(CCN2C[C@@H](c3ccccc3)OCC2=O)CC1]

In a 100-ml flask, 1.17 g (3.36 mmol) of (R)-tert-butyl 4-(2-((2-hydroxy-2-phenylethyl)amino)ethyl)piperidine-1-carboxylate previously prepared are solubilized in 5 ml of tetrahydrofuran and 3 ml of water. 2 ml of a 30% NaOH aqueous solution and 0.1 g (0.32 mmol) of benzyl tributyl ammonium chloride are added. The medium is then cooled to 0° C. and 0.53 ml (6.66 mmol) of 2-chloroacetyl chloride and a spatula tip of KI are added dropwise. The mixture is stirred at room temperature for 24 hours. Then 50 ml of water are added and it is extracted twice with dichloromethane. The organic phases are dried on MgSO₄, filtered and evaporated, and 1.15 g (2.96 mmol) of (R)-tert-butyl 4-(2-(5-oxo-2-phenylmorpholino)ethyl)piperidine-1-carboxylate are obtained in the form of a clear oil after purification by flash chromatography on silica gel (eluent: dichloromethane/ethyl acetate=70/30%)

Yield: 88%

¹H NMR (CDCl₃) δ: 1.12 (m, 2H); 1.45 (ls, 10H); 1.52 (m, 2H); 1.71 (m, 2H); 2.68 (m, 2H); 3.36 (m, 2H); 3.55 (m, 2H); 4.07 (m, 2H); 4.31 (d, 1H, J=16.6 Hz); 4.43 (d, 1H, J=16.6 Hz); 4.79 (dd, 1H, J=10.4 and 2.4 Hz); 7.36 (m, 5H).

### Step 5: Preparation of (R)-6-phenyl-4-(2-(piperidin-4-yl)ethyl)morpholin-3-one

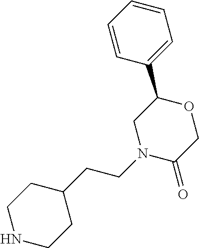 [SMILES: O=C1CO[C@H](c2ccccc2)CN1CCC1CCNCC1]

By proceeding as in Example 3.1 (step 3), but by replacing tert-butyl 4-(2-(2-oxooxazolidin-3-yl)ethyl)piperidine-1-carboxylate by (R)-tert-butyl 4-(2-(5-oxo-2-phenylmorpholino)ethyl)piperidine-1-carboxylate previously prepared, (R)-6-phenyl-4-(2-(piperidin-4-yl)ethyl)morpholin-3-one is obtained in the form of a clear oil.

Yield: 93%

¹H NMR (CDCl₃) δ: 1.18 (m, 2H); 1.40 (m, 1H); 1.53 (m, 2H); 1.72 (m, 2H); 2.23 (Is, 1H); 2.60 (m, 2H); 3.09 (m, 2H); 3.36 (m, 2H); 3.55 (m, 2H); 4.31 (d, 1H, J=16.6 Hz); 4.43 (d, 1H, J=16.6 Hz); 4.79 (dd, 1H, J=10.4 and 2.6 Hz); 7.37 (m, 5H).

### Intermediate 3.30 3-(3-(piperidin-4-yl)propyl)oxazolidin-2-one

### Step 1: tert-butyl 4-(3-hydroxypropyl)piperidine-1-carboxylate

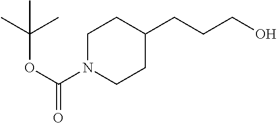 [SMILES: CC(C)(C)OC(=O)N1CCC(CCCO)CC1]

In a 500-ml flask swept with nitrogen, 5 g (17.53 mmol) of tert-butyl 4-(3-methoxy-3-oxopropyl)piperidine-1-carboxylate (prepared according to the method reported in J. Med. Chem. 1995, 38, p 3332-3341 by taking commercial tert-butyl 4-(hydroxymethyl)piperidine-1-carboxylate as the starting product) in 100 ml of dichloromethane. The medium is cooled to −78° C. and 52.6 ml (52.6 mmol) of a 1 M solution of DIBAL-H in dichloromethane are added dropwise. It is stirred for 2 h at 0° C. It is cooled to −78° C. and a saturated solution of potassium sodium tartrate is added. After return to room temperature, it is extracted and then the organic phase is dried on Na₂SO₄, filtered and evaporated, and 4.5 g of the crude oil are obtained, used as is in the next step.

### Step 1: tert-butyl 4-(3-iodopropyl)piperidine-1-carboxylate

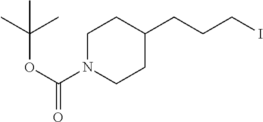 [SMILES: CC(C)(C)OC(=O)N1CCC(CCCI)CC1]

By proceeding as in Example 3.1 (step 1), but by replacing commercial tert-butyl 4-(2-hydroxyethyl)piperidine-1-carboxylate by tert-butyl 4-(3-hydroxypropyl)piperidine-1-carboxylate previously prepared, tert-butyl 4-(3-iodopropyl)piperidine-1-carboxylate is obtained in the form of a colorless oil.

Yield: 85%

¹H NMR (CDCl₃) δ: 1.10 (m, 2H); 1.35 (m, 3H); 1.45 (s, 9H); 1.64 (m, 2H); 1.85 (m, 2H); 2.67 (m, 2H); 3.17 (t, 2H, J=6.8 Hz); 4.06 (m, 2H).

### Step 2: Preparation tert-butyl 4-(3-(2-oxooxazolidin-3-yl)propyl)piperidine-1-carboxylate

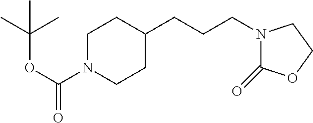 [SMILES: CC(C)(C)OC(=O)N1CCC(CCCN2CCOC2=O)CC1]

By proceeding as in Example 3.1 (step 2), but by replacing tert-butyl 4-(2-iodoethyl)piperidine-1-carboxylate by tert-butyl 4-(3-iodopropyl)piperidine-1-carboxylate previously prepared, tert-butyl 4-(3-(2-oxooxazolidin-3-yl)propyl)piperidine-1-carboxylate is obtained in the form of a white solid.

Yield: 81%

¹H NMR (CDCl₃) δ: 1.08 (m, 2H); 1.26 (m, 2H); 1.39 (m, 1H); 1.45 (s, 9H); 1.60 (m, 4H); 2.67 (m, 2H); 3.25 (t, 2H, J=7.6 Hz); 3.55 (m, 2H); 4.07 (m, 2H); 4.32 (m, 2H).

### Step 3: Preparation of 3-(3-(piperidin-4-yl)propyl)oxazolidin-2-one

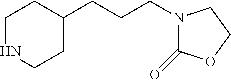 [SMILES: O=C1OCCN1CCCC1CCNCC1]

By proceeding as in Example 3.1 (step 3), but by replacing tert-butyl 4-(2-(2-oxooxazolidin-3-yl)ethyl)piperidine-1-carboxylate by tert-butyl 4-(3-(2-oxooxazolidin-3-yl)propyl)piperidine-1-carboxylate prepared previously, 3-(3-(piperidin-4-yl)propyl)oxazolidin-2-one is obtained in the form of a clear oil.

Yield: 90%

¹H NMR (CDCl₃) δ: 1.25 (m, 4H); 1.40 (m, 1H); 1.56 (m, 2H); 1.71 (m, 2H); 2.63 (m, 2H); 2.68 (ls, 1H); 3.14 (m, 2H); 3.25 (m, 2H); 3.56 (m, 2H); 4.33 (m, 2H).

### Intermediate 3.31 (S)-4-benzyl-3-(3-(piperidin-4-yl)propyl)oxazolidin-2-one

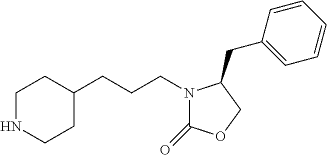 [SMILES: O=C1OC[C@H](Cc2ccccc2)N1CCCC1CCNCC1]

By proceeding as in Example 3.1, but replacing commercial 2-oxazolidinone by commercial (S)-4-benzyloxazolidin-2-one, (S)-4-benzyl-3-(3-(piperidin-4-yl)propyl)oxazolidin-2-one is obtained in the form of a pale yellow oil. The product is used in the crude form.

### Intermediate 3.32 (R)-4-phenyl-3-(3-(piperidin-4-yl)propyl)-oxazolidin-2-one

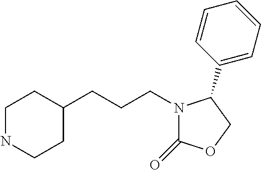 [SMILES: O=C1OC[C@@H](c2ccccc2)N1CCCC1CCNCC1]

By proceeding as in Example 3.1, but replacing commercial 2-oxazolidinone by commercial (R)-(−)-4-phenyl-oxazolidin-2-one, (R)-4-phenyl-3-(3-(piperidin-4-yl)propyl)oxazolidin-2-one is obtained in the form of a clear oil. The product is used in the crude form.

### Intermediate 3.33 4-(3-piperidin-4-yl-propyl)-morpholin-3-one

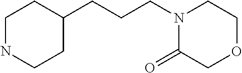 [SMILES: O=C1COCCN1CCCC1CCNCC1]

By proceeding as in Example 3.1, but replacing commercial 2-oxazolidinone by morpholin-3-one, 4-(3-piperidin-4-yl-propyl)-morpholin-3-one is obtained in the form of a clear yellow oil. The product is used in the crude form.

### Intermediate 3.34 3-(4-piperidin-4-yl-butyl)-oxazolidin-2-one

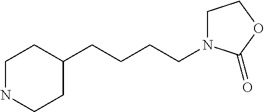 [SMILES: O=C1OCCN1CCCCC1CCNCC1]

By proceeding as in Example 3.1 where commercial 2-oxazolidinone is used, but replacing tert-butyl-4-(2-iodoethyl)piperidine-1-carboxylate in step 1 by tert-butyl-4-(4-iodobutyl)piperidine-1-carboxylate prepared by reduction of commercial ester with LiAlH₄ as described in WO2008/130718.

| Ex | structure | intermediates | reaction diagram |
|---|---|---|---|
| 1 | 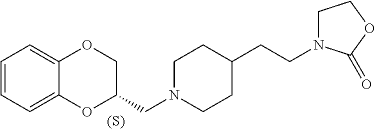 | 4.1/2.1 3.1 | diagram 2 diagram 3 |
| 2 | 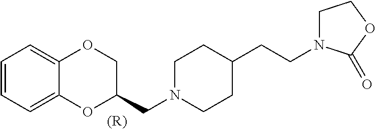 | 4.2/2.2 3.1 | diagram 2 diagram 3 |
| 3 | 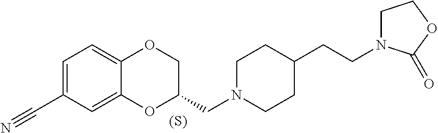 | 4.10/2.11 3.1 | diagram 2 diagram 3 |
| 4 | 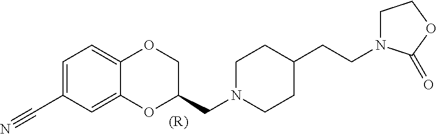 | 4.26/2.12 3.1 | diagram 2 diagram 3 |
| 5 | 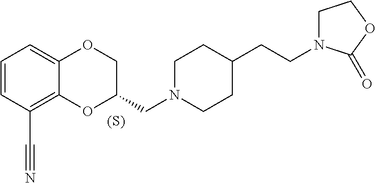 | 4.11/2.13 3.1 | diagram 2 diagram 3 |
| 6 | 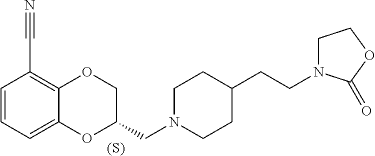 | 4.25/2.14 3.1 | diagram 2 diagram 3 |
| 7 | 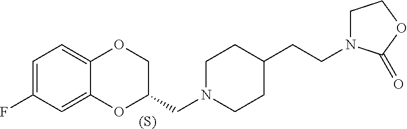 | 4.6/2.17 3.1 | diagram 2 diagram 3 |
| 8 | 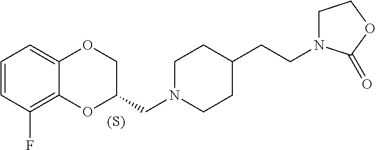 | 4.17/2.16 3.1 | diagram 2 diagram 3 |
| 9 | 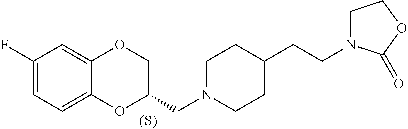 | 4.18/2.18 3.1 | diagram 2 diagram 3 |
| 10 | 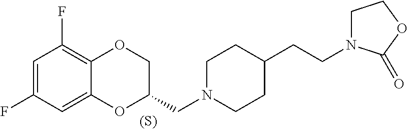 | 4.19/2.19 3.1 | diagram 2 diagram 3 |
| 11 | 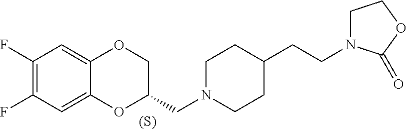 | 4.22/2.22 3.1 | diagram 2 diagram 3 |
| 12 | 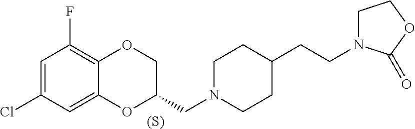 | 4.20/2.20 3.1 | diagram 2 diagram 3 |
| 13 | 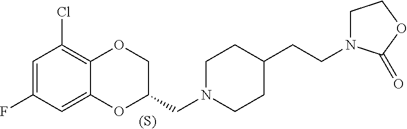 | 4.21/2.21 3.1 | diagram 2 diagram 3 |
| 14 | 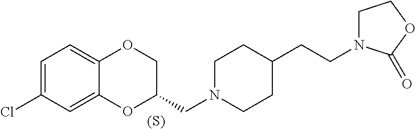 | 4.5/2.7 3.1 | diagram 2 diagram 3 |
| 15 | 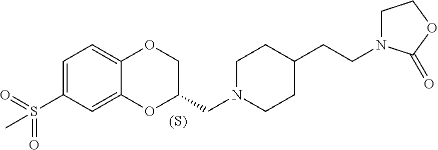 | 4.23/2.23 3.1 | diagram 2 diagram 3 |
| 16 | 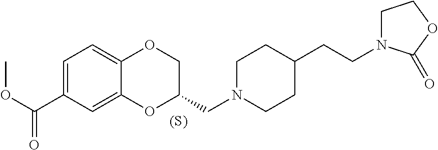 | 4.24/2.24 3.1 | diagram 2 diagram 3 |
| 17 | 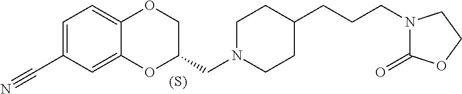 | 4.10/2.11 3.30 | diagram 2 diagrams 6 and 4 |
| 18 | 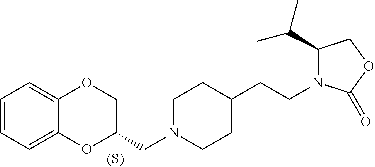 | 4.1/2.1 3.2 | diagram 2 diagram 3 |
| 19 | 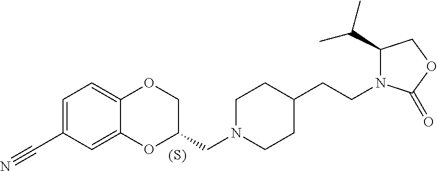 | 4.10/2.11 3.2 | diagram 2 diagram 3 |
| 20 | 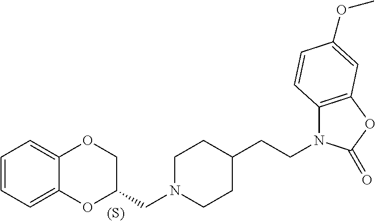 | 4.1/2.1 3.3 | diagram 2 diagram 3 |
| 21 | 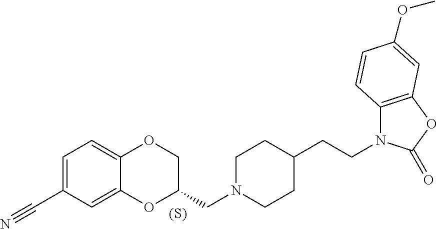 | 4.10/2.11 3.3 | diagram 2 diagram 3 |
| 22 | 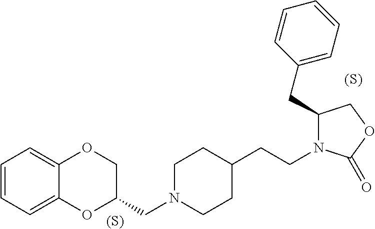 | 4.1/2.1 3.4 | diagram 2 diagram 3 |
| 23 | 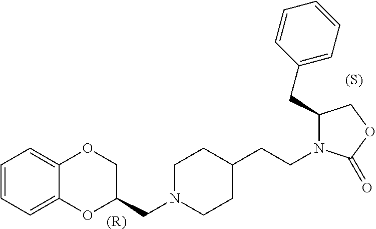 | 4.2/2.2 3.4 | diagram 2 diagram 3 |
| 24 | 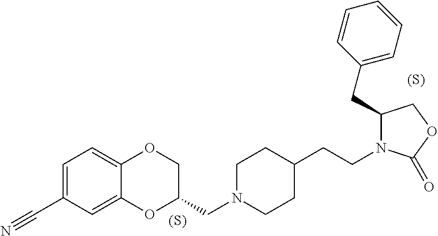 | 4.10/2.11 3.4 | diagram 2 diagram 3 |
| 25 | 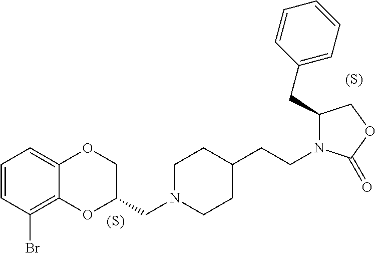 | 4.7/2.4 3.4 | diagram 2 diagram 3 |
| 26 | 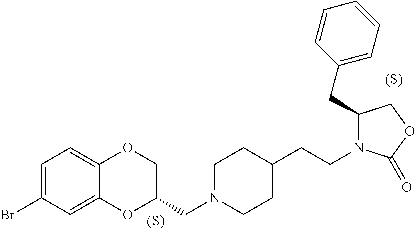 | 4.8/2.5 3.4 | diagram 2 diagram 3 |
| 27 | 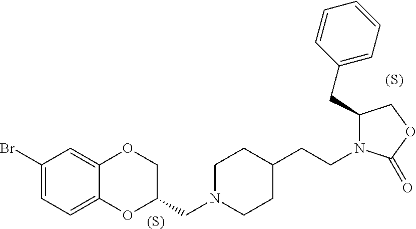 | 4.9/2.6 3.4 | diagram 2 diagram 3 |
| 28 | 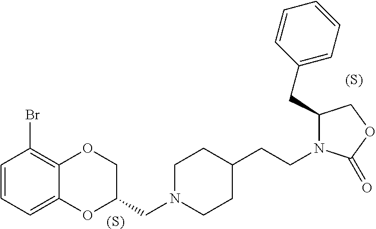 | 4.16/2.15 3.4 | diagram 2 diagram 3 |
| 29 | 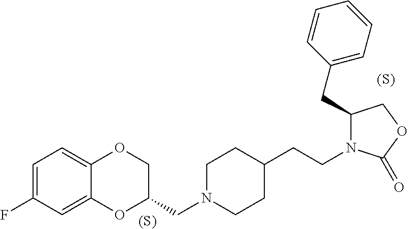 | 4.6/2.17 3.4 | diagram 2 diagram 3 |
| 30 | 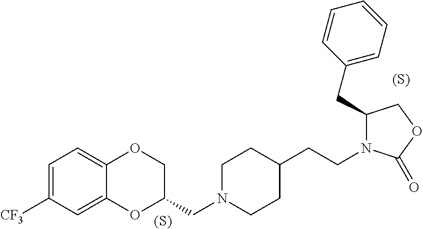 | 4.4/2.10 3.4 | diagram 2 diagram 3 |
| 31 | 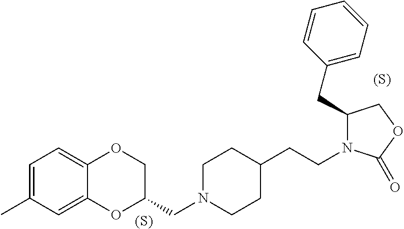 | 4.3/2.3 3.4 | diagram 2 diagram 3 |
| 32 | 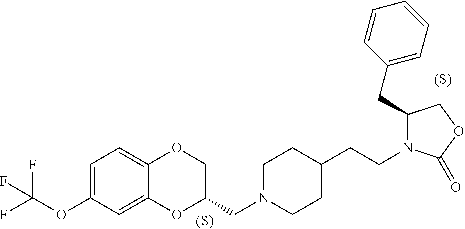 | 4.14/2.8 3.4 | diagram 2 diagram 3 |
| 33 | 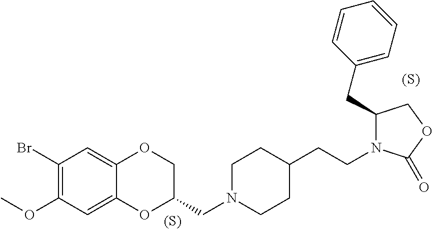 | 4.15/2.9 3.4 | diagram 2 diagram 3 |
| 34 | 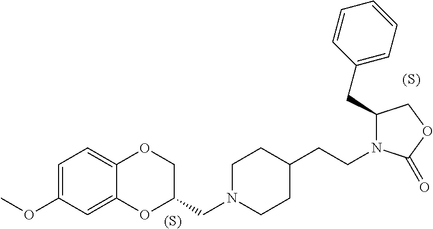 | 4.15/2.9 3.4 | diagram 2 diagram 3 |
| 35 | 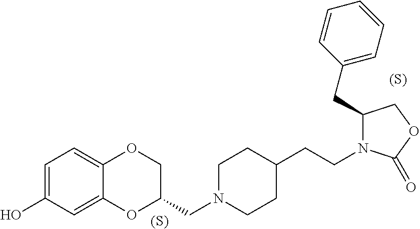 | 4.12/2.25 3.4 | diagram 2 diagram 3 |
| 36 | 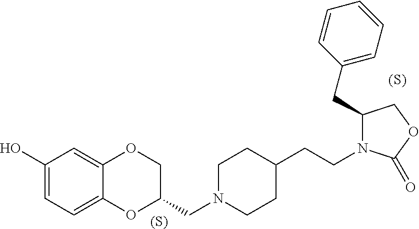 | 4.13/2.27 3.4 | diagram 2 diagram 3 |
| 37 | 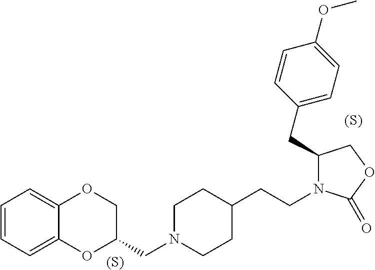 | 4.1/2.1 3.9 | diagram 2 diagrams 9 and 3 |
| 38 | 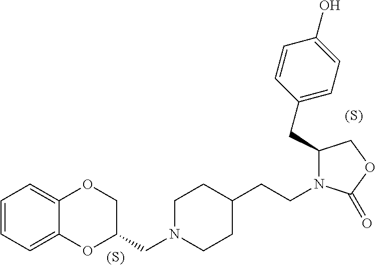 | 4.1/2.1 3.10 | diagram 2 diagrams 9 and 3 |
| 39 | 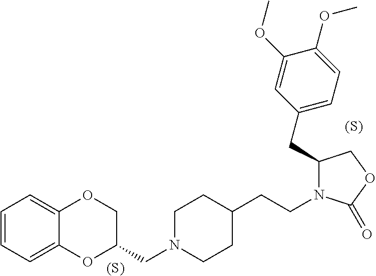 | 4.1/2.1 3.11 | diagram 2 diagrams 9 and 3 |
| 40 | 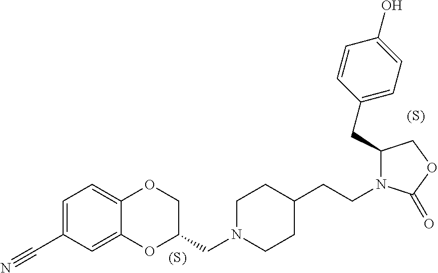 | 4.10/2.11 3.10 | diagram 2 diagrams 9 and 3 |
| 41 | 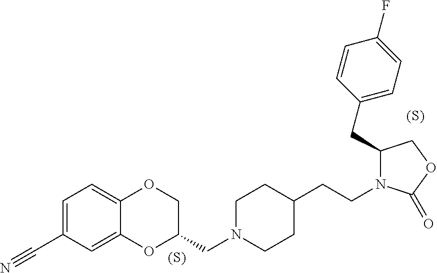 | 4.10/2.11 3.12 | diagram 2 diagrams 9 and 3 |
| 42 | 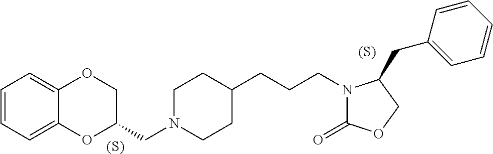 | 4.1/2.1 3.31 | diagram 2 diagram 3 |
| 43 | 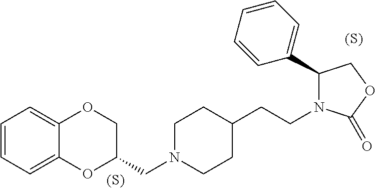 | 4.1/2.1 3.6 | diagram 2 diagram 3 |
| 44 | 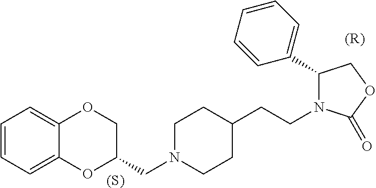 | 4.1/2.1 3.5 | diagram 2 diagram 3 |
| 45 | 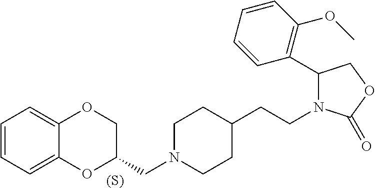 | 4.1/2.1 3.7 | diagram 2 diagrams 8 and 3 |
| 46 | 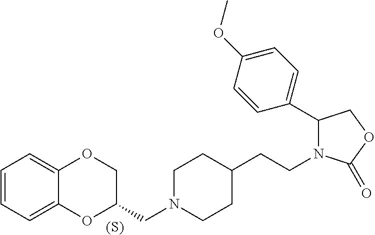 | 4.1/2.1 3.8 | diagram 2 diagrams 8 and 3 |
| 47 | 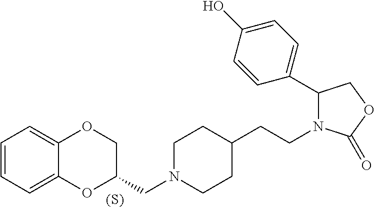 | 4.1/2.1 3.8 | diagram 2 diagrams 8 and 3 |
| 48 | 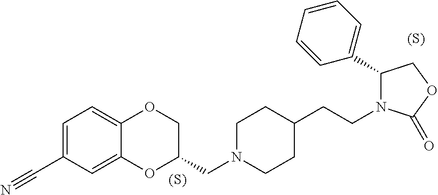 | 4.10/2.11 3.5 | diagram 2 diagram 3 |
| 49 | 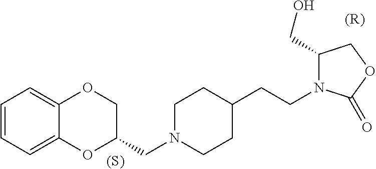 | 4.1/2.1 3.21 | diagram 2 diagrams 5 and 7 |
| 50 | 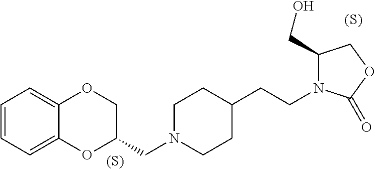 | 4.1/2.1 3.22 | diagram 2 diagrams 5 and 7 |
| 51 | 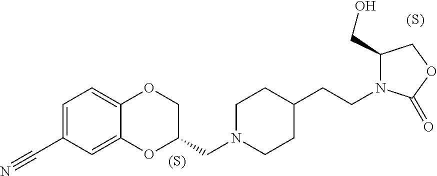 | 4.10/2.11 3.22 | diagram 2 diagrams 5 and 7 |
| 52 | 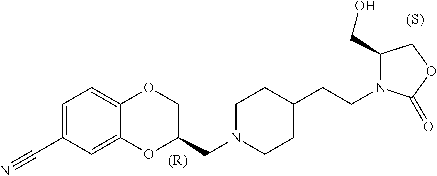 | 4.26/2.12 3.22 | diagram 2 diagrams 5 and 7 |
| 53 | 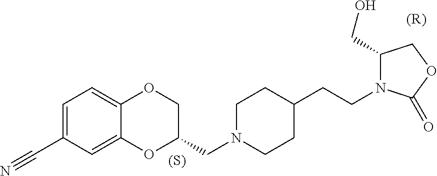 | 4.10/2.11 3.21 | diagram 2 diagrams 5 and 7 |
| 54 | 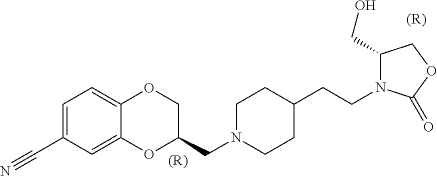 | 4.26/2.12 3.21 | diagram 2 diagrams 5 and 7 |
| 55 | 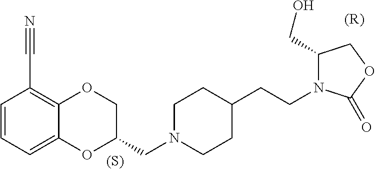 | 4.25/2.14 3.21 | diagram 2 diagrams 5 and 7 |
| 56 | 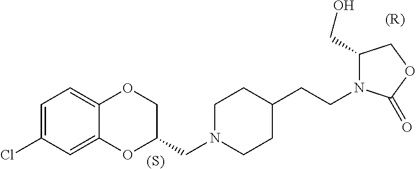 | 4.5/2.7 3.21 | diagram 2 diagrams 5 and 7 |
| 57 | 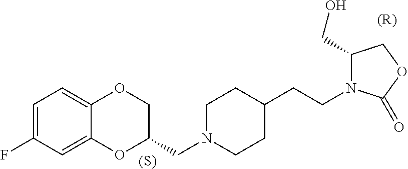 | 4.6/2.17 3.21 | diagram 2 diagrams 5 and 7 |
| 58 | 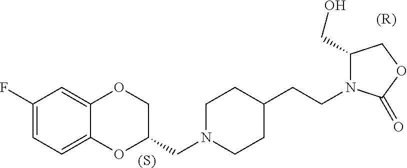 | 4.18/2.18 3.21 | diagram 2 diagrams 5 and 7 |
| 59 | 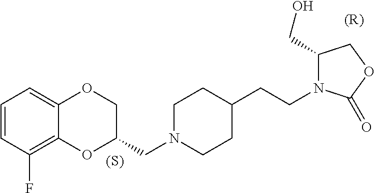 | 4.17/2.16 3.21 | diagram 2 diagrams 5 and 7 |
| 60 | 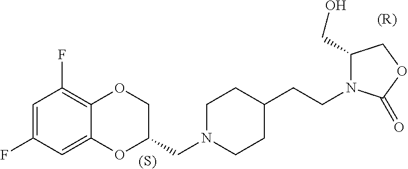 | 4.19/2.19 3.21 | diagram 2 diagrams 5 and 7 |
| 61 | 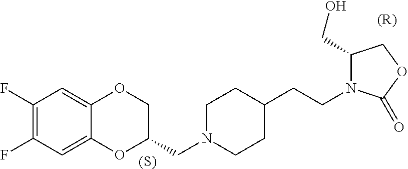 | 4.22/2.22 3.21 | diagram 2 diagrams 5 and 7 |
| 62 | 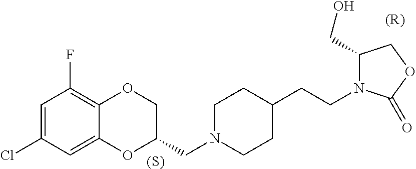 | 4.20/2.20 3.21 | diagram 2 diagrams 5 and 7 |
| 63 | 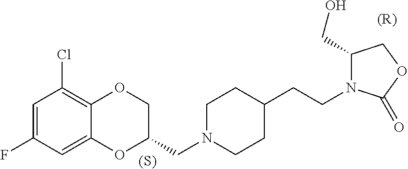 | 4.21/2.21 3.21 | diagram 2 diagrams 5 and 7 |
| 64 | 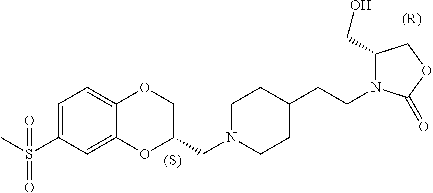 | 4.23/2.23 3.21 | diagram 2 diagrams 5 and 7 |
| 65 | 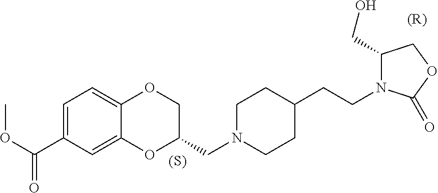 | 4.24/2.24 3.21 | diagram 2 diagrams 5 and 7 |
| 66 | 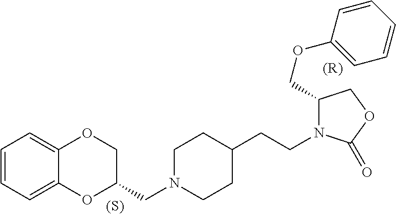 | 4.1/2.1 3.13 | diagram 2 diagrams 10 and 3 |
| 67 | 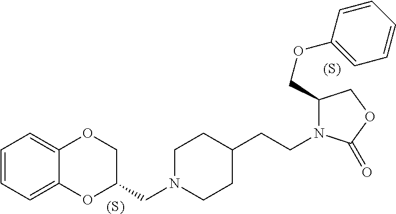 | 4.1/2.1 3.15 | diagram 2 diagrams 10 and 3 |
| 68 | 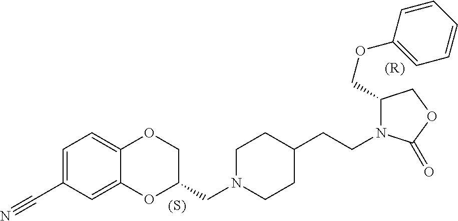 | 4.10/2.11 3.13 | diagram 2 diagrams 10 and 3 |
| 69 | 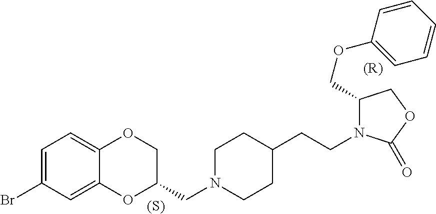 | 4.8/2.5 3.13 | diagram 2 diagrams 10 and 3 |
| 70 | 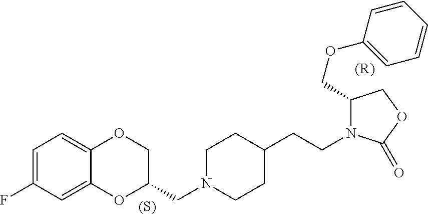 | 4.6/2.17 3.13 | diagram 2 diagrams 10 and 3 |
| 71 | 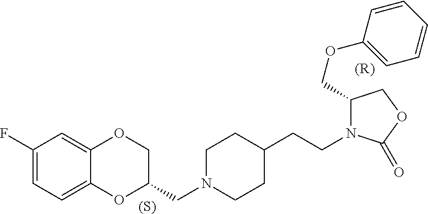 | 4.18/2.18 3.13 | diagram 2 diagrams 10 and 3 |
| 72 | 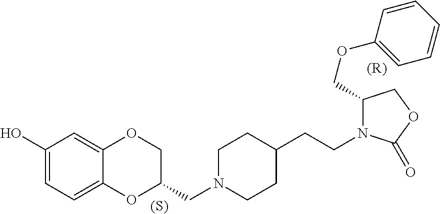 | 4.13/2.27 3.13 | diagram 2 diagrams 10 and 3 |
| 73 | 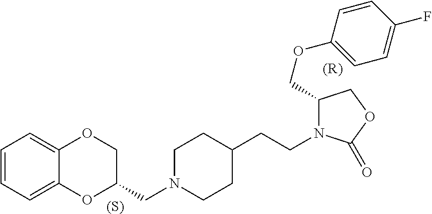 | 4.1/2.1 3.15 | diagram 2 diagrams 10 and 3 |
| 74 | 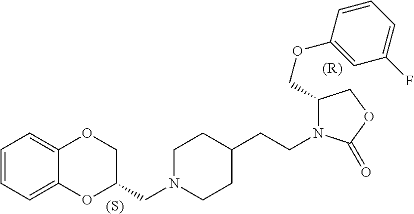 | 4.1/2.1 3.16 | diagram 2 diagrams 10 and 3 |
| 75 | 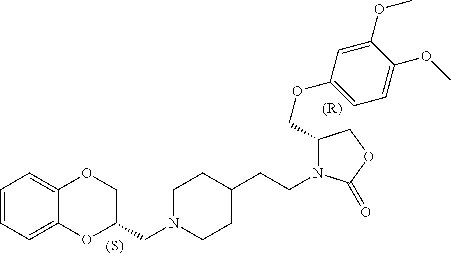 | 4.1/2.1 3.17 | diagram 2 diagrams 10 and 3 |
| 76 | 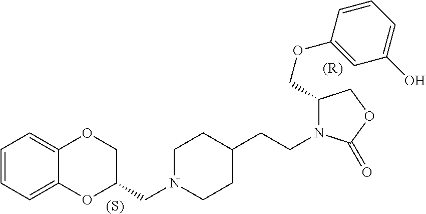 | 4.1/2.1 3.18 | diagram 2 diagrams 10 and 3 |
| 77 | 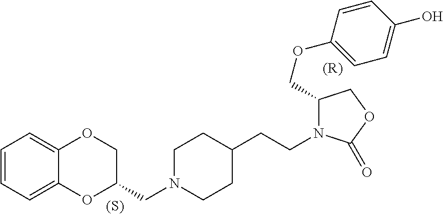 | 4.1/2.1 3.19 | diagram 2 diagrams 10 and 3 |
| 78 | 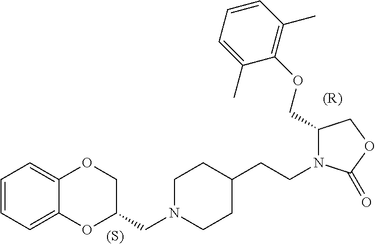 | 4.1/2.1 3.20 | diagram 2 diagrams 10 and 3 |
| 79 | 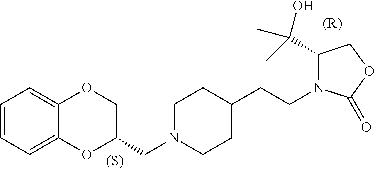 | 4.1/2.1 3.24 | diagram 2 diagrams 5 and 7 |
| 80 | 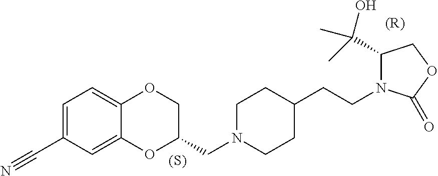 | 4.10/2.11 3.24 | diagram 2 diagrams 5 and 7 |
| 81 | 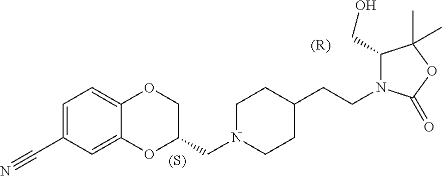 | 4.10/2.11 3.23 | diagram 2 diagrams 11 and 3 |
| 82 | 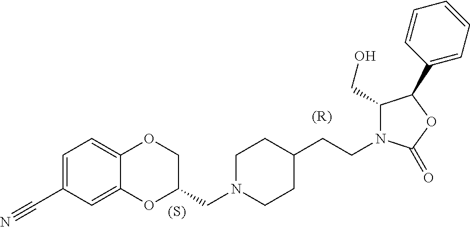 | 4.10/2.11 3.25 | diagram 2 diagrams 11 and 3 |
| 83 | 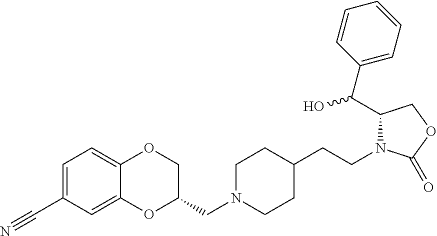 [SMILES: N#Cc1ccc2c(c1)O[C@@H](CN1CCC(CCN3C(=O)OC[C@H]3C(O)c3ccccc3)CC1)CO2] | 4.10/2.11 3.26 | diagram 2 diagrams 5 and 7 |
| 84 | 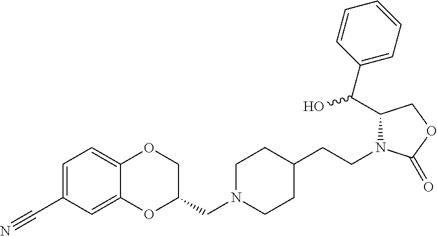 [SMILES: N#Cc1ccc2c(c1)O[C@@H](CN1CCC(CCN3C(=O)OC[C@H]3C(O)c3ccccc3)CC1)CO2] | 4.10/2.11 3.27 | diagram 2 diagrams 5 and 7 |
| 85 | 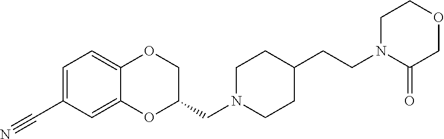 [SMILES: N#Cc1ccc2c(c1)O[C@@H](CN1CCC(CCN3CCOCC3=O)CC1)CO2] | 4.10/2.11 3.27 | diagram 2 diagram 3 |
| 86 | 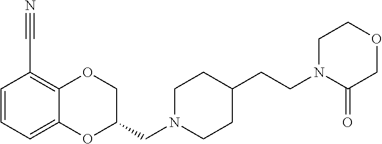 [SMILES: N#Cc1cccc2c1OC[C@H](CN1CCC(CCN3CCOCC3=O)CC1)O2] | 4.25/2.14 3.27 | diagram 2 diagram 3 |
| 87 | 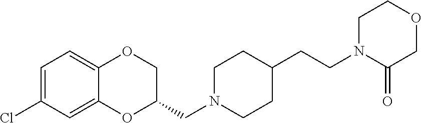 [SMILES: O=C1COCCN1CCC1CCN(C[C@H]2COc3ccc(Cl)cc3O2)CC1] | 4.5/2.7 3.27 | diagram 2 diagram 3 |
| 88 | 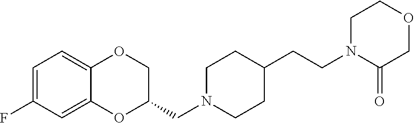 [SMILES: O=C1COCCN1CCC1CCN(C[C@H]2COc3ccc(F)cc3O2)CC1] | 4.6/2.17 3.27 | diagram 2 diagram 3 |
| 89 | 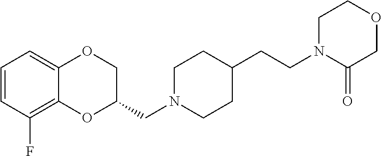 [SMILES: O=C1COCCN1CCC1CCN(C[C@H]2COc3cccc(F)c3O2)CC1] | 4.17/2.16 3.27 | diagram 2 diagram 3 |
| 90 | 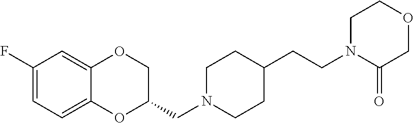 [SMILES: O=C1COCCN1CCC1CCN(C[C@H]2COc3cc(F)ccc3O2)CC1] | 4.18/2.18 3.28 | diagram 2 diagram 3 |
| 91 | 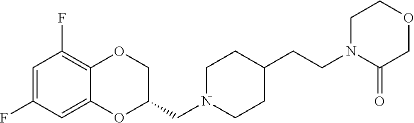 [SMILES: O=C1COCCN1CCC1CCN(C[C@H]2COc3c(F)cc(F)cc3O2)CC1] | 4.19/2.19 3.28 | diagram 2 diagram 3 |
| 92 | 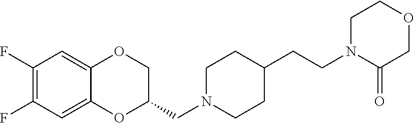 [SMILES: O=C1COCCN1CCC1CCN(C[C@H]2COc3cc(F)c(F)cc3O2)CC1] | 4.22/2.22 3.28 | diagram 2 diagram 3 |
| 93 | 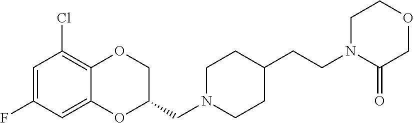 [SMILES: O=C1COCCN1CCC1CCN(C[C@H]2COc3c(Cl)cc(F)cc3O2)CC1] | 4.21/2.21 3.28 | diagram 2 diagram 3 |
| 94 | 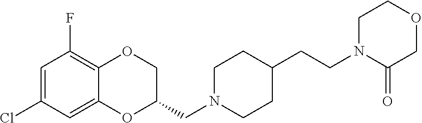 [SMILES: O=C1COCCN1CCC1CCN(C[C@H]2COc3c(F)cc(Cl)cc3O2)CC1] | 4.20/2.20 3.28 | diagram 2 diagram 3 |
| 95 | 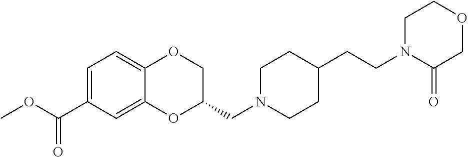 [SMILES: COC(=O)c1ccc2c(c1)O[C@@H](CN1CCC(CCN3CCOCC3=O)CC1)CO2] | 4.24/2.24 3.28 | diagram 2 diagram 3 |
| 96 | 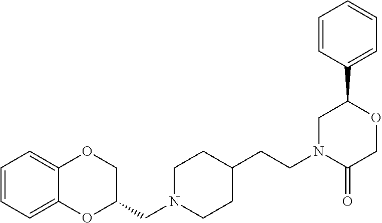 [SMILES: O=C1CO[C@H](c2ccccc2)CN1CCC1CCN(C[C@H]2COc3ccccc3O2)CC1] | 4.1/2.1 3.29 | diagram 2 diagrams 6 and 4 |
| 97 | 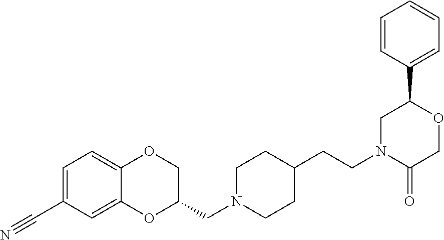 [SMILES: N#Cc1ccc2c(c1)O[C@@H](CN1CCC(CCN3C[C@@H](c4ccccc4)OCC3=O)CC1)CO2] | 4.10/2.11 3.29 | diagram 2 diagrams 6 and 4 |
| 98 | 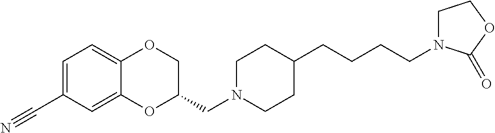 [SMILES: N#Cc1ccc2c(c1)O[C@@H](CN1CCC(CCCCN3CCOC3=O)CC1)CO2] | 4.10/2.11 3.34 | diagram 2 diagram 3 |
| 99 | 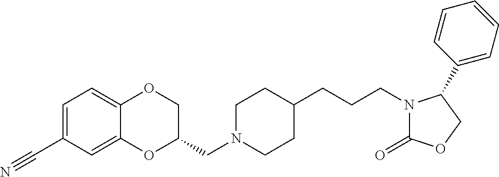 [SMILES: N#Cc1ccc2c(c1)O[C@@H](CN1CCC(CCCN3C(=O)OC[C@H]3c3ccccc3)CC1)CO2] | 4.10/2.11 3.32 | diagram 2 diagram 3 |
| 100 | 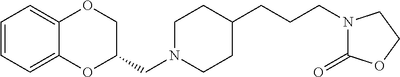 [SMILES: O=C1OCCN1CCCC1CCN(C[C@H]2COc3ccccc3O2)CC1] | 4.1/2.10 3.30 | diagram 2 diagram 3 |
| 101 | 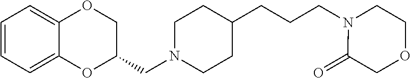 [SMILES: O=C1COCCN1CCCC1CCN(C[C@H]2COc3ccccc3O2)CC1] | 4.1/2.10 3.33 | diagram 2 diagram 3 |
| 102 | 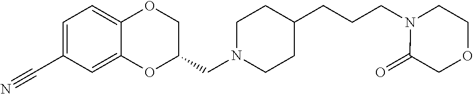 [SMILES: N#Cc1ccc2c(c1)O[C@@H](CN1CCC(CCCN3CCOCC3=O)CC1)CO2] | 4.10/2.11 3.33 | diagram 2 diagram 3 |
| 103 | 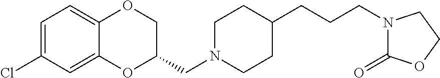 [SMILES: O=C1OCCN1CCCC1CCN(C[C@H]2COc3ccc(Cl)cc3O2)CC1] | 4.10/2.11 3.29 | diagram 2 diagram 3 |
| 104 | 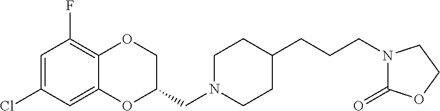 [SMILES: O=C1OCCN1CCCC1CCN(C[C@H]2COc3c(F)cc(Cl)cc3O2)CC1] | 4.20/2.20 3.30 | diagram 2 diagram 3 |
| 105 | 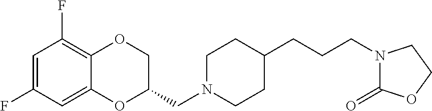 [SMILES: O=C1OCCN1CCCC1CCN(C[C@H]2COc3c(F)cc(F)cc3O2)CC1] | 4.19/2.19 3.30 | diagram 2 diagram 3 |
| 106 | 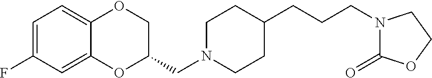 [SMILES: O=C1OCCN1CCCC1CCN(C[C@H]2COc3ccc(F)cc3O2)CC1] | 4.6/2.17 3.30 | diagram 2 diagram 3 |
| 107 | 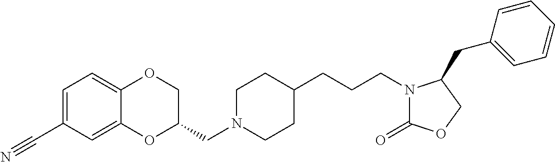 [SMILES: N#Cc1ccc2c(c1)O[C@@H](CN1CCC(CCCN3C(=O)OC[C@@H]3Cc3ccccc3)CC1)CO2] | 4.10/2.11 3.31 | diagram 2 diagram 3 |
| 108 | 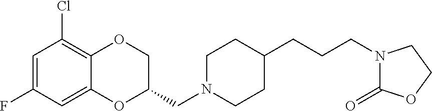 [SMILES: O=C1OCCN1CCCC1CCN(C[C@H]2COc3c(Cl)cc(F)cc3O2)CC1] | 4.21/2.21 3.30 | diagram 2 diagram 3 |
| 109 | 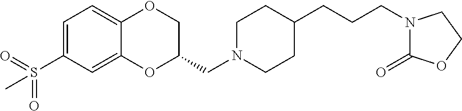 [SMILES: CS(=O)(=O)c1ccc2c(c1)O[C@@H](CN1CCC(CCCN3CCOC3=O)CC1)CO2] | 4.23/2.23 3.30 | diagram 2 diagram 3 |
| 110 | 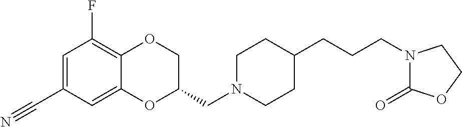 [SMILES: N#Cc1cc(F)c2c(c1)O[C@@H](CN1CCC(CCCN3CCOC3=O)CC1)CO2] | 4.27/2.26 3.30 | diagram 2 diagram 3 |

The invention also relates to a pharmaceutical composition comprising at least one compound of general formula (I) or one of its pharmaceutically-acceptable salts, and a pharmaceutically-acceptable excipient.

By considering the selective modulation of dopamine and norepinephrine transmissions that D2, D3 and alpha2C receptors exert in the brain, the compounds of the invention are suited to various therapeutic applications. The compounds of the invention can therefore be used for the preparation of compositions and pharmaceutical medicaments for the treatment of neurological or psychiatric diseases, conditions or disorders involving the D2, D3, and alpha2C receptors, such as schizophrenia, bipolar disorder, pervasive developmental disorders, depression and cognitive deficits associated with various neurological conditions.

The invention also concerns a treatment method for neurological or psychiatric conditions, diseases or disorders that consists of administrating a compound of formula 1 in a therapeutically effective quantity to a patient who requires treatment. It also concerns compounds of formula 1 for their use as a medicament and the use of a compound of formula 1 for the production of a medicament for the treatment of a neurological or psychiatric disease or disorder, erectile dysfunction or dependence on drugs and addictive substances.

The invention concerns compounds of general formula 1 for the production of a medicament for the treatment of schizophrenia, bipolar disorder, pervasive developmental disorders or autism, major depression and cognitive disorders related to age or associated with neurological disorders such as Parkinson's disease, Alzheimer's disease and epilepsy.

Compounds of formula 1 according to the invention may be orally, systemically, parenterally, nasally or rectally. In particular the compound may be administered orally in an appropriate formulation. The dosages of compounds of formula 1 in compositions of the invention can be adjusted to obtain a quantity of active substance that is effective to obtain a desired therapeutic response for a composition particular to the method of administration. The dosage level selected therefore depends on the desired therapeutic effect, the administration route, the desired treatment duration and other factors.

Compounds of formula 1 have been evaluated in vitro as ligands for dopaminergic and noradrenergic receptors and modulators of the activity of these receptors according to the invention in recombinant human cells expressing the receptors. The inhibition constants (Kᵢ) were measured for dopaminergic receptors D2 and D3, and for adrenergic receptors alpha2A, alpha2B and alpha2C, according to the described methods (Newman-Tancredi A, Assie M B, Martel J C, Cosi C, Slot L B, Palmier C, Rauly-Lestienne I, Colpaert F, Vacher B, Cussac D. F15063, a potential antipsychotic with D2/D3 antagonist, 5-HT 1A agonist and D4 partial agonist properties. I. In vitro receptor affinity and efficacy profile. Br J Pharmacol., 2007, 151:237-252). The intrinsic activity of the products for the D2 receptor was measured (Bruins Slot L A, De Vries L, Newman-Tancredi A, Cussac D. Differential profile of antipsychotics at serotonin 5-HT1A and dopamine D2S receptors coupled to extracellular signal-regulated kinase. Eur J Pharmacol, 2006, 534; 63-70. The results obtained for some of these compounds are presented in the following table.

Compounds of formula 1 were evaluated in vitro by the MK-801-induced hyperactivity test in mice (Leriche L. et al., Neuropharmacology 2003, 45, 174).

The in vitro and in vivo results are shown as examples in the following table:

| | pKi | | | | | | ED₅₀ |
|---|---|---|---|---|---|---|---|
| Example No. | D2 | D3 | α2A | α2B | α2C | I.A.* | (mg/kg)*** |
| 7 | 7.84 | 8.19 | 8.06 | 8.71 | 9.07 | 36 | 0.15 |
| 10 | 8.34 | 8.81 | 8.34 | 9.09 | 9.27 | 6 | 0.13 |
| 13 | 9.02 | 9.22 | 8.15 | 8.59 | 9.02 | 16 | 0.04 |
| 26 | 9.05 | 9.35 | 7.46 | 8.02 | 8.69 | 8 | 0.9 |
| 31 | 8.93 | 9.28 | 8.29 | 8.53 | 9.00 | 0 | 2.1 |
| 32 | 8.9 | 9.45 | 7.82 | 7.74 | 8.18 | ND | 2.4 |
| 36 | 8.67 | 8.89 | 8.22 | 8.93 | 9.25 | ND | 3.4 |
| 62 | 8.8 | 9.46 | 7.51 | 7.98 | 8.79 | 0 | 2.1 |
| 63 | 8.51 | 8.88 | 7.27 | 8.08 | 8.57 | 5 | 0.2 |
| 69 | 9.62 | 9.38 | 6.7 | 6.73 | 8.06 | 13 | 0.6 |
| 85 | 7.72 | 8.91 | 7.24 | 7.09 | 8.17 | ND** | 0.5 |
| 91 | 7.82 | 8.48 | 8.31 | 8.81 | 9.36 | 24 | 0.7 |
| 94 | 8.78 | 9.46 | 8.27 | 8.35 | 9.21 | 0 | 0.19 |

*I.A.: Intrinsic activity on receptor D2 in vitro in percentage of that of dopamine;
**ND; not determined;
***ED₅₀ in the MK-801-induced hyperactivity test.

Example 31 was evaluated in rats in a memory test, the passive avoidance test (Chopin P, Briley M. Effects of four non-cholinergic cognitive enhancers in comparison with tacrine and galanthamine on scopolamine-induced amnesia in rats, Psychopharmacology (Berl), 1992; 106:26-30). This test consists of teaching a rat not to enter a dark compartment (its naturally preferred place) where it had previously received an electric shock (0.8 mA). The animal's reticence (the time lapse or “latency” to enter the dark compartment) measured 48 h after its training is considered as an index of its explicit-declarative memory. Latency greatly increases when an amnesic product such as scopolamine is administered shortly after receiving the first shock. A product with promnesic properties opposes the effects of scopolamine. Example 31, administered at doses of 0.16, 0.63, 2.5 and 10 mg/kg, was opposed to the effect of scopolamine; latencies expressed in seconds were 24±12, 60±21 and 78±22, respectively, after treatment by the carrier and by Example 1 at doses of 0.63 and 2.5 mg/kg (P<0.05 for both doses of Example 1 vs. carrier by Mann-Whitney test)

The total daily dose of compounds used according to this invention administered in single or divided doses can be in quantities, for example, of around 0.001 to around 100 mg/kg of body weight per day.

The specific dose for any given patient will depend on a variety of factors including bodyweight, general health, sex, diet, period and route of administration, levels of intestinal absorption and excretion, combination with other medicaments and severity of the particular disorder being treated.

1. A compound of general formula 1 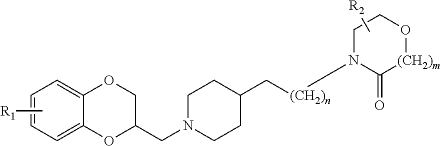
wherein:
R1 represents one or more identical or different substituent(s) on the benzene ring, each independently representing a hydrogen or halogen atom, or a C₁₋₄ alkyl group, or a C₁₋₄ alkoxy group or a C₁₋₄ hydroxyalkyl group or an alkylcarbonyl group or an alkoxycarbonyl group or an OH group or an SO2R group with R alkyl, or a CN group, or a CF3 group, or an OCF 3 group;
n=1, 2 or 3;
R2 represents one or more identical or different substituent(s) on the oxazolidinone or morpholinone ring, each independently representing:
a hydrogen atom or a C₁₋₄ alkyl group, or C₁₋₄ alkoxy group, or C₁₋₄ hydroxyalkyl group or an alkylcarbonyl group, or an alkoxycarbonyl group or an alkoxyphenyl group—optionally substituted by one or more identical or different substituent(s) each independently representing a C₁₋₄ alkoxy group or C₁₋₄ alkyl group or halogen, or a hydroxy—or an alkoxybenzyl group—optionally substituted by one or more identical or different substituent (s) each independently representing a C₁₋₄ alkoxy group or a C₁₋₄ alkyl group or a halogen, or a hydroxy—or a hydroxybenzyl group, or a benzyl group—optionally substituted with one or more identical or different substituent(s) each independently representing a C₁₋₄ alkoxy group or C₁₋₄ alkyl group or a halogen, or a hydroxy—or a phenyl group—optionally substituted with one or more identical or different substituent (s) each independently representing a C₁₋₄ alkoxy group or a C₁₋₄ alkyl group or halogen, or a hydroxy—while R2 is a ring fused with the oxazolidinone or morpholinone group that bears it, consisting of an unsubstituted benzene or benzene substituted by a C₁₋₄ alkoxy, hydroxy, halogen or cyano;
m=0 or 1.

2. Compound according to claim 1, characterized in that n=1 and m=0.

3. Compound according to claim 1, characterized in that n=1 and m=1.

4. Compound according to claim 1, characterized in that n=2 and m=0.

5. Compound according to claim 1, characterized in that n=2 and m=1.

6. Compound according to claim 1, characterized in that n=3 and m=0.

7. Compound according to claim 1, characterized in that n=3 and m=1.

8. Compound according to claim 1, characterized in that it is chosen from among the following compounds:

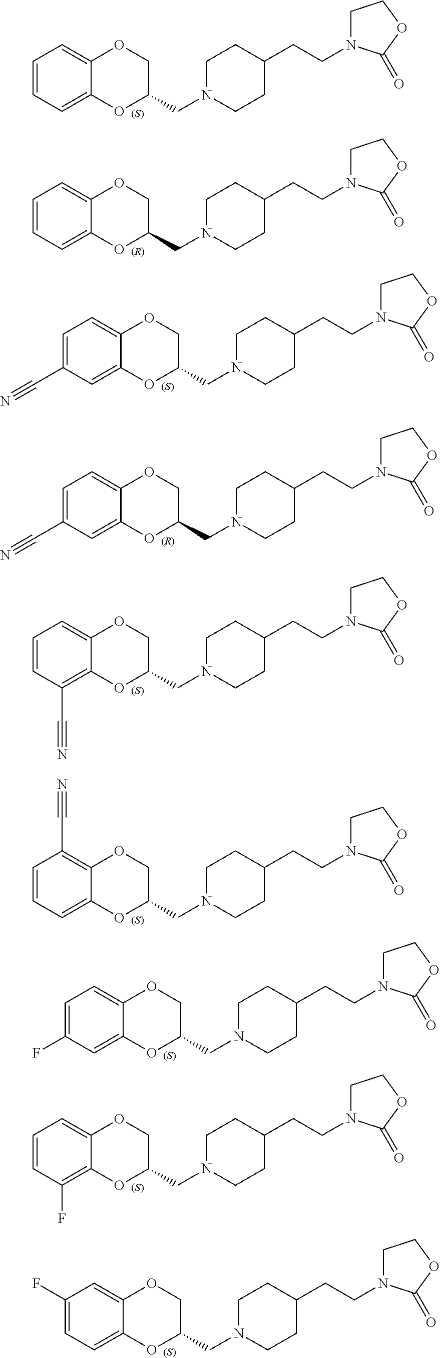

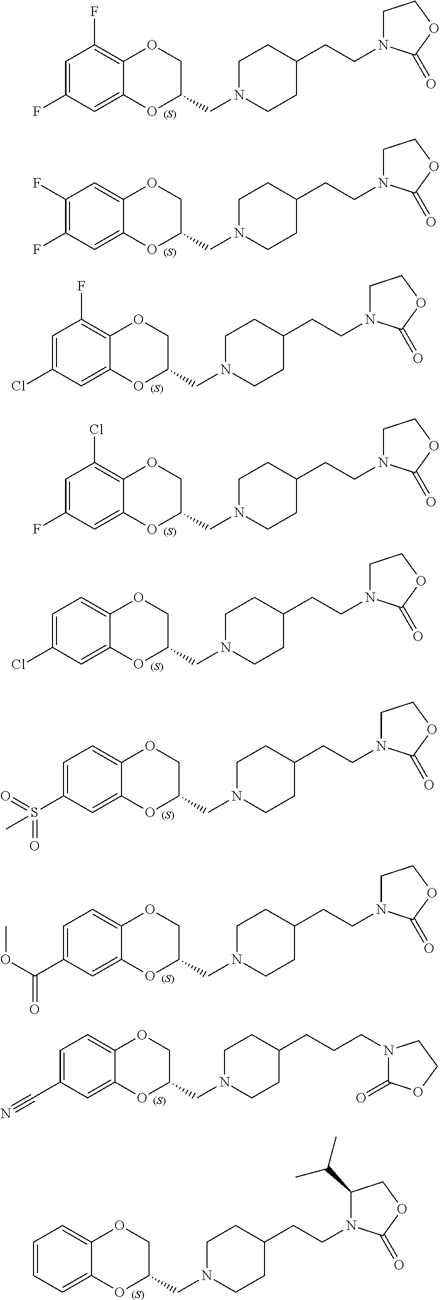

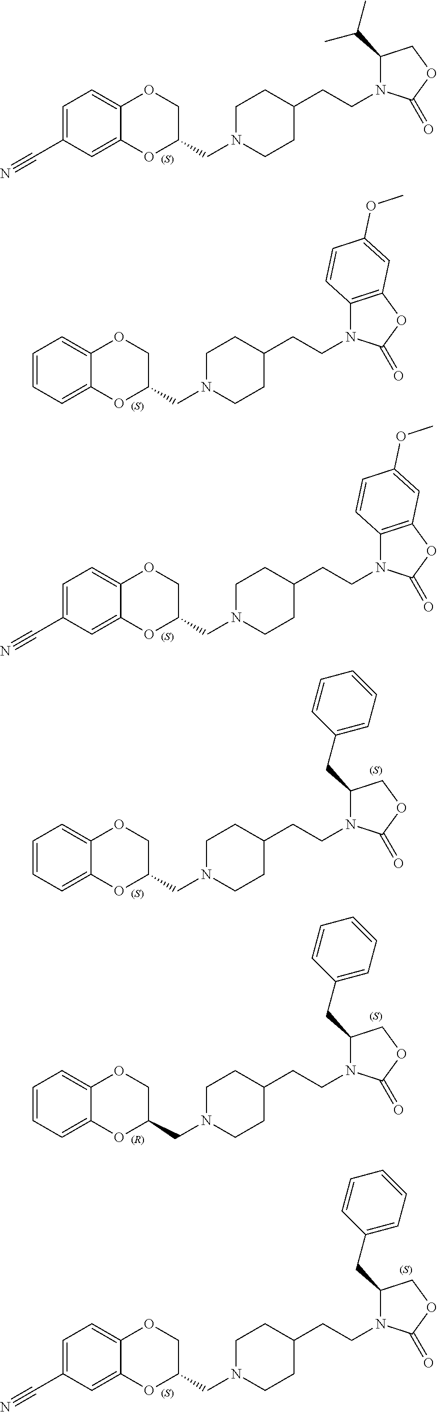

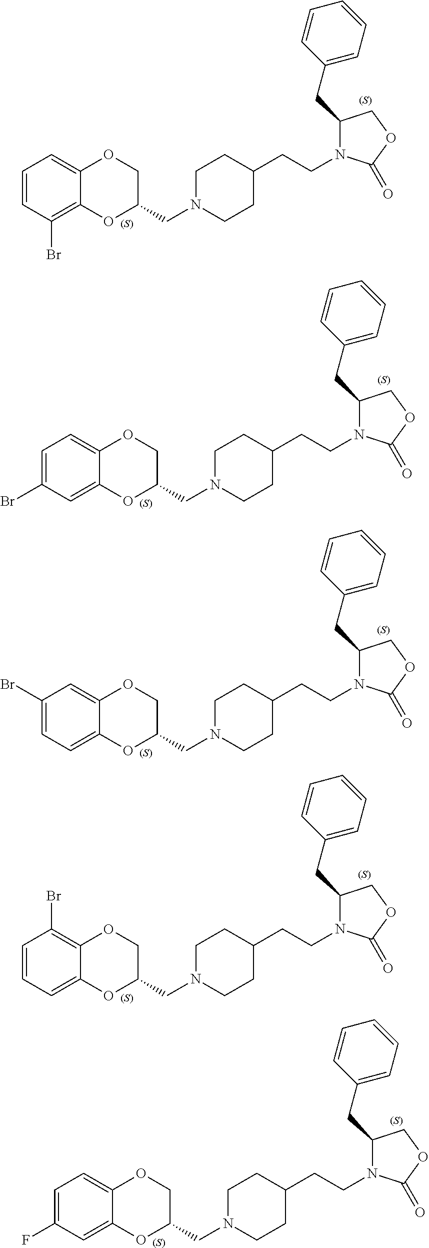

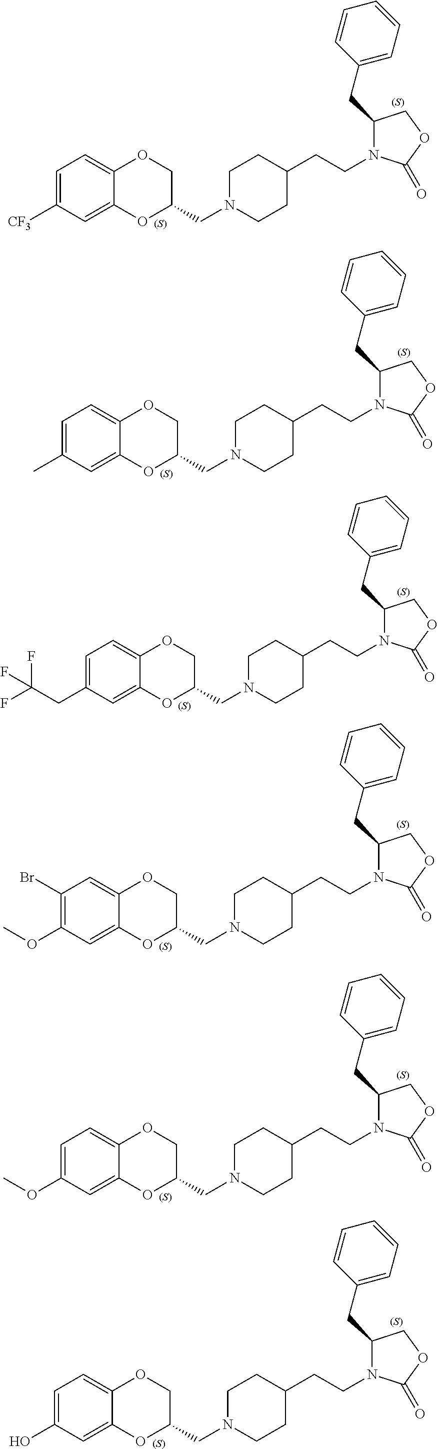

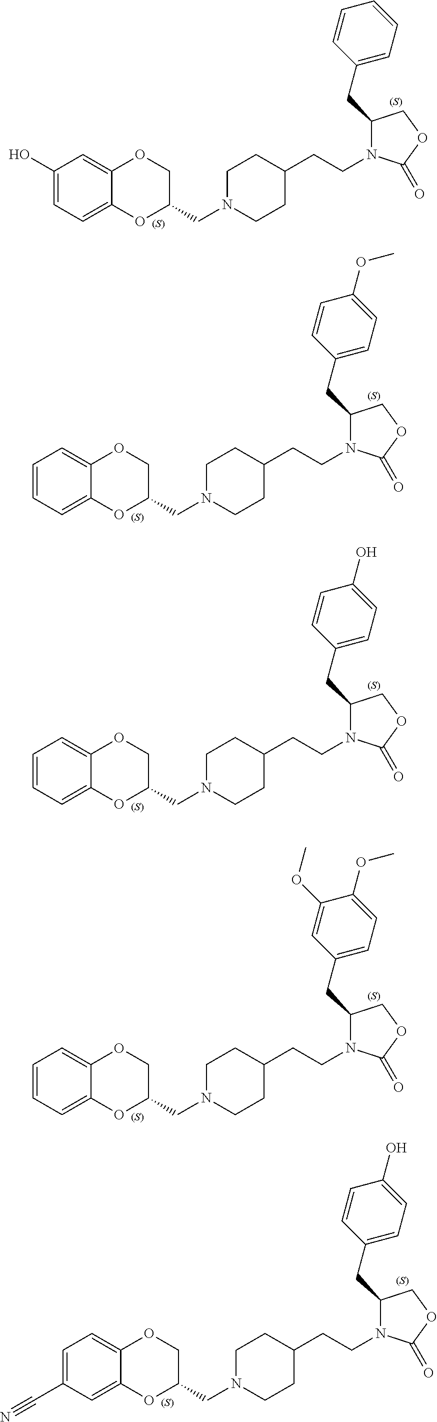

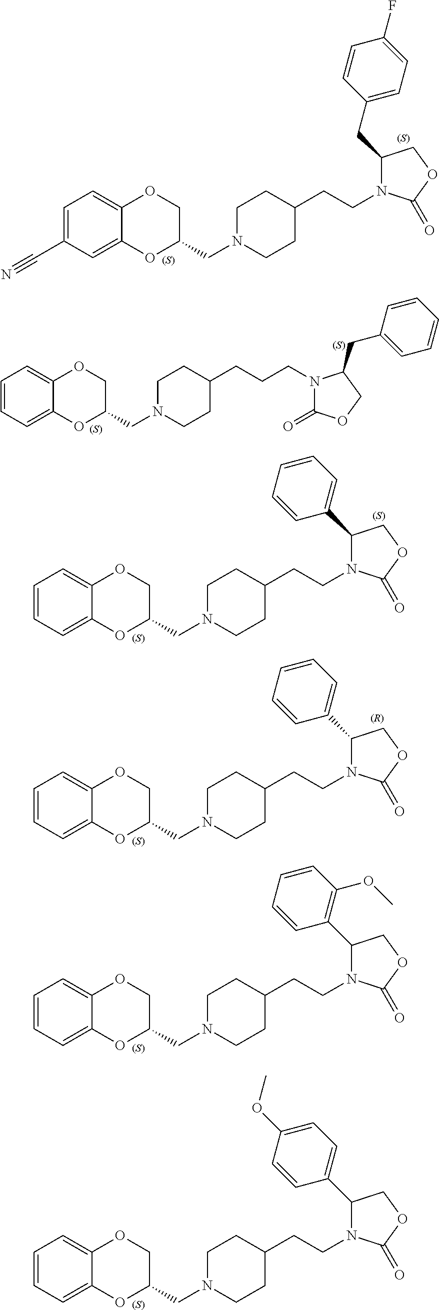

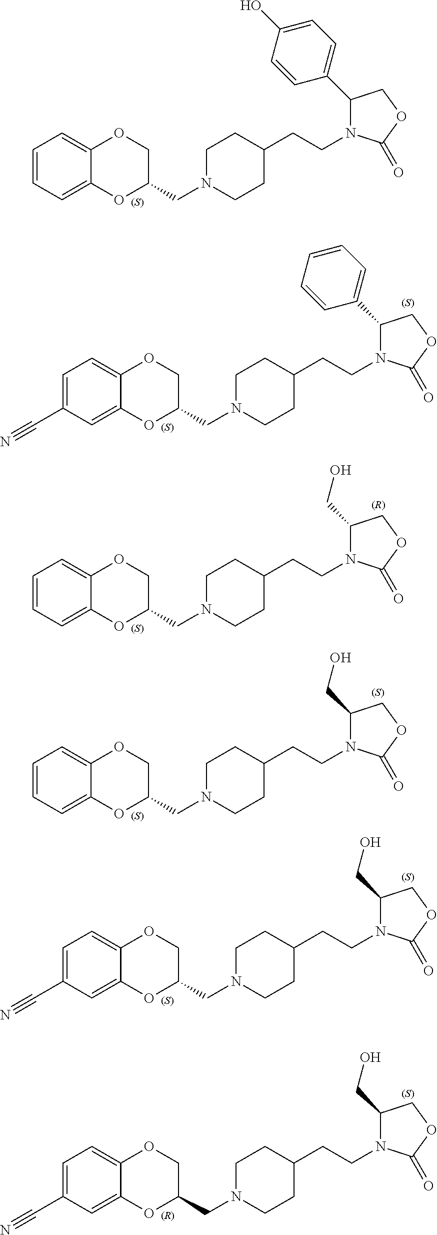

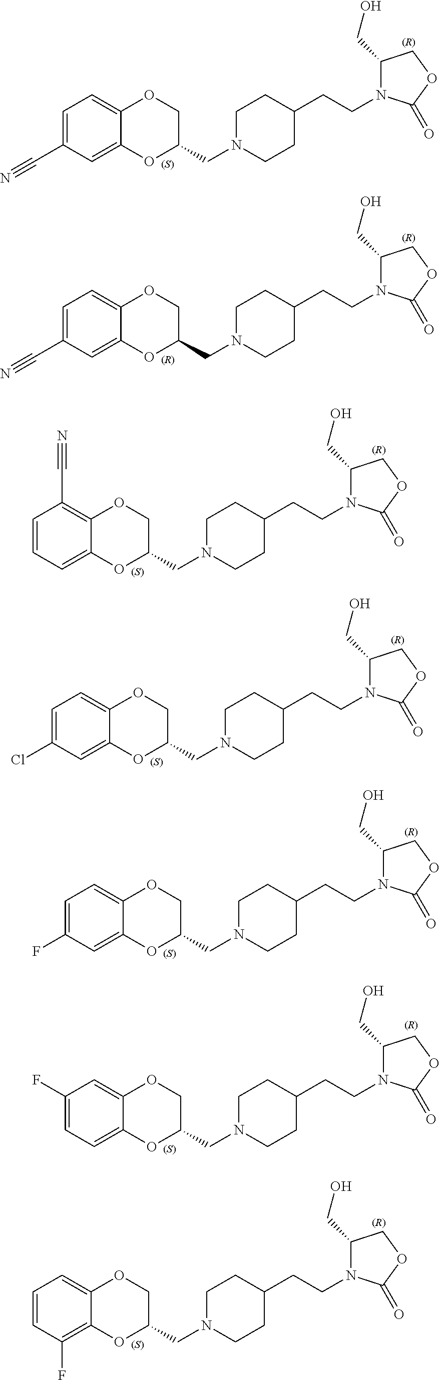

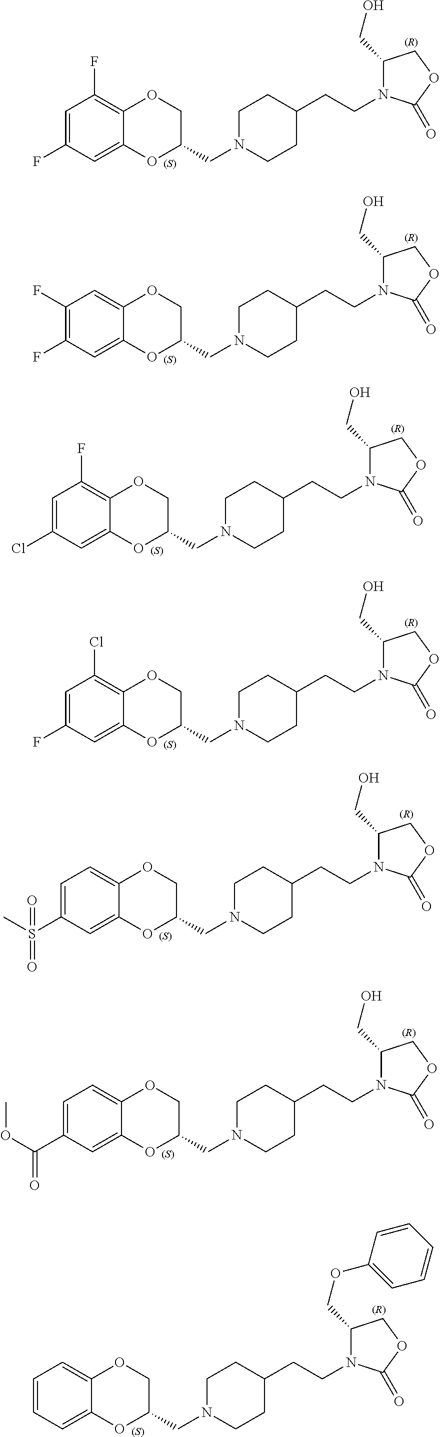

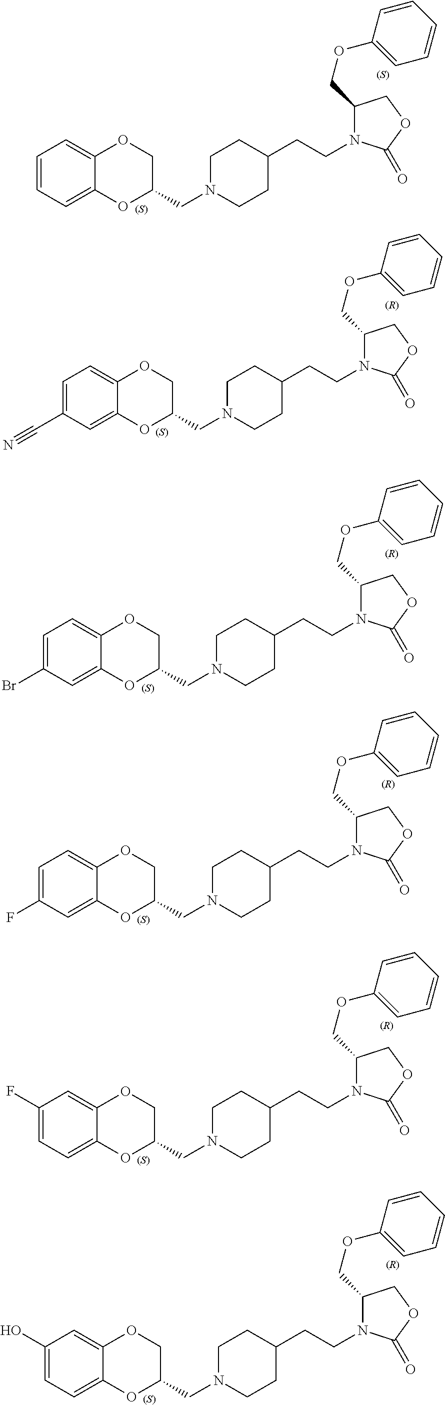

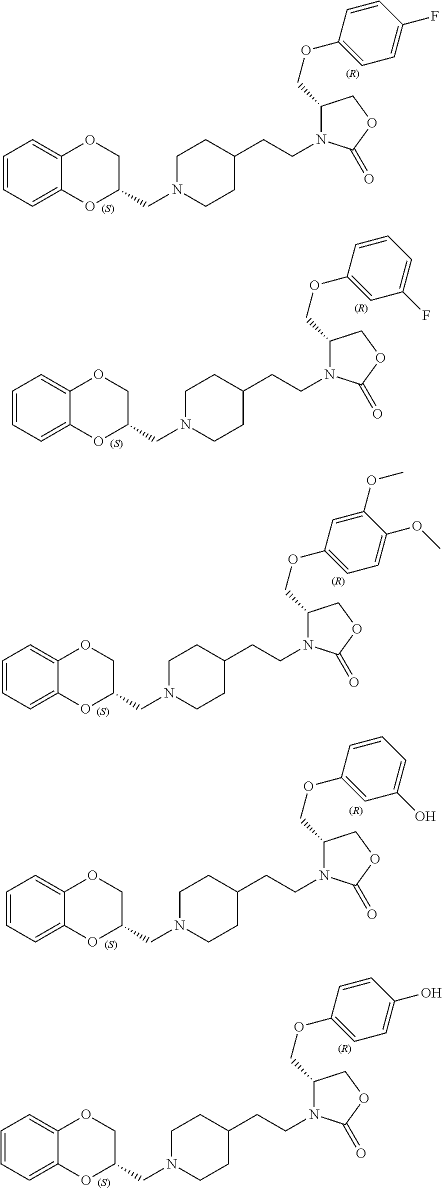

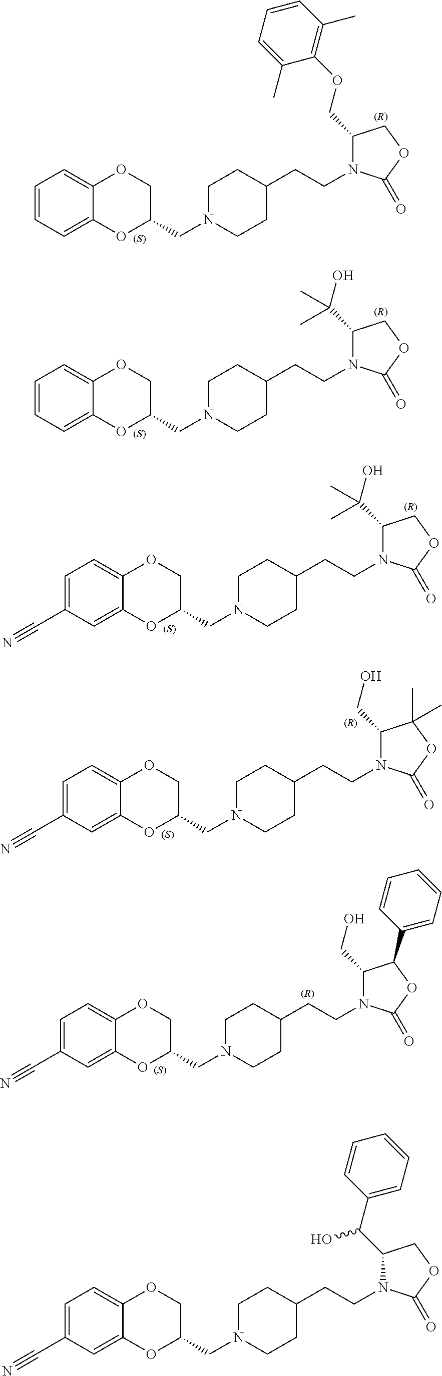

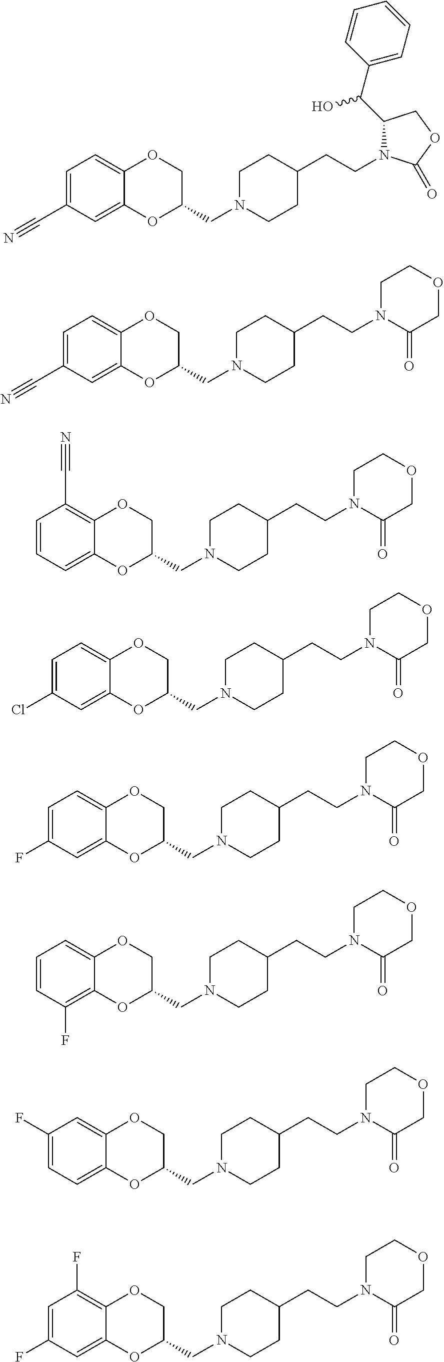

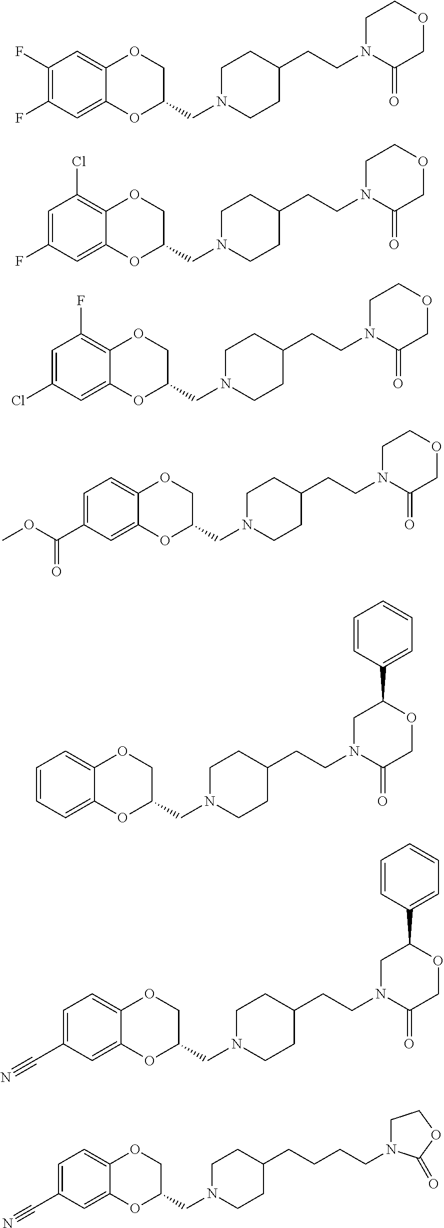

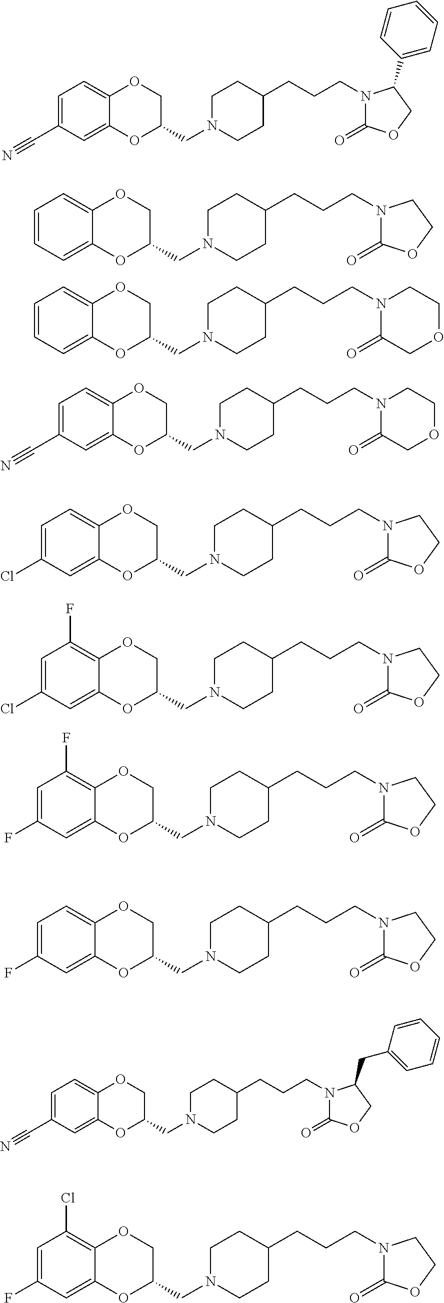

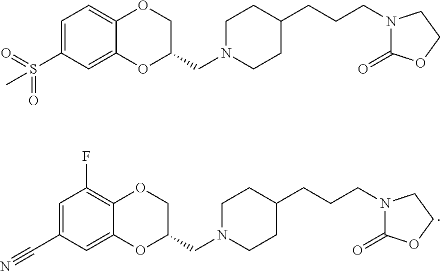 [SMILES: CS(=O)(=O)c1ccc2c(c1)O[C@@H](CN1CCC(CCCN3CCOC3=O)CC1)CO2 ; N#Cc1cc(F)c2c(c1)O[C@@H](CN1CCC(CCCN3CCOC3=O)CC1)CO2]

9. Stereoisomer forms of the compounds according to claim 1 and their salts.

10. Pharmaceutical composition comprising at least one compound according to claim 1 or one of its pharmaceutically acceptable salts and a pharmaceutically acceptable carrier.

11. (canceled)

12. A method of treating a disease or disorder selected from the group consisting of schizophrenia, bipolar disorder, pervasive development disorders and depression, which comprises administering a therapeutically effective amount of a compound according to claim 1 to a patient in need thereof.

13. A method of treating a disease or disorder selected from the group consisting of cognitive disorders associated with age, attention deficit/hyperactivity disorder, a neurodegenerative disease, strokes and their consequences, epilepsy, mood disorders, anxiety disorders and mental disorders propagated by stress, which comprises administering to a patient in need thereof, a therapeutically effective amount of a compound according to claim 1.
